SEQUENCE LISTING (44 sequences)

SEQ ID NO: 1 - DNA, 2088 bp, Lactococcus lactis
  CDS (279)..(2069)
gatctgtccttagtacgagaggaccgggatggacttaccgctggtgtaccagttgttccgccagagcacggctggatagctatgtagggaagggataagcgctgaaagcatctaagtgcgaagccacctcaagatgagattacccattcgagaattaagagcccagagagatgatcaagatgtcaataatttgcaaaaaatcttctttcagcaaaacgggatttgagtttttgctcgatttgtgggaatttaacagaaagtgatctgttgaaatcgcaagccctctcggtgtacttgctggtatcgttccaacgactaatccaacatcaacagcaatctttaaatctttattgactgcaaaaacacgtaatgctattgttttcgctttccaccctcaagctcaaaaatgttcaagccatgcagcaaaaattgtttacgatgctgcaattgaagctggtgcaccggaagactttattcaatggattgaagtaccaagccttgacatgactaccgccttgattcaaaaccgtggacttgcaacaatccttgcaactggtggcccaggaatggtaaacgccgcactcaaatctggtaacccttcactcggtgttggagctggtaatggtgctgtttatgttgatgcaactgcaaatattgaacgtgccgttgaagaccttttgctttcaaaacgttttgataatgggatgatttgtgccactgaaaattcagctgttattgatgcttcagtttatgatgaatttattgctaaaatgcaagaacaaggcgcttatatggttcctaaaaaagactacaaagctattgaaagtttcgtttttgttgaacgtgctggtgaaggttttggagtaactggtcctgttgccggtcgttctggtcaatggattgctgaacaagctggtgtcaaagttcctaaagataaagatgtccttctttttgaacttgataagaaaaatattggtgaagcactttcttctgaaaaactttctcctttgctttcaatctacaaagctgaaacacgtgaagaaggaattgagattgtacgtagcttacttgcttatcaaggtgctggacataatgctgcaattcaaatcggtgcaatggatgatccattcgttaaagaatatggcgaaaaagttgaagcttctcgtatcctcgttaaccaaccagattctattggtggggtcggagatatctatactgatgcaatgcgtccatcacttacacttggaactggttcatgggggaaaaattcactttcacacaatttgagtacatacgatctattgaatgttaaaacagtggctaaacgtcgtaatcgcccacaatgggttcgtttgccaaaagaaatttactacgaaaaaaatgcaatttcttacttacaagaattgccacacgtccacaaagctttcatcgttgctgaccctggtatggttaaatttggtttcgttgataaagttttggaacaacttgctatccgcccaactcaagttgaaacaagcatttatggctctgttcaacctgacccaactttgagcgaagcaattgcaatcgctcgtcaaatgaaacaatttgaacctgacactgtcatctgtcttggtggtggttctgctctcgatgccggtaagattggtcgtttgatttatgaatatgatgctcgtggtgaagctgacctttctgatgatgcaagtttgaaagaacttttccaagaattagctcaaaaatttgtcgatattcgtaaacgtattattaaattctaccatccacataaagcacaaatggttgcaattcctactacttctggtactggttctgaagtgactccatttgcagttatcactgatgatgaaactcatgttaagtacccacttgctgactaccaattaacaccacaagttgccattgttgaccctgagtttgttatgactgtaccaaaacgtactgtttcttggtctggtattgatgcgatgtcacacgcgcttgaatcttacgtttctgttatgtcttctgactatacaaaaccaatttcacttcaagcgatcccgggtctagattagggtaactttgaaagga

SEQ ID NO: 2 - protein, 597 aa, Lactococcus lactis
SPLGVLAGIVPTTNPTSTAIFKSLLTAKTRNAIVFAFHPQAQKCSSHAAKIVYDAAIEAGAPEDFIQWIEVPSLDMTTALIQNRGLATILATGGPGMVNAALKSGNPSLGVGAGNGAVYVDATANIERAVEDLLLSKRFDNGMICATENSAVIDASVYDEFIAKMQEQGAYMVPKKDYKAIESFVFVERAGEGFGVTGPVAGRSGQWIAEQAGVKVPKDKDVLLFELDKKNIGEALSSEKLSPLLSIYKAETREEGIEIVRSLLAYQGAGHNAAIQIGAMDDPFVKEYGEKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMKQFEPDTVICLGGGSALDAGKIGRLIYEYDARGEADLSDDASLKELFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIPGLD

SEQ ID NO: 3 - DNA, 3185 bp, Lactococcus lactis
  CDS (145)..(2853)
aagcttgttacaaaaccgttttctaaacttttgatgagtgtttttgtaaaaactatcacaatattgcttgacatctataaaaaactttgttaaactattcacgtaaaagaaagtgaatgaagtcacaaaggagaacctacaaatatggcaactaaaaaagccgctccagctgcaaagaaagttttaagcgctgaagaaaaagccgcaaaattccaagaagctgttgcttatactgacaaattagtcaaaaaagcacaagctgctgttcttaaatttgaaggatatacacaaactcaagtcgatactattgtcgctgcaatggctcttgcagcaagcaaacattctctagaactcgctcatgaagccgttaacgaaactggtcgtggtgttgtcgaagacaaagataccaaaaaccactttgcttctgaatctgtttataacgcaattaaaaatgacaaaactgttggtgtcatttctgaaaacaaggttgctggatctgttgaaatcgcaagccctctcggtgtacttgctggtatcgttccaacgactaatccaacatcaacagcaatctttaaatctttattgactgcaaaaacacgtaatgctattgttttcgctttccaccctcaagctcaaaaatgttcaagccatgcagcaaaaattgtttacgatgctgcaattgaagctggtgcaccggaagactttattcaatggattgaagtaccaagccttgacatgactaccgccttgattcaaaaccgtggacttgcaacaatccttgcaactggtggcccaggaatggtaaacgccgcactcaaatctggtaacccttcactcggtgttggagctggtaatggtgctgtttatgttgatgcaactgcaaatattgaacgtgccgttgaagaccttttgctttcaaaacgttttgataatgggatgatttgtgccactgaaaattcagctgttattgatgcttcagtttatgatgaatttattgctaaaatgcaagaacaaggcgcttatatggttcctaaaaaagactacaaagctattgaaagtttcgtttttgttgaacgtgctggtgaaggttttggagtaactggtcctgttgccggtcgttctggtcaatggattgctgaacaagctggtgtcaaagttcctaaagataaagatgtccttctttttgaacttgataagaaaaatattggtgaagcactttcttctgaaaaactttctcctttgctttcaatctacaaagctgaaacacgtgaagaaggaattgagattgtacgtagcttacttgcttatcaaggtgctggacataatgctgcaattcaaatcggtgcaatggatgatccattcgttaaagaatatggcgaaaaagttgaagcttctcgtatcctcgttaaccaaccagattctattggtggggtcggagatatctatactgatgcaatgcgtccatcacttacacttggaactggttcatgggggaaaaattcactttcacacaatttgagtacatacgatctattgaatgttaaaacagtggctaaacgtcgtaatcgcccacaatgggttcgtttgccaaaagaaatttactacgaaaaaaatgcaatttcttacttacaagaattgccacacgtccacaaagctttcatcgttgctgaccctggtatggttaaatttggtttcgttgataaagttttggaacaacttgctatccgcccaactcaagttgaaacaagcatttatggctctgttcaacctgacccaactttgagcgaagcaattgcaatcgctcgtcaaatgaaacaatttgaacctgacactgtcatctgtcttggtggtggttctgctctcgatgccggtaagattggtcgtttgatttatgaatatgatgctcgtggtgaagctgacctttctgatgatgcaagtttgaaagaacttttccaagaattagctcaaaaatttgtcgatattcgtaaacgtattattaaattctaccatccacataaagcacaaatggttgcaattcctactacttctggtactggttctgaagtgactccatttgcagttatcactgatgatgaaactcatgttaagtacccacttgctgactaccaattaacaccacaagttgccattgttgaccctgagtttgttatgactgtaccaaaacgtactgtttcttggtctggtattgatgcgatgtcacacgcgcttgaatcttacgtttctgttatgtcttctgactatacaaaaccaatttcacttcaagcgatcaaacttatctttgaaaacttgactgagtcttatcattatgacccagcgcatccaactaaagaaggacaaaaagcccgcgaaaacatgcacaatgctgcaacactcgctggtatggccttcgctaatgctttccttggaattaaccactcacttgctcataaaattggtggtgaatttggacttcctcatggtcttgccattgccatcgctatgccacatgtcattaaatttaacgctgtaacaggaaacgttaaacgtaccccttacccacgttatgaaacatatcgtgctcaagaggactacgctgaaatttcacgcttcatgggatttgctggtaaagatgattcagatgaaaaagctgtgcaagctctggttgctgaacttaagaaactgactgatagcattgatattaatatcaccctttcaggaaatggtatcgataaagctcaccttgaacgtgaacttgataaattggctgaccttgtttatgatgatcaatgtactcctgctaatcctcgtcaaccaagaattgatgagattaaacagttgttgttagatcaatactaataatctgttgataaaattattaaaacgctctgatcagagcattttttattatagcttatacaactatcaaaaggtataaatcaatttcgatataggctcttttcactccattgatttatgcatttctataaaaatcaataattaattagcgatagaagtcgagttcatgcatgctaataatgaaattgttttaaattctggtttttctttatgttctttgcgaacatctttcacagtttctttgttcatgaaaattcctccttattatggtactattttgagcccaaatagttatataagaatcctaaacttcggatatcttatcaaag

SEQ ID NO: 4 - protein, 903 aa, Lactococcus lactis
MATKKAAPAAKKVLSAEEKAAKFQEAVAYTDKLVKKAQAAVLKFEGYTQTQVDTIVAAMALAASKHSLELAHEAVNETGRGVVEDKDTKNHFASESVYNAIKNDKTVGVISENKVAGSVEIASPLGVLAGIVPTTNPTSTAIFKSLLTAKTRNAIVFAFHPQAQKCSSHAAKIVYDAAIEAGAPEDFIQWIEVPSLDMTTALIQNRGLATILATGGPGMVNAALKSGNPSLGVGAGNGAVYVDATANIERAVEDLLLSKRFDNGMICATENSAVIDASVYDEFIAKMQEQGAYMVPKKDYKAIESFVFVERAGEGFGVTGPVAGRSGQWIAEQAGVKVPKDKDVLLFELDKKNIGEALSSEKLSPLLSIYKAETREEGIEIVRSLLAYQGAGHNAAIQIGAMDDPFVKEYGEKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMKQFEPDTVICLGGGSALDAGKIGRLIYEYDARGEADLSDDASLKELFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIGGEFGLPHGLAIAIAMPHVIKFNAVTGNVKRTPYPRYETYRAQEDYAEISRFMGFAGKDDSDEKAVQALVAELKKLTDSIDINITLSGNGIDKAHLERELDKLADLVYDDQCTPANPRQPRIDEIKQLLLDQY

SEQ ID NO: 5 - protein, 835 aa, Lactococcus lactis
MATKKAAPAAKKVLSAEEKAAKFQEAVAYTDKLVKKAQAAVLKFEGYTQTQVDTIVAAMALAASKHSLELAHEAVNETGRGVVEDKDTKNHFASESVYNAIKNDKTVGVISENKVAGSVEIASPLGVLAGIVPTTNPTSTAIFKSLLTAKTRNAIVFAFHPQAQKCSSHAAKIVYDAAIEAGAPEDFIQWIEVPSLDMTTALIQNRGLATILATGGPGMVNAALKSGNPSLGVGAGNGAVYVDATANIERAVEDLLLSKRFDNGMICATENSAVIDASVYDEFIAKMQEQGAYMVPKKDYKAIESFVFVERAGEGFGVTGPVAGRSGQWIAEQAGVKVPKDKDVLLFELDKKNIGEALSSEKLSPLLSIYKAETREEGIEIVRSLLAYQGAGHNAAIQIGAMDDPFVKEYGEKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMKQFEPDTVICLGGGSALDAGKIGRLIYEYDARGEADLSDDASLKELFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIGGEFGLPHGLAIAIAMPHVIKFNAVTGNVKRTPYPRYETYRAQEDYAEISRFMGFAGKDDSDEKA

SEQ ID NO: 6 - protein, 797 aa, Escherichia coli
AVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKL

SEQ ID NO: 7 - DNA, 19 bp, Artificial Sequence
  Primer adhe-mg3
cttctttggttggatgagc

SEQ ID NO: 8 - protein, 490 aa, Lactococcus lactis
YQGAGHNAAIQIGAMDDPFVKEYGIKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMNHFEPDTVICLGGGSALDAGKIGRLIYEYDARGEADLSDDASLKEIFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIAGEFGLPHGLAIAIAMPHVIKFNAVTGNVKFTPYPRYETYRAQEDYAEISRFMGFAGKEDSDEKAVKAFVAELKKLTDSIDINITLSGNGVDKAHLERELDKLADLV

SEQ ID NO: 9 - protein, 903 aa, Lactococcus lactis; identical to SEQ ID NO: 4

SEQ ID NO: 10 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 11 - protein, 862 aa, Clostridium acetobutylicum
MKVTTVKELDEKLKVIKEAQKKFSCYSQEMVDEIFRNAAMAAIDARIELAKAAVLETGMGLVEDKVIKNHFAGEYIYNKYKDEKTCGIIERNEPYGITKIAEPIGVVAAIIPVTNPTSTTIFKSLISLKTRNGIFFSPHPRAKKSTILAAKTILDAAVKSGAPENIIGWIDEPSIELTQYLMQKADITLATGGPSLVKSAYSSGKPAIGVGPGNTPVIIDESAHIKMAVSSIILSKTYDNGVICASEQSVIVLKSIYNKVKDEFQERGAYIIKKNELDKVREVIFKDGSVNPKIVGQSAYTIAAMAGIKVPKTTRILIGEVTSLGEEEPFAHEKLSPVLAMYEADNFDDALKKAVTLINLGGLGHTSGIYADEIKARDKIDRFSSAMKTVRTFVNIPTSQGASGDLYNFRIPPSFTLGCGFWGGNSVSENVGPKHLLNIKTVAERRENMLWFRVPHKVYFKFGCLQFALKDLKDLKKKRAFIVTDSDPYNLNYVDSIIKILEHLDIDFKVFNKVGREADLKTIKKATEEMSSFMPDTIIALGGTPEMSSAKLMWVLYEHPEVKFEDLAIKFMDIRKRIYTFPKLGKKAMLVAITTSAGSGSEVTPFALVTDNNTGNKYMLADYEMTPNMAIVDAELMMKMPKGLTAYSGIDALVNSIEAYTSVYASEYTNGLALEAIRLIFKYLPEAYKNGRTNEKAREKMAHASTMAGMASANAFLGLCHSMAIKLSSEHNIPSGIANALLIEEVIKFNAVDNPVKQAPCPQYKYPNTIFRYARIADYIKLGGNTDEEKVDLLINKIHELKKALNIPTSIKDAGVLEENFYSSLDRISELALDDQCTGANPRFPLTSEIKEMYINCFKKQP

SEQ ID NO: 12 - DNA, 1470 bp, Lactococcus lactis
taccaaggagctggtcacaacgctgcaattcaaatcggtgcaatggacgacccatttgtcaaagaatacggaattaaagtcgaagcttctcgtatcctcgttaaccaacctgactctatcggtggggtcggagatatttatactgatgcaatgcgtccatcattgacgctcggaactggttcatgggggaaaaattcactttcacacaatttgagtacatacgatctattgaatgttaaaacagtggctaaacgtcgtaatcgccctcaatgggttcgtttgccaaaagaaatttactacgaaaaaaatgcaatttcttacttacaagaattgccacacgtccacaaagctttcattgttgccgaccctggtatggttaaattcggtttcgttgataaagttttggaacaacttgctatccgcccaactcaagttgaaacaagcatttatggctcagtccaacctgacccaactttgagtgaagcaattgcaatcgctcgtcaaatgaaccattttgaacctgacactgtcatctgtcttggtggtggttctgctctcgatgctggtaagattggtcgtttgatttatgaatatgatgctcgtggtgaggctgacctttccgatgacgcaagtttgaaagagatcttccaagagttagctcaaaaatttgttgatattcgtaaacgtattatcaaattctaccacccacacaaagcacaaatggttgctatccctactacttctggtactggttctgaagtgactccatttgcggttatcactgatgatgaaactcacgttaaatatccacttgctgactatcaattgacacctcaagttgccattgttgaccctgagtttgttatgactgtaccaaaacgtactgtttcttggtctgggattgatgctatgtcacacgcgcttgaatcttatgtttctgtcatgtcttctgactatacaaaaccaatttcacttcaagccatcaaactcatctttgaaaacttgactgagtcttatcattatgacccagctcatccaaccaaagaaggtcaaaaagctcgcgaaaacatgcacaatgctgcaacactcgctggtatggccttcgccaatgctttccttggaattaaccactcacttgctcataaaattgctggtgaatttgggcttcctcatggtcttgccattgctatcgctatgccacatgtcattaaatttaacgctgtaacaggaaacgttaaatttaccccttacccacgttatgaaacttatcgtgcgcaagaagactacgctgaaatttcacgcttcatgggatttgctggcaaagaagattcagatgaaaaagcggtcaaagcttttgttgctgaacttaaaaaattgactgatagtattgatattaatatcaccctttcaggaaatggtgtagataaagctcaccttgaacgtgagcttgataaattggctgaccttgtt

SEQ ID NO: 13 - DNA, 3193 bp, Lactococcus lactis
aagcttgttacaaaaccgttttctaaacttttgatgagtgtttttgtaaaaactatcacaatattgcttgacatctataaaaaactttgttaaactattcacgtaaaagaaagtgaatgaagtcacaaaggagaacctacaaatatggcaactaaaaaagccgctccagctgcaaagaaagttttaagcgctgaagaaaaagccgcaaaattccaagaagctgttgcttatactgacaaattagtcaaaaaagcacaagctgctgttcttaaatttgaaggatatacacaaactcaagtcgatactattgtcgctgcaatggctcttgcagcaagcaaacattctctagaactcgctcatgaagccgttaacgaaactggtcgtggtgttgtcgaagacaaagataccaaaaaccactttgcttctgaatctgtttataacgcaattaaaaatgacaaaactgttggtgtcatttctgaaaacaaggttgctggatctgttgaaatcgcaagccctctcggtgtacttgctggtatcgttccaacgactaatccaacatcaacagcaatctttaaatctttattgactgcaaaaacacgtaatgctattgttttcgctttccaccctcaagctcaaaaatgttcaagccatgcagcaaaaattgtttacgatgctgcaattgaagctggtgcaccggaagactttattcaatggattgaagtaccaagccttgacatgactaccgccttgattcaaaaccgtggacttgcaacaatccttgcaactggtggcccaggaatggtaaacgccgcactcaaatctggtaacccttcactcggtgttggagctggtaatggtgctgtttatgttgatgcaactgcaaatattgaacgtgccgttgaagaccttttgctttcaaaacgttttgataatgggatgatttgtgccactgaaaattcagctgttattgatgcttcagtttatgatgaatttattgctaaaatgcaagaacaaggcgcttatatggttcctaaaaaagactacaaagctattgaaagtttcgtttttgttgaacgtgctggtgaaggttttggagtaactggtcctgttgccggtcgttctggtcaatggattgctgaacaagctggtgtcaaagttcctaaagataaagatgtccttctttttgaacttgataagaaaaatattggtgaagcactttcttctgaaaaactttctcctttgctttcaatctacaaagctgaaacacgtgaagaaggaattgagattgtacgtagcttacttgcttatcaaggtgctggacataatgctgcaattcaaatcggtgcaatggatgatccattcgttaaagaatatggcgaaaaagttgaagcttctcgtatcctcgttaaccaaccagattctattggtggggtcggagatatctatactgatgcaatgcgtccatcacttacacttggaactggttcatgggggaaaaattcactttcacacaatttgagtacatacgatctattgaatgttaaaacagtggctaaacgtcgtaatcgcccacaatgggttcgtttgccaaaagaaatttactacgaaaaaaatgcaatttcttacttacaagaattgccacacgtccacaaagctttcatcgttgctgaccctggtatggttaaatttggtttcgttgataaagttttggaacaacttgctatccgcccaactcaagttgaaacaagcatttatggctctgttcaacctgacccaactttgagcgaagcaattgcaatcgctcgtcaaatgaaacaatttgaacctgacactgtcatctgtcttggtggtggttctgctctcgatgccggtaagattggtcgtttgatttatgaatatgatgctcgtggtgaagctgacctttctgatgatgcaagtttgaaagaacttttccaagaattagctcaaaaatttgtcgatattcgtaaacgtattattaaattctaccatccacataaagcacaaatggttgcaattcctactacttctggtactggttctgaagtgactccatttgcagttatcactgatgatgaaactcatgttaagtacccacttgctgactaccaattaacaccacaagttgccattgttgaccctgagtttgttatgactgtaccaaaacgtactgtttcttggtctggtattgatgcgatgtcacacgcgcttgaatcttacgtttctgttatgtcttctgactatacaaaaccaatttcacttcaagcgatcaaacttatctttgaaaacttgactgagtcttatcattatgacccagcgcatccaactaaagaaggacaaaaagcccgcgaaaacatgcacaatgctgcaacactcgctggtatggccttcgctaatgctttccttggaattaaccactcacttgctcataaaattggtggtgaatttggacttcctcatggtcttgccattgccatcgctatgccacatgtcattaaatttaacgctgtaacaggaaacgttaaacgtaccccttacccacgttatgaaacatatcgtgctcaagaggactacgctgaaatttcacgcttcatgggatttgctggtaaagatgattcagatgaaaaagctgtgcaagctctggttgctgaacttaagaaactgactgatagcattgatattaatatcaccctttcaggaaatggtatcgataaagctcaccttgaacgtgaacttgataaattggctgaccttgtttatgatgatcaatgtactcctgctaatcctcgtcaaccaagaattgatgagattaaacagttgttgttagatcaatactaataatctgttgataaaattattaaaacgctctgatgaattcgtcagagcattttttattatagcttatacaactatcaaaaggtataaatcaatttcgatataggctcttttcactccattgatttatgcatttctataaaaatcaataattaattagcgatagaagtcgagttcatgcatgctaataatgaaattgttttaaattctggtttttctttatgttctttgcgaacatctttcacagtttctttgttcatgaaaattcctccttattatggtactattttgagcccaaatagttatataagaatcctaaacttcggatatcttatcaaag

SEQ ID NO: 14 - protein, 758 aa, Escherichia coli
SELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQS

SEQ ID NO: 15 - DNA, 3412 bp, Lactococcus lactis
  CDS (80)..(2440)
gaattctgtttgctattctcaaactgtatgatataatgaagttgtaatttgaaacagaaagaacaaaggagatttcaaaatgaaaaccgaagttacggaaaatatctttgaacaagcttgggatggttttaaaggaaccaactggcgcgataaagcaagcgttactcgctttgtacaagaaaactacaaaccatatgatggtgatgaaagctttcttgctgggccaacagaacgtacacttaaagtaaagaaaattattgaagatacaaaaaatcactacgaagaagtaggatttcccttcgatactgaccgcgtaacctctattgataaaatccctgctggatatatcgatgctaatgataaagaacttgaactcatctatgggatgcaaaatagcgaacttttccgcttgaatttcatgccaagaggtggacttcgtgttgctgaaaagattttgacagaacacggtctctcagttgacccaggcttgcatgatgttttgtcacaaacaatgacttctgtaaatgatggaatctttcgtgcttatacttcagcaattcgtaaagcacgtcatgctcatactgtaacaggtttgccagatgcttactctcgtggacgtatcattggtgtctatgcacgtcttgccctttacggtgctgattaccttatgaaggaaaaagcaaaagaatgggatgcaatcactgaaattaacgaagaaaacattcgtcttaaagaagaaattaatatgcaataccaagctttgcaagaagttgtaaactttggtgccttatatggtcttgatgtttcacgtccagctatgaacgtaaaagaagcaatccaatgggttaacatcgcttatatggcagtatgtcgtgtcattaatggagctgcaacttcacttggacgtgttccaatcgttcttgatatctttgcagaacgtgaccttgctcgtggaacatttactgaacaagaaattcaagaatttgttgatgatttcgttttgaagcttcgtacaatgaaatttgctcgtgcagctgcttatgatgaactttattctggtgacccaacattcatcacaacatctatggctggtatgggtaatgacggacgtcaccgtgtcactaaaatggactaccgtttcttgaacacacttgatacaatcggaaatgctccagaaccaaacttgacagtcctttgggattctaaacttccttactcattcaaacgttattcaatgtctatgagccacaagcattcttctattcaatatgaaggtgttgaaacaatggctaaagatggatatggcgaaatgtcatgtatctcttgttgtgtctcaccacttgatccagaaaatgaagaaggacgtcataacctccaatactttggtgcgcgtgtaaacgtcttgaaagcaatgttgactggtttgaacggtggttatgatgacgttcataaagattataaagtattcgacatcgaacctgttcgtgacgaaattcttgactatgatacagttatggaaaactttgacaaatctctcgactggttgactgatacttatgttgatgcaatgaatatcattcattacatgactgataaatataactatgaagcagttcaaatggccttcttgcctactaaagttcgtgctaacatgggatttggtatctgtggattcgcaaatacagttgattcactttcagcaattaaatatgctaaagttaaaacattgcgtgatgaaaatggctatatctacgattacgaagtagaaggtgatttccctcgttatggtgaagatgatgatcgtgctgatgatattgctaaacttgtcatgaaaatgtaccatgaaaaattagcttcacacaaactttacaaaaatgctgaagctactgtttcacttttgacaattacatctaacgttgcttactctaaacaaactggtaattctccagtacataaaggagtattcctcaatgaagatggtacagtaaataaatctaaacttgaattcttctcaccaggtgctaacccatctaataaagctaagggtggttggttgcaaaaccttcgctcattggctaagttggaattcaaagatgcaaatgatggtatttcattgactactcaagtttcacctcgtgcacttggtaaaactcgtgatgaacaagtggataacttggttcaaattcttgatggatacttcacaccaggtgctttgattaatggtactgaatttgcaggtcaacacgttaacttgaacgtaatggaccttaaagatgtttacgataaaatcatgcgtggtgaagatgttatcgttcgtatctctggttactgtgtcaatactaaatacctcacaccagaacaaaaacaagaattaactgaacgtgtcttccatgaagttctttcaaacgatgatgaagaagtaatgcatacttcaaacatctaattcttaaaatttaatgaatattcggtctgtcagtactgacagacttttttttacgaaaaaattaatcataatagttaaaaactattgtttttagtttaagaaagttaaattttatgctaaaatagatgaatgaaaatggtaattggattgacaggcggaattgcgatgggaaatcaacggtggttgattttttgattctgagggttatcaagtgattgatgctgacaaagttgtccgtcaatttacaagaacctggcggaaaactttacaaggcaatattagaaacttacggtttagattttattgctgacaattggacagttaaatcgtgaaaaattaggagctttagttttttctgattcaaaagagcgcgagaaattatcaaacttacaagatgaaattattcgtacagaattatatgatagacgtgatgacttattaaaaaaaatgactgacaagtctgtcagtaaaaattttgattcaaagagtcaaggaaaaaatctgtcagtaaataagccaatatttatggatattccgttattaattgaatacaattataccggatttgatgaaatatggttggtcagcttacctgaaaaaatacaattagaaagactgatggcaagaaataagtttacggaagaagaagctaaaaaacgaatttcttcacaaatgccattgtcagaaaaacaaaaagtcgctgatgtcattctggataattctggaaagattgaagcactaaaaaaacaaatccagcgagaactagctaggatagaagaacagaaatagaggtgaatcgcacgaaaacagttaattggaaaggaatttatttataacatggattggctgcttttttgtaggttcatcattttcactcgtcatgcctttctccccttgtatattcaaggactgggtgaagcggtgggaatttgaactttactcagggttactttttctttgccagcctta

SEQ ID NO: 16 - protein, 787 aa, Lactococcus lactis
MKTEVTENIFEQAWDGFKGTNWRDKASVTRFVQENYKPYDGDESFLAGPTERTLKVKKIIEDTKNHYEEVGFPFDTDRVTSIDKIPAGYIDANDKELELIYGMQNSELFRLNFMPRGGLRVAEKILTEHGLSVDPGLHDVLSQTMTSVNDGIFRAYTSAIRKARHAHTVTGLPDAYSRGRIIGVYARLALYGADYLMKEKAKEWDAITEINEENIRLKEEINMQYQALQEVVNFGALYGLDVSRPAMNVKEAIQWVNIAYMAVCRVINGAATSLGRVPIVLDIFAERDLARGTFTEQEIQEFVDDFVLKLRTMKFARAAAYDELYSGDPTFITTSMAGMGNDGRHRVTKMDYRFLNTLDTIGNAPEPNLTVLWDSKLPYSFKRYSMSMSHKHSSIQYEGVETMAKDGYGEMSCISCCVSPLDPENEEGRHNLQYFGARVNVLKAMLTGLNGGYDDVHKDYKVFDIEPVRDEILDYDTVMENFDKSLDWLTDTYVDAMNIIHYMTDKYNYEAVQMAFLPTKVRANMGFGICGFANTVDSLSAIKYAKVKTLRDENGYIYDYEVEGDFPRYGEDDDRADDIAKLVMKMYHEKLASHKLYKNAEATVSLLTITSNVAYSKQTGNSPVHKGVFLNEDGTVNKSKLEFFSPGANPSNKAKGGWLQNLRSLAKLEFKDANDGISLTTQVSPRALGKTRDEQVDNLVQILDGYFTPGALINGTEFAGQHVNLNVMDLKDVYDKIMRGEDVIVRISGYCVNTKYLTPEQKQELTERVFHEVLSNDDEEVMHTSNI

SEQ ID NO: 17 - DNA, 2665 bp, Streptococcus mutans
aagcaagttctttcgcttgtgtaaccggttactgtatgatagaatataatcgtaaattgtaacagattaactgttactagaatagaggggaactcaattatggcaactgtcaaaactaacactgacgtttttgaaaaagcctgggaaggctttaaaggaactgactggaaagacagagcaagcatttctcgctttgttcaagacaactacactccatatgacggaggcgaaagttttcttgccggccctactgaacgttcacttcacatcaaaaaagtcgtagaagaaactaaagcgcattacgaagaaacacgttttccaatggatacacgtattacatctattgctgatatcccagcaggttatattgacaaggaaaatgaattgatttttggtatccaaaacgatgaactttttaagctgaacttcatgccaaaaggcggtattcgcatggctgaaacagctttgaaagaacatggttatgaaccagaccctgccgttcatgaaatctttaccaaatatgcaacaaccgttaatgatggtatctttcgtgcttacacttcaaacattcgccgtgcacgtcatgcccacactgtaactggtctcccagatgcatactctcgcggacgtattattggagtttatgcccgtcttgctctctatggtgctgactacttgatgcaagaaaaagtgaacgactggaactcaattgctgaaattgatgaagaatcaattcgtcttcgtgaagaaatcaatcttcaatatcaggcacttggcgaagtagtgcggttgggtgatctgtatggtcttgatgttcgcaaacctgctatgaatgttaaggaagctatccaatggattaatatcgcctttatggctgtctgccgcgttatcaatggtgctgcaacttctcttggacgtgtcccaatcgttcttgatatctttgcagaacgtgaccttgctcgtggcactttcactgaatcagaaatccaagaattcgttgatgacttcgttatgaaacttcgtacggttaaatttgcacgtactaaggcttatgacgaactttattcaggtgacccaacatttattacgacttctatggctggtatgggagctgatggacgtcaccgtgttactaagatggactaccgtttcttaaatacgcttgataatattggcaatgctccagaacctaacttaaccgttctttggtcaagtaaattgccttactctttccgtcattattgtatgtctatgagccacaagcattcttcaattcaatatgaaggtgtcacaactatggctaaagaaggttatggtgaaatgtcatgtatctcatgctgtgtatctccgcttgatcctgaaaacgaagatcgtcgccacaatctacaatactttggtgctcgtgttaacgttcttaaagcacttcttacaggtcttaatggcggttacgacgatgttcacaaagactacaaagtatttgatgtcgaacctatccgtgatgaagtccttgattttgaaacggttaaagctaattttgaaaaagcacttgattggttgactgatacttacgtggacgcaatgaatatcattcactatatgactgataaatataactatgaagccgttcaaatggccttcttaccaacacgtgttaaagccaatatgggatttggtatttgcggattctctaatacagttgattcattatcagctattaaatatgctactgtaaaacctattcgtgatgaagatggttacatttacgactatgaaactgttggtaacttccctcgttacggagaagatgatgaccgtgtagactcaatcgctgaatggttgcttgaagctttccatactcgtcttgcacgtcataaactgtacaaagattccgaagctactgtatcattgcttacaatcacttctaatgttgcttattctaaacaaactggtaattctccagttcacaagggtgtttacctcaatgaagatggttctgtgaacttgtctaaagtagaattcttctcaccaggtgctaacccatcaaataaagcttccggcggctggttgcaaaacttgaactcattgaagaaacttgactttgctcacgcaaatgatggtatctcattgacaactcaagtttcaccaaaagctcttggtaagacattcgatgaacaagttgctaacttagtaacaattcttgatggttactttgaaggcggcggtcaacacgttaacttgaacgttatggatcttaaagatgtttatgacaagatcatgaatggtgaagatgttatcgttcgtatctcaggttactgtgttaacactaaataccttactaaagaacaaaagactgaattgacacaacgtgttttccatgaagttctctcaatggatgatgcagctacagacttggttaacaacaagtaagagttaaacagtttagtttaaaagacctcactcataaaagtgaggtctttactttgctttcgggtacgatcaaagcagtgagagctttttatattctaaaaactcacaaattcagaaaaaaacagctcttgtgatttgaaaagcttttagctacaataatattatgaaaattaattatactcgcgacacactgtcatccacctatcttgatgcagtaaaaattagacaccttgtcttc

SEQ ID NO: 18 - DNA, 1993 bp, Haemophilus influenzae
gtccgaatggtgcaccagcacgacgaccatcaggggtgttacccgttttcttaccataaactacgttagaagtaatggttaatacagattgtgtaggcactgcattgcggtaagttttaagtttttgaattttcttcataaaacgttcaactaagtcacaagcgatgtcatcaacacggttatcattgttaccatattgtggatattcaccttcgatttcaaagtcgattgctacgttagttgcaacaacattgccatctttatctttgatgtcgccacgaactggtttaactttcgcatatttgattgctgaaagtgagtcagccgcaacagaaagacctgcgataccacaagccatagtacggtatacatcacgatcatgtaatgccattaatgcggcttcgtatgaatatttatcgtgcatatagtggattacgtttaaggcagtcacatattgttttgccaaccaatccataaagctatccatacgagtcattactgtatcgaaatctaatacttcatcagtaattggtgcagttttcggacctacttgcatacctaatttttcatcgataccgccgttgattgcgtataacaatgttttcgctaagtttgcacgtgcaccgaagaattgcatttgtttacccacaatcattggtgatacacaacatgcgattgcgtagtcatcgttgttgaagtctggacgcattaaatcatcgttttcgtattgaactgatgaggtatcaatcgatacttttgcacagaaacgtttgaagttttcaggtaattgttcagaccaaagaatggttaagtttggctctggagaagtacccatgttgtaaagggtgtgtaaaatacggaatgtatttttggttactaatgtacgaccatctaaacccatacctgcgatggtttcagttgcccacattgggtcaccagagaataattgatcgtattcaggtgtacgtaagaaacgaaccatacgaagtttcataactaagtggtcaactaattcttgcgcttcagtttcagtaatttttcctgcttttaaatcacgttcgatgtacacgtcaataaaggttgcggtacgaccgaatgacattgcagcaccattttgtgattttattgcagcaagataagcaaagtacatccattgaatggcttcttgagcattagttgctgggttagaaatatcataaccatagcttgctgccatttgttttaattgacctaatgcacggtgttgttctgcgatttcttcacgtaaacgaattgttgcttcaagatttacgccatcttctaaatctttttgtaaagaagagaattgtgcgtatttatctttcattaagaaatctacaccataaagtgctacacgacggtagtcaccgatgatacgaccacgaccataagcatctggaagaccagttaataccccagatttacggcaacgtaaaatatctggcgtgtaaacatcgaatacaccttggttatgtgttttacggtattcagtgaagatttttttcacttttggatcaagttcacgaccataaactttacaagaaccttccaccattttgataccaccgaatggcataatggcacgttttaaaggttcatcagtttgaagaccaacgattttttctaaatctttgttaatgtaaccaggtgcgtgagagataatggtagatggtgtatgttcatcaaaatctaatggcgcgtgagtacggttttcaattttaataccttccatcacagattcccaaagcttggttgttgcttcggttggacctgctaagaaagagtcatcgccttcataaggggtatagtttttttggataaagtcacgtacattgacattttcttgccaatcgccaccagcaaaaccagcccacgccaatttttgcatttcattaagttctgacatagtcatttcctttgttaattaataaataaatctttaatgtgttttggttaaataacgttggaatacacc

SEQ ID NO: 19 - protein, 746 aa, Escherichia coli
MKVDIDTSDKLYADAWLGFKGTDWKNEINVRDFIQHNYTPYEGDESFLAEATPATTELWEKVMEGIRIENATHAPVDFDTNIATTITAHDAGYINQPLEKIVGLQTDAPLKRALHPFGGINMIKSSFHAYGREMDSEFEYLFTDLRKTHNQGVFDVYSPDMLRCRKSGVLTGLPDGYGRGRIIGDYRRVALYGISYLVRERELQFADLQSRLEKGEDLEATIRLREELAEHRHALLQIQEMAAKYGFDISRPAQNAQEAVQWLYFAYLAAVKSQNGGAMSLGRTASFLDIYIERDFKAGVLNEQQAQELIDHFIMKIRMVRFLRTPEFDSLFSGDPIWATEVIGGMGLDGRTLVTKNSFRYLHTLHTMGPAPEPNLTILWSEELPIAFKKYAAQVSIVTSSLQYENDDLMRTDFNSDDYAIACCVSPMVIGKQMQFFGARANLAKTLLYAINGGVDEKLKIQVGPKTAPLMDDVLDYDKVMDSLDHFMDWLAVQYISALNIIHYMHDKYSYEASLMALHDRDVYRTMACGIAGLSVATDSLSAIKYARVKPIRDENGLAVDFEIDGEYPQYGNNDERVDSIACDLVERFMKKIKALPTYRNAVPTQSILTITSNVVYGQKTGNTPDGRRAGTPFAPGANPMHGRDRKGAVASLTSVAKLPFTYAKDGISYTFSIVPAALGKEDPVRKTNLVGLLDGYFHHEADVEGGQHLNVNVMNREMLLDAIEHPEKYPNLTIRVSGYACASTH

SEQ ID NO: 20 - protein, 769 aa, Haemophilus influenzae
SELNEMQKLAWAGFAGGDWQENVNVRDFIQKNYTPYEGDDSFLAGPTEATTKLWESVMEGIKIENRTHAPLDFDEHTPSTIISHAPGYINKDLEKIVGLQTDEPLKRAIMPFGGIKMVEGSCKVYGRELDPKVKKIFTEYRKTHNQGVFDVYTPDILRCRKSGVLTGLPDAYGRGRIIGDYRRVALYGVDFLMKDKYAQFSSLQKDLEDGVNLEATIRLREEIAEQHRALGQLKQMAASYGYDISNPATNAQEAIQWMYFAYLAAIKSQNGAAMSFGRTATFIDVYIERDLKAGKITETEAQELVDHLVMKLRMVRFLRTPEYDQLFSGDPMWATETIAGMGLDGRTLVTKNTFRILHTLYNMGTSPEPNLTILWSEQLPENFKRFCAKVSIDTSSVQYENDDLMRPDFNNDDYAIACCVSPMIVGKQMQFFGARANLAKTLLYAINGGIDEKLGMQVGPKTAPITDEVLDFDTVMTRMDSFMDWLAKQYVTALNVIHYMHDKYSYEAALMALHDRDVYRTMACGIAGLSVAADSLSAIKYAKVKPVRGDIKDKDGNVVATNVAIDFEIEGEYPQYGNNDNRVDDIACDLVERFMKKIQKLKTYRNAVPTQSVLTITSNVVYGKKTGNTPDGRRAGAPFGPGANPMHGRDQKGAVASLTSVAKLPFAYAKDGISYTFSIVPNALGKDAEAQRRNLAGLMDGYFHHEATVEGGQHLNVNVLNREMLLDAMENPDKYPQLTIRVSGYAVRFNSLTKEQQQDVITRTFTESM

SEQ ID NO: 21 - protein, 195 aa, Chlamydomonas reinhardtii
GSFPKYGNDDDRVDEIAEWVVSTFSSKLAKQHTYRNSVPTLSVLTITSNVVYGKKTGSTPDGRKKGEPFAPGANPLHGRDAHGALASLNSVAKLPYTMCLDGISNTFSLIPQVLGRGGEHERATNLASILDGYFANGGHHINVNVLNRSMLMDAVEHPEKYPNLTIRVSGYAVHFARLTREQQLEVIARTFHDTM

SEQ ID NO: 22 - DNA, 1006 bp, Lactococcus lactis
tgttacctggtttgaacggtggttacgttcataaagattataaagtattcgatattgaacctgttcgtgatgaaattcttgactatgatacagttatggaaaacttcgacaaatcactcaactggttgacagatacttatgttgatgcaatgaatatcattcactacatgactgacaaatataactatgaagcagttcaaatggccttcttgcctactaaagttcgtgctaacatgggatttggtatctgtggtttcgcaaatacagttgattcactttcagcgattaaatatgctaaagttaaaactttgcgtgatgaaaatggctacatctacgattatgaagtagaaggtgacttcccacgttatggtgaagatgatgaccgtgctgatgatatcgctaaacttgtcatgaaaatgtaccatgaaaaattagcttcacacaaactttacaaaaatgctgaagctactgtttcacttttgacaatcacatctaacgttgcttactctaaacaaactggtaactctccagttcataaaggagtattcctcaatgaagatggtacagtcaacaaatctaaacttgaattcttctcaccaggtgctaacccatctaacaaagctaaaggtggatggttgcaaaatcttcgttcattagctaaattggaattcaaagatgcaaatgacggtatttcattaactactcaagtttctcctcgtgcacttggtaaaactcgtgatgaacaagtagataacttggttcaaattcttgatggatacttcacaccaggagctttgattaatggtactgaatttgcaggtcaacacgttaacttgaacgttatggaccttaaagatgtttacgataaaatcatgcgtggtgaagatgttatcgttcgtatctctggatactgtgttaacactaaatacctcacacctgaacaaaaacaagaattgactgaacgtgtcttccatgaagtactttcaaacgatgatgaagaagtaat

SEQ ID NO: 23 - protein, 334 aa, Lactococcus lactis
LPGLNGGYVHKDYKVFDIEPVRDEILDYDTVMENFDKSLNWLTDTYVDAMNIIHYMTDKYNYEAVQMAFLPTKVRANMGFGICGFANTVDSLSAIKYAKVKTLRDENGYIYDYEVEGDFPRYGEDDDRADDIAKLVMKMYHEKLASHKLYKNAEATVSLLTITSNVAYSKQTGNSPVHKGVFLNEDGTVNKSKLEFFSPGANPSNKAKGGWLQNLRSLAKLEFKDANDGISLTTQVSPRALGKTRDEQVDNLVQILDGYFTPGALINGTEFAGQHVNLNVMDLKDVYDKIMRGEDVIVRISGYCVNTKYLTPEQKQELTERVFHEVLSNDDEEV

SEQ ID NO: 24 - protein, 776 aa, Streptococcus mutans
MATVKTNTDVFEKAWEGFKGTDWKDRASISRFVQDNYTPYDGGESFLAGPTERSLHIKKVVEETKAHYEETRFPMDTRITSIADIPAGYIDKENELIFGIQNDELFKLNFMPKGGIRMAETALKEHGYEPDPAVHEIFTKYATTVNDGIFRAYTSNIRRARHAHTVTGLPDAYSRGRIIGVYARLALYGADYLMQEKVNDWNSIAEIDEESIRLREEINLQYQALGEVVRLGDLYGLDVRKPAMNVKEAIQWINIAFMAVCRVINGAATSLGRVPIVLDIFAERDLARGTFTESEIQEFVDDFVMKLRTVKFARTKAYDELYSGDPTFITTSMAGMGADGRHRVTKMDYRFLNTLDNIGNAPEPNLTVLWSSKLPYSFRHYCMSMSHKHSSIQYEGVTTMAKEGYGEMSCISCCVSPLDPENEDRRHNLQYFGARVNVLKALLTGLNGGYDDVHKDYKVFDVEPIRDEVLDFETVKANFEKALDWLTDTYVDAMNIIHYMTDKYNYEAVQMAFLPTRVKANMGFGICGFSNTVDSLSAIKYATVKPIRDEDGYIYDYETVGNFPRYGEDDDRVDSIAEWLLEAFHTRLARHKLYKDSEATVSLLTITSNVAYSKQTGNSPVHKGVYLNEDGSVNLSKVEFFSPGANPSNKASGGWLQNLNSLKKLDFAHANDGISLTTQVSPKALGKTFDEQVANLVTILDGYFEGGGQHVNLNVMDLKDVYDKIMNGEDVIVRISGYCVNTKYLTKEQKTELTQRVFHEVLSMDDAATDLVNNKZ

SEQ ID NO: 25 - protein, 740 aa, Clostridium pasteurianum
LFKQWEGFQDGEWTNDVNVRDFIQKNYKEYTGDKSFLKGPTEKTKKVWDKAVSLILEELKKGILDVDTETISGINSFKPGYLDKDNEVIVGFQTDAPLKRITNPFGGIRMAEQSLKEYGFKISDEMHNIFTNYRKTHNQGVFDAYSEETRIARSAGVLTGLPDAYGRGRIIGDYRRVALYGIDFLIQEKKKDLSNLKGDMLDELIRLREEVSEQIRALDEIKKMALSYGVDISRPAVNAKEAAQFLYFGYLAGVKENNGAAMSLGRTSTFLDIYIERDLEQGLITEDEAQEVIDQFIIKLRLVRHLRTPEYNELFAGDPTWVTESIAGVGIDGRSLVTKNSFRYLHTLINLGSAPEPNMTVLWSENLPESFKKFCAEMSILTDSIQYENDDIMRPIYGDDYAIACCVSAMRVGKDMQFFGARCNLAKCLLLAINGGVDEKKGIKVVPDIEPITDEVLDYEKVKENYFKVLEYMAGLYVNTMNIIHFMHDKYAYEASQMALHDTKVGRLMAFGIAGFSVAADSLSAIRYAKVKPIRENGITVDFVKEGDFPKYGNDDDRVDSIAVEIVEKFSDELKKHPTYRNAKHTLSVLTITSNVMYGKKTGTTPDGRKVGEPLAPGANPMHGRDMEGALASLNSVAKVPYVCCEDGVSNTFSIVPDALGNDHDVRINNLVSIMGGYFGQGAHHLNVNVLNRETLIDAMNNPDKYPTLTIRVSGYAVNFNRLSKDHQKEVISRTFHEKL

SEQ ID NO: 26 - DNA, 1848 bp, Lactococcus lactis
  CDS (591)..(1613)
ctgcagcttgttttttagtaccaacaaaaaggactactgcaccttcttgtgaagcgttttttacatagttgtaagcatcgtcaacaagttttacagttttttgaaggtcgataacgtggataccattacgttctgtgaagatgtatggtttcatttttgggttccaacgacgagtttggtgaccgaagtgaacaccagcttcaagaagttgtttcattgaaataactgacatgttaatgtctccttttaaaatagtttttcctctttcatctgtcatccgcagccgcaatacttgcgtacactacgactttgtcgagacgaaatgcgagatggttgcatagcaactctatcattatacattgtttgacctatttttgcaagtatctattcatgcttctattgttcagtaaatctatttttctaaccactcctattatctgacaaatttaattgttaatttaggctctataatcactaaaagagtaagtttttaaatttttttctaagaaaaaaattaatatttttgctgaaaccgctttttttgtgataaaataattatagtaaataaattagtttgtgaggagagaaatatgaaagaaaaaatccttttaggcggctatacaaaacgtgtatctaaaggcgtatatagtgttcttttggacactaaagctgctgaattatcatcattaaatgaagtcgctgcggttcaaaaccctacttatatcactctcgatgaaaagggacacctctatacttgtgcagcagatagtaatggtggaggaatcgccgcctttgattttgatggcgaaactgctactcatctcggaaatgtcacaaccacgggagctccactctgctatgttgccgtggacgaagcgcgacaattagtttacggagcgaactatcatcttggagaagttcgtgtttataagattcaagctaatggctcactccgattaacggatacagtaaaacataccggttctggaccacgtcctgaacaagctagctcacacgttcattattctgatttgactcctgacggacgacttgtcacctgtgatttgggaacagatgaagtcactgtttatgatgtcattggtgaaggtaaactcaatattgctacaatttatcgggcagaaaaaggaatgggtgctcgtcatattactttccatccaaatggtaaaatcgcttatttggttggagagttaaattcaacaattgaagttttaagttacaatgaagaaaaaggacgctttgctcgtcttcaaacaattagcaccctacctgaagattatcatggagcaaatggtgttgctgccatccgtatttcatctgacggtaaattcctctatacttctaatcgtggacatgattctttgacaacttacaaagtaagtcctcttggtacaaaacttgaaactattggctggacaaatactgaaggtcatatccctcgcgattttaatttcaacaaaactgaagattatatcattgtcgctcatcaagaatctgataatttatctcttttcttgcgagataaaaaaaccggtactttaactttggaacaaaaagatttttacgctcctgaaatcacttgtgttttaccactataaaaatttattttttcacaaagtttgactgataaactaaaaaagattgctaatttctctcaaagaattagcaatctttttttcttcagtaaagcttgttacaaaaccgttttctaaacttttgatgagtgtttttgtaaaaactatcacaatattgcttgacatctataaaaaactttgttaaactattcacgtaaaagaaagtgaatgaagtcacaaaggagaacctacaaat

SEQ ID NO: 27 - protein, 341 aa, Lactococcus lactis
MKEKILLGGYTKRVSKGVYSVLLDTKAAELSSLNEVAAVQNPTYITLDEKGHLYTCAADSNGGGIAAFDFDGETATHLGNVTTTGAPLCYVAVDEARQLVYGANYHLGEVRVYKIQANGSLRLTDTVKHTGSGPRPEQASSHVHYSDLTPDGRLVTCDLGTDEVTVYDVIGEGKLNIATIYRAEKGMGARHITFHPNGKIAYLVGELNSTIEVLSYNEEKGRFARLQTISTLPEDYHGANGVAAIRISSDGKFLYTSNRGHDSLTTYKVSPLGTKLETIGWTNTEGHIPRDFNFNKTEDYIIVAHQESDNLSLFLRDKKTGTLTLEQKDFYAPEITCVLPL

SEQ ID NO: 28 - DNA, 4741 bp, Lactococcus lactis
  CDS (453)..(1475)
tttggtgaccgaagtgaacaccagcttcaagaagttgtttcattgaaataactgacatgttaatgtctccttttaaaatagtttttcctctttcatctgtcatccgcagccgcaatacttgcgtacactacgactttgtcgagacgaaatgcgagatggttgcatagcaactctctcattatacattgtttaagctacttttgcaagcatctattcatttatttcttttatcaatatgagtaaatgaaagctatcctacccccctttctttttattctgttttttatatctcaatgttgtctgacaaatttaacgaatatttttgcctatataatccccataagggagatttttacatttttttctaagaataaaattaatatttttgctgaaaacgctttttttgtgataaaataattatagtaaataaaatagtttgtgaggagagaaatatgaaagaaaaaatccttttaggcggttatactaaacgtgtatctaaaggcgtttacagtgttctattagatagcaagaaagctgaattgtcggctttaactgaagttgcagcggttcaaaatccaacttatatcactcttgatcaaaaagggcacctctacacttgtgctgctgatggaaatggtggtggaattgctgcctttgatttcgatggtcaaaatacaactcacctagggaatgtaacgagtactggagcccctttgtgttatgtggctgttgatgaagcacgtcaactcgtttatggtgccaactatcacttgggtgaagttcgtgtgtacaaaattcaagctgatggttcccttagattaaccgatacagttaaacataatggttctggccctcgacctgagcaagcaagttctcatgtccattactctgatttaactccagatggtcgtcttgttacttgtgatttaggtacagatgaagtgactgtttacgatgttattggtgaaggtaaactcaatatcgttacgatttatcgtgccgaaaaaggaatgggagctcgtcacatcagcttccatcctaatggaaaaattgcttatctcgtcggagaattaaattcaactattgaagttctaagctataatgaagaaaaaggacgattcgctcgtcttcaaacaatcagtactttacctgaagactatcacggagccaatggagtagctgctattcgaatttcttctgatggtaagttcctctatgcttctaatcgtgggcacgactctttagcaatttacaaggtaagtcctctcggaacaaaattagaatctattggttggacaaagactgaatatcatattccacgcgattttaattttaataaaaccgaagattatatcattgtcgctcatcaagaatctgataatttaactcttttcttgagagataaaaatacagggtcattaacgttagaacaaaaagacttttacgctcctgaaattacttgtgttttacctttgtaaaaactaaactttagtaaatcttgcttttgttttttcacaaagttttactaaatcagacaaaaaaatattgccaaatctttaaaaggattggcaatatttttttgtctgaaacccttgcttataaagcgatttctaaaagtttgatgagtttttttgtaaatttcatcacaatatcgcttgacttctttaaaaaactttgttaaactattcacgtaaaagaaagtgaatggaatcacaaaggagaacgtacacatatggcaactaaaaaagccgctccagctgcaaagaaagttttaagcgctgaagaaaaagccgcaaaattccaaggaagtgtcgcttatactgatcaattagtcaaaaaagctcaagctgcagttcttaaatttgaaggatacacacaaactcaagttgatactattgttgctgcaatggctcttgcagcaagcaaacattctctggaactcgctcacgaagccgttaatgaaactggccgtggagttgttgaggacaaagatacaaaaaaccattttgcttctgaatctgtttataatgcaatcaaaaatgataaaacagttggcgttatcgctgaaaacaaagttgctggttctgttgaaatcgcaagcccccttggagtacttgctggtattgtcccaacaactaatccaacatcaacagccatctttaaatcattattaactgcaaagacacgtaatgctattgtctttgcctttcacccacaagcacaaaaatgctcaagccatgcggcaaaaattgtttatgatgctgcgattgaagctggtgcacctgaagactttattcaatggattgaagtacccagtcttgatatgacgactgctttgattcaaaatagaggaattgctacaattcttgcaactggtggtccaggtatggtcaatgccgcgcttaagtctggtaatccttcacttggtgtaggtgctggtaatggtgcagtttatgttgatgcaactgcaaatatcgatcgtgctgttgaagatcttttgctttcaaaacgttttgataacggaatgatttgtgcgactgaaaactctgcagttattgatgcatcaatctatgatgaatttgtcgctaaaatgccaacgcaaggcgcttatatggttcctaaaaaagattacaaggcaattgaaagttttgttttcgttgaacgtgctggtgaaggttttggtgtaactggtcctgttgctggtcgttctggtcaatggattgctgaacaagctggtgttaacgtccctaaagataaagatgttcttctttttgaacttgataagaaaaatattggggaagctctttcttctgaaaaactttctcctttgctttcaatctacaaatcagaaacacgtgaagaaggaattgaaattgtacgtagcttacttgcttaccaaggagctggtcacaacgctgccattcaaatcggtgcaatggacgacccatttgtcaaagaatacggaattaaagtcgaagcttctcgtatcctcgttaaccaacctgactctatcggtggggtcggagatatttatactgatgcaatgcgtccatcattgacgctcggaactggttcatgggggaaaaattcactttcacacaatttgagtacatacgatctattgaatgttaaaacagtggctaaacgtcgtaatcgccctcaatgggttcgtttgccaaaagaaatttactacgaaaaaaatgcaatttcttacttacaagaattgccacacgtccacaaagctttcattgttgccgaccctggtatggttaaattcggtttcgttgataaagttttggaacaacttgctatccgcccaactcaagttgaaacaagcatttatggctcagtccaacctgacccaactttgagtgaagcaattgcaatcgctcgtcaaatgaaccattttgaacctgacactgtcatctgtcttggtggtggttctgctctcgatgctggtaagattggtcgtttgatttatgaatatgatgctcgtggtgaggctgacctttccgatgacgcaagtttgaaagagatcttccaagagttagctcaaaaatttgttgatattcgtaaacgtattatcaaattctaccacccacacaaagcacaaatggttgctatccctactacttctggtactggttctgaagtgactccatttgcggttatcactgatgatgaaactcacgttaaatatccacttgctgactatcaattgacacctcaagttgccattgttgaccctgagtttgttatgactgtaccaaaacgtactgtttcttggtctgggattgatgctatgtcacacgcgcttgaatcttatgtttctgtcatgtcttctgactatacaaaaccaatttcacttcaagccatcaaactcatctttgaaaacttgactgagtcttatcattatgacccagctcatccaaccaaagaaggtcaaaaagctcgcgaaaacatgcacaatgctgcaacactcgctggtatggccttcgccaatgctttccttggaattaaccactcacttgctcataaaattgctggtgaatttgggcttcctcatggtcttgccattgctatcgctatgccacatgtcattaaatttaacgctgtaacaggaaacgttaaatttaccccttacccacgttatgaaacttatcgtgcgcaagaagactacgctgaaatttcacgcttcatgggatttgctggcaaagaagattcagatgaaaaagcggtcaaagctttggttgctgaacttaaaaaattgactgatagtattgatattaatatcaccctttcaggaaatggtgtagataaagctcatcttgaacgtgagcttgataaattggctgaccttgtttacgatgaccaatgtacacctgctaatccacgtcaaccaagaattgatgagattaaacaactcttgttagaccaatattaatatattaattatagtatttggaaccgaacgatatccatgctcgctaacctgctaaagcaggaagtcgcaatggtacgtcaaccaagaattgatgagattaaacaactcttgttagatcaatactaataatctgttgataaaaataattaaaacgctctgatgaattcgtcagagcgttttttattatagcttatacaactatcaaaaggtataaatcaatttcgatataggctcttttcactccattgatttatatttatataaaaatcaataattaattagcgatagaagtgatcc

SEQ ID NO: 29 - protein, 341 aa, Lactococcus lactis
MKEKILLGGYTKRVSKGVYSVLLDSKKAELSALTEVAAVQNPTYITLDQKGHLYTCAADGNGGGIAAFDFDGQNTTHLGNVTSTGAPLCYVAVDEARQLVYGANYHLGEVRVYKIQADGSLRLTDTVKHNGSGPRPEQASSHVHYSDLTPDGRLVTCDLGTDEVTVYDVIGEGKLNIVTIYRAEKGMGARHISFHPNGKIAYLVGELNSTIEVLSYNEEKGRFARLQTISTLPEDYHGANGVAAIRISSDGKFLYASNRGHDSLAIYKVSPLGTKLESIGWTKTEYHIPRDFNFNKTEDYIIVAHQESDNLTLFLRDKNTGSLTLEQKDFYAPEITCVLPL

SEQ ID NO: 30 - DNA, 4741 bp, Lactococcus lactis; identical to SEQ ID NO: 28
  CDS (1733)..(4441)

SEQ ID NO: 31 - protein, 903 aa, Lactococcus lactis
MATKKAAPAAKKVLSAEEKAAKFQGSVAYTDQLVKKAQAAVLKFEGYTQTQVDTIVAAMALAASKHSLELAHEAVNETGRGVVEDKDTKNHFASESVYNAIKNDKTVGVIAENKVAGSVEIASPLGVLAGIVPTTNPTSTAIFKSLLTAKTRNAIVFAFHPQAQKCSSHAAKIVYDAAIEAGAPEDFIQWIEVPSLDMTTALIQNRGIATILATGGPGMVNAALKSGNPSLGVGAGNGAVYVDATANIDRAVEDLLLSKRFDNGMICATENSAVIDASIYDEFVAKMPTQGAYMVPKKDYKAIESFVFVERAGEGFGVTGPVAGRSGQWIAEQAGVNVPKDKDVLLFELDKKNIGEALSSEKLSPLLSIYKSETREEGIEIVRSLLAYQGAGHNAAIQIGAMDDPFVKEYGIKVEASRILVNQPDSIGGVGDIYTDAMRPSLTLGTGSWGKNSLSHNLSTYDLLNVKTVAKRRNRPQWVRLPKEIYYEKNAISYLQELPHVHKAFIVADPGMVKFGFVDKVLEQLAIRPTQVETSIYGSVQPDPTLSEAIAIARQMNHFEPDTVICLGGGSALDAGKIGRLIYEYDARGEADLSDDASLKEIFQELAQKFVDIRKRIIKFYHPHKAQMVAIPTTSGTGSEVTPFAVITDDETHVKYPLADYQLTPQVAIVDPEFVMTVPKRTVSWSGIDAMSHALESYVSVMSSDYTKPISLQAIKLIFENLTESYHYDPAHPTKEGQKARENMHNAATLAGMAFANAFLGINHSLAHKIAGEFGLPHGLAIAIAMPHVIKFNAVTGNVKFTPYPRYETYRAQEDYAEISRFMGFAGKEDSDEKAVKALVAELKKLTDSIDINITLSGNGVDKAHLERELDKLADLVYDDQCTPANPRQPRIDEIKQLLLDQY

SEQ ID NO: 32 - DNA, 31 bp, Artificial Sequence
  Primer adhP1-XhoI
ggccgctcgaggttgaacgtgctggtgaagg

SEQ ID NO: 33 - DNA, 31 bp, Artificial Sequence
  Primer adhP2-BamHI
tagtaggatccgggtcaggttggactgagcc

SEQ ID NO: 34 - DNA, 1750 bp, Lactococcus lactis
  CDS (464)..(1378)
gcgcctagataagaaacagcaacagctaaaagataggtatcaaaagcacttgatttaaaaataatgactttatccgattttttgattcccaactcagataagagacttgccttatcaacaattgcttgatgagtcttttggtaagtcgtttcaagagctagttcggggaaagctccaacagcctcatcaaagataattgggctatcaggaaactgttcagctgattttttaaagtttagatacaaatttaggggttcgtgtttgaatttcaaaaaaaatctcctcaagttaataagtttattatatcacaaagtattctttagaccaatagttaatgtaaatgttttcttaagtcgtagagaataaaattctcggaaaaaaagtctaaaatctgctacaattaaagggacactaagaggattccaatcctcttttatcaggaaaagaagggatagataggaaaatgattaaaaattatgaactatccaacgaaaaaaaattaatttcaacctctgaaatgaagaatttcacctatgttctcaatccaacacgtgaagaaattgggaatatttctgaatactatgacttcccttttgactatttatcaggaattttggatgactatgaaaatgcccgttttgaaacagatgataatgataataatctgattctcttacaatatcctccactctctaattatggagaagtggcgacttttccatattctttggtttggactaaaaatgaatcggttattttagcacttaatcatgagattgataatggcttaattttcgagcgtgaatatgattataaacgctacaaacatcaagttatttttcaagtgatgtatcaaatgacacacactttccatgattatttgagagatttccgaacaaggcgtcgcagacttgaacagggaatcaaaaattcaacaaagaacgaccaaattgttgatttgattgccattcaagcaagtttaatttattttgaagatgccttgcacaataatatgcaagtacttcaggattttattgattacttgagagaagatgatgaagacggttttgctgaaaagatttatgatatttttgtcgaaacagaccaagcttatacagaaaccaagattcagctcaagttactagaaaatctccgagatttgttctcaaacaatgtctctaataacttgaacattgtcatgaaaatcatgacatcagctactttcgttctagggattcctgcagtaattgttggtttttacggaatgaatgttccaattcctggtcaaaattttaattggatggtttggcttattttagttctaggaattttattatgtgtttgggtcacttggtggttacataaaaaagatatgttataaaatggagaaaaatctccatttttttgctctttgtgaaaaaattaattagtgattgcagattatgaagttagcaatgtttgttaaaactattttgtgaattatttatgaaaacgttttaaaaaagtataacagatattaaaataattggaactgtattagtaaagaatctgtaatttctcttgaattctgtttgctattctcaaactgtatgatataatgaagttgtaatttgaaacagaaagaacaaaggagatttcaaaatgaaaaccgaagttacggaaaatatctttgaacaagcttgggatggttttaaaggaaccaactggcgcgataaagcaagcgttactcgctttgtacaagaaaactaca

SEQ ID NO: 35 - protein, 305 aa, Lactococcus lactis
MIKNYELSNEKKLISTSEMKNFTYVLNPTREEIGNISEYYDFPFDYLSGILDDYENARFETDDNDNNLILLQYPPLSNYGEVATFPYSLVWTKNESVILALNHEIDNGLIFEREYDYKRYKHQVIFQVMYQMTHTFHDYLRDFRTRRRRLEQGIKNSTKNDQIVDLIAIQASLIYFEDALHNNMQVLQDFIDYLREDDEDGFAEKIYDIFVETDQAYTETKIQLKLLENLRDLFSNNVSNNLNIVMKIMTSATFVLGIPAVIVGFYGMNVPIPGQNFNWMVWLILVLGILLCVWVTWWLHKKDML

SEQ ID NO: 36 - DNA, 4191 bp, Lactococcus lactis
  CDS (270)..(1184)
ttgggctataaggaaattgttctgctgattttttaaagtttagatataggtttaggggttcatgtttgaatttcaaaaaaagtctcctcaagttaataagtttattatatcacaaagtattatttagaccaacttccttcaaaaaacttttcgttaaggctttgaaataaaataatgagaaaaaaataggaaaatctgctacaattagaaggagaagaagaggatttaaatccttttttattaggaaaagaagggatagataggctgatatgataaaaaattatgaactatccaatgaaaaaaaattgatctcaacttctgagatgaagaatttcacttatgtcctcaatccaacacgtgaagaaattgggaatatctcagaacactatgattttccttttgactatctatctggaattttagatgactatgaaaatgcccgttttgaaacagatgataatgacaataatctgattcttttgcaatatcccgccttgtccaactatggagaagtggccacttttccatattctttggtttggactaagaatgaatcggttattttggcccttaaccatgaaattgataatggtctcatttttgaacgagaatatgattataaacgctataaacaccaattgatttttcaagtgatgtaccaaatgactcatacttttcatgattatttgagagactttagaacaaggcgccgccggcttgaagttggtatcaaaaattcaacaaaaaatgaccaaattgttgacttaattgccattcaagcgagtttgatttattttgaagatgcgctgcacaataatatgcaagttctccagaattttattgattacttacgagaagatgatgaagatggttttgccgaaaaaatctatgatatttttgtcgaaacagaccaagcttatacagaaaccaagattcagctcaagttactagaaaatctccgagatttgttctcaaacattgtctctaataatttgaatatcgtcatgaaaattatgacctcagcaacatttgttctaggtattccggcggttattgtcggcttttatggaatgaatgttccgattcctggtcaaaattttaattggatggtctggctcattttggtgtttggaattttattatgtgtttgggttacttggtggctacacaaaaaagatatgttatgaatggagaaaatttctccgtttttttatctttgtgaaaaaattaattagtgataataaatcatgaagttagcaatgtttgtcaaagctatttagtgaattaattatgaaaacgttttaaaaaagtataacagatattaaaataattgaaactgtattagtaaagaatctgtaatttctcttgaattctgtttgctattatcaaactgtatgatataatgaagttgtaatttgaaacagaaagaacaaaggagatttcaaaatgaaaaccgaagttacggaaaatatctttgaacaagcttgggatggttttaaaggaactaactggcgcgataaagcaagcgttactcgctttgtacaagaaaactacaaaccatatgatggtgatgaaagctttcttgctgggccaacagaacgtacacttaaagtaaagaaaattattgaagatacaaaaaatcactacgaagaagtaggatttccctttgatactgaccgcgtaacctctatcgataaaattcctgctggatatattgatgctaatgataaagaacttgaactcatctatgggatgcaaaatagcgaacttttccgcttaaacttcatgccaagaggtggtcttcgtgttgctgaaaagattttgacagaacacggtctttcagttgacccaggtttgcatgatgttttgtcacaaacaatgacttctgtaaatgatggaatcttccgtgcttatacttcagcaattcgtaaagcacgtcacgctcacactgtaacaggtttgcctgatgcatactctcgtggacgtatcatcggggtatatgcacgtcttgctctttatggagctgactaccttatgaaggaaaaagcaaaagaatgggatgcaatcactgaaattaatgatgataacattcgtcttaaagaagaaattaacatgcaataccaagctttgcaagaagttgtaaactttggtgctttgtatggtcttgacgtttctcgtccagcgatgaacgtaaaagaagcaatccaatgggttaatattgcatacatggcagtttgtcgtgttatcaatggtgctgcaacttcacttggacgtgtgccaatcgttcttgacatctttgcagaacgtgaccttgctcgtggaacatttactgagcaagaaatccaagaatttgttgatgatttcattttaaaacttcgtacaatgaaatttgctcgtgctgctgcttatgatgaactttattctggtgaccccacgttcatcacaacatctatggctggtatgggtaatgacggacgccaccgtgtcactaaaatggactatcgtttcttgaacacacttgatacaatcggaaatgctccagaaccaaacttgacagttctttgggactctaaactcccatattcattcaaacgttattcaatgtctatgagtcacaaacactcatctatccaatatgaaggtgttgaaacaatggctaaagatggatatggcgaaatgtcatgtatctcttgttgtgtctcaccacttgacccagaaaatgaagaaggacgtcataatctccaatactttggtgcgcgtgtaaacgtcttgaaagcaatgttgactggtttgaacggtggttacgatgacgttcataaagattataaagtattcgatattgaacctgttcgtgatgaaattcttgactatgatacagttatggaaaacttcgacaaatcactcaactggttgacagatacttatgttgatgcaatgaatatcattcactacatgactgacaaatataactatgaagcagttcaaatggccttcttgcctactaaagttcgtgctaacatgggatttggtatctgtggtttcgcaaatacagttgattcactttcagcgattaaatatgctaaagttaaaactttgcgtgatgaaaatggctacatctacgattatgaagtagaaggtgacttcccacgttatggtgaagatgatgaccgtgctgatgatatcgctaaacttgtcatgaaaatgtaccatgaaaaattagcttcacacaaactttacaaaaatgctgaagctactgtttcacttttgacaatcacatctaacgttgcttactctaaacaaactggtaactctccagttcataaaggagtattcctcaatgaagatggtacagtcaacaaatctaaacttgaattcttctcaccaggtgctaacccatctaacaaagctaaaggtggatggttgcaaaatcttcgttcattagctaaattggaattcaaagatgcaaatgacggtatttcattaactactcaagtttctcctcgtgcacttggtaaaactcgtgatgaacaagtagataacttggttcaaattcttgatggatacttcacaccaggagctttgattaatggtactgaatttgcaggtcaacacgttaacttgaacgttatggaccttaaagatgtttacgataaaatcatgcgtggtgaagatgttatcgttcgtatctctggatactgtgttaacactaaatacctcacacctgaacaaaaacaagaattgactgaacgtgtcttccatgaagtactttcaaatgatgatgaagaagtaatgcacacttcaaatatctaattcttagtattaaaaaatataaggtctgtcagttctactgacagactttttttctataaattaattataatagttaaaaactattatttttagtttaagaaaaataaaatttgtgctaaaatagatgaatgataaaggtaattggattaacaggcggaattgcgagtgggaaatcaacggtggttgattttttgatttctgaaggttatcaagtaattgatgctgacaaagttgttcgtcagttgcaagaacctgatgggaaactttttaatgcaataatggaaactttcggttcagattttactgacgaaaatgggaaattaaaccgatgcaaaattgagtgcttaagttttgctgacccaaatcaacgtcaaaaattat

SEQ ID NO: 37 - protein, 305 aa, Lactococcus lactis
MIKNYELSNEKKLISTSEMKNFTYVLNPTREEIGNISEHYDFPFDYLSGILDDYENARFETDDNDNNLILLQYPALSNYGEVATFPYSLVWTKNESVILALNHEIDNGLIFEREYDYKRYKHQLIFQVMYQMTHTFHDYLRDFRTRRRRLEVGIKNSTKNDQIVDLIAIQASLIYFEDALHNNMQVLQNFIDYLREDDEDGFAEKIYDIFVETDQAYTETKIQLKLLENLRDLFSNIVSNNLNIVMKIMTSATFVLGIPAVIVGFYGMNVPIPGQNFNWMVWLILVFGILLCVWVTWWLHKKDML

SEQ ID NO: 38 - DNA, 4191 bp, Lactococcus lactis; identical to SEQ ID NO: 36
  CDS (1447)..(3807)

SEQ ID NO: 39 - protein, 787 aa, Lactococcus lactis
MKTEVTENIFEQAWDGFKGTNWRDKASVTRFVQENYKPYDGDESFLAGPTERTLKVKKIIEDTKNHYEEVGFPFDTDRVTSIDKIPAGYIDANDKELELIYGMQNSELFRLNFMPRGGLRVAEKILTEHGLSVDPGLHDVLSQTMTSVNDGIFRAYTSAIRKARHAHTVTGLPDAYSRGRIIGVYARLALYGADYLMKEKAKEWDAITEINDDNIRLKEEINMQYQALQEVVNFGALYGLDVSRPAMNVKEAIQWVNIAYMAVCRVINGAATSLGRVPIVLDIFAERDLARGTFTEQEIQEFVDDFILKLRTMKFARAAAYDELYSGDPTFITTSMAGMGNDGRHRVTKMDYRFLNTLDTIGNAPEPNLTVLWDSKLPYSFKRYSMSMSHKHSSIQYEGVETMAKDGYGEMSCISCCVSPLDPENEEGRHNLQYFGARVNVLKAMLTGLNGGYDDVHKDYKVFDIEPVRDEILDYDTVMENFDKSLNWLTDTYVDAMNIIHYMTDKYNYEAVQMAFLPTKVRANMGFGICGFANTVDSLSAIKYAKVKTLRDENGYIYDYEVEGDFPRYGEDDDRADDIAKLVMKMYHEKLASHKLYKNAEATVSLLTITSNVAYSKQTGNSPVHKGVFLNEDGTVNKSKLEFFSPGANPSNKAKGGWLQNLRSLAKLEFKDANDGISLTTQVSPRALGKTRDEQVDNLVQILDGYFTPGALINGTEFAGQHVNLNVMDLKDVYDKIMRGEDVIVRISGYCVNTKYLTPEQKQELTERVFHEVLSNDDEEVMHTSNI

SEQ ID NO: 40 - DNA, 14 bp, Artificial Sequence
  Consensus FNR box
ttgatnnnnatcaa

SEQ ID NO: 41 - DNA, 14 bp, Artificial Sequence
  Consensus FNR box
ggagtnnnnatcaa

SEQ ID NO: 42 - DNA, 14 bp, Artificial Sequence
  Consensus FNR box
tttgcnnnnatcaa

SEQ ID NO: 43 - DNA, 32 bp, Artificial Sequence
  Primer pfl-P1MG1363
ggccgctcgagttgtgtctcaccacttgaccc

SEQ ID NO: 44 - DNA, 33 bp, Artificial Sequence
  Primer pfl-P2MG1363
tagtaggatcccatcatcttcaccataacgtgg

(19) United States
(12) Patent Application Publication
(10) Pub. No.: US 2003/0199035 A1
(43) Pub. Date: Oct. 23, 2003

(54) Metabolically engineered lactic acid bacteria and means for providing same

(76) Inventors: Jose Arnau, Hellerup (DK); Hans Israelsen, Alleroed (DK); Astrid Vrang, Lyngby (DK); Flemming Joergensen, Lyngby (DK); Soeren Michael Madsen, Copenhagen (DK)

Correspondence Address: IVER COOPER, Browdy and Neimark, Suite 300, 624 Ninth St., N.W., Washington, DC, 20001, US

(21) Appl. No.: 10/267,989

(22) Filed: Oct. 10, 2002

Related U.S. Application Data
(63) Continuation of application No. 08/981,097, filed on Dec. 17, 1997, now abandoned.
Application No. 08/981,097 is a 371 of international application No. PCT/DK97/00336, filed on Aug. 20, 1997.

(51) Int. Cl. C12P 21/02; C12P 7/06; C12N 9/02; C07H 21/04; C12N 1/21; C12N 15/74

(52) U.S. Cl. 435/69.1; 435/189; 435/252.3; 435/320.1; 536/23.2; 435/161

(57) ABSTRACT

The complete DNA sequences for adhE and pfl genes of Lactococcus lactis, recombinant replicons comprising one or both of these genes or comprising mutants or variants hereof including mutants in which the genes are inactivated, and recombinant lactic acid bacteria comprising such a replicon are provided. The gene sequences and/or sequences regulating the expression of the genes can be modified to provide metabolically engineered lactic acid bacteria which have an enhanced or reduced production of one or more metabolites resulting from citrate and/or sugar fermentation. Such metabolically modified cell are useful as starter cultures in the manufacturing of food products and animal feed having improved flavour and/or shelf life, including dairy products, or they can be used directly in the manufacturing of a lactic acid bacterial metabolite.

### FIELD OF INVENTION

The present invention pertains to the field of lactic acid bacterial starter cultures which are useful in the production of food products, animal feed or aroma compounds, and specifically there is provided means for metabolically engineering such lactic acid bacteria which are thereby modified in their production of metabolic end products including aroma or flavour compounds and/or compounds having antimicrobial effects.

### TECHNICAL BACKGROUND AND PRIOR ART

Lactic acid bacteria are used extensively as starter cultures in the food industry in the manufacture of fermented products including milk products such as e.g. yoghurt and cheese, meat products, bakery products, wine and vegetable products. Lactococcus lactis is one of the most commonly used lactic acid bacteria in dairy starter cultures. However, several other lactic acid bacteria such as Leuconostoc species, Lactobacillus species and Streptococcus species are also commonly used in food starter cultures. In the art, species of the obligate anaerobic bacteria belonging to Bifidobacterium which are taxonomically different from the group of bacteria generally referred to as lactic acid bacteria, are frequently included in the group of lactic acid bacteria due to their application as dairy starter cultures. Lactic acid bacteria are also commonly used as inoculants in feedstuffs of plant and animal origin, i.a. for preservation purposes.

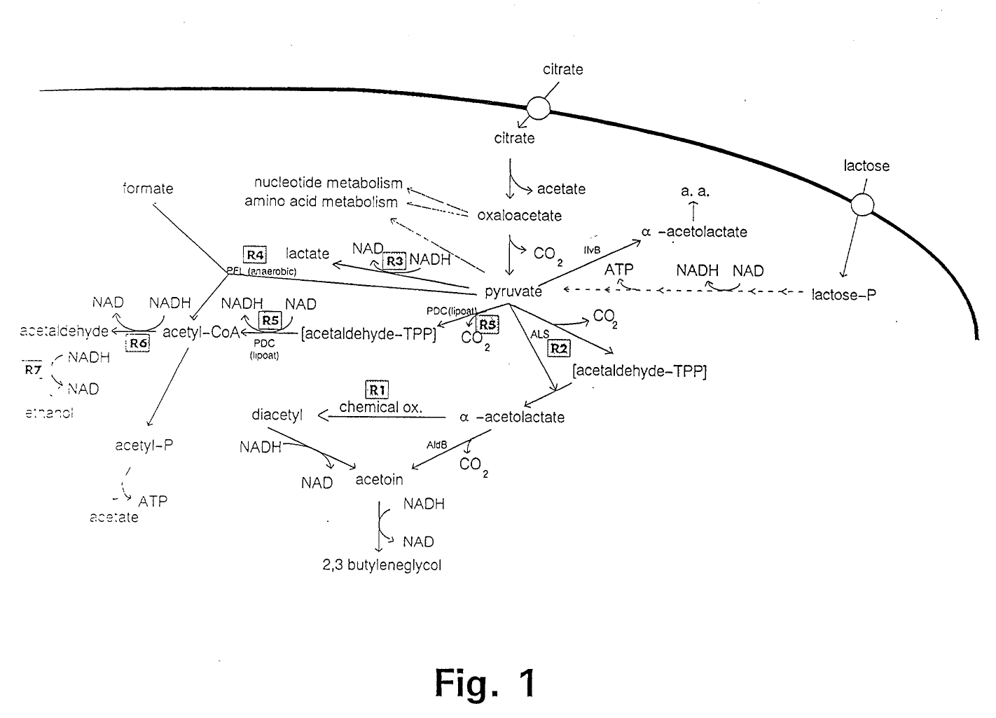

When a lactic acid bacterial starter culture is added to a substrate including milk or any other food or feed product starting material under appropriate conditions, the bacteria grow rapidly with concomitant conversion of lactose or other sugars to lactic acid/lactate and minor amount of acetate resulting in a pH decrease. In addition, several other metabolites are produced during the growth of lactic acid bacteria. Among these metabolites, diacetyl is one essential flavour compound which is formed during fermentation of the citrate-utilizing species of e.g. Lactococcus, Leuconostoc, and Lactobacillus. Diacetyl is formed by an oxidative decarboxylation (R1, FIG. 1) of α-acetolactate which is formed from two molecules of pyruvate by the action of α-acetolactate synthase (R2, FIG. 1).

Pyruvate is a key intermediate of several lactic acid bacterial metabolic pathways including the citrate metabolism and the degradation of lactose or glucose to lactate. The pool of pyruvate in the cells is critical for the flux through the pathway leading to diacetyl, acetoin and 2,3 butylene glycol due to α-acetolactate synthase affinity for pyruvate. Overproduction of α-acetolactate synthase in Lactococcus lactis as an approach for increased production of diacetyl has been disclosed by Platteuw et al. 1995.

An alternative metabolic engineering approach to providing an increased pool of pyruvate in lactic acid bacteria is to block one or several pyruvate degrading pathways. As an example hereof, a Lactococcus lactis mutant defective in the lactate dehydrogenase (R3, FIG. 1) has been disclosed by Gasson et al. (ref. 8, unpublished data, in Platteuw et al. 1995). Under aerobic conditions pyruvate is accumulated in this mutant leading to the formation of increased levels of acetoin and 2,3 butylene glycol. However, formate and ethanol were the major metabolic end products obtained under anaerobic conditions, but the formation of the latter end products in high amounts is generally undesired in fermented dairy products typically being produced under anaerobic conditions.

The reaction whereby pyruvate is converted to formate and acetyl coenzyme A (acetyl CoA) (R4, FIG. 1) by the action of pyruvate formate-lyase (Pfl) takes place only under anaerobic conditions (Frey et al. 1994). An alternative pathway for the formation of acetyl CoA from pyruvate (R5, FIG. 1) in a lactic acid bacterium is by the activity of the pyruvate dehydrogenase complex (PDC). In contrast to Pfl, the activity of PDC appears to be optimal under aerobic conditions (Snoep et al. 1992). Consequently, the pyruvate pool assumingly will be increased under anaerobic conditions by partially or completely blocking the Pfl activity. As mentioned above, an increased pyruvate pool may in turn lead to an increased flux from pyruvate towards acetoin and diacetyl via the intermediate α-acetolactate. Fermented foods or feed products produced by using a starter culture with reduced Pfl activity therefore may contain an increased amount of diacetyl or other products derived from conversion of α-acetolactate. In contrast, starter cultures with increased Pfl activity should result in enhanced production of the antimicrobially active metabolite formate and the use of such cultures in the production of feed or food products having increased shelf life can therefore be contemplated.

The pfl gene has been isolated from several microorganisms including Escherichia coli, Haemophilus influenzae, Clostridium pasteurianum and Streptococcus mutans. The Pfl enzyme is post-translationally activated by the Pfl activase via formation of an organic free radical into a glycine residue located at the C-terminal of Pfl (Frey et al. 1994). This modification of Pfl occurs only in the absence of oxygen. Although the activation gene, act encoding the Pfl activase flanks the pfl gene in E. coli, H. influenzae and C. pasteurianum, the act gene is transcribed from its own promoter, and the expression is essentially constitutive (Weidner et al. 1996). In contrast, the pfl expression is induced 12 to 15 fold by anaerobiosis (Sauter and Sawers 1990). The free radical enzyme, i.e. the activated Pfl, is destroyed by oxygen with concomitant fragmentation of the polypeptide chain (ref. 2 in Kessler 1992). However, in E. coli a Pfl deactivase activity has been found which under anaerobic conditions reverts the active radical form to the native non-radical form of Pfl (Kessler et al. 1992). By this activity, Pfl deactivase protects Pfl against being irreversibly destroyed by oxygen.

The AdhE protein of E. coli has acetaldehyde dehydrogenase activity, catalyzing the conversion of acetyl CoA to acetaldehyde (R6, FIG. 1), and ethanol dehydrogenase activity, catalyzing the conversion of acetaldehyde to ethanol (R7, FIG. 1). Additionally, the E. coli AdhE protein is responsible for the Pfl deactivase activity.

In the strict anaerobe, Clostridium acetobutylicum an adhE analogue, aad, has been cloned and characterized. However, the presence of Pfl deactivase activity could not be verified for the Aad protein, since no evidence exists for the presence of Pfl in C. acetobutylicum (Nair et al. 1994).

Lactic acid bacteria including Lactococcus lactis species are facultatively anaerobic organisms like E. coli, indicating that the occurrence of Pfl activase and deactivase activities in these organisms is to be expected. Analysis of the expression of adhE in E. coli has shown an eight fold increase under anaerobic growth (Chen and Lin 1991). The facts that the regulation of expression of pfl and adhE under anaerobic conditions is similar and that expression of act in E. coli is constitutive suggest that an equilibrium is formed between activated and deactivated Pfl under anaerobic conditions. If the deactivase activity of the AdhE protein is partially or completely blocked in lactic acid bacteria, an increased Pfl activity is expected to occur while, on the other hand, a reduced Pfl activity is expected to occur if the deactivase activity is overexpressed. If the Pfl activase is blocked, a decreased Pfl activity is contemplated.

The acetaldehyde dehydrogenase and the ethanol dehydrogenase activities of the AdhE protein are also potential targets for metabolic engineering in lactic acid bacterial food starter cultures and cultures used in feed production or as cultures for the production of aroma compounds or antimicrobially active compounds. Thus, it can be contemplated that a block or modification of the ethanol dehydrogenase activity of such cultures may result in the overproduction of acetaldehyde which is an important flavour compound in yoghurt. Alternatively, a block of the acetaldehyde dehydrogenase activity could give rise to an increased production of acetate which in turn may result in improved preservation of fermented foods or feed products in whose production such modified cultures are used. Additionally, it is contemplated that such modifications of starter cultures would increase the pyruvate pool and consequently, the formation of diacetyl or other compounds derived from the conversion of α-acetolactate. Increasing one or both dehydrogenase activities will most likely direct the conversion of acetyl CoA from acetate to acetaldehyde or ethanol.

Based on the above analysis of the potential means of regulating the size of the pyruvate pool in lactic acid bacteria and the intracellular fluxes from this metabolic intermediate pool towards desirable end products, a novel approach has been developed for metabolically engineering lactic acid bacteria allowing the provision of useful lactic acid bacterial starter cultures either having an enhanced production of desirable flavour compounds or an increased production of antimicrobially active compounds which can be used to increase the shelf life of food or feed products.

In particular, the starting point for the invention is the achievement of the isolation and sequencing of the entire adhE and pfl genes of Lactococcus lactis. Based on these findings, it has become possible, by appropriate modifications of the genes and their expression and/or activity of one or more of the enzyme activities encoded by these genes, to provide in a goal-directed manner lactic acid bacterial starter cultures having the above desirable characteristics, including cultures of strains having reduced or enhanced production of particular metabolites.

### SUMMARY OF THE INVENTION

Accordingly, the present invention provides novel means for metabolically engineering lactic acid bacteria, and lactic acid bacteria being modified by such means. Specifically, the invention relates in a first aspect to an isolated DNA sequence comprising a sequence derived from a lactic acid bacterium, said sequence coding for a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

In further aspects, the invention pertains to a recombinant replicon comprising the above DNA sequence and to a recombinant lactic acid bacterial cell comprising such a replicon.

In still further aspects, there is provided an-isolated DNA sequence comprising a sequence derived from a lactic acid bacterium, said sequence coding for a polypeptide having pyruvate formate-lyase activity, subject to the limitation that the sequence is not derived from oral Streptococcus species, a recombinant replicon comprising such a DNA sequence and a recombinant lactic acid bacterial cell comprising such a replicon.

In another aspect, the invention relates to a method of producing a lactic acid bacterial metabolite, the method comprising cultivating a lactic acid bacterium comprising a DNA sequence as defined above which is modified so as to inactivate or reduce or enhance the expression of at least one of the enzymatic activities selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity, or a lactic acid bacterium comprising a DNA sequence which is modified whereby its production of pyruvate formate-lyase is reduced or inhibited, or whereby the enzyme is expressed in a modified form having a reduced pyruvate formate-lyase activity, or wherein the DNA sequence is modified whereby the expression of pyruvate formate-lyase is enhanced or whereby the enzyme is expressed in a modified form having an increased pyruvate formate-lyase activity, and isolating the metabolite from the culture.

The invention also pertains to methods of producing a food product or an animal feed, the method comprising the step of admixing to the food product or feed starting materials a starter culture of a lactic acid bacterium according to the invention and keeping the mixture under conditions allowing the starter culture to be metabolically active.

There is also provided an isolated DNA sequence derived from a lactic acid bacterium, said sequence coding for a product having a formate transporter activity.

### DETAILED DISCLOSURE OF THE INVENTION

The facultative anaerobe Escherichia coli is capable of carrying out mixed-acid fermentation during anaerobic growth in the absence of exogenous electron acceptors. In this connection, a major fermentation product is ethanol which is synthesized from acetyl CoA by two consecutive NADH-dependent reductions catalyzed by a single polypeptide, AdhE, with an acetaldehyde dehydrogenase (ACDH) domain and alcohol dehydrogenase (ADH) domain. It has also been found that this polypeptide is responsible for pyruvate formate-lyase deactivase activity.

It has now been found that a DNA sequence showing significant homology to the E. coli gene, adhE which codes for a polypeptide showing substantial similarity with the above multi-functional E. coli AdhE polypeptide is present in lactic acid bacteria which are also facultative anaerobes, such as in Lactococcus lactis. It was therefore hypothesized that the gene product of the thus identified and isolated lactic acid bacterial DNA sequence might have similar enzymatic activities as the corresponding E. coli gene. This was found to be the case.

Accordingly, the present invention provides, as mentioned above, in its first aspect an isolated DNA sequence which comprises a sequence derived from a lactic acid bacterium, which sequence codes for a multi-functional polypeptide having at least one of the following enzymatic activities: (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity. The coding sequence for the multifunctional polypeptide is also referred to herein as the adhE gene, and the polypeptide encoded by the gene as the AdhE polypeptide.

In accordance with the invention, the DNA sequence coding for the multi-functional polypeptide may be derived from any lactic acid bacterium. In the present context, the term “lactic acid bacterium” designates gram-positive, microaerophilic or facultatively anaerobic bacteria which ferment sugars with the production of acids including lactic acid as the predominantly produced acid, acetic acid and propionic acid. The industrially most useful lactic acid bacteria are found among Lactococcus species, Streptococcus species, Lactobacillus species, Leuconostoc species and Pediococcus species. Additionally, the strict anaerobic Bifidobacterium species, which are commonly used in the manufacture of dairy products, are included in the group of lactic acid bacteria. The group of lactic acid bacteria comprises so-called mesophilic species which have optimum growth temperatures in the range of 15-30° C. and which in many cases do not grow at temperatures exceeding 35-40° C. Other groups of lactic acid bacteria have higher growth temperatures, in particular species for which humans and/or animals are the natural habitat, e.g. Enterococcus species, oral streptococci and pathogenic streptococci.

In certain preferred embodiments, the above DNA sequence is derived from Lactococcus lactis including Lactococcus lactis subspecies lactis, Lactococcus lactis subspecies diacetylactis (also frequently referred to as Lactococcus lactis subspecies lactis biovar diacetylactis) and Lactococcus lactis subspecies cremoris.

In useful embodiments of the invention, the lactic acid bacterium-derived DNA sequence codes for a multifunctional polypeptide that is at least 30% identical with the gene products of the adhE gene of E. coli (FASTA, GCG Wisconsin accession No. P17547) or the aad gene of Clostridium acetobutylicum (FASTA, GCG Wisconsin accession No. P33744) or the gene product of the sequence of Table 1.4 herein (SEQ ID NO:3). In other useful embodiments, the identity to such other gene products is at least 40%, such as at least 50%, such as at least 60% identity or even at least 70% identity. The homology between the .above gene products may also be expressed in terms of amino acid similarity in which case the similarity suitably is at least 60%, such as at least 70%, e.g. at least 80% similarity. In this context, the expression “amino acid similarity” indicates that a particular amino acid in a polypeptide sequence can be replaced by another amino acid having similar physical/chemical characteristics such as charge or polarity characteristics.

The sequence according to the invention which codes for the AdhE protein also includes such a coding sequence of lactic acid bacterial origin which hybridizes to the adhE coding sequence from L. lactis strain DB1341 under the following conditions: hybridization overnight at 65° C. followed by washing the filters twice in 5×SSC at room temperature for 30 minutes and subsequently once in 3×SSC; 0.1% SDS at 65° C. for 30 minutes.

In one specific embodiment, the DNA sequence according to the invention comprises the sequence as shown herein in Table 1.4 (SEQ ID NO:3) or the sequence designated adhemg1363 as shown in the below Table 1.8 (SEQ ID NO:12) or the sequence shown in Table 1.9 (SEQ ID NOS:28/30), or a mutant or variant hereof which codes at least in part for a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

In the present context, the above term “mutant or variant” is used to designate any naturally occurring or constructed nucleotide modification of the above DNA sequence which still allows a polypeptide having at least one of the defined activities to be expressed by the thus modified sequence. Accordingly, the modification may consist in one or more nucleotide substitutions in one or more codons, resulting in the translation of the same or different amino acid(s), or the modification may be in the form of the insertion or deletion of one or more nucleotides/codons. The modifications can be provided by any conventional method including, where appropriate, modifications hereof, such as e.g. the use of restriction enzymes or random or site-directed mutagenesis, e.g. by means of transposable elements. It will be understood that the above DNA sequence according to the invention may also be provided-as a synthetically produced sequence or it may be a hybrid sequence comprising in part a native sequence and in part a synthetically prepared sequence. Additionally, the above term “mutant and variant” includes any mutein of the sequence.

The above lactic acid bacterial DNA sequence whether in its native form or in a modified mutant or variant form may further comprise one or more sequences that regulate the expression of the coding sequence. Such regulatory sequences may be located upstream and/or downstream of the coding sequence or they can be placed on a different replicon, i.e. in trans. The regulatory sequences may be sequences which are natively associated with the coding sequence or they may be inserted or modified promoter sequences not natively associated with the coding sequence, which can be operably linked to the coding sequence. Such sequences which are not natively associated with the coding sequence may be derived from the bacterial strain which is the source of the coding sequence or from a different organism. In this context, a regulatory sequence includes a promoter/operator sequence, a ribosome binding site, a sequence coding for a gene product which either enhances or inhibits the expression the coding sequence, such as a repressor or activator substance including e.g. a RNA sequence including an antisense RNA, a terminator sequence or a leader sequence regulating the excretion of the above multifunctional enzyme product. A promoter which is derived from a different organism or from the same organism may, depending on the desired characteristics of the resulting bacterial cell, have a stronger or a weaker promoter activity than the promoter with which the coding sequence is natively associated.

In a useful embodiment, the coding sequence is under the control of a regulatable promoter. As used herein, the term “regulatable promoter” is used to describe a promoter sequence, the activity of which is dependent on physical or chemical factors present in the medium where organisms comprising the above coding sequence and its regulatory sequences are cultivated. Such factors include the cultivation temperature, the pH and/or the arginine content of the medium, a temperature shift eliciting the expression of heat shock genes, the composition of the growth medium including the ionic strength/NaCl content and the growth phase/growth rate of the host cell and stringent response.

A promoter sequence as defined above may further comprise sequences whereby the activity of the promoter becomes regulated. Thus, in lactic acid bacterial cultures for which it is advantageous to have a gradually decreasing activity of the coding sequence under control of the promoter sequence such further sequences may provide a regulation by a stochastic event and may e.g. be sequences, the presence of which results in a recombinational excision of the promoter or of genes coding for substances which are positively needed for the promoter function.

It has been found that in e.g. Lactococcus lactis there may be, upstream of the sequence coding for the above multifunctional polypeptide, DNA sequences coding for one or more open reading frames. Thus, such open reading frames were identified in both L. lactis strain DB1341 and strain MG1363. These open reading frames were designated orfB.

In a further aspect, the invention relates, as it is mentioned above, to a recombinant replicon comprising the above DNA sequence coding for the multifunctional polypeptide. As used herein, the term “replicon” designates a DNA sequence which is capable of autonomous replication in a lactic acid bacterium. Such a replicon can be selected from a plasmid capable of replicating in a lactic acid bacterium, a lactic acid bacterial chromosome and a bacteriophage derived from a lactic acid bacterium.

The replicon may comprise further sequences including marker sequences and linker sequences for the insertion of genes coding for desirable gene products. Thus, in useful embodiments, the replicon may comprise a gene coding for a lipase, a peptidase, a gene coding for a gene product involved in carbohydrate or citrate metabolism, a gene coding for a gene product involved in bacteriophage resistance or a gene coding for a lytic enzyme or a gene coding for a bacteriocin such as e.g. nisin or pediocin. The gene may also be one which codes for a gene product conferring resistance to an antibiotic.

The gene coding for a desired gene product may be a homologous gene, i.e. a gene isolated from the same species as the host cell for the replicon, or a heterologous gene including a gene isolated from a lactic acid bacterial species which is of a species different from the host cell.

The invention also provides a recombinant lactic acid bacterial cell comprising the above replicon. Such a host cell may be derived from any species of lactic acid bacteria as defined herein, such as a Lactococcus species, a Lactobacillus species, a Streptococcus species, a Pediococcus species, a Bifidobacterium species and a Leuconostoc species.

The above lactic acid bacterial cell is useful in starter culture compositions for the manufacturing of food products including dairy products, meat products, wine, vegetables and bakery products, or in the preservation of animal feed. In the latter context, the present recombinant lactic acid bacterial cells are particularly useful as inoculants in field crops which are to be ensiled or as preserving agents in feedstuff components of animal origin such as waste products from the slaughtering and fish processing industries.

When the cells are to be used for these purposes they are conveniently provided in the form of freeze-dried or frozen concentrates typically containing 10⁸ to 10¹² colony forming units (CFUs) per g of concentrate. Such concentrates may be provided as starter culture compositions comprising further suitable components such as e.g. preserving agents, stabilizing agent, cryoprotectants, nutrients, bacterial growth factors or further active components including enzymes.

An interesting use of the above lactic acid bacterial cell is in the manufacturing of a probiotically active composition. In the present context, the term “probiotically active” indicates that the bacteria selected for this purpose have characteristics which enable them to colonize in the gastrointestinal tract and hereby exert a beneficial regulatory effect on the microbial flora in this habitat. Such an effect may be recognizable as an improved food or feed conversion in humans or animals to which the cells are administered, or as an increased resistance against invading pathogenic microorganisms.

The above lactic acid bacterial cell can also be provided in the form of a culture for the production of an aroma or antimicrobially active compound.

In a particularly useful embodiment, the above lactic acid bacterial cell is one wherein the DNA sequence comprising the sequence coding for the multifunctional polypeptide is modified so as to inactivate or reduce the production of or the activity of at least one of the enzymatic activities selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

Such a modification can be made by methods which are known per se in the art. Thus, as typical examples, a DNA modification can be in the form of deletion, insertion or substitution of one or more nucleotides in the coding sequence possibly leading to the translation of a polypeptide having a modified amino acid composition. Such a modified polypeptide may have lost one or more of the above enzymatic activities or it/they may be reduced. An inactivation of the coding sequence may also be obtained by random or site-directed mutagenesis, e.g. using a transposable element which is integratable in the replicon comprising the coding sequence. Another useful means of providing inactivated mutants is Campbell-like homologous integration as it is described in the below examples.

The level of production of the multi-functional polypeptide can also be reduced by modifying or regulating regulatory sequences controlling the expression of the gene coding for the polypeptide. Thus, as one example, a native constitutive promoter can be replaced by a regulatable promoter, the function of which can be reduced or inhibited under appropriate conditions such as those physical and chemical promoter regulating factors as mentioned above. Alternatively, a native promoter which is in itself regulatable by certain factors may be replaced by another regulatable promoter which is negatively regulatable by other factors present in the cultivation medium for the recombinant cell.

Generally, the term “metabolic engineering” in relation to lactic acid bacteria covers manipulations of the bacteria themselves or of the conditions under which they are cultivated whereby the production of metabolites from the fermentation of sugars or citrate is modulated quantitatively or qualitatively. Accordingly, a lactic acid bacterial cell which is modified as described above in one or more of its glycolytic pathways can be characterized as a metabolically engineered cell. Dependent on the type and the site of the DNA modification such a cell will be at least partially blocked in one or more of the above pathways catalyzed by the multi-functional polypeptide (R6/R7 in FIG. 1) and/or the pyruvate formate-lyase deactivase activity will be reduced or blocked. Accordingly, such a metabolically engineered cell may as a result of these modifications produce increased amounts of i.a. acetaldehyde, ethanol and/or acetate.

In a further useful embodiment, the above lactic acid bacterial cell is one wherein the DNA sequence comprising the sequence coding for the multi-functional polypeptide is modified so as to enhance the production of or the activity of at least one of its native enzymatic activities as defined above. It is contemplated that such a modification can be provided by appropriate modifications of the coding sequence itself which result in an enhanced production level of the polypeptide and/or the production of a modified polypeptide having an enhanced activity of at least one of its native activities. Such modification can be made by substitution, deletion or insertion of one or more nucleotides using any conventional methods for such DNA modifications, including random or site-directed mutagenesis followed by selection of the desired mutants.

Alternatively, a lactic acid bacterial cell having enhanced production of and/or enhanced activity of at least one of its native enzymatic activities can be provided by suitable modifications of sequences regulating the production and/or the activity of the multifunctional polypeptide. One suitable manner whereby this can be obtained is by operably linking the coding sequence to a promoter sequence having a stronger promoter activity than the native promoter for the coding sequence.

In suitable embodiments such an inserted promoter is regulatable by a factor as mentioned above and the expression of the polypeptide can then be enhanced by cultivating the cell in the presence of a factor which mediates a strong promoter activity. It is contemplated that an enhanced production of the AdhE polypeptide in a host cell can be obtained by using a replicon which occurs in a high copy number in that host cell.

It is aimed at that such a metabolically engineered lactic acid bacterial cell having enhanced production of and/or enhanced activity of at least one of its native enzymatic activities will result in that the cell produces increased amounts of at least one metabolite selected from the group consisting of acetaldehyde, ethanol, formate, acetate, acetoin, diacetyl and 2,3 butylene glycol. Thus, in preferred embodiments, such metabolically engineered have a production of one or more of these metabolites which, in comparison with a wild type strain, is at least 2-fold higher such as at last 5-fold higher, e.g. at least 10-fold higher or even at least 20-fold higher.

The present invention relates in a still further aspect to an isolated lactic acid bacterial DNA sequence that comprises a sequence coding for a polypeptide having pyruvate formate-lyase activity, i.e. a pfl gene. In useful embodiments, such a DNA sequence further comprises at least one regulatory sequence operably linked to the coding sequence and regulating the production of the pyruvate formate-lyase polypeptide or coding for a gene product regulating the pyruvate formate-lyase activity of the polypeptide. In the following, the gene product of pfl will also be referred to as a Pfl polypeptide.

Such regulatory sequences may be located upstream and/or downstream of the coding sequence. The regulatory sequences may be sequences which are natively associated with the coding sequence or they may be inserted or modified promoter sequences not natively associated with the coding sequence, but which can be operably linked to the coding sequence. Such sequences which are not natively associated with the coding sequence may be derived from the bacterial strain which is the source of the coding sequence or from a different organism. In this context, regulatory sequences include a promoter sequence, a ribosome binding site, a sequence coding for a gene product which either enhances or inhibits the expression of the coding sequence, such as a repressor or activator substance including e.g an antisense RNA, a transcription terminator sequence or a leader sequence directing the excretion of the Pfl polypeptide. In a useful embodiment, the coding sequence is under the control of a regulatable promoter as defined hereinbefore and being regulatable as also described above.

The activity of the pyruvate formate-lyase enzyme can be regulated or modulated under anaerobic conditions by the presence or absence of an activase and a deactivase, respectively. Accordingly, the DNA sequence comprising the sequence coding for the Pfl polypeptide preferably comprises sequences coding for a pyruvate formate-lyase activase (act gene) and/or a pyruvate formate-lyase deactivase. In preferred embodiments, such a deactivase is a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity as defined hereinbefore.

In accordance with the invention, the Pfl-encoding DNA sequence can be derived from any lactic acid bacterium including a Lactobacillus species, a Streptococcus species, a Pediococcus species a Bifidobacterium species, a Leuconostoc species and a Lactococcus species such as Lactococcus lactis including Lactococcus lactis subspecies lactis, Lactococcus lactis subspecies lactis biovar diacetylactis and Lactococcus lactis subspecies cremoris.

It has been found that the Pfl polypeptide as encoded by the pfl gene of Lactococcus lactis subspecies lactis biovar diacetylactis strain DB1341 comprises 787 amino acids (Table 3.2 below) (SEQ ID NO:15) and has a deduced molecular weight of 89.1 kDa. This polypeptide shows considerable identity with known pfl gene products (Table 3.1). Furthermore, it has been found that the corresponding pfl gene in Lactococcus lactis subspecies lactis MG1363 differs from the DB1341 gene in only about 50 of the nucleotides.

In specific embodiments, the DNA sequence comprising a Pfl encoding sequence comprises the coding sequence as shown in Table 3.2 below (SEQ ID NO:15), the sequence designated mg1363-pfl as shown in Table 3.6 (SEQ ID NO:22) and the sequence shown in Table 5.3 (SEQ ID NOS:36 and 38), or a DNA sequence which is a mutant or variant hereof which codes for a polypeptide having pyruvate formate-lyase activity, the term “mutant or variant” being used in the same manner as defined hereinbefore.

In accordance with the invention, a pfl gene as defined herein encompasses any of the specific sequences as exemplified in the following and a lactic acid bacterial sequence coding for a polypeptide having the enzymatic activity of the gene products of such isolated sequences which has a DNA homology of at least 50% with the coding sequence of the plf of L. lactis strains DB1341 or MG1363 such as at least 60% homology including at least 70% homology or at least 80% homology, e.g. at least 90% homology.

In useful embodiments of the invention, the lactic acid bacterium-derived DNA sequence codes for a Pfl protein that is at least 30% identical with the gene products of the pfl gene of Streptococcus mutans (FASTA, GCG Wisconsin, Accession No. D50491) or the pfl gene of Hemophilus influenzae (FASTA, GCG Wisconsin, Accession Nos. U32812 and L42023) or the gene product of the sequence of Table 3.2 herein (SEQ ID NO:15). In other useful embodiments, the identity to such gene products is at least 40%, such as at least 50%, such as at least 60% identity or even at least 70% identity. The homology between the above gene products may also be expressed in terms of amino acid similarity in which case the similarity suitably is at least 60%, such as at least 70%, e.g. at least 80% similarity.

In accordance with the invention, the DNA sequence coding for the Pfl polypeptide may also be a coding sequence of lactic acid bacterial origin that hybridizes to the pfl encoding sequence isolated from L. lactis strain MG1363, under the following conditions: hybridization overnight at 65° C. followed by washing the filter twice in 5×SSC at room temperature for 30 minutes and subsequently once in 3×SSC; 0.1% SDS at 65° C. for 30 minutes.

It was found that e.g. in L. lactis open reading frames may be identified upstream of the coding region for the Pfl polypeptide. Such open reading frames were designated orfA and it was found that the gene products hereof has a function in transport across cell membranes of formate. Thus, it was found that a mutant strain of L. lactis wherein the open reading had been disrupted showed an increased tolerance to the toxic formate analogue, hypophosphite.

In accordance with the invention there is also provided herein a recombinant replicon comprising the above Pfl-encoding DNA sequence. Such a replicon can be derived from a plasmid, a lactic acid bacterial bacteriophage or a lactic acid bacterial chromosome.

In one aspect the invention relates to a recombinant lactic acid bacterial host cell comprising such a replicon. The cell can be selected from the group consisting of a Lactococcus species, a Lactobacillus species, a Streptococcus species, a Pediococcus species a Bifidobacterium species and a Leuconostoc species.

The lactic acid bacterial cell may conveniently be provided in the form of a starter culture composition for use in the manufacturing of food products as described above. It is also contemplated that the above cells may be used as probiotically active cultures or as inoculants in animal feed preservation. In this connection, a particular use is as inoculants in field crops or animal waste materials which are subjected to an ensiling process.

In particularly useful embodiments, the above lactic acid bacterial cell is one wherein the DNA sequence coding for pyruvate formate-lyase activity is modified whereby the production of the pyruvate formate-lyase is reduced or eliminated or whereby the enzyme is produced in a modified form having a reduced pyruvate formate-lyase activity.

Such a modification can, as it has been described above for a cell comprising a sequence coding for the AdhE polypeptide, be made by methods which are known per se in the art. Thus, as typical examples, a DNA modification can e.g. be made by deletion, insertion or substitution of one or more nucleotides in the coding sequence possibly leading to the expression of a polypeptide having a modified amino acid composition. An inactivation of the coding sequence can also be obtained by random or site-directed mutagenesis, e.g. by using a transposable element which is integratable in the replicon comprising the coding sequence. Another possible means of providing Pfl-inactivated (pfl⁻)mutants is Campbell-like homologous integration.

The level of expression of the Pfl polypeptide can also be reduced by modifying or regulating regulatory sequences controlling the production of the polypeptide. Thus, as one example, a native constitutive promoter can be replaced by a regulatable promoter, the function of which can be reduced or inhibited under appropriate conditions such as those physical and chemical promoter regulating factors as mentioned hereinbefore. Alternatively, a native promoter which is in itself regulatable by certain factors may be replaced by another regulatable promoter which is negatively regulatable by other factors present in the cultivation medium for the recombinant cell.

A cell being modified in this manner will be a metabolically engineered cell, since under conditions where the pyruvate formate-lyase is normally metabolically active as shown in FIG. 1 such a modified cell will lack one of the major pathways whereby the pyruvate pool in normally consumed. This will result in a modification of the metabolic pathways based on pyruvate including an enhanced flux towards α-acetolactate which is a precursor substance for diacetyl, acetoin and 2,3 butylene glycol. Such a cell is particularly useful in dairy starter cultures where such flavour compounds are generally desirable.

In further useful embodiments, the lactic acid bacterial cell according to the invention is a cell wherein the DNA sequence comprising the sequence coding for pyruvate formate-lyase is modified so that the production of the pyruvate formate-lyase is enhanced or so that the enzyme is produced in a modified form having an increased pyruvate formate-lyase activity.

Analogously with what is described above with respect to the modifications leading to an enhanced expression or activity of the AdhE polypeptide, it is contemplated that such a modification can be provided by appropriate modifications of the coding sequence itself which result in an enhanced production level of the Pfl polypeptide and/or the production of a modified polypeptide having an enhanced activity of at least one of its native activities. Such modifications can be made by substitution, deletion or insertion of one or more nucleotides using any conventional methods for such DNA modifications, including random or site-directed mutagenesis followed by selection of the desired mutants.

Alternatively, a lactic acid bacterial cell having enhanced production of and/or enhanced activity of pyruvate formate-lyase can be provided by suitable modifications of sequences regulating the expression of the pfl gene and/or the activity of the enzyme. One suitable manner whereby this can be obtained is by operably linking the coding sequence to a promoter sequence having a stronger promoter activity than the native promoter for the coding sequence. In suitable embodiments such an inserted promoter is regulatable by a factor as mentioned above and the production of the polypeptide can then be enhanced by cultivating the cell in the presence of a factor which confers a strong promoter activity. It is contemplated that a thus modified lactic acid bacterial cell produces increased amounts of formate and/or acetate. Enhanced production of the Pfl polypeptide may also be obtained in a host by using a replicon which occurs in a high copy number in that host cell or by chromosomal amplification.

In accordance with the invention, there is also provided a recombinant lactic acid bacterial cell comprising both the DNA sequence comprising the above sequence coding for an AdhE polypeptide, and the above sequence comprising a sequence coding for pyruvate formate-lyase, in both instances including sequences regulating the production and/or the activity of the enzyme activities. As used herein, the term “recombinant” implies that at least one of the coding sequences or regulatory sequences is not a naturally occurring sequence. The sequences may be located on the same replicon or they may be on separate replicons.

Preferably, at least one of the sequences of the above cell is modified so as to modify the production of the pyruvate formate-lyase or the activity hereof, or the distribution of the amounts of end products resulting from the lactose and/or citrate metabolism of the cell.

It will be understood that a lactic acid bacterium which is metabolically engineered in accordance with the invention so that it has an enhanced production of one or more metabolites is useful in a method of producing such a metabolite or such metabolites. In general, such a the method comprises cultivating a lactic acid bacterium which is metabolically engineered in accordance with the invention under conditions where the metabolite is produced, and isolating the metabolite from the culture. The isolation of the metabolite may be carried out according to any conventional methods of recovering the particular substance, such as e.g. distillation.

As it is also mentioned above, the lactic acid bacterial cells according to the invention are useful as food starter cultures. In accordance herewith, the invention also provides a method of producing a food product, the method comprising the step of admixing to the food product starting materials a starter culture of a lactic acid bacterium as defined above and keeping the mixture under conditions allowing the starter culture to be metabolically active. Such a method where a starter culture which is metabolically engineered in accordance with the invention is used will, dependent on the type of metabolite modifications, result in a food product having an improved flavour and/or a product which has an improved shelf life due to an enhanced production of antimicrobially active metabolites by the starter culture.

The invention will now be further illustrated in the below examples and the drawing wherein:

FIG. 1 illustrates selected metabolic pathways in citrate fermenting lactic acid bacteria;

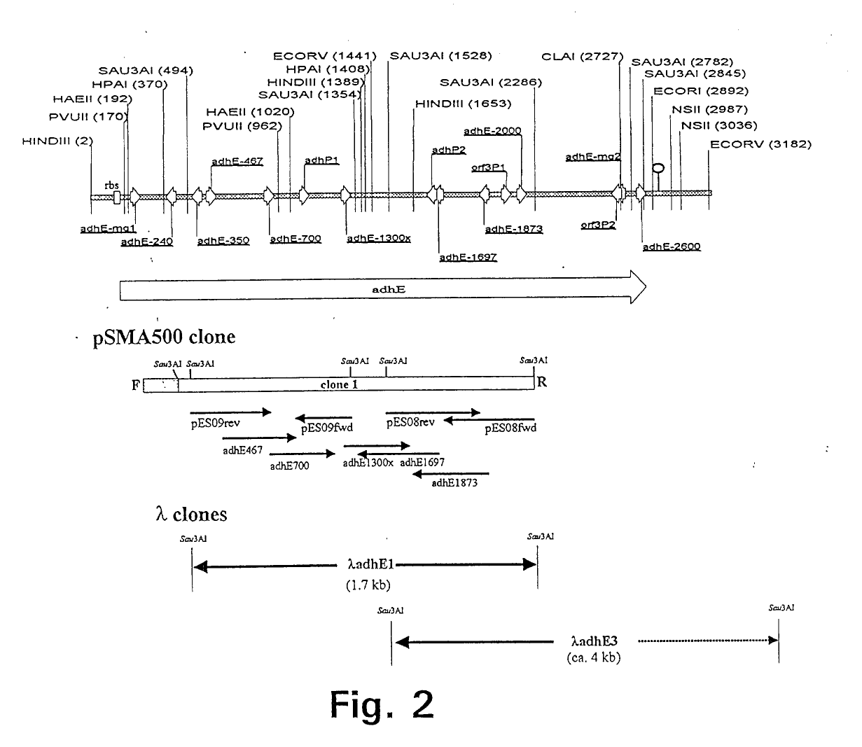

FIG. 2 shows an overview of the cloned L. lactis DB1341 adhE gene (open arrow), the sequence strategy for clone 1 (box in middle) and the regions covered by the λZAP clones adhE1 and adhE3 (bottom). The nucleotide position of relevant restriction sites is shown (top). The position of PCR and sequencing primers is shown as small open arrows. A putative transcription terminator present downstream of the stop codon is shown as a circle. The rbs box shows the position of a consensus lactococcal ribosome binding site. Arrows show the sequencing strategy for clone 1 (middle);

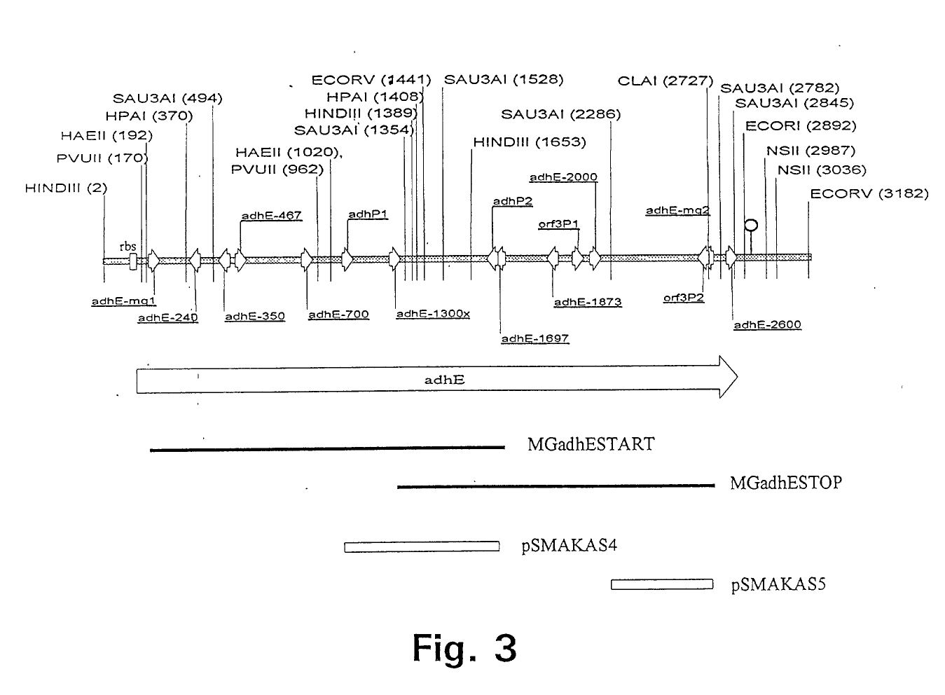

FIG. 3 shows an overview of the cloned L. lactis DB1341 adhE gene fragment (open arrow). The nucleotide position of relevant restriction sites is shown (top). The position of PCR and sequencing primers is shown as small open arrows. A putative transcription terminator present downstream of the stop codon is shown as a circle. The rbs box shows the position of a consensus lactococcal ribosome binding site. The cloned PCR fragments of the L. lactis MG1363 adhE gene are shown as lines (MGadhESTART and MGadhESTOP). The PCR fragments used to clone into pSMA500 for gene inactivation in strain DB1341 are shown as open boxes (pSMAKAS4 and pSMAKAS5);

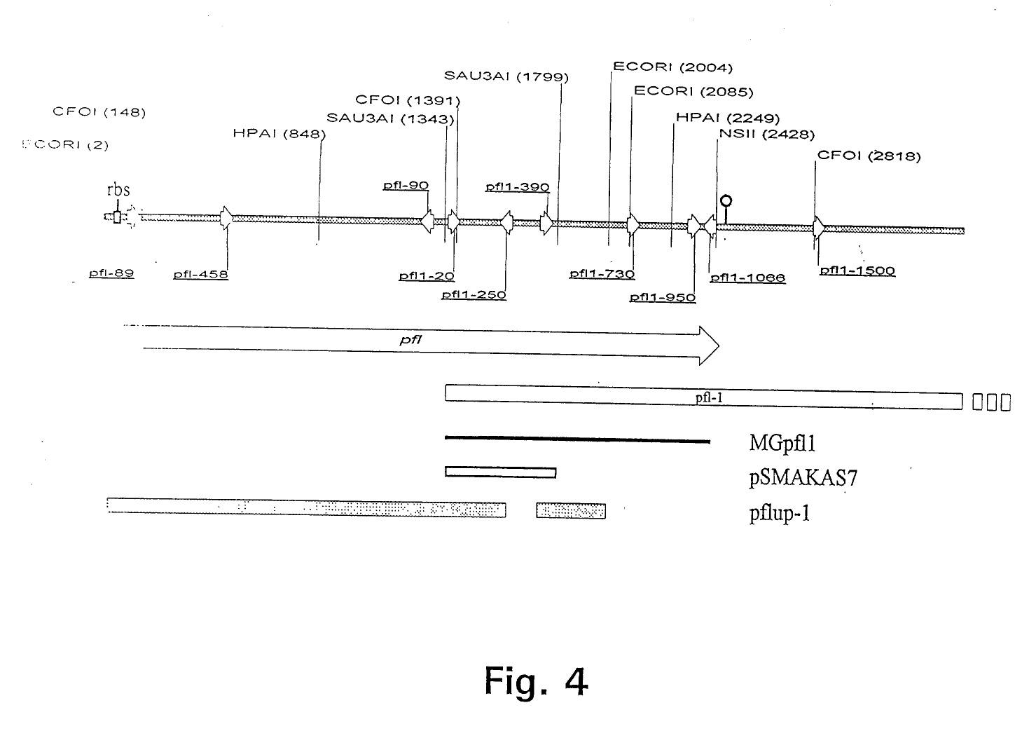

FIG. 4 is an overview of the cloned Lactococcus lactis DB1341 strain (L. lactis subspecies lactis biovar diacetylactis) pfl gene (open arrow box). The nucleotide positions of relevant restriction sites are shown (top). The position of PCR and sequencing primers is shown as small open arrows. A putative ribosome binding site (rbs box) and a transcription terminator present downstream of the stop codon is shown as a circle. The plf1 (open box) shows the fragment of the λZAP clone of the DB1341 genomic library containing a pfl gene fragment. The cloned PCR fragment of the L. lactis subspecies lactis MG1363 pfl fragment is shown as a line (MGpfl1). A Sau3AI fragment used for gene inactivation in strain DB1341 is shown as an open box (pSMAKAS7). The pfl region included in the fragment as obtained by inverse PCR from DB1341 using EcoRI digestion and primers pfl1-250 and pfl1-390 is shown as a dotted box (pflup-1);

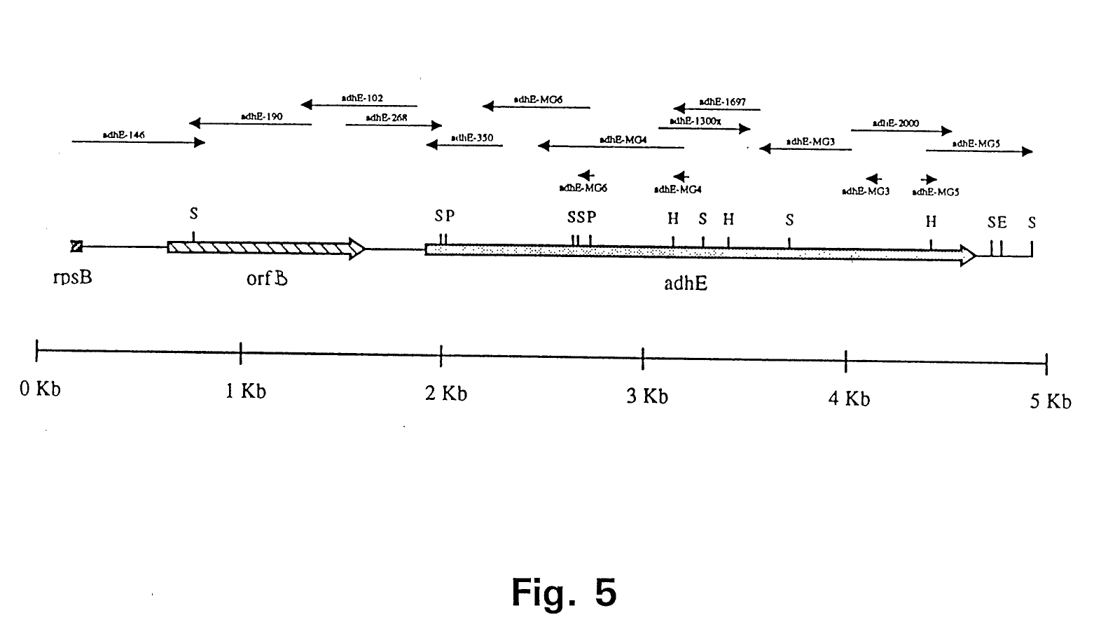

FIG. 5 is a genetic map of the L. lactis MG1363 adhE locus including the orfB open reading frame. In the upper part are indicated primer sequences;

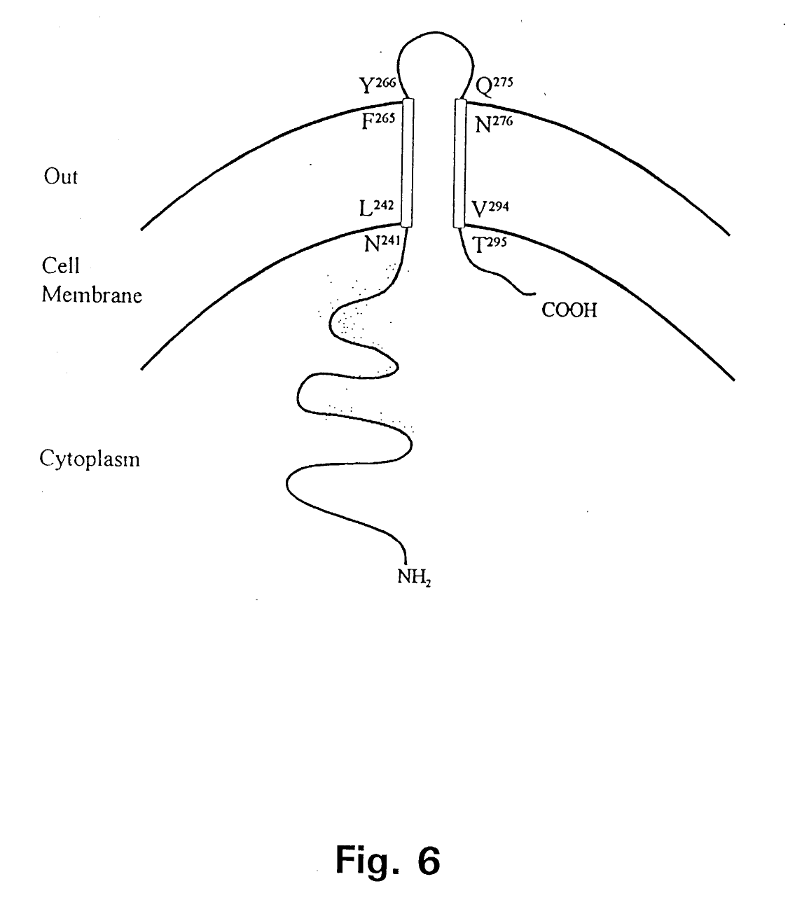

FIG. 6 illustrates the structure of the L. lactis OrfA protein. The shadowed box at the terminal region of OrfA depicts the area covered by the internal orfA fragment used for gene inactivation. The two transmembrane regions were identified using the PredictProtein server at the EMBL, Heidelberg, Germany;

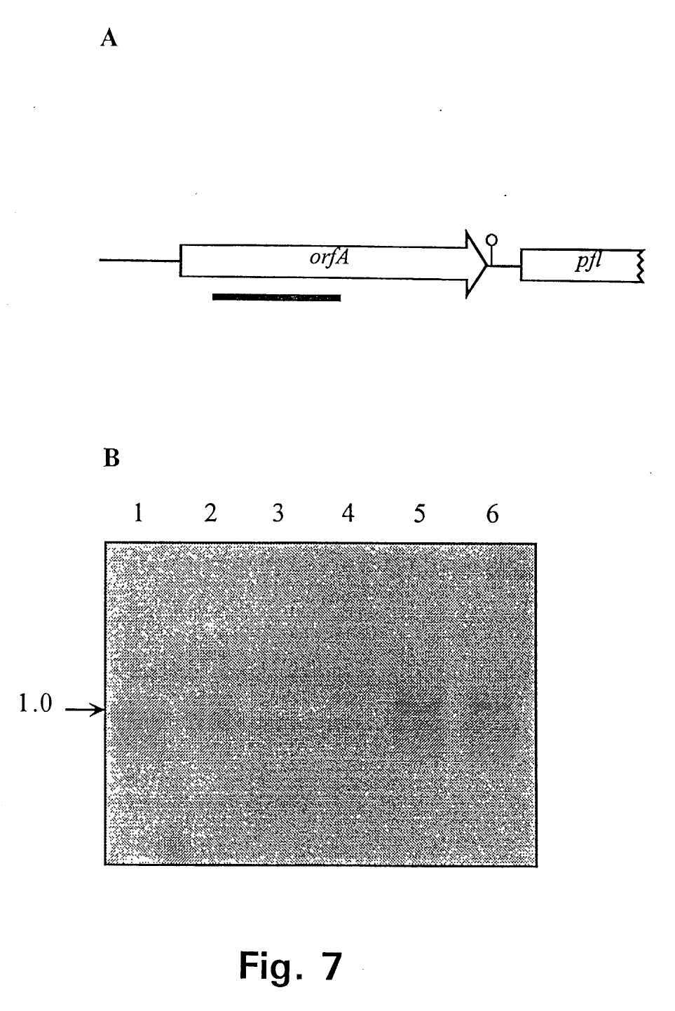

FIG. 7 illustrates expression of orfA in L. lactis. A: genetic map of orfA showing the region covered by the probe (thick line below orfA) used in expression studies and in the construction of a null mutant strain. B: Northern blot analysis. RNA isolated from MG1363 was hybridized to the orfA probe. Lane 1: exponential culture in GM17 aerobic; lane 2: same, anaerobic; lane 3: stationary culture in GM17, aerobic; lane 4: same, anaerobic; lane 5: exponential culture i GalM17, aerobic; lane 6: same, anaerobic. The transcript size is shown in kb to the left. The autoradiogram was exposed for 14 days;

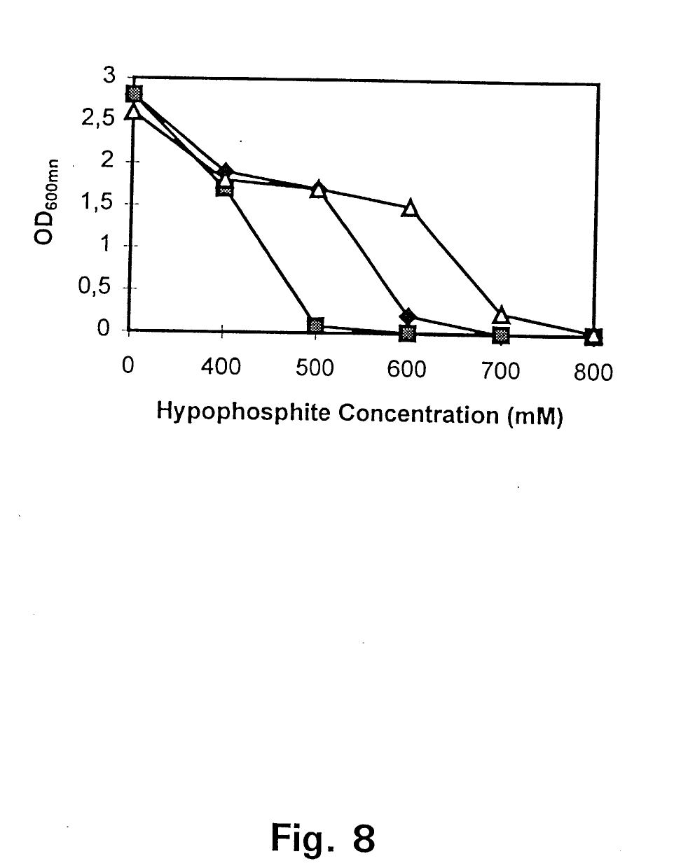

FIG. 8 illustrates inhibition of growth by hypophosphite in strains of L. lactis. Strains were grown anaerobically overnight in GM17 supplemented with different concentrations of hypophosphite. At the end of the incubation period (about 18 hours), OD₆₀₀ was measured. Symbols: (♦) MG1363; (▴) MG1363Δo-rfA; (▪) MG1363 pAK80::orfA;

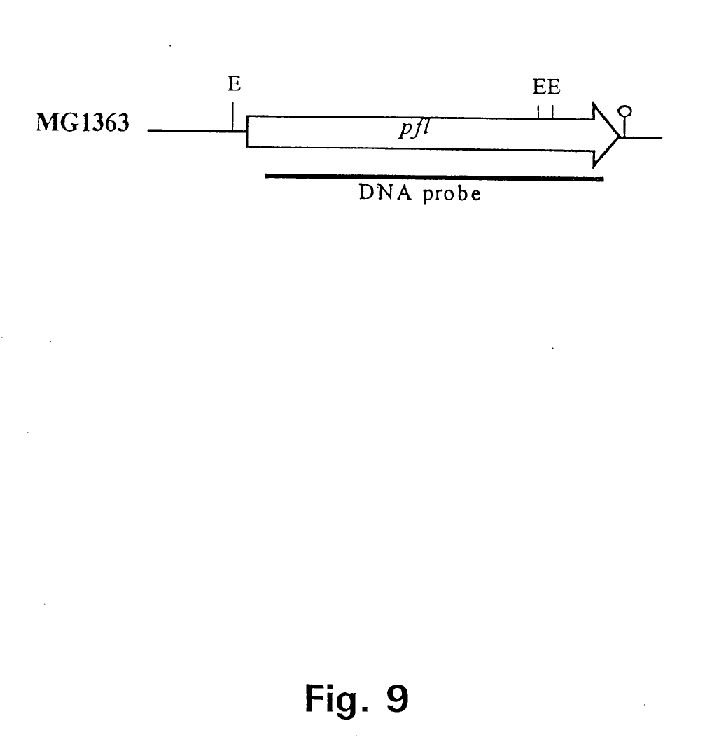

FIG. 9 shows a genetic map of the L. lactis MG1363 pfl gene, showing the region used as a probe in the identification of pfl homologues in other lactic acid bacteria, including the position of EcoR1 sites;

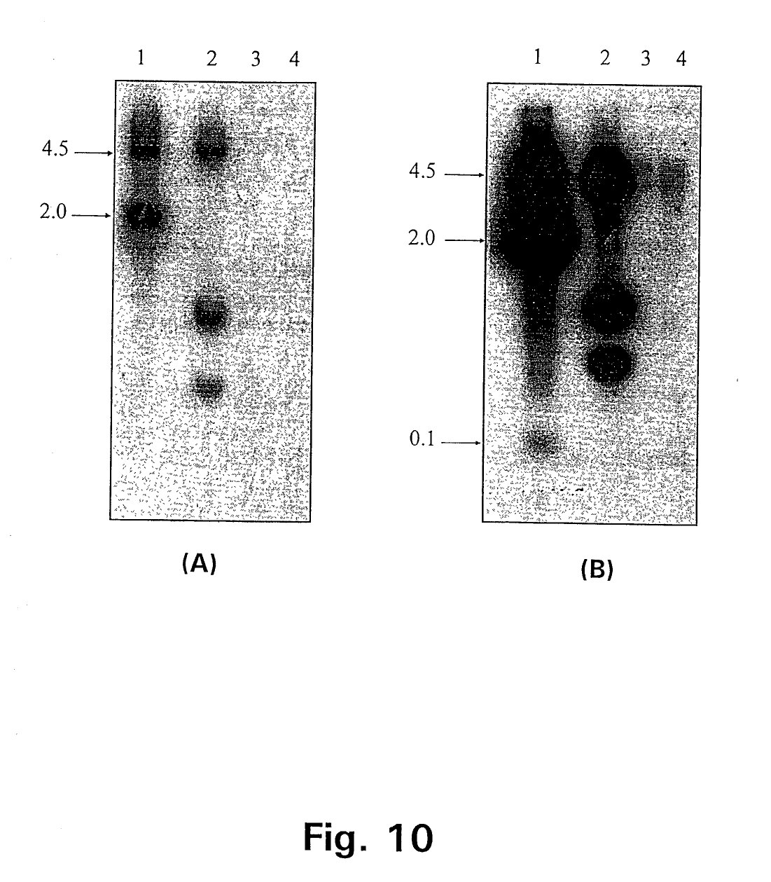

FIG. 10 shows autoradiograms from Southern hybridization of genomic DNA from non-Lactococcus lactic acid bacteria to a L. lactis pfl probe; Lane 1: L. lactis MG1363; lane 2: Streptococcus thermophilus; lane 3: Leuconostoc mesenteroides; lane 4 Lactobacillus acidophilus. Bands are shown in kb. Filters were exposed 2 h (A) or overnight (B);

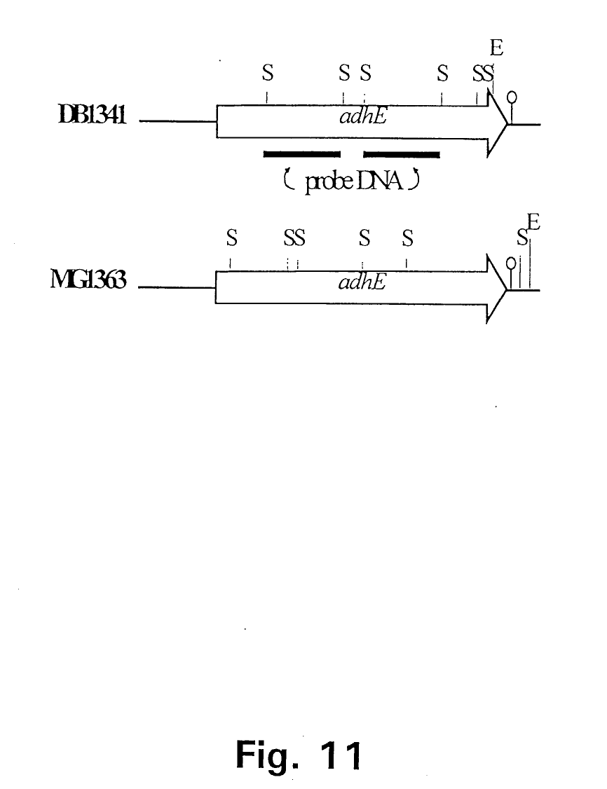

FIG. 11 illustrates two Sau3AI fragments including most of the L. lactis strain DB1341 adhE coding sequence used in Southern hybridization experiments with EcoRI-digested genomic DNA from non-Lactococcus lactic acid bacteria;

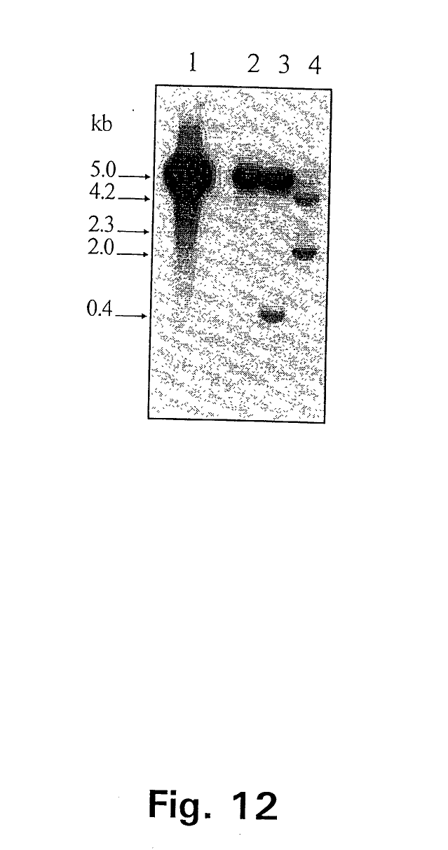

FIG. 12 illustrates detection of adhE homologues in other lactic acid bacteria by Southern hybridization experiments with EcoRI-digested genomic DNA from non-Lactococcus lactic acid bacteria. Lane 1: L. lactis MG1363; lane 2: S. thermophilus; lane 3: L. mesenteroides; lane 4 L. acidophilus. Bands are shown in kb. Filters were exposed overnight;

### EXAMPLE 1

### Cloning of the L. lactis adhE Gene

1. Construction of a L. lactis ssp. lactis Biovar diacetylactis DB1341 Genomic Library for Genetic Complementation

A genomic library was constructed by cloning partially Sau3AI-digested chromosomal DNA from strain DB1341 into BamHI-digested pSMA500 (Madsen et al. 1996) and transforming into E. coli MC1000 by electroporation (Sambrook et al., 1989). Strain DB1341 was kindly provided by Chr. Hansen A/S, Hørsholm, Denmark. The genomic library consisted of about 10,000 independent recombinant clones with an average insert size of 4 kb. A mixed culture, containing all clones obtained, was grown in LB+erythromycin term, 50 μg/ml) and plasmid DNA was isolated for genetic complementation.

2. Genetic Complementation in E. coli NZN111 Using the pSMA500 Library

E. coli strain NZN111 (pfl⁻; ldb::Tn5; kan^{R}) is unable to grow in the absence of O₂ due to the accumulation of NADH derived from the lack of fermentative enzyme activities encoded by the pfl and ldh genes (Mat-Jan et al., 1989).

Genetic complementation was attempted by transformation of NZN111 using 200 ng plasmid DNA from the library (see above). Transformation mixtures were plated on LB+erm (50 μg/ml)+Kanamycin (kan; 50 μg/ml) and incubated at 37° C. in anaerobic jars. As a control, pSMA500-transformed strain NZN111 was used. After two days, transformation plates were incubated aerobically for another two days to allow weak complementing clones to grow. A clone was identified (clone 1) in the library-transformed plates, and no growth was observed in the pSMA500 control.

In a preliminary screening, protein extracts of clone 1 were used in a modified “Ldh” assay (Crow and Pritchard 1977), where the pyruvate-dependent conversion of NADH to NAD is monitored, to ensure that complementation of the fermentative defects in strain NZN111 had occurred. Protein extraction was carried out adding 100 μl 100 mM MOPS buffer (pH 6.5); 2% Triton X-100 to the cell pellet from 1.5 ml stationary cultures grown in LB+erm (50 μg/ml) which had been washed in fresh ice cold LB, and frozen at −80° C. for 15 min. Pellets were dissolved and transferred to Eppendorf tubes. Lysozyme (5 mg) was added and samples were incubated on ice for 30 min. Subsequently, glass beads (100 μM, Sigma; 100 μl) were added and samples were vortexed for 30 sec and kept on ice for 30 sec. This step was repeated 10-15 times, and samples were centrifuged at maximum speed for 2 min. Supernatants were transferred to a new Eppendorf tube and kept at −80° C. until assayed. To measure NADH oxidation, the following components were mixed in a quartz cuvette: 700 μl 100 mM MOPS, pH 6.5; 100 μl 120 mM Na-Pyruvate; 50 μl 2.56 mM NADH and 50 μl H₂O. The decrease in OD₃₄₀ as a result of the oxidation of NADH to NAD was monitored after the addition of 100 μl sample. As control reaction, pyruvate was omitted. No significant decrease in OD was observed in the control. A relatively high conversion rate (approximately 2-fold as compared to the NZN111::pSMA500 control) was observed in clone 1.

Plasmid DNA was isolated from clone 1 and used to retransform E. coli NZN111. Duplicate LB+erm plates were incubated (i) aerobically for 4 days or (ii) anaerobically for 2 days and then 2 days aerobically at 37° C. A similar number of transformants was obtained in both procedures (see Table 1.1 below) Thus, clone 1 did not result from artifact cloning and can indeed complement the defect in strain NZN111.

TABLE 1.1
Retransformation of clone 1 into E. coil NZN111

No. of colonies per 10 ng DNA

| Plasmid | anaerobic growth | aerobic growth |
|---|---|---|
| clone 1 | 600 | 800 |
| pSMA500 | 0 | 1000 |

NZN111 competent cells were electroporated with the corresponding plasmid, and one half of the cell mixture was plated onto LB+kan+erm and incubated without O₂ (anaerobic growth), and the other half was plated onto the same medium and incubated with O₂ (aerobic growth). Transformants were scored after 4 days (see main text).

A sample of clone 1 in E. coli was deposited under the Budapest Treaty with the German Collection of Microorganisms and Cell Cultures, Mascheroder Weg 1b, D-38 124 Braunschweig, Germany on 18 Jul. 1996 under the accession No. DSM 11093.

3. Sequence Analysis of Clone 1 and Identification of an adhE Fragment

Clone 1 was further characterized by restriction enzyme analysis and included a 2.2 kb insert. Sequence analysis determined that it included a 1.7 kb fragment of an open reading frame (ORF) showing homology to the E. coli adhE gene disclosed by Goodlove et al., 1989. The sequence of the 2.2 kb insert is shown in Table 1.2 below (SEQ ID NO:1).

TABLE 1.2
Sequence of the insert in clone 1

| | | | |
|---|---|---|---|
| | Sau3AI | | |
| 1 | GATCTGTCCTTAGTACGAGAGGACCGGGATGGACTTACCGCTGGTGTACC | | (SEQ ID NO:1) |
| 51 | AGTTGTTCCGCCAGAGCACGGCTGGATAGCTATGTAGGGAAGGGATAAGC | | |
| 101 | GCTGAAAGCATCTAAGTGCGAAGCCACCTCAAGATGAGATTACCCATTCG | | |
| | Sau3AI | | |
| 151 | AGAATTAAGAGCCCAGAGAGATGATCAAGATGTCAATAATTTGCAAAAAA | | |
| 201 | TCTTCTTTCAGCAAAACGGGATTTGAGTTTTTGCTCGATTTGTGGGAATT | | |
| | Sau3AI | | |
| 251 | TAACAGAAAGTGATCTGTTGAAATCGCAAGCCCTCTCGGTGTACTTGCTG | | |
| 301 | GTATCGTTCCAACGACTAATCCAACATCAACAGCAATCTTTAAATCTTTA | | |
| 351 | TTGACTGCAAAAACACGTAATGCTATTGTTTTCGCTTTCCACCCTCAAGC | | |
| 401 | TCAAAAATGTTCAAGCCATGCAGCAAAAATTGTTTACGATGCTGCAATTG | | |
| 451 | AAGCTGGTGCACCGGAAGACTTTATTCAATGGATTGAAGTACCAAGCCTT | | |
| 501 | GACATGACTACCGCCTTGATTCAAAACCGTGGACTTGCAACAATCCTTGC | | |
| 551 | AACTGGTGGCCCAGGAATGGTAAACGCCGCACTCAAATCTGGTAACCCTT | | |
| 601 | CACTCGGTGTTGGAGCTGGTAATGGTGCTGTTTATGTTGATGCAACTGCA | | |
| 651 | AATATTGAACGTGCCGTTGAAGACCTTTTGCTTTCAAAACGTTTTGATAA | | |
| | −35 | | |
| 701 | TGGGATGATTTGTGCCACTGAAAATTCAGCTGTTATTGATGCTTCAGTTT | | |
| | −10 SD → | | |
| 751 | ATGATGAATTTATTGCTAAAATGCAAGAACAAGGCGCTTATATGGTTCCT | | |
| | \overline{M }V P | 3 | (SEQ ID NO:2) |
| 801 | AAAAAAGACTACAAAGCTATTGAAAGTTTCGTTTTTGTTGAACGTGCTGG | | |
| | K K D Y K A I E S F V F V E R A G | 20 | |
| 851 | TGAAGGTTTTGGAGTAACTGGTCCTGTTGCCGGTCGTTCTGGTCAATGGA | | |
| | E G F G V T G P V A G R S G Q W I | 37 | |
| 901 | TTGCTGAACAAGCTGGTGTCAAAGTTCCTAAAGATAAAGATGTCCTTCTT | | |
| | A E Q A G V K V P K D K D V L L | 53 | |
| 951 | TTTGAACTTGATAAGAAAAATATTGGTGAAGCACTTTCTTCTGAAAAACT | | |
| | F E L D K K N I G E A L S S E K L | 70 | |
| 1001 | TTCTCCTTTGCTTTCAATCTACAAAGCTGAAACACGTGAAGAAGGAATTG | | |
| | S P L L S I Y K A E T R E E G I E | 87 | |
| 1051 | AGATTGTACGTAGCTTACTTGCTTATCAAGGTGCTGGACATAATGCTGCA | | |
| | I V R S L L A Y Q G A G H N A A | 103 | |
| | Sau3AI | | |
| 1101 | ATTCAAATCGGTGCAATGGATGATCCATTCGTTAAAGAATATGGCGAAAA | | |
| | I Q I G A M D D P F V K E Y G E K | 120 | |
| 1151 | AGTTGAAGCTTCTCGTATCCTCGTTAACCAACCAGATTCTATTGGTGGGG | | |
| | V E A S R I L V N Q P D S I G G V | 137 | |
| 1201 | TCGGAGATATCTATACTGATGCAATGCGTCCATCACTTACACTTGGAACT | | |
| | G D I Y T D A M R P S L T L G T | 153 | |
| | Sau3AI | | |
| 1251 | GGTTCATGGGGGAAAAATTCACTTTCACACAATTTGAGTACATACGATCT | | |
| | G S W G K N S L S H N L S T Y D L | 170 | |
| 1301 | ATTGAATGTTAAAACAGTGGCTAAACGTCGTAATCGCCCACAATGGGTTC | | |
| | L N V K T V A K R R N R P Q W V R | 187 | |
| 1351 | GTTTGCCAAAAGAAATTTACTACGAAAAAAATGCAATTTCTTACTTACAA | | |
| | L P K E I Y Y E K N A I S Y L Q | 203 | |
| 1401 | GAATTGCCACACGTCCACAAAGCTTTCATCGTTGCTGACCCTGGTATGGT | | |
| | E L P H V H K A F I V A D P G M V | 220 | |
| 1451 | TAAATTTGGTTTCGTTGATAAAGTTTTGGAACAACTTGCTATCCGCCCAA | | |
| | K F G F V D K V L E Q L A I R P T | 237 | |
| 1501 CTCAAGTTGAAACAAGCATTTATGGCTCTGTTCAACCTGACCCAACTTTG | | | |
| | Q V E T S I Y G S V Q P D P T L | 253 | |
| 1551 | AGCGAAGCAATTGCAATCGCTCGTCAAATGAAACAATTTGAACCTGACAC | | |
| | S E A I A I A R Q M K Q F E P D T | 270 | |
| 1601 | TGTCATCTGTCTTGGTGGTGGTTCTGCTCTCGATGCCGGTAAGATTGGTC | | |
| | V I C L G G G S A L D A G K I G R | 287 | |
| 1651 | GTTTGATTTATGAATATGATGCTCGTGGTGAAGCTGACCTTTCTGATGAT | | |
| | L I Y E Y D A R G E A D L S D D | 303 | |
| 1701 | GCAAGTTTGAAAGAACTTTTCCAAGAATTAGCTCAAAAATTTGTCGATAT | | |
| | A S L K E L F Q E L A Q K F V D I | 320 | |
| 1751 | TCGTAAACGTATTATTAAATTCTACCATCCACATAAAGCACAAATGGTTG | | |
| | R K R I I K F Y F Y H P H K A Q M V A | 337 | |
| 1801 | CAATTCCTACTACTTCTGGTACTGGTTCTGAAGTGACTCCATTTGCAGTT | | |
| | I P T T S G T G S E V T P F A V | 353 | |
| 1851 | ATCACTGATGATGAAACTCATGTTAAGTACCCACTTGCTGACTACCAATT | | |
| | I T D D E T H V K Y P L A D Y Q L | 370 | |
| 1901 | AACACCACAAGTTGCCATTGTTGACCCTGAGTTTGTTATGACTGTACCAA | | |
| | T P Q V A I V D P E F V M T V P K | 387 | |
| 1951 | AACGTACTGTTTCTTGGTCTGGTATTGATGCGATGTCACACGCGCTTGAA | | |
| | R T V S W S G I D A M S H A L E | 403 | |
| 2001 | TCTTACGTTTCTGTTATGTCTTCTGACTATACAAAACCAATTTCACTTCA | | |
| | S Y V S V M S S D Y T K P I S L Q | 420 | |
| | Sau3AI | | |
| 2051 | AGCGATCCCGGGTCTAGATTAGGGTAACTTTGAAAGGA | | |
| | A I P G L D * | | |

Sau3AI recognition sites are indicated above the sequence. DNA homology to the E. coli adhE starts at nucleotide position 262 (data not shown). A Sau3AI fragment with 100% homology to the 23S rRNA of L. lactis is shown doubly underlined at the top (positions 1-173). Putative expression signals functional in E. coli are shown: −35, −10 promoter regions (underlined); Shine Dalgarno (SD, doubly underlined) and putative start codon (bold, discontinuous underline). The amino acid sequence of the open reading frame is given in one-letter-code. The open reading frame ends in the multiple cloning site of vector pSMA500 (doubly underlined at bottom) (Madsen et al., 1996).

E. coli AdhE is a multi-functional protein consisting of 890 amino acids that catalyzes the conversion of acetyl CoA into ethanol and has acetaldehyde-DHase (ACDH) and alcohol-DHase (ADH) activities. Additionally, AdhE shows Pfl deactivase activity involved in the inactivation of pyruvate-formate lyase, a key enzyme in anaerobic metabolism (Knappe et al. 1991).

As shown in the above Table 1.2 and Table 1.3 below, clone 1 includes the ADH domain of a L. lactis AdhE homologue, and it contains expression signals necessary for expression in E. coli (Shine Dalgarno and −35 and −10 regions). The putative gene product of 427 amino acids is highly homologous to a number of other iron-dependent ADHs. Comparison at the protein level showed a 41.4% identity (78% similarity) with E. coli AdhE, in addition to significant homology to other ADHs of both eukaryotic and prokaryotic origin (Table 1.3).

TABLE 1.3

Homology search (FASTA, GCG Wisconsin package version 8,
Genetics Computer Group) using the 427 amino acid putative
protein encoded by clone 1 (see also TABLE 1.2)
The region of homology to AdhE corresponds to the central
region, where the ADH domain is possibly located. Only homology
to the best score is shown.
(Peptide) FASTA of: clone1.pep from: 1 to: 427
TRANSLATE of: clone1.seq check: 2521 from: 792 to: 2072

| The best scores are: | init1 | initn | opt.. |
|---|---|---|---|
| sw:adhe_ecoli P17547 escherichia coli. alcohol dehydroge. | 276 | 736 | 768 |
| sw:adhe_cloab P33744 clostridium acetobutylicum. alcoh.. | 256 | 600 | 703 |
| sw:adh1_cloab P13604 clostridium acetobutylicum. nadph.. | 256 | 357 | 279 |
| sw:medh_bacmt P31005 bacillus methanolicus. nad-depend.. | 169 | 224 | 173 |
| sw:adh4_yeast P10127 saccharomyces cerevisiae (baker's.. | 146 | 224 | 165 |
| sw:adhf_schpo Q09669 schizosaccharomyces pombe (fission. | 146 | 219 | 162 |
| sw:yiay_ecoli P37686 escherichia coli. hypothetical 40.. | 158 | 218 | 187 |
| sw:sucd_clok1 P38947 clostriclium kluyveri. succinate-s.. | 132 | 186 | 179 |
| sw:adh2_zymmo P06758 zymomonas mobilis. alcohol dehydr.. | 129 | 180 | 169 |
| sw:fuco_ecoli P11549 escherichia coli. lactaldehyde re.. | 141 | 175 | 147 |
| sw:adha_cloab Q04944 clostridium acetobutylicum. nadh-.. | 136 | 153 | 145 |

clone1.pep
sw:adhe_ecoli
ID ADHE_ECOLI STANDARD; PRT; 890 AA.
AC P17547;
DE ALCOHOL DEHYDROGENASE (EC 1.1.1.1) (ADH)/ACETALDEHYDE DEHYDROGENASE . . .
SCORES Init1: 276 Initn: 736 Opt: 768
41.4% identity in 430 aa overlap

| | | |
|---|---|---|
| | 10 20 30 | |
| clone 1 | MVPKKDYKAIESFVFVERAGEGFGVTGPVA | (SEQ ID NO:2) |
| | ::: \|: \|\|::: :: ::\| ::::::: | |
| adhe_e | GVICASEQSVVVVDSVYDAVRERFATHGGYLLQGKELKAVQDVIL--KNG---ALNAAIV | |

(corresponding to a.a. residues 43-762 of SEQ ID NO:6)

| | |
|---|---|
| | 250 260 270 280 290 |
| | 40 50 60 70 80 90 |
| clone 1 | GRSGQWIAEQAGVKVPKDKDVLLFELDKKNIGEALSSEKLSPLLSIYKAETREEGIEIVR |
| | \|::: \|\|\| \|\| :\|\|:::::\|: \|:: : :\|::: \|\|\|\|\| \|::\|:\|:: \|:::\| : |
| adhe_e | GQPAYKIAELAGFSVPENTKILIGEVTVVDESEPFAHEKLSPTLAMYRAKDFEDAVEKAE |
| | 300 310 320 330 340 350 |
| | 100 110 120 130 140 149 |
| clone1 | SLLAYQGAGHNAAIQIGAMDDP-FVKEYGEKVEASRILVNQPDSIGGVGDIYTDAMRPSL |
| | :\|:\| \| \|\|:: : ::: ::\| \|: :\|:\|::::\|\|\|:\| \|:\| \|\|:\|\|:\|: : \|\|\| |
| adhe_e | KLVAMGGIGHTSCLYTDQDNQPARVSYFGQKMKTARILINTPASQGGIGDLYNFKLAPSL |
| | 360 370 380 390 400 410 |
| | 150 160 170 180 190 200 |
| clone1 | TLGTGSWGKNSLSHNLSTYDLLNVKTVAKRRNRPQWVRLPKEIYYEKNAISY-LQE-LPH |
| | \|\|\| \|\|\|\| \|\|:\|:\|::: :\|:\| \|\|\|\|\|\| :: \| :\|\|\|:\|\|: ::::: \|:\| ::: |
| adhe_e | TLGCGSWGGNSISENVGPKHLINKKTVAKRAENMLWHKLPKSIYFRRGSLPIALDEVITD |
| | 420 430 440 450 460 470 |
| | 210 220 230 240 250 260 |
| clone1 | VHK-AFIVADPGMVKFGFVDKVLEQLAIRPTQVETSIYGSVQPDPTLSEAIAIARQMKQF |
| | \|\| \|:\|\|:\|: : : \|::\|:: : \| ::: \|\|\|::: :\|::\|\|\|\|\| : \| : \| |
| adhe_e | GHKRALIVTDRFLFNNGYADQITSVL--KAAGVETEVFFEVEADPTLSIVRKGAELANSF |
| | 480 490 500 510 520 530 |
| | 270 280 290 300 310 320 |
| clone1 | EPDTVICLGGGSALDAGKIGRLIYEYDARGEADLSDDASLKELFQELAQKFVDIRKRIIK |
| | :\|\|::\| \|\|\|\|\|::\|\|:\|\| :::\|\| : \|:: \|:\|\|\| :\|:\|\|\|\|\|\| \| |
| adhe_e | KPDVIIALGGGSPMDAAKIMWVMYE---HPETH----------FEELALRFMDIRKRIYK |
| | 540 550 560 570 580 |
| | 330 340 350 360 370 380 |
| clone1 | FYH-PHKAQMVAIPTTSGTGSEVTPFAVITDDETHVKYPLADYQLTPQVAIVDPEFVMTV |
| | \| : \|\|:\|:\|::\|\|\|\|\|\|\|\|\|\|\|\|\|\|:\|\|\|:\| \|\|\|\|\|\|\|:\|\|\|::\|\|\|\|:::\|\|:: |
| adhe_e | FPKMGVKAKMIAVTTTSGTGSEVTPFAVVTDDATGQKYPLADYALTPDMAIVDANLVMDM |
| | 590 600 610 620 630 640 |
| | 390 400 410 420 |
| clone1 | PKRTVSWSGIDAMSHALESYVSVMSSDYTKPISLQAIPGLD |
| | \|\|: :::\|:\|\|::\|\|:\|:\|\|\|\|::\|:::: :\|\|\|: \|: |
| adhe_e | PKSLCAFGGLDAVTHAMEAYVSVLASEFSDGQALQALKLLKEYLPASYHEGSKNPVARER |
| | 650 660 670 680 690 700 |
| adhe_e | VHSAATIAGIAFANAFLGVCHSMAHKLGSQFHIPHGLANALLICNVIRYNANDNPTKQTA |
| | 710 720 730 740 750 760 |

4. DNA Hybridization of the DB1341 λZAP Library Using an adhE Fragment

Sequence comparison of clone 1 with the previously cloned adhE gene indicated that the first 500 bp and the last 600 bp of the putative L. lactis adhE homologue were not present in clone 1. Therefore, a λZAP genomic library of strain DB1341 was constructed according to manufacturer's instructions (Stratagene). The average insert size was estimated to be approx. 3 kb, with 80% recombinant clones. Approximately 2×10⁵ pfu were screened using a 0.8 kb Sau3AI fragment (position 1296-2054 in Table 1.2) and 10 positive clones (named adhE-1 to 10 were selected for characterization.

5. Sequencing of Positive λZAP adhE Clones

Following ‘in vivo’ excision of the pBK plasmid version (Stratagene) of the clones, restriction mapping and sequencing of clones adhE-1 and adhE-3 was carried out as shown in FIG. 2. Clone adhE-1 included a 1.7 kb insert that was identical to the adhE fragment of clone 1 (position 262-2054 in Table 1.2). Clone adhE-3 contained a 4 kb insert spanning from the Sau3AI site at position 1296 in Table 1.2. This fragment could harbour the 3′-end of the L. lactis adhE gene. Sequence analysis of this clone confirmed that it included the 3′-end of the L. lactis adhE gene, which ends with a double stop codon (TAATAA, position 2854-2859 in Table 1.4 below). Downstream from this position, a possible transcription terminator was found (position 2883-2905 in Table 1.4).

A sample of clones adhE-1 and adhE-3, respectively in E. coli was deposited under the Budapest Treaty with the German Collection of Microorganisms and Cell Cultures, Mascheroder Weg 1b, D-38 124 Braunschweig, Germany on 25 Jul. 1996 under the accession Nos DSM 11101 and DSM 11102, respectively.

TABLE 1.4
Sequence of the L. lactis DB1341 adhE gene (SEQ ID NO:3)

| | | | |
|---|---|---|---|
| 1 | AAGCTTGTTACAAAACCGTTTTCTAAACTTTTGATGAGTGTTTTTGTAAA | | (SEQ ID NO:3) |
| 1 | ---------+---------+---------+---------+---------+ | 50 | |
| | AACTATCACAATATTGCTTGACATCTATAAAAAACTTTGTTAAACTATTC | | |
| 51 | ---------+---------+---------+---------+---------+ | 100 | |
| | ACGTAAAAGAAAGTGAATGAAGTCACAAAGGAGAACCTACAAATATGGCA | | |
| 101 | ---------+---------+---------+---------+---------+ | 150 | |
| | MetAla | — | (SEQ ID NO:4) |
| | ACTAAAAAAGCCGCTCCAGCTGCAAAGAAAGTTTTAAGCGCTGAAGAAAA | | |
| 151 | ---------+---------+---------+---------+---------+ | 200 | |
| | ThrLysLysAlaAlaProAlaAlaLysLysValLeuSerAlaGluGluLys | — | |
| | AGCCGCAAAATTCCAAGAAGCTGTTGCTTATACTGACAAATTAGTCAAAA | | |
| 201 | ---------+---------+---------+---------+---------+ | 250 | |
| | AlaAlaLysPheGlnGluAlaValAlaTyrThrAspLysLeuValLysLys | — | |
| | AAGCACAAGCTGCTGTTCTTAAATTTGAAGGATATACACAAACTCAAGTC | | |
| 251 | ---------+---------+---------+---------+---------+ | 300 | |
| | AlaGlnAlaAlaValLeuLysPheGluGlyTyrThrGlnThrGlnVal | — | |
| | GATACTATTGTCGCTGCAATGGCTCTTGCAGCAAGCAAACATTCTCTAGA | | |
| 301 | ---------+---------+---------+---------+---------+ | 350 | |
| | AspThrIleValAlaAlaMetAlaLeuAlaAlaSerLysHisSerLeuGlu | — | |
| | ACTCGCTCATGAAGCCGTTAACGAAACTGGTCGTGGTGTTGTCGAAGACA | | |
| 351 | ---------+---------+---------+---------+---------+ | 400 | |
| | LeuAlaHisGluAlaValAsnGluThrGlyArgGlyValValGluAspLys | — | |
| | AATGACAAAACTGTTGGTGTCATTTCTGAAAACAAGGTTGCTGGATCTGT | | |
| 401 | ---------+---------+---------+---------+---------+ | 450 | |
| | AspThrLysAsnHisPheAlaSerGluSerValTyrAsnAlaIleLys | — | |
| | AATGACAAAACTGTTGGTGTCATTTCTGAAAACAAGGTTGCTGGATCTGT | | |
| 451 | ---------+---------+---------+---------+---------+ | 500 | |
| | AsnAspLysThrValGlyValIleSerGluAsnLysValAlaGlySerVal | — | |
| | TGAAATCGCAAGCCCTCTCGGTGTACTTGCTGGTATCGTTCCAACGACTA | | |
| 501 | ---------+---------+---------+---------+---------+ | 550 | |
| | GluIleAlaSerProLeuGlyValLeuAlaGlyIleValProThrThrAsn | — | |
| | ATCCAACATCAACAGCAATCTTTAAATCTTTATTGACTGCAAAAACACGT | | |
| 551 | ---------+---------+---------+---------+---------+ | 600 | |
| | ProThrSerThrAlaIlePheLysSerLeuLeuThrAlaLysThrArg | — | |
| | AATGCTATTGTTTTCGCTTTCCACCCTCAAGCTCAAAAATGTTCAAGCCA | | |
| 601 | ---------+---------+---------+---------+---------+ | 650 | |
| | AsnAlaIleValPheAlaPheHisProGlnAlaGlnLysCysSerSerHis | — | |
| | TGCAGCAAAAATTGTTTACGATGCTGCAATTGAAGCTGGTGCACCGGAAG | | |
| 651 | ---------+---------+---------+---------+---------+ | 700 | |
| | AlaAlaLysIleValTyrAspAlaAlaIleGluAlaGlyAlaProGluAsp | — | |
| | ACTTTATTCAATGGATTGAAGTACCAAGCCTTGACATGACTACCGCCTTG | | |
| 701 | ---------+---------+---------+---------+---------+ | 750 | |
| | PheIleGlnTrpIleGluValProSerLeuAspMetThrThrAlaLeu | — | |
| | ATTCAAAACCGTGGACTTGCAACAATCCTTGCAACTGGTGGCCCAGGAAT | | |
| 751 | ---------+---------+---------+---------+---------+ | 800 | |
| | IleGlnAsnArgGlyLeuAlaThrIleLeuAlaThrGlyGlyProGlyMet — | | |
| | GGTAAACGCCGCACTCAAATCTGGTAACCCTTCACTCGGTGTTGGAGCTG | | |
| 801 | ---------+---------+---------+---------+---------+ | 850 | |
| | ValAsnAlaAlaLeuLysSerGlyAsnProSerLeuGlyValGlyAlaGly | — | |
| | GTAATGGTGCTGTTTATGTTGATGCAACTGCAAATATTGAACGTGCCGTT | | |
| 851 | ---------+---------+---------+---------+---------+ | 900 | |
| | AsnGlyAlaValTyrValAspAlaThrAlaAsnIleGluArgAlaVal | — | |
| | GAAGACCTTTTGCTTTCAAAACGTTTTGATAATGGGATGATTTGTGCCAC | | |
| 901 | ---------+---------+---------+---------+---------+ | 950 | |
| | GluAspLeuLeuLeuserLysArgPheAspAsnGlyMetIleCysAlaThr | — | |
| | TGAAAATTCAGCTGTTATTGATGCTTCAGTTTATGATGAATTTATTGCTA | | |
| 951 | ---------+---------+---------+---------+---------+ | 1000 | |
| | GluAsnSerAlaValIleAspAlaSerValTyrAspGluPheIleAlaLys | — | |
| | AAATGCAAGAACAAGGCGCTTATATGGTTCCTAAAAAAGACTACAAAGCT | | |
| 1001 | ---------+---------+---------+---------+---------+ | 1050 | |
| | MetGlnGluGlnGlyAlaTyrMetValProLysLysAspTyrLysAla | — | |
| | ATTGAAAGTTTCGTTTTTGTTGAACGTGCTGGTGAAGGTTTTGGAGTAAC | | |
| 1051 | ---------+---------+---------+---------+---------+ | 1100 | |
| | IleGluSerPheValPheValGluArgAlaGlyGluGlyPheGlyValThr | — | |
| | TGGTCCTGTTGCCGGTCGTTCTGGTCAATGGATTGCTGAACAAGCTGGTG | | |
| 1101 | ---------+---------+---------+---------+---------+ | 1150 | |
| | GlyProValAlaGlyArgSerGlyGlnTrpIleAlaGluGluAlaGlyVal | — | |
| | TCAAAGTTCCTAAAGATAAAGATGTCCTTCTTTTTGAACTTGATAAGAAA | | |
| 1151 | ---------+---------+---------+---------+---------+ | 1200 | |
| | LysValProLysAspLysAspValLeuLeuPheGluLeuAspLysLys | — | |
| | AATATTGGTGAAGCACTTTCTTCTGAAAAACTTTCTCCTTTGCTTTCAAT | | |
| 1201 | ---------+---------+---------+---------+---------+ | 1250 | |
| | AsnIleGlyGluAlaLeuSerSerGluLysLeuSerProLeuLeuSerIle | — | |
| | CTACAAAGCTGAAACACGTGAAGAAGGAATTGAGATTGTACGTAGCTTAC | | |
| 1251 | ---------+---------+---------+---------+---------+ | 1300 | |
| | TyrLysAlaGluThrArgGluGluGlyIleGluIleValArgSerLeuLeu | — | |
| | TTGCTTATCAAGGTGCTGGACATAATGCTGCAATTCAAATCGGTGCAATG | | |
| 1301 | ---------+---------+---------+---------+---------+ | 1350 | |
| | AlaTyrGlnGlyAlaGlyHisAsnAlaAlaIleGlnIleGlyAlaMet | — | |
| | GATGATCCATTCGTTAAAGAATATGGCGAAAAAGTTGAAGCTTCTCGTAT | | |
| 1351 | ---------+---------+---------+---------+---------+ | 1400 | |
| | AspAspProPheValLysGluTyrGlyGluLysValGluAlaSerArgIle | — | |
| | CCTCGTTAACCAACCAGATTCTATTGGTGGGGTCGGAGATATCTATACTG | | |
| 1401 | ---------+---------+---------+---------+---------+ | 1450 | |
| | LeuValAsnGlnProAspSerIleGlyGlyValGlyAspIleTyrThrAsp | — | |
| | ATGCAATGCGTCCATCACTTACACTTGGAACTGGTTCATGGGGGAAAAAT | | |
| 1451 | ---------+---------+---------+---------+---------+ | 1500 | |
| | AlaMetArgProSerLeuThrLeuGlyThrGlySerTrpGlyLysAsn | — | |
| | TCACTTTCACACAATTTGAGTACATACGATCTATTGAATGTTAAAACAGT | | |
| 1501 | ---------+---------+---------+---------+---------+ | 1550 | |
| | SerLeuSerHisAsnLeuSerThrTyrAspLeuLeuAsnValLysThrVal | — | |
| | GGCTAAACGTCGTAATCGCCCACAATGGGTTCGTTTGCCAAAAGAAATTT | | |
| 1551 | ---------+---------+---------+---------+---------+ | 1600 | |
| | AlaLysArgArgAsnArgProGlnTrpValArgLeuProLysGluIleTyr | — | |
| | ACTACGAAAAAAATGCAATTTCTTACTTACAAGAATTGCCACACGTCCAC | | |
| 1601 | ---------+---------+---------+---------+---------+ | 1650 | |
| | TyrGluLysAsnAlaIleSerTyrLeuGlnGluLeuProHisValHis | — | |
| | AAAGCTTTCATCGTTGCTGACCCTGGTATGGTTAAATTTGGTTTCGTTGA | | |
| 1651 | ---------+---------+---------+---------+---------+ | 1700 | |
| | LysAlaPheIleValAlaAspProGlyMetValLysPheGlyPheValAsp | — | |
| | TAAAGTTTTGGAACAACTTGCTATCCGCCCAACTCAAGTTGAAACAAGCA | | |
| 1701 | ---------+---------+---------+---------+---------+ | 1750 | |
| | LysValLeuGluGlnLeuAlaIleArgProThrGlnValGluThrSerIle | — | |
| | TTTATGGCTCTGTTCAACCTGACCCAACTTTGAGCGAAGCAATTGCAATC | | |
| 1751 | ---------+---------+---------+---------+---------+ | 1800 | |
| | TyrGlySerValGlnProAspProThrLeuSerGluAlaIleAlaIle | — | |
| | GCTCGTCAAATGAAACAATTTGAACCTGACACTGTCATCTGTCTTGGTGG | | |
| 1801 | ---------+---------+---------+---------+---------+ | 1850 | |
| | AlaArgGlnMetLysGlnPheGluProAspThrValIleCysLeuGlyGly | — | |
| | TGGTTCTGCTCTCGATGCCGGTAAGATTGGTCGTTTGATTTATGAATATG | | |
| 1851 | ---------+---------+---------+---------+---------+ | 1900 | |
| | GlySerAlaLeuAspAlaGlyLysIleGlyArgLeuIleTyrGluTyrAsp | — | |
| | ATGCTCGTGGTGAAGCTGACCTTTCTGATGATGCAGGTTTGAAAGAACTT | | |
| 1901 | ---------+---------+---------+---------+---------+ | 1950 | |
| | AlaArgGlyGluAlaAspLeuSerAspAspAlaSerLeuLysGluLeu | — | |
| | TTCCAAGAATTAGCTCAAAAATTTGTCGATATTCGTAAACGTATTATTAA | | |
| 1951 | ---------+---------+---------+---------+---------+ | 2000 | |
| | PheGlnGluLeuAlaGlnLysPheValAspIleArgLysArgIleIleLys | — | |
| | ATTCTACCATCCACATAAAGCACAAATGGTTGCAATTCCTACTACTTCTG | | |
| 2001 | ---------+---------+---------+---------+---------+ | 2050 | |
| | PheTyrHisProHisLysAlaGlnMetValAlaIleProThrThrSerGly | — | |
| | GTACTGGTTCTGAAGTGACTCCATTTGCAGTTATCACTGATGATGAAACT | | |
| 2051 | ---------+---------+---------+---------+---------+ | 2100 | |
| | ThrGlySerGluValThrProPheAlaValIleThrAspAspGluThr | — | |
| | CATGTTAAGTACCCACTTGCTGACTACCAATTAACACCACAAGTTGCCAT | | |
| 2101 | ---------+---------+---------+---------+---------+ | 2150 | |
| | HisValLysTyrProLeuAlaAspTyrGlnLeuThrProGlnValAlaIle | — | |
| | TGTTGACCCTGAGTTTGTTATGACTGTACCAAAACGTACTGTTTCTTGGT | | |
| 2151 | ---------+---------+---------+---------+---------+ | 2200 | |
| | ValAspProGluPheValMetThrValProLysArgThrValSerTrpSer | — | |
| | CTGGTATTGATGCGATGTCACACGCGCTTGAATCTTACGTTTCTGTTATG | | |
| 2201 | ---------+---------+---------+---------+---------+ | 2250 | |
| | GlyIleAspAlaMetSerHisAlaLeuGluSerTyrValSerValMet | — | |
| | TCTTCTGACTATACAAAACCAATTTCACTTCAAGCGATCAAACTTATCTT | | |
| 2251 | ---------+---------+---------+---------+---------+ | 2300 | |
| | SerSerAspTyrThrLysProIleSerLeuGlnAlaIleLysLeuIlePhe | — | |
| | TGAAAACTTGACTGAGTCTTATCATTATGACCCAGCGCATCCAACTAAAG | | |
| 2301 | ---------+---------+---------+---------+---------+ | 2350 | |
| | GluAsnLeuThrGluSerTyrHisTyrAspProAlaHisProThrLysGlu | — | |
| | AAGGACAAAAAGCCCGCGAAAACATGCACAATGCTGCAACACTCGCTGGT | | |
| 2351 | ---------+---------+---------+---------+---------+ | 2400 | |
| | GlyGlnLysAlaArgGluAsnMetHisAsnAlaAlaThrLeuAlaGly | — | |
| | ATGGCCTTCGCTAATGCTTTCCTTGGAATTAACCACTCACTTGCTCATAA | | |
| 2401 | ---------+---------+---------+---------+---------+ | 2450 | |
| | MetAlaPheAlaAsnAlaPheLeuGlyIleAsnHisSerLeuAlaHisLys | — | |
| | AATTGGTGGTGAATTTGGACTTCCTCATGGTCTTGCCATTGCCATCGCTA | | |
| 2451 | ---------+---------+---------+---------+---------+ | 2500 | |
| | IleGlyGlyGluPheGlyLeuProHisGlyLeuAlaIleAlaIleAlaMet | — | |
| | TGCCACATGTCATTAAATTTAACGCTGTAACAGGAAACGTTAAACGTACC | | |
| 2501 | ---------+---------+---------+---------+---------+ | 2550 | |
| | ProHisValIleLysPheAsnAlaValThrGlyAsnValLysArgThr | — | |
| | CCTTACCCACGTTATGAAACATATCGTGCTCAAGAGGACTACGCTGAAAT | | |
| 2551 | ---------+---------+---------+---------+---------+ | 2600 | |
| | ProTyrProArgTyrGluThrTyrArgAlaGlnGluAspTyrAlaGluIle | — | |
| | TTCACGCTTCATGGGATTTGCTGGTAAAGATGATTCAGATGAAAAAGCTG | | |
| 2601 | ---------+---------+---------+---------+---------+ | 2650 | |
| | SerArgPheMetGlyPheAlaGlyLysAspAspSerAspGluLysAlaVal | — | |
| | TGCAAGCTCTGGTTGCTGAACTTAAGAAACTGACTGATAGCATTGATATT | | |
| 2651 | ---------+---------+---------+---------+---------+ | 2700 | |
| | GlnAlaLeuValAlaGluLeuLysLysLeuThrAspSerIleAspIle | — | |
| | AATATCACCCTTTCAGGAAATGGTATCGATAAAGCTCACCTTGAACGTGA | | |
| 2701 | ---------+---------+---------+---------+---------+ | 2750 | |
| | AsnIleThrLeuSerGlyAsnGlyIleAspLysAlaHisLeuGluArgGlu | — | |
| | ACTTGATAAATTGGCTGACCTTGTTTATGATGATCAATGTACTCCTGCTA | | |
| 2751 | ---------+---------+---------+---------+---------+ | 2800 | |
| | LeuAspLysLeuAlaAspLeuValTyrAspAspGlnCysThrProAlaAsn | — | |
| | ATCCTCGTCAACCAAGAATTGATGAGATTAAACAGTTGTTGTTAGATCAA | | |
| 2801 | ---------+---------+---------+---------+---------+ | 2850 | |
| | ProArgGlnProArgIleAspGluIleLysGlnLeuLeuLeuAspGln | — | |
| | TACTAATAATCTGTTGATAAAATTATTAAAACGCTCTGATGAATTCGTCA | | |
| 2851 | ---------+---------+---------+---------+---------+ | 2900 | |
| | TyrEndEnd | | |
| | GAGCATTTTTTATTATAGCTTATACAACTATCAAAAGGTATAAATCAATT | | |
| 2901 | ---------+---------+---------+---------+---------+ | 2950 | |
| | TCGATATAGGCTCTTTTCACTCCATTGATTTATGCATTTCTATAAAAATC | | |
| 2951 | ---------+---------+---------+---------+---------+ | 3000 | |
| | AATAATTAATTAGCGATAGAAGTCGAGTTCATGCATGCTAATAATGAAAT | | |
| 3001 | ---------+---------+---------+---------+---------+ | 3050 | |
| | TGTTTTAAATTCTGGTTTTTCTTTATGTTCTTTGCGAACATCTTTCACAG | | |
| 3051 | ---------+---------+---------+---------+---------+ | 3100 | |
| | TTTCTTTGTTCATGAAAATTCCTCCTTATTATGGTACTATTTTGAGCCCA | | |
| 3101 | ---------+---------+---------+---------+---------+ | 3150 | |
| | AATAGTTATATAAGAATCCTAAACTTCGGATATCTTATCAAAG | | |
| 3151 | ---------+---------+---------+---------+--- 3193 | | |

In this Table a putative ribosome binding site is shown in bold (position 127-133), 12 bp upstream the putative start codon (position 145-147), deduced from homology comparisons (FIGS. 2 and 3).
Two adjacent stop codons, located at position 2854-2859) are shown (double underline).
A putative rho-independent transcription terminator (de Vos and Simons, 1994) is also shown downstream of the stop codons at position 2883-2904 (single and dotted underline show stem and loop sequences, respectively).

The L. lactis adhE gene of strain DB1341 encodes a 903 amino acid long protein, as deduced from the DNA sequence (Table 1.5), with an estimated molecular weight of 98.2 KDa. A putative ribosome binding site (AAAGGAG, position 127-133 in Table 1.4 is found 11 bp upstream of the start codon (de Vos and Simmons 1994).

Homology comparisons have shown a 44% identity (81% similarity) of the L. lactis AdhE to the E. coli protein and 42.4% identity (80% similarity) to the Clostridium acetobutylicum Aad protein throughout an approx. 750 amino acids fragment (Tables 1.4 and 1.5). A significantly lower homology is observed at the C-terminal region of these three proteins.

TABLE 1.5

Protein homology search (FASTA. GCG Wisconsin pack
age version 8. Genetics Computer Group) using the deduced
sequence of the AdhE Protein encoded by the L. lactis DB1341
adhE gene
In this Table only alignment of the best two scores (E. coli
AdhE and C. acetobutylicum Aad) is shown.
(Peptide) FASTA of: adhedb1341.pep from: 1 to: 904
TRANSLATE of: adhedb246.seq check: 3519 from: 145 to: 2856

| The best scores are: | init1 | initn | opt |
|---|---|---|---|
| sw:adhe_ecoli P17547 escherichia coli. alcohol dehydr.... | 708 | 1819 | 1507 |
| sw:adhe_cloab P33744 clostridium acetobutylicum. alcoh... | 404 | 1297 | 1053 |
| sw:adh1_cloab P13604 clostridiuxn acetobutylicum. nadph... | 283 | 581 | 434 |
| sw:sucd_clok1 P38947 clostridium kluyveri. succinate-s... | 290 | 460 | 621 |
| sw:medh_bacmt P31005 bacillus methanolicus. nad-depend... | 187 | 389 | 298 |
| sw:adh2_zymmo P06758 zymomonas mobilis. alcohol dehydr... | 170 | 376 | 299 |
| sw:adh4_yeast P10127 saccharomyces cerevisiae (baker's .. | 173 | 368 | 295 |
| sw:dhat_citfr P45513 citrobacter freundii. 1,3-Propan.... | 163 | 329 | 295 |
| sw:eute_salty P41793 salmonella typhimurium. ethanolam... | 150 | 309 | 372 |

adhedb1341.pep
sw:adhe_ecoli
ID ADHE_ECOLI STANDARD; PRT; 890 AA.
AC P17547;
DT 01 AUG. 1990 (REL. 15, CREATED)
DT 01 AUG. 1990 (REL. 15, LAST SEQUENCE UPDATE)
DT 01 NOV. 1995 (REL. 32, LAST ANNOTATION UPDATE)
DE ALCOHOL DEHYDROGENASE (EC 1.1.1.1) (ADH)/ACETALDEHYDE DEHYDROGENASE . . .
SCORES Init1: 708 Initn: 1819 Opt: 1507
44.3% identity in 757 aa overlap

| | |
|---|---|
| | 10 20 30 40 50 60 |
| adhe24 | MATKKAAPAAKKVLSAEEKAAKFQEAVAYTDKLVKKAQAAVLKFEGYTQTQVDTIVAAMA |
| | : \|\|:::: \| :::::\|\|:\|\|\|:\| \| \| |
| adhe_e | AVTNVAELNALVERVKKAQREYASFTQEQVDKIFRAAA |
| | 10 20 30 |
| | 70 80 90 100 110 120 |
| adhe24 | LAASKHSLELAHEAVNETGRGVVEDKDTKNHFASESVYNAIKNDKTVGVISENKVAGSVE |
| | \|\|\|:: :: \|\|: \|\|:\|:\|:\|:\|\|\|\| :\|\|\|\|\|\|\| :\|\|\| \|::\|\| \|\|:\|\|::: \|::: |
| adhe_e | LAAADARIPLAKMAVAESGMGIVEDKVIKNHFASEYIYNAYKDEKTCGVLSEDDTFGTIT |
| | 40 50 60 70 80 90 |
| | 130 140 150 160 170 180 |
| adhe24 | IASPLGVLAGIVPTTNPTSTAIFKSLLTAKTRNAIVFAFHPQAQKCSSHAAKIVYDAAIE |
| | \|\|:\|:\|:: \|\|\|\|\|\|\|\|\|\|\|\|\|\|\|\|\|:: \|\|\|\|\|\|:\|: \|\|:\|:: :::\|\|:\|\| :\|\|\| |
| adhe_e | IAEPIGIICGIVPTTNPTSTAIFKSLISLKTRNAIIFSPHPRAKDATNKAADIVLQAAIA |
| | 100 110 120 130 140 150 |
| | 190 200 210 220 230 240 |
| adhe24 | AGAPEDFIQWIEVPSLDMTTALIQNRGLATILATGGPGMVNAALKSGNPSLGVGAGNGAV |
| | \|\|\|\|:\|:\| \|\|: \|\|:::::\|\|::::::: \|\|\|\|\|\|\|\|\|\|:\|\| :\|\|:\|::\|\|\|\|\|\|::\| |
| adhe_e | AGAPKDLIGWIDQPSVELSNALMHHPDINLILATGGPGMVKAAYSSGKPAIGVGAGNTPV |
| | 160 170 180 190 200 210 |
| | 250 260 270 280 290 300 |
| adhe24 | YVDATANIERAVEDLLLSKRFDNGMICATENSAVIDASVYDEFIAKMQEQGAYMVPKKDY |
| | :\|:\|\|:\|:\|\|\|:::\|:\|\| \|\|\|\|:\|\|\|:\|:\|:\|: :\|\|\|\|: ::::::\|:\|::: \|: |
| adhe_e | VIDETADIKRAVASVLMSKTFDNGVICASEQSVVVVDSVYDAVRERFATHGGYLLQGKEL |
| | 220 230 240 250 260 270 |
| | 310 320 330 340 350 360 |
| adhe24 | KAIESFVFVERAGEGFGVTGPVAGRSGQWIAEQAGVKVPKDKDVLLFELDKKNIGEALSS |
| | \|\|::: :: ::\| :::::::\|::: \|\|\| \|\| :\|\|:::::\|: \|:: : :\|::: |
| adhe_e | KAVQDVIL--KNG---ALNAAIVGQPAYKIAELAGFSVPENTKILIGEVTVVDESEPFAH |
| | 280 290 300 310 320 330 |
| | 370 380 390 400 410 419 |
| adhe24 | EKLSPLLSIYKAETREEGIEIVRSLLAYQGAGHNAAIQIGAMDDP-FVKEYGEKVEASRI |
| | \|\|\|\|\| \|::\|:\|:: \|:::\| : :\|:\| \| \|\|:: : ::: ::\| \|: :\|:\|::::\|\| |
| adhe_e | EKLSPTLAMYRAKDFEDAVEKAEKLVAMGGIGHTSCLYTDQDNQPARVSYFGQKMKTARI |
| | 340 350 360 370 380 390 |
| | 420 430 440 450 460 470 479 |
| adhe24 | LVNQPDSIGGVGDIYTDAMRPSLTLGTGSWGKNSLSHNLSTYDLLNVKTVAKRRNRPQWV |
| | \|:\| \|:\| \|\|:\|\|:\|: : \|\|\|\|\|\| \|\|\|\| \|\|:\|:\|::: :\|:\| \|\|\|\|\|\| :: \| |
| adhe_e | LINTPASQGGIGDLYNFKLAPSLTLGCGSWGGNSISENVGPKHLINKKTVAKRAENMLWH |
| | 400 410 420 430 440 450 |
| | 480 490 500 510 520 530 |
| adhe24 | RLPKEIYYEKNAISY-LQE-LPHVHK-AFIVADPGMVKFGFVDKVLEQLAIRPTQVETSI |
| | :\|\|\|:\|\|: ::::: \|:\| ::: \|\| \|:\|\|:\|: : : \|::\|:: : \| ::: \|\|\| |
| adhe_e | KLPKSIYFRRGSLPIALDEVITDGHKRALIVTDRFLFNNGYADQITSVL--KAAGVETEV |
| | 460 470 480 490 500 510 |
| | 540 550 560 570 580 590 |
| adhe24 | YGSVQPDPTLSEAIAIARQMKQFEPDTVICLGGGSALDAGKIGRLIYEYDARGEADLSDD |
| | : :\|::\|\|\|\|\| : \| : \|:\|\|::\| \|\|\|\|\|::\|\|:\|\| :::\|\| : \|:: |
| adhe_e | FFEVEADPTLSIVRKGAELANSFKPDVIIALGGGSPMDAAKIMWVMYE---HPETH---- |
| | 520 530 540 550 560 |
| | 600 610 620 630 640 650 |
| adhe24 | ASLKELFQELAQKFVDIRKRIIKFYH-PHKAQMVAIPTTSGTGSEVTPFAVITDDETHVK |
| | \|:\|\|\| :\|:\|\|\|\|\|\| \|\| : \|\|:\|:\|::\|\|\|\|\|\|\|\|\|\|\|\|\|\|:\|\|\|:\| \| |
| adhe_e | ------FEELALRFMDIRKRIYKFPKMGVKAKMIAVTTTSGTGSEVTPFAVVTDDATGQK |
| | 570 580 590 600 610 |
| | 660 670 680 690 700 710 |
| adhe24 | YPLADYQLTPQVAIVDPEFVMTVPKRTVSWSGIDAMSHALESYVSVMSSDYTKPISLQAI |
| | \|\|\|\|\|\|:\|\|\|::\|\|\|\|:::\|\|::\|\|: :::\|:\|\|::\|\|:\|:\|\|\|\|::\|:::: :\|\|\| |
| adhe_e | YPLADYALTPDMAIVDANLVMDMPKSLCAFGGLDAVTHAMEAYVSVLASEFSDGQALQAL |
| | 620 630 640 650 660 670 |
| | 720 730 740 750 760 770 |
| adhe24 | KLIFENITESYHYDPAHPTKEGQKARENMHNAATLAGMAFANAFLGINHSLAHKIGGEFG |
| | \|\|: \| \|::\|\|\| :: :\|: :: ::: :: ::\|: \|\| :: :::\|:\|: \|\|::::\| |
| adhe_e | KLLKEYLPASYHEGSKNPVARERVHSAATIAGIAFAN-AFLGVCHSMAHKLGSQFHIPHG |
| | 680 690 700 710 720 730 |
| | 780 790 800 810 820 830 |
| adhe24 | LPHGLAIAIAMPHVIKFNAVTGNVKRTPYPRYETYRAQEDYAEISRFMGFAGKDDSDEKA |
| | \|:::\| \| |
| adhe_e | LANALLICNVIRYNANDNPTKQTAFSQYDRPQARRRYAEIADHLGLSAPGDRTAAKIEKL |
| | 740 750 760 770 780 790 |
| adhe24: SEQ ID NO:5; adh_e: SEQ ID NO:6 | |

| | |
|---|---|
| adhedb1341.pep | |
| sw:adhe_cloab | |
| ID ADHE_CLOAB STANDARD; PRT; 862 AA. | |
| AC | P33744; |
| DT 01 FEB. 1994 (REL. 28, CREATED) | |
| DT 01 FEB. 1994 (REL. 28, LAST SEQUENCE UPDATE) | |
| DT 01 FEB. 1995 (REL. 31, LAST ANNOTATION UPDATE) | |
| DE ALCOHOL DEHYDROGENASE (EC 1.1.1.1) (ADH)/ACETALDEHYDE DEHYDROGENASE | |
| SCORES Init1: 404 Initn: 1297 Opt: 1053 | |
| 38.6% identity in 568 aa overlap | |

| | | |
|---|---|---|
| | 10 20 30 40 50 60 | |
| adhe24 | MATKKAAPAAKKVLSAEEKAAKFQEAVAYTDKLVKKAQAAVLKFEGYTQTQVDTIVAAMA | |
| | \|: :\| ::\| \|\|: \|:\|: \|\|:\| : \| | |
| adhe_c | MKVTTVKELDEKLKVIKEAQKKFSCYSQEMVDEIFRNAA | |
| | 10 20 30 | |
| | 70 80 90 100 110 120 | |
| adhe24 | LAASKHSLELAHEAVNETGRGVVEDKDTKNHFASESVYNAIKNDKTVGVISENKVAGSVE | |
| | :\|\| : ::\|\|\|::\|\| \|\|\|:\|:\|\|\|\| :\|\|\|\|\|:\| :\|\| \|::\|\| \|:\|: \|: \| :: | |
| adhe_c | MAAIDARIELAKAAVLETGMGLVEDKVIKNHFAGEYIYNKYKDEKTCGIIERNEPYGITK | |
| | | 40 50 60 70 80 90 |
| | 130 140 150 160 170 180 | |
| adhe24 | IASPLGVLAGIVPTTNPTSTAIFKSLLTAKTRNAIVFAFHPQAQKCSSHAAKIVYDAAIE | |
| | \|\|:\|:\|\|:\|:\|:\|:\|\|\|\|\|\|:\|\|\|\|\|:: \|\|\|\|:\| \|: \|\|:\|:\|:: \|\|\|:: \|\|\|:: | |
| adhe_c | IAEPIGVVAAIIPVTNPTSTTIFKSLISLKTRNGIFFSPHPRAXKSTILAAKTILDAAVK | |
| | 100 110 120 130 140 150 | |
| | 190 200 210 220 230 240 | |
| adhe24 | AGAPEDFIQWIEVPSLDMTTALIQNRGLATILATGGPGMVNAALKSGNPSLGVGAGNGAV | |
| | :\|\|\|\|::\| \|\|: \|\|:::\| \|:\|: ::: \|\|\|\|\|\|::\|::\| :\|\|:\|::\|\|\|:\|\|::\| | |
| adhe_c | SGAPENIIGWIDEPSIELTQYLMQKADIT--LATGGPSLVKSAYSSGKPAIGVGPGNTPV | |
| | 160 170 180 190 200 210 | |
| | 250 260 270 280 290 300 | |
| adhe24 | YVDATANIERAVEDLLLSKRFDNGMICATENSAVIDASVYDEFIAKMQEQGAYMVPKKDY | |
| | :\|::\|:\|::\|\|::::\|\|\| :\|\|\|:\|\|\|:\|:\|::: \|:\|:: :::\|\|:\|\|\|:: \|:: | |
| adhe_c | IIDESAHIKMAVSSIILSKTYDNGVICASEQSVIVLKSIYNKVKDEFQERGAYIIKKNEL | |
| | 220 230 240 250 260 270 | |
| | 310 320 330 340 350 360 | |
| adhe24 | KAIESFVFVERAGEGFGVTGPVAGRSGQWIAEQAGVKVPKDKDVLLFELDKKNIGEALSS | |
| | : : : :\| ::\| :\|: ::\|:\|: \|\|: \|\|:\|\|\|\|:: :\|: \|::: : :\|::: | |
| adhe_c | DKVREVIF--KDG---SVNPKIVGQSAYTIAAMAGIKVPKTTRILIGEVTSLGEEEPFAH | |
| | 280 290 300 310 320 330 | |
| | 370 380 390 400 410 419 | |
| adhe24 | EKLSPLLSIYKAETREEGIEIVRSLLAYQGAGHNAAIQIGAMDDP-FVKEYGEKVEASRI | |
| | \|\|\|\|\|:\|::\|:\|:: ::::: : :\|:: \| \|\|:::\| :::::: :: ::: ::: \|: | |
| adhe_c | EKLSPVLAMYEADNFDDALKKAVTLINLGGLGHTSGIYADEIKARDKIDRFSSAMKTVRT | |
| | 340 350 360 370 380 390 | |
| | 420 430 440 450 460 470 479 | |
| adhe24 | LVNQPDSIGGVGDIYTDAMRPSLTLGTGSWGKNSLSHNLSTYDLLNVKTVAKRRNRPQWV | |
| | :\|\| \|:\| \|: \|\|:\|: ::\|\|:\|\|\| \| \|\| \|\|:\|:\|::: :\|\|\|:\|\|\|\|:\|\|:: \| | |
| adhe_c | FVNIPTSQGASGDLYNFRIPPSFTLGCGFWGGNSVSENVGPKHLLNIKTVAERRENMLWF | |
| | 400 410 420 430 440 450 | |
| | 480 490 500 510 520 530 | |
| adhe24 | RLPKEIYYEKNAISY-LQELPHVHK--AFIVADPGMVKFGFVDKVLEQLAIRPTQVETSI | |
| | \|:\|:::\|:: : : : \|::\| :::\| \|\|\|\|:\|:: ::::\|\|:::: \|: : ::: :: | |
| adhe_c | RVPHKVYFKFGCLQFALKDLKDLKKKRAFIVTDSDPYNLNYVDSIIKILE--HLDIDFKV | |
| | 460 470 480 490 500 510 | |
| | 540 550 560 570 580 590 | |
| adhe24 | YGSVQPDPTLSEAIAIARQMKQFEPDTVICLGGGSALDAGKIGRLIYEYDARGEADLSDD | |
| | :::\| ::::\|:: : :\|: \| \|\|\|:\| \|\|\|:::::::\|: :::\|\|: \| :\|\|: | |
| adhe_c | FNKVGREADLKTIKKATEEMSSFMPDTIIALGGTPEMSSAKLMWVLYEHPEVKFEDLAIK | |
| | 520 530 540 550 560 570 | |
| | 600 610 620 630 640 650 | |
| adhe24 | ASLKELFQELAQKFVDIRKRIIKFYHPHKAQMVAIPTTSGTGSEVTPFAVITDDETHVKY | |
| adhe_c | FMDIRKRIYTFPKLGKKAMLVAITTSAGSGSEVTPFALVTDNNTGNKYMLADYEMTPNMA | |
| | 580 590 600 610 620 630 | |

adhe24: Corresponding to amino acid residues 1-656 of SEQ ID NO:5
adh_c: corresponding to amino acid residues 1-630 of SEQ ID NO:11

6. Inverse PCR to Obtain Sequences Upstream of the L. lactis DB1341 adhE Coding Sequence and Cloning of PCR Fragments

Inverse PCR was used to obtain additional sequences from the upstream region of the L. lactis DB1341 adhE gene. HindIII-, HpaI- or PvuII-digested genomic DNA of strain DB1341 was ligated at low concentration and PCR was carried out using primers adhE-350 and adhE-700 (or adhE1300x) (see FIG. 2). Sequence analysis of the obtained PCR products, using primers adhE-240 (or adhE-1300x), allowed the identification of the upstream region of the adhE gene. A 0.6 kb PCR product obtained from HindIII inverse PCR amplification was subsequently cloned into pSMA500 resulting in E. coli DH5α strain adhEup-1.

A sample of adhEup-1 was deposited under the Budapest Treaty with the German Collection of Microorganisms and Cell Cultures, Mascheroder Weg 1b, D-38 124 Braunschweig, Germany on 18 Jul. 1996 under the accession No. DSM 11091.

Further inverse PCR was carried out using PstI-digested and religated chromosomal DNA of strain DB1341, using primers derived from the above sequence. An about 5 kb PCR product was obtained which in addition to the entire coding sequence of the adhE gene comprises about 1800 bp upstream of the coding sequence. This upstream sequence includes an open reading frame, designated orfB that encodes a putative 341 aa protein having no homology to in available databases.

TABLE 1.6
DNA sequence upstream of the coding sequence of the
L. lactis DB1341 adhE gene

| | PstI | | |
|---|---|---|---|
| 1 | CTGCAGCTTGTTTTTTAGTACCAACAAAAAGGACTACTGCACCTTCTTGT | 50 | (SEQ ID NO:26) |
| 51 | GAAGCGTTTTTTACATAGTTGTAAGCATCGTCAACAAGTTTTACAGTTTT | 100 | |
| 101 | TTGAAGGTCGATAACGTGGATACCATTACGTTCTGTGAAGATGTATGGTT | 150 | |
| 151 | TCATTTTTGGGTTCCAACGACGAGTTTGGTGACCGAAGTGAACACCAGCT | 200 | |
| 201 | TCAAGAAGTTGTTTCATTGAAATAACTGACATGTTAATGTCTCCTTTTAA | 250 | |
| 251 | AATAGTTTTTCCTCTTTCATCTGTCATCCGCAGCCGCAATACTTGCGTAC | 300 | |
| 301 | ACTACGACTTTGTCGAGACGAAATGCGAGATGGTTGCATAGCAACTCTAT | 350 | |
| 351 | CATTATACATTGTTTGACCTATTTTTGCAAGTATCTATTCATGCTTCTAT | 400 | |
| 401 | TGTTCAGTAAATCTATTTTTCTAACCACTCCTATTATCTGACAAATTTAA | 450 | |
| 451 | TTGTTAATTAGGCTCTATAATCACTAAAAGAGTAAGTTTTTTAAATTTTT | 500 | |
| 501 | TTCTAAGAAAAAAATTAATATTTTTGCTGAAACCGCTTTTTTTGTGATAA | 550 | |
| 551 | AATAATTATAGTAAATAAATTAGTTTGTGAGGAGAGAAATATGAAAGAAA | 600 | |
| | orfB M K E K | | (SEQ ID NO:27) |
| 601 | AAATCCTTTTAGGCGGCTATACAAAACGTGTATCTAAAGGCGTATATAGT | 650 | |
| | I L L G G Y T K R V S K G V Y S | | |
| 651 | GTTCTTTTGGACACTAAAGCTGCTGAATTATCATCATTAAATGAAGTCGC | 700 | |
| | V L L D T K A A E L S S L N E V A | | |
| 701 | TGCGGTTCAAAACCCTACTTATATCACTCTCGATGAAAAGGGACACCTCT | 750 | |
| | A V Q N P T Y I T L D E K G H L Y | | |
| 751 | ATACTTGTGCAGCAGATAGTAATGGTGGAGGAATCGCCGCCTTTGATTTT | 800 | |
| | T C A A D S N G G G I A A F D F | | |
| 801 | GATGGCGAAACTGCTACTCATCTCGGAAATGTCACAACCACGGGAGCTCC | 850 | |
| | D G E T A T H L G N V T T T G A P | | |
| 851 | ACTCTGCTATGTTGCCGTGGACGAAGCGCGACAATTAGTTTACGGAGCGA | 900 | |
| | L C Y V A V D E A R Q L V Y G A N | | |
| 901 | ACTATCATCTTGGAGAAGTTCGTGTTTATAAGATTCAAGCTAATGGCTCA | 950 | |
| | Y H L G E V R V Y K I Q A N G S | | |
| 951 | CTCCGATTAACGGATACAGTAAAACATACCGGTTCTGGACCACGTCCTGA | 1000 | |
| | L R L T D T V K H T G S G P R P E | | |
| 1001 | ACAAGCTAGCTCACACGTTCATTATTCTGATTTGACTCCTGACGGACGAC | 1050 | |
| | Q A S S H V H Y S D L T P D G R L | | |
| 1051 | TTGTCACCTGTGATTTGGGAACAGATGAAGTCACTGTTTATGATGTCATT | 1100 | |
| | V T C D L G T D E V T V Y D V I | | |
| 1101 | GGTGAAGGTAAACTCAATATTGCTACAATTTATCGGGCAGAAAAAGGAAT | 1150 | |
| | G E G K L N I A T I Y R A E K G M | | |
| 1151 | GGGTGCTCGTCATATTACTTTCCATCCAAATGGTAAAATCGCTTATTTGG | 1200 | |
| | G A R H I T F H P N G K I A Y L V | | |
| 1201 | TTGGAGAGTTAAATTCAACAATTGAAGTTTTAAGTTACAATGAAGAAAAA | 1250 | |
| | G E L N S T I E V L S Y N E E K | - | |
| 1251 | GGACGCTTTGCTCGTCTTCAAACAATTAGCACCCTACCTGAAGATTATCA | 1300 | |
| | G R F A R L Q T I S T L P E D Y H | | |
| 1301 | TGGAGCAAATGGTGTTGCTGCCATCCGTATTTCATCTGACGGTAAATTCC | 1350 | |
| | G A N G V A A I R I S S D G K F L | | |
| 1351 | TCTATACTTCTAATCGTGGACATGATTCTTTGACAACTTACAAAGTAAGT | 1400 | |
| | Y T S N R G H D S L T T Y K V S | | |
| 1401 | CCTCTTGGTACAAAACTTGAAACTATTGGCTGGACAAATACTGAAGGTCA | 1450 | |
| | P L G T K L E T I G W T N T E G H | | |
| 1451 | TATCCCTCGCGATTTTAATTTCAACAAAACTGAAGATTATATCATTGTCG | 1500 | |
| | I P R D F N F N K T E D Y I I V A | | |
| 1501 | CTCATCAAGAATCTGATAATTTATCTCTTTTCTTGCGAGATAAAAAAACC | 1550 | |
| | H Q E S D N L S L F L R D K K T | | |
| 1551 | GGTACTTTAACTTTGGAACAAAAAGATTTTTACGCTCCTGAAATCACTTG | 1600 | |
| | G T L T L E Q K D F Y A P E I T C | | |
| 1601 | TGTTTTACCACTATAAAAATTTATTTTTTCACAAAGTTTGACTGATAAAC | 1650 | |
| | V L P L Stop | | |
| 1651 | TAAAAAAGATTGCTAATTTCTCTCAAAGAATTAGCAATCTTTTTTTCTTC | 1700 | |
| 1701 | AGTAAAGCTTGTTACAAAACCGTTTTCTAAACTTTTGATGAGTGTTTTTG | 1750 | |
| 1751 | TAAAAACTATCACAATATTGCTTGACATCTATAAAAAACTTTGTTAAACT | 1800 | |
| 1801 | ATTCACGTAAAAGAAAGTGAATGAAGTCACAAAGGAGAACCTACAAAT | | |

7. Sequence of a Fragment of the L. lactis Strain MG1363 adhE Gene

PCR was used to characterize the adhE homologue of strain MG1363. Primers adhE-mg1 and adhE-1697 were used to amplify a 1.5 kb fragment from this strain, named MGadhESTART. Primers adhE-1300x and adhE-mg2 were used to amplify an overlapping 1.5 kb fragment, named MGadhESTOP (FIG. 3).

The above fragments were subsequently cloned into the plasmid pGEM and transformed into E. coli DH5α resulting in strains MGadhESTART and MGadhESTOP, respectively. Using the relevant primers a sequence was obtained that spans from position 1306-2775 shown in Table 1.2. An additional primer adhE-mg3 (5′-CTTCTTTGGTTGGATGAGC-3′) (SEQ ID NO:7), derived from the MG1363 adhE sequence and corresponding to position 2359-2335 of the DB1341 adhE sequence (Table 1.4) was used to fill a sequence gap. A limited sequence variation at the DNA level (84 base changes, no insertion/deletions in the 1470 bp MG1363 adhE fragment, corresponding to 5.7% variation; Table 1.7 below), resulting in only 8 amino acid substitutions (or 1.6% variation; Table 1.7).

A sample of E. coli DH5α strain MGadhESTART and strain MGadhESTOP, respectively were deposited under the Budapest Treaty with the German Collection of Microorganisms and Cell Cultures, Mascheroder Weg 1b, D-38 124 Braunschweig, Germany on 18 Jul. 1996 under the accession Nos DSM 11089 and DSM 11090, respectively.

TABLE 1.7
Multialignment of the deduced L. lactis AdhE protein
from strain MG1363 (fragment, adhemg1363) and DB1341 (adhedb13-
41) with the E. coli (adhe_ec) and C. acetobutylicum (aad_ca)
AdhE homologues
The Program lineup (GCG Wisconsin package version 8, Genetics
Computer Group) was used for the alignment. The consensus
sequence (bold type at bottom) shows only conserved residues
for all proteins. The differences between the two L. lactis
AdhE proteins are shown as bold, underlined in adhemg1363.

| | |
|---|---|
| | 1 50 |
| adhemg1363 | .......... .......... .......... .......... .......... |
| adhedb1341 | MATKKAAPAA KKVLSAEEKA AKF.QEAVAY TDKLVKKAQA AVLK.FEGYT |
| adhe_ec | MAVTNVA... ..ELNALVER VKKAQREYAS FT......QE QVDKIFRA.. |
| aad_ca | MKVTTVK... ..ELDEKLKV IKEAQKKFSC YS......QE MVDEIFRN.. |
| consensus | M......... ...L...... .K..Q..... ........Q. .V...F.... |
| | 51 100 |
| adhemg1363 | .......... .......... .......... .......... .......... |
| adhedb1341 | QTQVDTIVAA MALAASKHSL ELAHEAVNET GRGVVEDKDT KNHFASESVY |
| adhe_ec | .......... AALAAADARI PLAKMAVAES GMGIVEDKVI KNHFASEYIY |
| aad_ca | .......... AAMAAIDARI ELAKAAVLET GMGLVEDKVI KNHFAGEYIY |
| consensus | .......... .A.AA..... .LA..AV.E. G.G.VEDK.. KNHFA.E..Y |
| | 101 150 |
| adhemg1363 | .......... .......... .......... .......... .......... |
| adhedb1341 | NAIKNDKTVG VISENKVAGS VEIASPLGVL AGIVPTTNPT STAIFKSLLT |
| adhe_ec | NAYKDEKTCG VLSEDDTFGT ITIAEPIGII CGIVPTTNPT STAIFKSLIS |
| aad_ca | NKYKDEKTCG IIERNEPYGI TKIAEPIGVV AAIIPVTNPT STTIFKSLIS |
| consensus | N..K..KT.G ........G. ..IA.P.G.. ..I.P.TNPT ST.IFKSLI. |
| | 151 200 |
| adhemg1363 | .......... .......... .......... .......... .......... |
| adhedb1341 | AKTRNAIVFA FHPQAQKCSS HAAKIVYDAA IEAGAPEDFI QWIEVPSLDM |
| adhe_ec | LKTRNAIIFS PHPRAKDATN KAADIVLQAA IAAGAPKDLI GWIDQPSVEL |
| aad_ca | LKTRNGIFFS PHPRAKKSTI LAAKTILDAA VKSGAPENII GWIDEPSIEL |
| consensus | .KTRN..... .HP.A..... .AA.....AA ...GAP...I .WI..PS... |
| | 201 250 |
| adhemg1363 | .......... .......... .......... .......... .......... |
| adhedb1341 | TTALIQNRGL ATILATGGPG MVNAALKSGN PSLGVGAGNG AVYVDATANI |
| adhe_ec | SNALMHHPDI NLILATGGPG MVKAAYSSGK PAIGVGAGNT PVVIDETADI |
| aad_ca | TQYLMQKADI T..LATGGPS LVKSAYSSGK PAIGVGPGNT PVIIDESAHI |
| consensus | ...L...... ...LATGGP. .VK.A..SG. P.IGVG.GN. .V..D..A.I |
| | 251 300 |
| adhemg1363 | .......... .......... .......... .......... .......... |
| adhedb1341 | ERAVEDLLLS KRFDNGMICA TENSAVIDAS VYDEFIAKMQ EQGAYMVPKK |
| adhe_ec | KRAVASVLMS KTFDNGVICA SEQSVVVVDS VYDAVRERFA THGGYLLQGK |
| aad_ca | KMAVSSIILS KTYDNGVICA SEQSVIVLKS IYNKVKDEFQ ERGAYIIKKN |
| consensus | ..AV.....S ...DNG.ICA .E.S.....S .Y.......G ..G.Y..... |
| | 301 350 |
| adhemg1363 | .......... .......... .......... .......... .......... |
| adhedb1341 | DYKAIESFVF VERAGEGFGV TGPVAGRSGQ WIAEQAGVKV PKDKDVLLFE |
| adhe_ec | ELKAVQDVIL ..KNG...AL NAAIVGQPAY KIAELAGFSV PENTKILIGE |
| aad_ca | ELDKVREVIF ..KDG...SV NPKIVGQSAY TIAAMAGIKV PKTTRILIGE |
| consensus | .......... ....G..... .....G.... .IA..AG..V P.....LIGE |
| | 351 400 |
| adhemg1363 | .......... .......... .......... .........Y QGAGHNAAIQ |
| adhedb1341 | LDKKNIGEAL SSEKLSPLLS IYKAETREEG IEIVRSLLAY QGAGHNAAIQ |
| adhe_ec | VTVVDESEPF AHEKLSPTLA MYRAKDFEDA VEKAEKLVAM GGIGHTSCLY |
| aad_ca | VTSLGEEEPF AHEKLSPVLA MYEADNFDDA LKKAVTLINL GGLGHTSGIY |
| consensus | .......E.. ..EKLSP.L. .Y.A...... ......L... .G.GH..... |
| | 401 450 |
| adhemg1363 | IGAMDDP.FV KEYGIKVEAS RILVNQPDSI GGVGDIYTDA MRPSLTLGTG |
| adhedb1341 | IGAMDDP.FV KEYGEKVEAS RILVNQPDSI GGVGDIYTDA MRPSLTLGTG |
| adhe_ec | TDQDNQPARV SYFGQKMKTA RILINTPASQ GGIGDLYNFK LAPSLTLGCG |
| aad_ca | ADEIKARDKI DRFSSAMKTV RTFVNIPTSQ GASGDLYNFR IPPSFTLGCG |
| consensus | .......... .......... R...N.P.S. G..GD.Y... ..PS.TLG.G |
| | 451 500 |
| adhemg1363 | SWGKNSLSHN LSTYDLLNVK TVAKRRNRPQ WVRLPKEIYY EKNAISYLQE |
| adhedb1341 | SWGKNSLSHN LSTYDLLNVK TVAKRRNRPQ WVRLPKEIYY EKNAISYLQE |
| adhe_ec | SWGGNSISEN VGPKHLINKK TVAKRAENML WHKLPKSIYF RRGSLPIALD |
| aad_ca | FWGGNSVSEN VGPKHLLNIK TVAERRENML WFRVPHKVYF KFGCLQFALK |
| consensus | .WG.NS.S.N .....L.N.K TVA.R..... W...P...Y. .......... |
| | 501 550 |
| adhemg1363 | LPHVHK...A FIVADPGMVK FGFVDKVLEQ LAIRPTQVET SIYGSVQPDP |
| adhedb1341 | LPHVHK...A FIVADPGMVK FGFVDKVLEQ LAIRPTQVET SIYGSVQPDP |
| adhe_ec | EVITDGHKRA LIVTDRFLFN NGYADQITSV L..KAAGVET EVFFEVEADP |
| aad_ca | DLKDLKKKRA FIVTDSDPYN LNYVDSIIKI L..EHLDIDF KVFNKVGREA |
| consensus | .......... .IV.D..... ....D..... L......... .....V.... |
| | 551 600 |
| adhemg1363 | TLSEAIAIAR QMNHFEPDTV ICLGGGSALD AGKIGRLIYE YDARGEADLS |
| adhedb1341 | TLSEAIAIAR QMKQFEPDTV ICLGGGSALD AGKIGRLIYE YDARGEADLS |
| adhe_ec | TLSIVRKGAE LANSFKPDVI IALGGGSPMD AAKIMWVMYE ...HPETH.. |
| aad_ca | DLKTIKKATE EMSSFMPDTI IALGGTPEMS SAKLMWVLYE ...HPEVK.. |
| consensus | .S........ ....F.PD.. I.LGG..... ..K.....YE .....E.... |
| | 601 650 |
| adhemg1363 | DDASLKEIFQ ELAQKFVDIR KRIIKFYH.P HKAQMVAIPT TSGTGSEVTP |
| adhedb1341 | DDASLKELFQ ELAQKFVDIR KRIIKFYH.P HKAQMVAIPT TSGTGSEVTP |
| adhe_ec | ........FE ELALRFMDIR KRIYKFPKMG VKAKMIAVTT TSGTGSEVTP |
| aad_ca | ........FE DLAIKFMDIR KRIYTFPKLG KKAMLVAITT SAGSGSEVTP |
| consensus | ........F. .LA..F.DIR KRI..F.... .KA...A..T ....GSEVTP |
| | 651 700 |
| adhemg1363 | FAVITDDETH VKYPLADYQL TPQVAIVDPE FVMTVPKRTV SWSGIDAMSH |
| adhedb1341 | FAVITDDETH VKYPLADYQL TPQVAIVDPE FVMTVPKRTV SWSGIDAMSH |
| adhe_ec | FAVVTDDATG QKYPLADYAL TPDMAIVDAN LVMDMPKSLC AFGGLDAVTH |
| aad_ca | FALVTDNNTG NKYMLADYEM TPNMAIVDAE LMMKMPKGLT AYSGIDALVN |
| consensus | FA..TD..T. .KY.LADY.. TP..AIVD.. ..M..PK... ...G.DA... |
| | 701 750 |
| adhemg1363 | ALESYVSVMS SDYTKPISLQ AIKLIFENLT ESYEYDPAHP TKEGQKAREN |
| adhedb1341 | ALESYVSVMS SDYTKPISLQ AIKLIFENLT ESYHYDPAHP TKEGQKAREN |
| adhe_ec | AMEAYVSVLA SEFSDGQALQ ALKLLKEYLP ASYHEGSKNP .....VARER |
| aad_ca | SIEAYTSVYA SEYTNGLALE AIRLIFKYLP EAYKNGRTNE .....KAREK |
| consensus | ..E.Y.SV.. S.......L. AI.L....L. ..Y....... ......ARE. |
| | 751 800 |
| adhemg1363 | MHNAATLAGM AFANAFLGIN HSLAHKIAGE FGLPHGLAIA IAMPHVIKFN |
| adhedb1341 | MHNAATLAGM AFANAFLGIN HSLAHKIGGE FGLPHGLAIA IAMPHVIKFN |
| adhe_ec | VHSAATIAGI AFANAFLGVC HSMAHKLGSQ FHIPHGLANA LLICNVIRYN |
| aad_ca | MAHASTMAGM ASANAFLGLC HSMAIKLSSE HNIPSGIANA LLIEEVIKFN |
| consensus | ...A.T.AG. A.ANAFLG.. HSMA.K.... ...P.G.A.A .....VI..N |
| | 801 850 |
| adhemg1363 | AVTGNVKTP YPRYETYRAQ EDYAEISRFM GFAGKEDSDE KAVKAFVAEL |
| adhedb1341 | AVTGNVKRTP YPRYETYRAQ EDYAEISRFM GFAGKDDSDE KAVQALVAEL |
| adhe_ec | ANDNPTKQTA FSQYDRPQAR RRYAEIADHL GLSAPGDRTA AKIEKLLAWL |
| aad_ca | AVDNPVKQAP CPQYKYPNTI FRYARIADYI KLGGNTDEEK VDLLINKIHE |
| consensus | A.....K... ...Y...... ..YA.I.... ......D... .......... |
| | 851 900 |
| adhemg1363 | KKLTDSIDIN ITLSGN..GV DKAHLERELD KLADLV |
| adhedb1341 | KKLTDSIDIN ITLSGN..GI DKAHLERELD KLADLVYDDQ CTPANPRQPR |
| adhe_ec | ETLKA..ELG IPKSIREAGV QEADFLANVD KLSEDAFDDQ CTGANPRYPL |
| aad_ca | LKKAL....N IPTSIKDAGV LEENFYSSLD RISELALDDQ CTGANPRFPL |
| consensus | .......... I..S....G. .........D .......DDQ CT.ANPR.P. |
| | 901 941 |
| adhedb1341 | IDEIKQLLLD QY* |
| adhe_ec | ISELKQILLD TYYGPDYVEG ETAAKKEAAP AKAEKKAKKS A |
| aad_ca | TSEIKEMYIN CFKKQP |
| consensus | ..E.K..... .......... .......... .......... . |

adhemg1363: SEQ ID NO:8; adhedb1341: SEQ ID NO:9; adhe_ec: SEQ ID NO:10; aad_ca: SEQ ID NO:11

TABLE 1.8

Alignment of the adhE sequences from L. lactis
DB1341 and MG1363
The complete sequence of the adhE gene of strain DB1341 is
compared to the sequence obtained via PCR amplification of
MG1363 adhE fragments (see FIG. 2).

| | | |
|---|---|---|
| | 1 50 | |
| adhemg1363 | .......... .......... .......... .......... .......... | (SEQ ID NO:12) |
| adhedb1341 | AAGCTTGTTA CAAAACCGTT TTCTAAACTT TTGATGAGTG TTTTTGTAAA | (SEQ ID NO:13) |
| consensus | .......... .......... .......... .......... .......... | |
| | 51 100 | |
| adhemg1363 | .......... .......... .......... .......... .......... | |
| adhedb1341 | AACTATCACA ATATTGCTTG ACATCTATAA AAAACTTTGT TAAACTATTC | |
| consensus | .......... .......... .......... .......... .......... | |
| | 101 150 | |
| adhemg1363 | .......... .......... .......... .......... .......... | |
| adhedb1341 | ACGTAAAAGA AAGTGAATGA AGTCACAAAG GAGAACCTAC AAATATGGCA | |
| consensus | .......... .......... .......... .......... .......... | |
| | 151 200 | |
| adhemg1363 | .......... .......... .......... .......... .......... | |
| adhedb1341 | ACTAAAAAAG CCGCTCCAGC TGCAAAGAAA GTTTTAAGCG CTGAAGAAAA | |
| consensus | .......... .......... .......... .......... .......... | |
| | 201 250 | |
| adhemg1363 | .......... .......... .......... .......... .......... | |
| adhedb1341 | AGCCGCAAAA TTCCAAGAAG CTGTTGCTTA TACTGACAAA TTAGTCAAAA | |
| consensus | .......... .......... .......... .......... .......... | |
| | 251 300 | |
| adhemg1363 | .......... .......... .......... .......... .......... | |
| adhedb1341 | AAGCACAAGC TGCTGTTCTT AAATTTGAAG GATATACACA AACTCAAGTC | |
| consensus | .......... .......... .......... .......... .......... | |
| | 301 350 | |
| adhemg1363 | .......... .......... .......... .......... .......... | |
| adhedb1341 | GATACTATTG TCGCTGCAAT GGCTCTTGCA GCAAGCAAAC ATTCTCTAGA | |
| consensus | .......... .......... .......... .......... .......... | |
| | 351 400 | |
| adhemg1363 | .......... .......... .......... .......... .......... | |
| adhedb1341 | ACTCGCTCAT GAAGCCGTTA ACGAAACTGG TCGTGGTGTT GTCGAAGACA | |
| consensus | .......... .......... .......... .......... .......... | |
| | 401 450 | |
| adhemg1363 | .......... .......... .......... .......... .......... | |
| adhedb1341 | AAGATACCAA AAACCACTTT GCTTCTGAAT CTGTTTATAA CGCAATTAAA | |
| consensus | .......... .......... .......... .......... .......... | |
| | 451 500 | |
| adhemg1363 | .......... .......... .......... .......... .......... | |
| adhedb1341 | AATGACAAAA CTGTTGGTGT CATTTCTGAA AACAAGGTTG CTGGATCTGT | |
| consensus | .......... .......... .......... .......... .......... | |
| | 501 550 | |
| adhemg1363 | .......... .......... .......... .......... .......... | |
| adhedb1341 | TGAAATCGCA AGCCCTCTCG GTGTACTTGC TGGTATCGTT CCAACGACTA | |
| consensus | .......... .......... .......... .......... .......... | |
| | 551 600 | |
| adhemg1363 | .......... .......... .......... .......... .......... | |
| adhedb1341 | ATCCAACATC AACAGCAATC TTTAAATCTT TATTGACTGC AAAAACACGT | |
| consensus | .......... .......... .......... .......... .......... | |
| | 601 650 | |
| adhemg1363 | .......... .......... .......... .......... .......... | |
| adhedb1341 | AATGCTATTG TTTTCGCTTT CCACCCTCAA GCTCAAAAAT GTTCAAGCCA | |
| consensus | .......... .......... .......... .......... .......... | |
| | 651 700 | |
| adhemg1363 | .......... .......... .......... .......... .......... | |
| adhedb1341 | TGCAGCAAAA ATTGTTTACG ATGCTGCAAT TGAAGCTGGT GCACCGGAAG | |
| consensus | .......... .......... .......... .......... .......... | |
| | 701 750 | |
| adhemg1363 | .......... .......... .......... .......... .......... | |
| adhedb1341 | ACTTTATTCA ATGGATTGAA GTACCAAGCC TTGACATGAC TACCGCCTTG | |
| consensus | .......... .......... .......... .......... .......... | |
| | 751 800 | |
| adhemg1363 | .......... .......... .......... .......... .......... | |
| adhedb1341 | ATTCAAAACC GTGGACTTGC AACAATCCTT GCAACTGGTG GCCCAGGAAT | |
| consensus | .......... .......... .......... .......... .......... | |
| | 801 850 | |
| adhemg1363 | .......... .......... .......... .......... .......... | |
| adhedb1341 | GGTAAACGCC GCACTCAAAT CTGGTAACCC TTCACTCGGT GTTGGAGCTG | |
| consensus | .......... .......... .......... .......... .......... | |
| | 851 900 | |
| adhemg1363 | .......... .......... .......... .......... .......... | |
| adhedb1341 | GTAATGGTGC TGTTTATGTT GATGCAACTG CAAATATTGA ACGTGCCGTT | |
| consensus | .......... .......... .......... .......... .......... | |
| | 901 950 | |
| adhemg1363 | .......... .......... .......... .......... .......... | |
| adhedb1341 | GAAGACCTTT TGCTTTCAAA ACGTTTTGAT AATGGGATGA TTTGTGCCAC | |
| consensus | .......... .......... .......... .......... .......... | |
| | 951 1000 | |
| adhemg1363 | .......... .......... .......... .......... .......... | |
| adhedb1341 | TGAAAATTCA GCTGTTATTG ATGCTTCAGT TTATGATGAA TTTATTGCTA | |
| consensus | .......... .......... .......... .......... .......... | |
| | 1001 1050 | |
| adhemg1363 | .......... .......... .......... .......... .......... | |
| adhedb1341 | AAATGCAAGA ACAAGGCGCT TATATGGTTC CTAAAAAAGA CTACAAAGCT | |
| consensus | .......... .......... .......... .......... .......... | |
| | 1051 1100 | |
| adhemg1363 | .......... .......... .......... .......... .......... | |
| adhedb1341 | ATTGAAAGTT TCGTTTTTGT TGAACGTGCT GGTGAAGGTT TTGGAGTAAC | |
| consensus | .......... .......... .......... .......... .......... | |
| | 1101 1150 | |
| adhemg1363 | .......... .......... .......... .......... .......... | |
| adhedb1341 | TGGTCCTGTT GCCGGTCGTT CTGGTCAATG GATTGCTGAA CAAGCTGGTG | |
| consensus | .......... .......... .......... .......... .......... | |
| | 1151 1200 | |
| adhemg1363 | .......... .......... .......... .......... .......... | |
| adhedb1341 | TCAAAGTTCC TAAAGATAAA GATGTCCTTC TTTTTGAACT TGATAAGAAA | |
| consensus | .......... .......... .......... .......... .......... | |
| | 1201 1250 | |
| adhemg1363 | .......... .......... .......... .......... .......... | |
| adhedb1341 | AATATTGGTG AAGCACTTTC TTCTGAAAAA CTTTCTCCTT TGCTTTCAAT | |
| consensus | .......... .......... .......... .......... .......... | |
| | 1251 1300 | |
| adhemg1363 | .......... .......... .......... .......... .......... | |
| adhedb1341 | CTACAAAGCT GAAACACGTG AAGAAGGAAT TGAGATTGTA CGTAGCTTAC | |
| consensus | .......... .......... .......... .......... .......... | |
| | 1301 1350 | |
| adhemg1363 | .....TACCA AGGAGCTGGT CACAACGCTG CAATTCAAAT CGGTGCAATG | |
| adhedb1341 | TTGCTTATCA AGGTGCTGGA CATAATGCTG CAATTCAAAT CGGTGCAATG | |
| consensus | .....TA.CA AGG.GCTGG. CA.AA.GCTG CAATTCAAAT CGGTGCAATG | |
| | 1351 1400 | |
| adhemg1363 | GACGACCCAT TTGTCAAAGA ATACGGAATT AAAGTCGAAG CTTCTCGTAT | |
| adhedb1341 | GATGATCCAT TCGTTAAAGA ATATGGCGAA AAAGTTGAAG CTTCTCGTAT | |
| consensus | GA.GA.CCAT T.GT.AAAGA ATA.GG.... AAAGT.GAAG CTTCTCGTAT | |
| | 1401 1450 | |
| adhemg1363 | CCTCGTTAAC CAACCTGACT CTATCGGTGG GGTCGGAGAT ATTTATACTG | |
| adhedb1341 | CCTCGTTAAC CAACCAGATT CTATTGGTGG GGTCGGAGAT ATCTATACTG | |
| consensus | CCTCGTTAAC CAACC.GA.T CTAT.GGTGG GGTCGGAGAT AT.TATACTG | |
| | 1451 1500 | |
| adhemg1363 | ATGCAATGCG TCCATCATTG ACGCTCGGAA CTGGTTCATG GGGGAAAAAT | |
| adhedb1341 | ATGCAATGCG TCCATCACTT ACACTTGGAA CTGGTTCATG GGGGAAAAAT | |
| consensus | ATGCAATGCG TCCATCA.T. AC.CT.GGAA CTGGTTCATG GGGGAAAAAT | |
| | 1501 1550 | |
| adhemg1363 | TCACTTTCAC ACAATTTGAG TACATACGAT CTATTGAATG TTAAAACAGT | |
| adhedb1341 | TCACTTTCAC ACAATTTGAG TACATACGAT CTATTGAATG TTAAAACAGT | |
| consensus | TCACTTTCAC ACAATTTGAG TACATACGAT CTATTGAATG TTAAAACAGT | |
| | 1551 1600 | |
| adhemg1363 | GGCTAAACGT CGTAATCGCC CTCAATGGGT TCGTTTGCCA AAAGAAATTT | |
| adhedb1341 | GGCTAAACGT CGTAATCGCC CACAATGGGT TCGTTTGCCA AAAGAAATTT | |
| consensus | GGCTAAACGT CGTAATCGCC C.CAATGGGT TCGTTTGCCA AAAGAAATTT | |
| | 1601 1650 | |
| adhemg1363 | ACTACGAAAA AAATGCAATT TCTTACTTAC AAGAATTGCC ACACGTCCAC | |
| adhedb1341 | ACTACGAAAA AAATGCAATT TCTTACTTAC AAGAATTGCC ACACGTCCAC | |
| consensus | ACTACGAAAA AAATGCAATT TCTTACTTAC AAGAATTGCC ACACGTCCAC | |
| | 1651 1700 | |
| adhemg1363 | AAAGCTTTCA TTGTTGCCGA CCCTGGTATG GTTAAATTCG GTTTCGTTGA | |
| adhedb1341 | AAAGCTTTCA TCGTTGCTGA CCCTGGTATG GTTAAATTTG GTTTCGTTGA | |
| consensus | AAAGCTTTCA T.GTTGC.GA CCCTGGTATG GTTAAATT.G GTTTCGTTGA | |
| | 1701 1750 | |
| adhemg1363 | TAAAGTTTTG GAACAACTTG CTATCCGCCC AACTCAAGTT GAAACAAGCA | |
| adhedb1341 | TAAAGTTTTG GAACAACTTG CTATCCGCCC AACTCAAGTT GAAACAAGCA | |
| consensus | TAAAGTTTTG GAACAACTTG CTATCCGCCC AACTCAAGTT GAAACAAGCA | |
| | 1751 1800 | |
| adhemg1363 | TTTATGGCTC AGTCCAACCT GACCCAACTT TGAGTGAAGC AATTGCAATC | |
| adhedb1341 | TTTATGGCTC TGTTCAACCT GACCCAACTT TGAGCGAAGC AATTGCAATC | |
| consensus | TTTATGGCTC .GT.CAACCT GACCCAACTT TGAG.GAAGC AATTGCAATC | |
| | 1801 1850 | |
| adhemg1363 | GCTCGTCAAA TGAACCATTT TGAACCTGAC ACTGTCATCT GTCTTGGTGG | |
| adhedb1341 | GCTCGTCAAA TGAAACAATT TGAACCTGAC ACTGTCATCT GTCTTGGTGG | |
| consensus | GCTCGTCAAA TGAA.CA.TT TGAACCTGAC ACTGTCATCT GTCTTGGTGG | |
| | 1851 1900 | |
| adhemg1363 | TGGTTCTGCT CTCGATGCTG GTAAGATTGG TCGTTTGATT TATGAATATG | |
| adhedb1341 | TGGTTCTGCT CTCGATGCCG GTAAGATTGG TCGTTTGATT TATGAATATG | |
| consensus | TGGTTCTGCT CTCGATGC.G GTAAGATTGG TCGTTTGATT TATGAATATG | |
| | 1901 1950 | |
| adhemg1363 | ATGCTCGTGG TGAGGCTGAC CTTTCCGATG ACGCAAGTTT GAAAGAGATC | |
| adhedb1341 | ATGCTCGTGG TGAAGCTGAC CTTTCTGATG ATGCAAGTTT GAAAGAACTT | |
| consensus | ATGCTCGTGG TGA.GCTGAC CTTTC.GATG A.GCAAGTTT GAAAGA..T. | |
| | 1951 2000 | |
| adhemg1363 | TTCCAAGAGT TAGCTCAAAA ATTTGITGAT ATTCGTAAAC GTATTATCAA | |
| adhedb1341 | TTCCAAGAAT TAGCTCAAAA ATTTGTCGAT ATTCGTAAAC GTATTATTAA | |
| consensus | TTCCAAGA.T TAGCTCAAAA ATTTGT.GAT ATTCGTAAAC GTATTAT.AA | |
| | 2001 2050 | |
| adhemg1363 | ATTCTACCAC CCACACAAAG CACAAATGGT TGCTATCCCT ACTACTTCTG | |
| adhedb1341 | ATTCTACCAT CCACATAAAG CACAAATGGT TGCAATTCCT ACTACTTCTG | |
| consensus | ATTCTACCA. CCACA.AAAG CACAAATGGT TGC.AT.CCT ACTACTTCTG | |
| | 2051 2100 | |
| adhemg1363 | GTACTGGTTC TGAAGTGACT CCATTTGCGG TTATCACTGA TGATGAAACT | |
| adhedb1341 | GTACTGGTTC TGAAGTGACT CCATTTGCAG TTATCACTGA TGATGAAACT | |
| consensus | GTACTGGTTC TGAAGTGACT CCATTTGC.G TTATCACTGA TGATGAAACT | |
| | 2101 2150 | |
| adhemg1363 | CACGTTAAAT ATCCACTTGC TGACTATCAA TTGACACCTC AAGTTGCCAT | |
| adhedb1341 | CATGTTAAGT ACCCACTTGC TGACTACCAA TTAACACCAC AAGTTGCCAT | |
| consensus | CA.GTTAA.T A.CCACTTGC TGACTA.CAA TT.ACACC.C AAGTTGCCAT | |
| | 2151 2200 | |
| adhemg1363 | TGTTGACCCT GAGTTTGTTA TGACTGTACC AAAACGTACT GTTTCTTGGT | |
| adhedb1341 | TGTTGACCCT GAGTTTGTTA TGACTGTACC AAAACGTACT GTTTCTTGGT | |
| consensus | TGTTGACCCT GAGTTTGTTA TGACTGTACC AAAACGTACT GTTTCTTGGT | |
| | 2201 2250 | |
| adhemg1363 | CTGGGATTGA TGCTATGTCA CACGCGCTTG AATCTTATGT TTCTGTCATG | |
| adhedb1341 | CTGGTATTGA TGCGATGTCA CACGCGCTTG AATCTTACGT TTCTGTTATG | |
| consensus | CTGG.ATTGA TGC.ATGTCA CACGCGCTTG AATCTTA.GT TTCTGT.ATG | |
| | 2251 2300 | |
| adhemg1363 | TCTTCTGACT ATACAAAACC AATTTCACTT CAAGCCATCA AACTCATCTT | |
| adhedb1341 | TCTTCTGACT ATACAAAACC AATTTCACTT CAAGCGATCA AACTTATCTT | |
| consensus | TCTTCTGACT ATACAAAACC AATTTCACTT CAAGC.ATCA AACT.ATCTT | |
| | 2301 2350 | |
| adhemg1363 | TGAAAACTTG ACTGAGTCTT ATCATTATGA CCCAGCTCAT CCAACCAAAG | |
| adhedb1341 | TGAAAACTTG ACTGAGTCTT ATCATTATGA CCCAGCGCAT CCAACTAAAG | |
| consensus | TGAAAACTTG ACTGAGTCTT ATCATTATGA CCCAGC.CAT CCAAC.AAAG | |
| | 2351 2400 | |
| adhemg1363 | AAGGTCAAAA AGCTCGCGAA AACATGCACA ATGCTGCAAC ACTCGCTGGT | |
| adhedb1341 | AAGGACAAAA AGCCCGCGAA AACATGCACA ATGCTGCAAC ACTCGCTGGT | |
| consensus | AAGG.CAAAA AGC.CGCGAA AACATGCACA ATGCTGCAAC ACTCGCTGGT | |
| | 2401 2450 | |
| adhemg1363 | ATGGCCTTCG CCAATGCTTT CCTTGGAATT AACCACTCAC TTGCTCATAA | |
| adhedb1341 | ATGGCCTTCG CTAATGCTTT CCTTGGAATT AACCACTCAC TTGCTCATAA | |
| consensus | ATGGCCTTCG C.AATGCTTT CCTTGGAATT AACCACTCAC TTGCTCATAA | |
| | 2451 2500 | |
| adhemg1363 | AATTGCTGGT GAATTTGGGC TTCCTCATGG TCTTGCCATT GCTATCGCTA | |
| adhedb1341 | AATTGGTGGT GAATTTGGAC TTCCTCATGG TCTTGCCATT GCCATCGCTA | |
| consensus | AATTG.TGGT GAATTTGG.C TTCCTCATGG TCTTGCCATT GC.ATCGCTA | |
| | 2501 2550 | |
| adhemg1363 | TGCCACATGT CATTAAATTT AACGCTGTAA CAGGAAACGT TAAATTTACC | |
| adhedb1341 | TGCCACATGT CATTAAATTT AACGCTGTAA CAGGAAACGT TAAACGTACC | |
| consensus | TGCCACATGT CATTAAATTT AACGCTGTAA CAGGAAACGT TAAA..TACC | |
| | 2551 2600 | |
| adhemg1363 | CCTTACCCAC GTTATGAAAC TTATCGTGCG CAAGAAGACT ACGCTGAAAT | |
| adhedb1341 | CCTTACCCAC GTTATGAAAC ATATCGTGCT CAAGAGGACT ACGCTGAAAT | |
| consensus | CCTTACCCAC GTTATGAAAC .TATCGTGC. CAAGA.GACT ACGCTGAAAT | |
| | 2601 2650 | |
| adhemg1363 | TTCACGCTTC ATGGGATTTG CTGGCAAAGA AGATTCAGAT GAAAAAGCGG | |
| adhedb1341 | TTCACGCTTC ATGGGATTTG CTGGTAAAGA TGATTCAGAT GAAAAAGCTG | |
| consensus | TTCACGCTTC ATGGGATTTG CTGG.AAAGA .GATTCAGAT GAAAAAGC.G | |
| | 2651 2700 | |
| adhemg1363 | TCAAAGCTTT TGTTGCTGAA CTTAAAAAAT TGACTGATAG TATTGATATT | |
| adhedb1341 | TGCAAGCTCT GGTTGCTGAA CTTAAGAAAC TGACTGATAG CATTGATATT | |
| consensus | T..AAGCT.T .GTTGCTGAA CTTAA.AAA. TGACTGATAG .ATTGATATT | |
| | 2701 2750 | |
| adhemg1363 | AATATCACCC TTTCAGGAAA TGGTGTAGAT AAAGCTCACC TTGAACGTGA | |
| adhedb1341 | AATATCACCC TTTCAGGAAA TGGTATCGAT AAAGCTCACC TTGAACGTGA | |
| consensus | AATATCACCC TTTCAGGAAA TGGT.T.GAT AAAGCTCACC TTGAACGTGA | |
| | 2751 2800 | |
| adhemg1363 | GCTTGATAAA TTGGCTGACC TTGTT | |
| adhedb1341 | ACTTGATAAA TTGGCTGACC TTGTTTATGA TGATCAATGT ACTCCTGCTA | |
| consensus | .CTTGATAAA TTGGCTGACC TTGTT..... .......... .......... | |
| | 2801 2850 | |
| adhedb1341 | ATCCTCGTCA ACCAAGAATT GATGAGATTA AACAGTTGTT GTTAGATCAA | |
| consensus | .......... .......... .......... .......... .......... | |
| | 2851 2900 | |
| adhedb1341 | TACTAATAAT CTGTTGATAA AATTATTAAA ACGCTCTGAT GAATTCGTCA | |
| consensus | .......... .......... .......... .......... .......... | |
| | 2901 2950 | |
| adhedb1341 | GAGCATTTTT TATTATAGCT TATACAACTA TCAAAAGGTA TAAATCAATT | |
| consensus | .......... .......... .......... .......... .......... | |
| | 2951 3000 | |
| adhedb1341 | TCGATATAGG CTCTTTTCAC TCCATTGATT TATGCATTTC TATAAAAATC | |
| consensus | .......... .......... .......... .......... .......... | |
| | 3001 3050 | |
| adhedb1341 | AATAATTAAT TAGCGATAGA AGTCGAGTTC ATGCATGCTA ATAATGAAAT | |
| consensus | .......... .......... .......... .......... .......... | |
| | 3051 3100 | |
| adhedb1341 | TGTTTTAAAT TCTGGTTTTT CTTTATGTTC TTTGCGAACA TCTTTCACAG | |
| consensus | .......... .......... .......... .......... .......... | |
| | 3101 3150 | |
| adhedb1341 | TTTCTTTGTT CATGAAAATT CCTCCTTATT ATGGTACTAT TTTGAGCCCA | |
| consensus | .......... .......... .......... .......... .......... | |
| | 3151 3193 | |
| adhedb1341 | AATAGTTATA TAAGAATCCT AAACTTCGGA TATCTTATCA AAG | |
| consensus | .......... .......... .......... .......... ... | |

8. Obtaining and Sequencing the Entire adhE Locus from L. lactis Strain MG1363

Inverse PCR was carried out on digested and religated chromosomal DNA of strain MG1363, using primers adhE-146 and adhE-MG5 (see FIG. 5). A PCR fragment was obtained which in addition to the above fragment of the MG1363 adhE sequence comprised an about 2.9 kb sequence upstream of that fragment including the 5′-end of the adhE coding sequence and and open reading frame, designated orfB showing a high homology with the corresponding open reading frame from strain DB1341.

The entire sequence of the adhE locus of Lactococcus lactis strain MG1363 is shown in Table 1.9 below.

TABLE 1.9
The adhE locus of strain MG1363

| | |
|---|---|
| 1 TTTGGTGACCGAAGTGAACACCAGCTTCAAGAAGTTGTTTCATTGAAATA 50 | (SEQ ID NOS:28/30) |
| 51 ACTGACATGTTAATGTCTCCTTTTAAAATAGTTTTTCCTCTTTCATCTGT 100 | |
| 101 CATCCGCAGCCGCAATACTTGCGTACACTACGACTTTGTCGAGACGAAAT 150 | |
| 151 GCGAGATGGTTGCATAGCAACTCTCTCATTATACATTGTTTAAGCTACTT 200 | |
| 201 TTGCAAGCATCTATTCATTTATTTCTTTTATCAATATGAGTAAATGAAAG 250 | |
| 251 CTATCCTACCCCCCTTTCTTTTTATTCTGTTTTTTATATCTCAATGTTGT 300 | |
| 301 CTGACAAATTTAACGAATATTTTTGCCTATATAATCCCCATAAGGGAGAT 350 | |
| 351 TTTTACATTTTTTTCTAAGAATAAAATTAATATTTTTGCTGAAAACGCTT 400 | |
| 401 TTTTTGTGATAAAATAATTATAGTAAATAAAATAGTTTGTGAGGAGAGAA 450 | |
| 451 ATATGAAAGAAAAAATCCTTTTAGGCGGTTATACTAAACGTGTATCTAAA 500 | |
| orfB M K E K I L L G G Y T K R V S K | (SEQ ID NO:29) |
| 501 GGCGTTTACAGTGTTCTATTAGATAGCAAGAAAGCTGAATTGTCGGCTTT 550 | |
| G V Y S V L L D S K K A E L S A L | |
| Sau3AI | |
| 551 AACTGAAGTTGCAGCGGTTCAAAATCCAACTTATATCACTCTTGATCAAA 600 | |
| T E V A A V Q N P T Y I T L D Q K | |
| 601 AAGGGCACCTCTACACTTGTGCTGCTGATGGAAATGGTGGTGGAATTGCT 650 | |
| G H L Y T C A A D G N G G G I A | |
| 651 GCCTTTGATTTCGATGGTCAAAATACAACTCACCTAGGGAATGTAACGAG 700 | |
| A F D F D G Q N T T H L G N V T S | |
| 701 TACTGGAGCCCCTTTGTGTTATGTGGCTGTTGATGAAGCACGTCAACTCG 750 | |
| T G A P L C Y V A V D E A R Q L V | |
| 751 TTTATGGTGCCAACTATCACTTGGGTGAAGTTCGTGTGTACAAAATTCAA 800 | |
| Y G A N Y H L G E V R V Y K I Q | |
| 801 GCTGATGGTTCCCTTAGATTAACCGATACAGTTAAACATAATGGTTCTGG 850 | |
| A D G S L R L T D T V K H N G S G | |
| 851 CCCTCGACCTGAGCAAGCAAGTTCTCATGTCCATTACTCTGATTTAACTC 900 | |
| P R P E Q A S S H V H Y S D L T P | |
| 901 CAGATGGTCGTCTTGTTACTTGTGATTTAGGTACAGATGAAGTGACTGTT 950 | |
| D G R L V T C D L G T D E V T V | |
| 951 TACGATGTTATTGGTGAAGGTAAACTCAATATCGTTACGATTTATCGTGC 1000 | |
| Y D V I G E G K L N I V T I Y R A | |
| 1001 CGAAAAAGGAATGGGAGCTCGTCACATCAGCTTCCATCCTAATGGAAAAA 1050 | |
| E K G M G A R H I S F H P N G K I | |
| 1051 TTGCTTATCTCGTCGGAGAATTAAATTCAACTATTGAAGTTCTAAGCTAT 1100 | |
| A Y L V G E L N S T I E V L S Y | |
| 1101 AATGAAGAAAAAGGACGATTCGCTCGTCTTCAAACAATCAGTACTTTACC 1150 | |
| N E E K G R F A R L Q T I S T L P | |
| 1151 TGAAGACTATCACGGAGCCAATGGAGTAGCTGCTATTCGAATTTCTTCTG 1200 | |
| E D Y H G A N G V A A I R I S S D | |
| 1201 ATGGTAAGTTCCTCTATGCTTCTAATCGTGGGCACGACTCTTTAGCAATT 1250 | |
| G K F L Y A S N R G H D S L A I | |
| 1251 TACAAGGTAAGTCCTCTCGGAACAAAATTAGAATCTATTGGTTGGACAAA 1300 | |
| Y K V S P L G T K L E S I G W T K | |
| 1301 GACTGAATATCATATTCCACGCGATTTTAATTTTAATAAAACCGAAGATT 1350 | |
| T E Y H I P R D F N F N K T E D Y | |
| 1351 ATATCATTGTCGCTCATCAAGAATCTGATAATTTAACTCTTTTCTTGAGA 1400 | |
| I I V A H Q E S D N L T L F L R | |
| 1401 GATAAAAATACAGGGTCATTAACGTTAGAACAAAAAGACTTTTACGCTCC 1450 | |
| D K N T G S L T L E Q K D F Y A P | |
| 1451 TGAAATTACTTGTGTTTTACCTTTGTAAAAACTAAACTTTAGTAAATCTT 1500 | |
| E I T C V L P L Stop | |
| 1501 GCTTTTGTTTTTTCACAAAGTTTTACTAAATCAGACAAAAAAATATTGCC 1550 | |
| 1551 AAATCTTTAAAAGGATTGGCAATATTTTTTTGTCTGAAACCCTTGCTTAT 1600 | |
| 1601 AAAGCGATTTCTAAAAGTTTGATGAGTTTTTTTGTAAATTTCATCACAAT 1650 | |
| 1651 ATCGCTTGACTTCTTTAAAAAACTTTGTTAAACTATTCACGTAAAAGAAA 1717 | |
| 1701 GTGAATGGAATCACAAAGGAGAACGTACACATATGGCAACTAAAAAAGCC 1750 | |
| adhE M A T K K A | (SEQ ID NO:31) |
| 1751 GCTCCAGCTGCAAAGAAAGTTTTAAGCGCTGAAGAAAAAGCCGCAAAATT 1800 | |
| A P A A K K V L S A E E K A A K F | |
| Sau3AI | |
| 1801 CCAAGGAAGTGTCGCTTATACTGATCAATTAGTCAAAAAAGCTCAAGCTG 1850 | |
| Q G S V A Y T D Q L V K K A Q A A | |
| 1851 CAGTTCTTAAATTTGAAGGATACACACAAACTCAAGTTGATACTATTGTT 1900 | |
| V L K F E G Y T Q T Q V D T I V | |
| 1901 GCTGCAATGGCTCTTGCAGCAAGCAAACATTCTCTGGAACTCGCTCACGA 1950 | |
| A A M A L A A S K H S L E L A H E | |
| 1951 AGCCGTTAATGAAACTGGCCGTGGAGTTGTTGAGGACAAAGATACAAAAA 2000 | |
| A V N E T G R G V V E D K D T K N | |
| 2001 ACCATTTTGCTTCTGAATCTGTTTATAATGCAATCAAAAATGATAAAACA 2050 | |
| H F A S E S V Y N A I K N D K T | |
| 2051 GTTGGCGTTATCGCTGAAAACAAAGTTGCTGGTTCTGTTGAAATCGCAAG 2100 | |
| V G V I A E N K V A G S V E I A S | |
| 2101 CCCCCTTGGAGTACTTGCTGGTATTGTCCCAACAACTAATCCAACATCAA 2150 | |
| P L G V L A G I V P T T N P T S T | |
| 2151 CAGCCATCTTTAAATCATTATTAACTGCAAAGACACGTAATGCTATTGTC 2200 | |
| A I F K S L L T A K T R N A I V | |
| 2201 TTTGCCTTTCACCCACAAGCACAAAAATGCTCAAGCCATGCGGCAAAAAT 2250 | |
| F A F H P Q A Q K C S S H A A K I | |
| 2251 TGTTTATGATGCTGCGATTGAAGCTGGTGCACCTGAAGACTTTATTCAAT 2300 | |
| V Y D A A I E A G A P E D F I Q W | |
| 2301 GGATTGAAGTACCCAGTCTTGATATGACGACTGCTTTGATTCAAAATAGA 2350 | |
| I E V P S L D M T T A L I Q N R | |
| 2351 GGAATTGCTACAATTCTTGCAACTGGTGGTCCAGGTATGGTCAATGCCGC 2400 | |
| G I A T I L A T G G P G M V N A A | |
| 2401 GCTTAAGTCTGGTAATCCTTCACTTGGTGTAGGTGCTGGTAATGGTGCAG 2450 | |
| L K S G N P S L G V G A G N G A V | |
| Sau3AI Sau3AI | |
| 2451 TTTATGTTGATGCAACTGCAAATATCGATCGTGCTGTTGAAGATCTTTTG 2500 | |
| Y V D A T A N I D R A V E D L L | |
| 2501 CTTTCAAAACGTTTTGATAACGGAATGATTTGTGCGACTGAAAACTCTGC 2550 | |
| L S K R F D N G M I C A T E N S A | |
| 2551 AGTTATTGATGCATCAATCTATGATGAATTTGTCGCTAAAATGCCAACGC 2600 | |
| V I D A S I Y D E F V A K M P T Q | |
| 2601 AAGGCGCTTATATGGTTCCTAAAAAAGATTACAAGGCAATTGAAAGTTTT 2650 | |
| G A Y M V P K K D Y K A I E S F | |
| 2651 GTTTTCGTTGAACGTGCTGGTGAAGGTTTTGGTGTAACTGGTCCTGTTGC 2700 | |
| V F V E R A G E G F G V T G P V A | |
| 2701 TGGTCGTTCTGGTCAATGGATTGCTGAACAAGCTGGTGTTAACGTCCCTA 2750 | |
| G R S G Q W I A E Q A G V N V P K | |
| 2751 AAGATAAAGATGTTCTTCTTTTTGAACTTGATAAGAAAAATATTGGGGAA 2800 | |
| D K D V L L F E L D K K N I G E | |
| 2801 GCTCTTTCTTCTGAAAAACTTTCTCCTTTGCTTTCAATCTACAAATCAGA 2850 | |
| A L S S E K L S P L L S I Y K S E | |
| 2851 AACACGTGAAGAAGGAATTGAAATTGTACGTAGCTTACTTGCTTACCAAG 2900 | |
| T R E E G I E I V R S L L A Y Q G | |
| 2901 GAGCTGGTCACAACGCTGCCATTCAAATCGGTGCAATGGACGACCCATTT 2950 | |
| A G H N A A I Q I G A M D D P F | |
| 2951 GTCAAAGAATACGGAATTAAAGTCGAAGCTTCTCGTATCCTCGTTAACCA 3000 | |
| V K E Y G I K V E A S R I L V N Q | |
| 3001 ACCTGACTCTATCGGTGGGGTCGGAGATATTTATACTGATGCAATGCGTC 3050 | |
| P D S I G G V G D I Y T D A M R P | |
| 3051 CATCATTGACGCTCGGAACTGGTTCATGGGGGAAAAATTCACTTTCACAC 3100 | |
| S L T L G T G S W G K N S L S H | |
| Sau3AI | |
| 3101 AATTTGAGTACATACGATCTATTGAATGTTAAAACAGTGGCTAAACGTCG 3150 | |
| N L S T Y D L L N V K T V A K R R | |
| 3151 TAATCGCCCTCAATGGGTTCGTTTGCCAAAAGAAATTTACTACGAAAAAA 3200 | |
| N R P Q W V R L P K E I Y Y E K N | |
| 3201 ATGCAATTTCTTACTTACAAGAATTGCCACACGTCCACAAAGCTTTCATT 3250 | |
| A I S Y L Q E L P H V H K A F I | |
| 3251 GTTGCCGACCCTGGTATGGTTAAATTCGGTTTCGTTGATAAAGTTTTGGA 3300 | |
| V A D P G M V K F G F V D K V L E | |
| 3301 ACAACTTGCTATCCGCCCAACTCAAGTTGAAACAAGCATTTATGGCTCAG 3350 | |
| Q L A I R P T Q V E T S I Y G S V | |
| 3351 TCCAACCTGACCCAACTTTGAGTGAAGCAATTGCAATCGCTCGTCAAATG 3400 | |
| Q P D P T L S E A I A I A R Q M | |
| 3401 AACCATTTTGAACCTGACACTGTCATCTGTCTTGGTGGTGGTTCTGCTCT 3450 | |
| N H F E P D T V I C L G G G S A L | |
| 3451 CGATGCTGGTAAGATTGGTCGTTTGATTTATGAATATGATGCTCGTGGTG 3500 | |
| D A G K I G R L I Y E Y D A R G E | |
| Sau3AI | |
| 3501 AGGCTGACCTTTCCGATGACGCAAGTTTGAAAGAGATCTTCCAAGAGTTA 3550 | |
| A D L S D D A S L K E I F Q E L | |
| 3551 GCTCAAAAATTTGTTGATATTCGTAAACGTATTATCAAATTCTACCACCC 3600 | |
| A Q K F V D I R K R I I K F Y H P | |
| 3601 ACACAAAGCACAAATGGTTGCTATCCCTACTACTTCTGGTACTGGTTCTG 3650 | |
| H K A Q M V A I P T T S G T G S E | |
| 3651 AAGTGACTCCATTTGCGGTTATCACTGATGATGAAACTCACGTTAAATAT 3700 | |
| V T P F A V I T D D E T H V K Y | |
| 3701 CCACTTGCTGACTATCAATTGACACCTCAAGTTGCCATTGTTGACCCTGA 3750 | |
| P L A D Y Q L T P Q V A I V D P E | |
| 3751 GTTTGTTATGACTGTACCAAAACGTACTGTTTCTTGGTCTGGGATTGATG 3800 | |
| F V M T V P K R T V S W S G I D A | |
| 3801 CTATGTCACACGCGCTTGAATCTTATGTTTCTGTCATGTCTTCTGACTAT 3850 | |
| M S H A L E S Y V S V M S S D Y | |
| 3851 ACAAAACCAATTTCACTTCAAGCCATCAAACTCATCTTTGAAAACTTGAC 3900 | |
| T K P I S L Q A I K L I F E N L T | |
| 3901 TGAGTCTTATCATTATGACCCAGCTCATCCAACCAAAGAAGGTCAAAAAG 3950 | |
| E S Y H Y D P A H P T K E G Q K A | |
| 3951 CTCGCGAAAACATGCACAATGCTGCAACACTCGCTGGTATGGCCTTCGCC 4000 | |
| R E N M H N A A T L A G M A F A | |
| 4001 AATGCTTTCCTTGGAATTAACCACTCACTTGCTCATAAAATTGCTGGTGA 4050 | |
| N A F L G I N H S L A H K I A G E | |
| 4051 ATTTGGGCTTCCTCATGGTCTTGCCATTGCTATCGCTATGCCACATGTCA 4100 | |
| F G L P H G L A I A I A M P H V I | |
| 4101 TTAAATTTAACGCTGTAACAGGAAACGTTAAATTTACCCCTTACCCACGT 4150 | |
| K F N A V T G N V K F T P Y P R | |
| 4151 TATGAAACTTATCGTGCGCAAGAAGACTACGCTGAAATTTCACGCTTCAT 4200 | |
| Y E T Y R A Q E D Y A E I S R F M | |
| 4201 GGGATTTGCTGGCAAAGAAGATTCAGATGAAAAAGCGGTCAAAGCTTTGG 4250 | |
| G F A G K E D S D E K A V K A L V | |
| 4251 TTGCTGAACTTAAAAAATTGACTGATAGTATTGATATTAATATCACCCTT 4300 | |
| A E L K K L T D S I D I N I T L | |
| 4301 TCAGGAAATGGTGTAGATAAAGCTCATCTTGAACGTGAGCTTGATAAATT 4350 | |
| S G N G V D K A H L E R E L D K L | |
| 4351 GGCTGACCTTGTTTACGATGACCAATGTACACCTGCTAATCCACGTCAAC 4400 | |
| A D L V Y D D Q C T P A N P R Q P | |
| 4401 CAAGAATTGATGAGATTAAACAACTCTTGTTAGACCAATATTAATATATT 4450 | |
| R I D E I K Q L L L D Q Y Stop | |
| 4451 AATTATAGTATTTGGAACCGAACGATATCCATGCTCGCTAACCTGCTAAA 4500 | |
| 4501 GCAGGAAGTCGCAATGGTACGTCAACCAAGAATTGATGAGATTAAACAAC 4550 | |
| Sau3AI | |
| 4551 TCTTGTTAGATCAATACTAATAATCTGTTGATAAAAATAATTAAAACGCT 4600 | |
| 4601 CTGATGAATTCGTCAGAGCGTTTTTTATTATAGCTTATACAACTATCAAA 4650 | |
| 4651 AGGTATAAATCAATTTCGATATAGGCTCTTTTCACTCCATTGATTTATAT 4700 | |
| Sau3AI | |
| 4701 TATATAAAAATCAATAATTAATTAGCGATAGAAGTGATCC 4741 | |

### EXAMPLE 2

1. Construction of L. lactis DB1341 and MG1363 adhE Mutant Strains by Gene Inactivation

Inactivation of the adhE gene of strain DB1341 was carried out by Campbell-like integration (Leenthous et al., 1991) of pSMA-500 derivatives into the DB1341 chromosome. The adhE gene of strain DB1341 was inactivated at two different positions by cloning of PCR fragments (see FIG. 2) into the integration vector pSMA500 (Madsen et al., 1996). A 706 bp internal adhE fragment was amplified from the DB1341 chromosome using-primer adhP1 (position 1069-1088 in Table 1.4) and primer adhP2 (position 1775-1756 in Table 1.4). These primers contain a XhoI and a BamHI recognition site at the 5′ end. The PCR fragment was digested with XhoI and BamHI followed by cloning into pSMA500. The resulting plasmid, pSMAKAS4 (FIG. 3), was introduced into E. coli MC1000 by electroporation (Sambrook et al., 1989). Plasmid pSMAKAS4 was purified and subsequently introduced into strain DB1341 by electroporation (Holo and Nes 1989) and transformants were selected on SGM17 plates containing 1 μg/ml erythromycin and 80 μg/ml X-gal (Madsen et al., 1996). Homologous integration leads to an adhE gene which is interrupted after amino acid residue Asp⁵⁴³. About 100 blue transformants were obtained, indicating that a transcriptional fusion of the adhE gene to the lacLM reporter gene of pSMA500 had occurred. Eight blue transformants were restreaked and the integration point was verified by PCR analysis. One strain, DBKAS4, was selected for further studies.

Another integration further downstream in the adhE gene was constructed by a similar strategy. A 616 bp adhE fragment was amplified from the DB1341 chromosome using primer orf3P1 (position 2112-2138 in Table 1.4) and primer orf3P2 (position 2728-2708 in Table 1.4). The cloning of this fragment into pSMA500 resulted in plasmid pSMAKAS5 (FIG. 3). Introduction of pSMAKAS5 into DB1341 and subsequent integration into the adhE gene leads to an adhE gene, which is interrupted after amino acid residue Ile⁸⁶¹. About 400 blue transformants were obtained, which again indicated that a transcriptional fusion of the adhE gene to the lacLM reporter gene of pSMA500 had occurred. Eight blue transformants were restreaked and the integration point was verified by PCR analysis. One strain, DBKAS5, was selected for further studies.

pSMAKAS4 and pSMAKAS5 were used also to inactivate the MG1363 adhE gene. One transformant from each transformation that turned blue on X-gal plates (MGKAS4 and MGKAS5), and therefore contained a translational fusion of the lacLM reporter gene of pSMA500 to the MG1363 adhE gene, was isolated for further studies.

A sample of Lactococcus lactis subspecies lactis biovar diacetylactis strains DBKAS4 and DBKAS5, respectively and of Lactococcus lactis subspecies lactis strains MGKAS4 and MGKAS5, respectively were deposited under the Budapest Treaty with the German Collection of Microorganisms and Cell Cultures, Mascheroder Weg 1b, D-38 124 Braunschweig, Germany on 18 Jul. 1996 under the accession Nos DSM 11084, DSM 11085, DSM 11081 and DSM 11082, respectively.

A further adhE mutant strain was obtained by PCR using MG1363 DNA as template and primers adhP1-XhoI (sequence 5′-GGCCGCTCGAGGTTGAACGTGCTGGTGAAGG-3′; spanning position 2657-2676 in the MG1363 adhE sequence) (SEQ ID NO:32) and adhP2-BamHI (sequence 5′-TAGTAGGATCCGGGTCAGGTTGGACTGAGCC-3′; spanning position 3363-3344 in the MG1363 adhE sequence) (SEQ ID NO:33). A 700 bp fragment was digested with XhoI and BamHI, cloned into likewise digested pSMA500 and transformed into E. coli MC1000. The new construction, pSMAKAS14 was introduced into L. lactis MG1363 via electroporation. Integration led to disruption of the resident adhE gene and one transformant that turned blue on X-gal plates (integration results in transcriptional fusion to lacLM, a reporter gene) was selected for further analysis and was named MGKAS14. This integrant should express an AdhE protein truncated at position Asp⁵⁴³.

A sample of MGKAS14 was deposited under the Budapest Treaty with the German Collection of Microorganisms and Cell Cultures, Mascheroder Weg 1b, D-38 124 Braunschweig, Germany on 10 Jul. 1997 under the accession No. DSM 11654.

2. Physiological Characterization of MGKAS14

Physiological studies of MGKAS14 was carried by cultivating the strain in anaerobiosis in M17 medium supplemented with either glucose (GM17) or galactose (GalM17). The production under these conditions of the metabolites formate, acetaldehyde and pyruvate, respectively was measured and compared to corresponding measurement for the wild type strain, cultivated under similar conditions. In GM17 the production of formate in the mutant strain was reduced (4.86 in GM1363 vs. 1.67 in MGKAS14), the production of acetaldehyde was increased (0.52 in MG1363 vs. 0.67 in MGKAS14). No pyruvate was detected with any of the test strains. In the GalM17 medium, the production of formate was reduced substantially in the mutant strain (39.11 in GM1363 vs. 4.39 in MGKAS14) and that of acetaldehyde increased (0.67 in MG1363 vs. 1.12 in MGKAS14). None of the strains produced pyruvate.

### EXAMPLE 3

### Cloning of the L. lactis pfl Gene

The sequence of the pfl gene encoding pyruvate formate-lyase, a key enzyme in anaerobic metabolism, has only been reported in a few bacteria. DNA sequence homology between the different bacterial pfl genes is limited, making it difficult to clone this gene from other organisms (Table 3.1). Recently, this gene has been cloned in Streptococcus mutans (Yamamoto et al., 1996). The S. mutans pfl gene encodes a 775 amino acid protein as deduced from the published DNA sequence.

TABLE 3.1
Homology (DNA and protein level) of the L. lactis
pfl with other bacterial pfl genes

| | Homology to the | | | |
|---|---|---|---|---|
| | L. lactis Pfl protein | | | |
| Pfl protein | Identity | Similarity | DNA homology^{a} | Organism |
| 759 aa | 42.2% | 73% | 55.1% | E. coli |
| 769 aa | 42.1% | 76% | 55.4% | H. influenzae |
| 740 aa | NA | NA | 52.6% | C. pasteurianum |
| 775 aa | NA | NA | 71.8% | S. mutans |

^{a}DNA homology through the L. lactis pfl sequence obtained.
NA: not submitted to the databases;
NF: not found in database searches.

1. Construction of Lactococcus lactis λZAP Genomic Libraries

λZAP genomic libraries of L. lactis strains DB1341 and MG1363 were constructed according to the manufacturer's instructions (Stratagene) using partially Sau3AI-digested chromosomal DNA (average size about 5 kb) cloned into λ vector BamHI arms. Average insert size was estimated to be 3 kb.

2. Screening of a λZAP Genomic Library of Strain DB1341 with a S. mutans pfl Probe

A 1 kb EcoRI fragment from the S. mutans pfl gene, encompassing positions 1190-2213 of the published S. mutans sequence (codons 298-639 of the pfl gene) was randomly labelled and used for screening the λZAP genomic library of strain DB1341 (approximately 2×10⁵ pfu; Sambrook et al., 1989). Filters were washed at low stringency (2×30 min at room temperature in 5×SSC, then 1×30 min at 65° C. in 3×SSC; 0.1% SDS), and two positive clones, pfl1 and pfl2 were identified.

A sample of an E. coli strain transformed with clone pfl1 was deposited under the Budapest Treaty with the German Collection of Microorganisms and Cell Cultures, Mascheroder Weg 1b, D-38 124 Braunschweig, Germany on 25 Jul. 1996 under the accession Nos DSM 11103.

3. Sequencing Positive λZAP Clones and Identification of Clones Containing a pfl Fragment

Following in vivo excision (Stratagene) and plasmid DNA isolation, sequence analysis (ALF sequenator, Pharmacia) was carried out for pfl1 using T7 and T3 primers (Stratagene). Approximately 2.1 kb was sequenced from one end of clone pfl1 (from position 1342 in Table 3.2 below), and a truncated, uninterrupted ORF spanning 1.1 kb was found that showed significant homology to other pfl genes, both at the DNA and protein level (Tables 3.3 and 3.4). A putative rho-independent transcription terminator (de Vos and Simons 1994) is located 26 bp downstream of the stop codon (positions 2468-2490 in Table 3.2).

TABLE 3.2

Sequence of the L. lactis DB1341 pf1 gene
The coding sequence starts at position 80 and ends at position 2443.
A putative ribosome binding site is shown in bold, double underline
(positions 65-71). A putative rho-independent transcriptional terminator
(de Vos and Simons 1994) is found at positions 2468-2490 and
is shown in bold, underline (stem) or dotted underline (loop).

| | | | |
|---|---|---|---|
| | E | | |
| | c | | |
| | o | | |
| | R | | |
| | I | | |
| | GAATTCTGTTTGCTATTCTCAAACTGTATGATATAATGAAGTTGTAATTT | (SEQ ID NO:15) | |
| 1 | ---------+---------+---------+---------+---------+ | 50 | |
| | GAAACAGAAAGAACAAAGGAGATTTCAAAATGAAAACCGAAGTTACGGAA | | |
| 51 | ---------+---------+---------+---------+---------+ | 100 | |
| | MetLysThrGluValThrGlu | - | (SEQ ID NO:16) |
| | AATATCTTTGAACAAGCTTGGGATGGTTTTAAAGGAACCAACTGGCGCGA | | |
| 1 | ---------+---------+---------+---------+---------+ | 150 | |
| | AsnIlePheGluGlnAlaTrpAspGlyPheLysGlyThrAsnTrpArgAsp | - | |
| | TAAAGCAAGCGTTACTCGCTTTGTACAAGAAAACTACAAACCATATGATG | | |
| 151 | ---------+---------+---------+---------+---------+ | 200 | |
| | LysAlaSerValThrArgPheValGlnGluAsnTyrLysProTyrAspGly | - | |
| | GTGATGAAAGCTTTCTTGCTGGGCCAACAGAACGTACACTTAAAGTAAAG | | |
| 201 | ---------+---------+---------+---------+---------+ | 250 | |
| | AspGluSerPheLeuAlaGlyProThrGluArgThrLeuLysValLys | - | |
| | AAAATTATTGAAGATACAAAAAATCACTACGAAGAAGTAGGATTTCCCTT | | |
| 251 | ---------+---------+---------+---------+---------+ | 300 | |
| | LysIleIleGluAspThrLysAsnHisTyrGluGluValGlyPheProPhe | - | |
| | CGATACTGACCGCGTAACCTCTATTGATAAAATCCCTGCTGGATATATCG | | |
| 301 | ---------+---------+---------+---------+---------+ | 350 | |
| | AspThrAspArgValThrSerIleAspLysIleProAlaGlyTyrIleAsp | - | |
| | ATGCTAATGATAAAGAACTTGAACTCATCTATGGGATGCAAAATAGCGAA | | |
| 351 | ---------+---------+---------+---------+---------+ | 400 | |
| | AlaAsnAspLysGluLeuGluLeuIleTyrGlyMetGlnAsnSerGlu | - | |
| | CTTTTCCGCTTGAATTTCATGCCAAGAGGTGGACTTCGTGTTGCTGAAAA | | |
| 401 | ---------+---------+---------+---------+---------+ | 450 | |
| | LeuPheArgLeuAsnPheMetProArgGlyGlyLeuArgValAlaGluLys | - | |
| | GATTTTGACAGAACACGGTCTCTCAGTTGACCCAGGCTTGCATGATGTTT | | |
| 451 | ---------+---------+---------+----------+--------+ | 500 | |
| | IleLeuThrGluHisGlyLeuSerValAspProGlyLeuHisAspValLeu | - | |
| | TGTCACAAACAATGACTTCTGTAAATGATGGAATCTTTCGTGCTTATACT | | |
| 501 | ---------+---------+---------+---------+---------+ | 550 | |
| | SerGlnThrMetThrSerValAsnAspGlyIlePheArgAlaTyrThr | - | |
| | TCAGCAATTCGTAAAGCACGTCATGCTCATACTGTAACAGGTTTGCCAGA | | |
| 551 | ---------+---------+---------+---------+---------+ | 600 | |
| | SerAlaIleArgLysAlaArgHisAlaHisThrValThrGlyLeuProAsp | - | |
| | TGCTTACTCTCGTGGACGTATCATTGGTGTCTATGCACGTCTTGCCCTTT | | |
| 601 | ---------+---------+---------+---------+---------+ | 650 | |
| | AlaTyrSerArgGlyArgIleIleGlyValTyrAlaArgLeuAlaLeuTyr | - | |
| | ACGGTGCTGATTACCTTATGAAGGAAAAAGCAAAAGAATGGGATGCAATC | | |
| 651 | ---------+---------+---------+---------+---------+ | 700 | |
| | GlyAlaAspTyrLeuMetLysGluLysAlaLysGluTrpAspAlaIle | - | |
| | ACTGAAATTAACGAAGAAAACATTCGTCTTAAAGAAGAAATTAATATGCA | | |
| 701 | ---------+---------+---------+---------+---------+ | 750 | |
| | ThrGluIleAsnGluGluAsnIleArgLeuLysGluGluIleAsnMetGln | - | |
| | ATACCAAGCTTTGCAAGAAGTTGTAAACTTTGGTGCCTTATATGGTCTTG | | |
| 751 | ---------+---------+---------+---------+---------+ | 800 | |
| | TyrGlnAlaLeuGlnGluValValAsnPheGlyAlaLeuTyrGlyLeuAsp | - | |
| | ATGTTTCACGTCCAGCTATGAACGTAAAAGAAGCAATCCAATGGGTTAAC | | |
| 801 | ---------+---------+---------+---------+---------+ | 850 | |
| | ValSerArgProAlaMetAsnValLysGluAlaIleGlnTrpValAsn | - | |
| | ATCGCTTATATGGCAGTATGTCGTGTCATTAATGGAGCTGCAACTTCACT | | |
| 851 | ---------+---------+---------+---------+---------+ | 900 | |
| | IleAlaTyrMetAlaValCysArgValIleAsnGlyAlaAlaThrserLeu - | | |
| | TGGACGTGTTCCAATCGTTCTTGATATCTTTGCAGAACGTGACCTTGCTC | | |
| 902 | ---------+---------+---------+---------+---------+ | 950 | |
| | GlyArgValProIleValLeuAspIlePheAlaGluArgAspLeuAlaArg | - | |
| | GTGGAACATTTACTGAACAAGAAATTCAAGAATTTGTTGATGATTTCGTT | | |
| 951 | ---------+---------+---------+---------+---------+ | 1000 | |
| | GlyThrPheThrGluGlnGluIleGlnGluPheValAspAspPheVal | - | |
| | TTGAAGCTTCGTACAATGAAATTTGCTCGTGCAGCTGCTTATGATGAACT | | |
| 1001 | ---------+---------+---------+---------+---------+ | 1050 | |
| | LeuLysLeuArgThrMetLysPheAlaArgAlaAlaAlaTyrAspGluLeu | - | |
| | TTATTCTGGTGACCCAACATTCATCACAACATCTATGGCTGGTATGGGTA | | |
| 1051 | ---------+---------+---------+---------+---------+ | 1100 | |
| | TyrSerGlyAspProThrPheIleThrThrSerMetAlaGlyMetGlyAsn | - | |
| | ATGACGGACGTCACCGTGTCACTAAAATGGACTACCGTTTCTTGAACACA | | |
| 1101 | ---------+---------+---------+---------+---------+ | 1150 | |
| | AspGlyArgHisArgValThrLysMetAspTyrArgPheLeuAsnThr | - | |
| | CTTGATACAATCGGAAATGCTCCAGAACCAAACTTGACAGTCCTTTGGGA | | |
| 1151 | ---------+---------+---------+---------+---------+ | 1200 | |
| | LeuAspThrIleGlyAsnAlaProGluProAsnLeuThrValLeuTrpAsp | - | |
| | TTCTAAACTTCCTTACTCATTCAAACGTTATTCAATGTCTATGAGCCACA | | |
| 1201 | ---------+---------+---------+---------+---------+ | 1250 | |
| | SerLysLeuProTyrSerPheLysArgTyrSerMetSerMetSerHisLys | - | |
| | AGCATTCTTCTATTCAATATGAAGGTGTTGAAACAATGGCTAAAGATGGA | | |
| 1251 | ---------+---------+---------+---------+---------+ | 1300 | |
| | HisSerSerIleGlnTyrGluGlyValGluThrMetAlaLysAspGly | - | |
| | S | | |
| | a | | |
| | u | | |
| | 3 | | |
| | A | | |
| | I | | |
| | TATGGCGAAATGTCATGTATCTCTTGTTGTGTCTCACCACTTGATCCAGA | | |
| 1301 | ---------+---------+---------+---------+---------+ | 1350 | |
| | TyrGlyGluMetSerCysIleSerCysCysValSerProLeuAspProGlu | - | |
| | AAATGAAGAAGGACGTCATAACCTCCAATACTTTGGTGCGCGTGTAAACG | | |
| 1351 | ---------+---------+---------+---------+---------+ | 1400 | |
| | AsnGluGluGlyArgHisAsnLeuGlnTyrPheGlyAlaArgValAsnVal | - | |
| | TCTTGAAAGCAATGTTGACTGGTTTGAACGGTGGTTATGATGACGTTCAT | | |
| 1401 | ---------+---------+---------+---------+---------+ | 1450 | |
| | LeuLysAlaMetLeuThrGlyLeuAsnGlyGlyTyrAspAspValHis | - | |
| | AAAGATTATAAAGTATTCGACATCGAACCTGTTCGTGACGAAATTCTTGA | | |
| 1451 | ---------+---------+---------+---------+---------+ | 1500 | |
| | LysAspTyrLysValPheAspIleGluProValArgAspGluIleLeuAsp | - | |
| | CTATGATACAGTTATGGAAAACTTTGACAAATCTCTCGACTGGTTGACTG | | |
| 1501 | ---------+---------+---------+---------+---------+ | 1550 | |
| | TyrAspThrValMetGluAsnPheAspLysSerLeuAspTrpLeuThrAsp | - | |
| | ATACTTATGTTGATGCAATGAATATCATTCATTACATGACTGATAAATAT | | |
| 1551 | ---------+---------+---------+---------+---------+ | 1600 | |
| | ThrTyrValAspAlaMetAsnIleIleHisTyrMetThrAspLysTyr | - | |
| | AACTATGAAGCAGTTCAAATGGCCTTCTTGCCTACTAAAGTTCGTGCTAA | | |
| 1601 | ---------+---------+---------+---------+---------+ | 1650 | |
| | AsnTyrGluAlaValGlnMetAlaPheLeuProThrLysValArgAlaAsn | - | |
| | CATGGGATTTGGTATCTGTGGATTCGCAAATACAGTTGATTCACTTTCAG | | |
| 1651 | ---------+---------+---------+---------+---------+ | 1700 | |
| | MetGlyPheGlyIleCysGlyPheAlaAsnThrValAspSerLeuSerAla | - | |
| | CAATTAAATATGCTAAAGTTAAAACATTGCGTGATGAAAATGGCTATATC | | |
| 1701 | ---------+---------+---------+---------+---------+ | 1750 | |
| | IleLysTyrAlaLysValLysThrLeuArgAspGluAsnGlyTyrIle | - | |
| | S | | |
| | a | | |
| | u | | |
| | 3 | | |
| | A | | |
| | I | | |
| | TACGATTACGAAGTAGAAGGTGATTTCCCTCGTTATGGTGAAGATGATGA | | |
| 1751 | ---------+---------+---------+---------+---------+ | 1800 | |
| | TyrAspTyrGluValGluGlyAspPheProArgTyrGlyGluAspAspAsp | - | |
| | TCGTGCTGATGATATTGCTAAACTTGTCATGAAAATGTACCATGAAAAAT | | |
| 1801 | ---------+---------+---------+---------+---------+ | 1850 | |
| | ArgAlaAspAspIleAlaLysLeuValMetLysMetTyrHisGluLysLeu | - | |
| | TAGCTTCACACAAACTTTACAAAAATGCTGAAGCTACTGTTTCACTTTTG | | |
| 1851 | ---------+---------+---------+---------+---------+ | 1900 | |
| | AlaSerHisLysLeuTyrLysAsnAlaGluAlaThrValSerLeuLeu | - | |
| | ACAATTACATCTAACGTTGCTTACTCTAAACAAACTGGTAATTCTCCAGT | | |
| 1901 | ---------+---------+---------+---------+---------+ | 1950 | |
| | ThrIleThrSerAsnValAlaTyrSerLysGlnThrGlyAsnSerProVal | - | |
| | ACATAAAGGAGTATTCCTCAATGAAGATGGTACAGTAAATAAATCTAAAC | | |
| 1951 | ---------+---------+---------+---------+---------+ | 2000 | |
| | HisLysGlyValPheLeuAsnGluAspGlyThrValAsnLysSerLysLeu | - | |
| | E | | |
| | c | | |
| | o | | |
| | R | | |
| | I | | |
| | TTGAATTCTTCTCACCAGGTGCTAACCCATCTAATAAAGCTAAGGGTGGT | | |
| 2001 | ---------+---------+---------+---------+---------+ | 2050 | |
| | GluPhePheSerProGlyAlaAsnProSerAsnLysAlaLysGlyGly | - | |
| | E | | |
| | c | | |
| | o | | |
| | R | | |
| | I | | |
| | TGGTTGCAAAACCTTCGCTCATTGGCTAAGTTGGAATTCAAAGATGCAAA | | |
| 2051 | ---------+---------+---------+---------+---------+ | 2100 | |
| | TrpLeuGlnAsnLeuArgSerLeuAlaLysLeuGluPheLysAspAlaAsn | - | |
| | TGATGGTATTTCATTGACTACTCAAGTTTCACCTCGTGCACTTGGTAAAA | | |
| 2101 | ---------+---------+---------+---------+---------+ | 2150 | |
| | AspGlyIleSerLeuThrThrGlnValSerProArgAlaLeuGlyLysThr | - | |
| | CTCGTGATGAACAAGTGGATAACTTGGTTCAAATTCTTGATGGATACTTC | | |
| 2151 | ---------+---------+---------+---------+---------+ | 2200 | |
| | ArgAspGluGlnValAspAsnLeuValGlnIleLeuAspGlyTyrPhe | - | |
| | ACACCAGGTGCTTTGATTAATGGTACTGAATTTGCAGGTCAACACGTTAA | | |
| 2201 | ---------+---------+---------+---------+---------+ | 2250 | |
| | ThrProGlyAlaLeuIleAsnGlyThrGluPheAlaGlyGlnHisValAsn | - | |
| | CTTGAACGTAATGGACCTTAAAGATGTTTACGATAAAATCATGCGTGGTG | | |
| 2252 | ---------+---------+---------+---------+---------+ | 2300 | |
| | LeuAsnValMetAspLeuLysAspValTyrAspLysIleMetArgGlyGlu | - | |
| | AAGATGTTATCGTTCGTATCTCTGGTTACTGTGTCAATACTAAATACCTC | | |
| 2301 | ---------+---------+---------+---------+---------+ | 2350 | |
| | AspValIleValArgIleSerGlyTyrCysValAsnThrLysTyrLeu | - | |
| | ACACCAGAACAAAAACAAGAATTAACTGAACGTGTCTTCCATGAAGTTCT | | |
| 2351 | ---------+---------+---------+---------+---------+ | 2400 | |
| | ThrProGluGlnLysGlnGluLeuThrGluArgValPheHisGluValLeu | - | |
| | TTCAAACGATGATGAAGAAGTAATGCATACTTCAAACATCTAATTCTTAA | | |
| 2401 | ---------+---------+---------+---------+---------+ | 2450 | |
| | SerAsnAspAspGluGluValMetHisThrSerAsnIleEnd | | |
| | AATTTAATGAATATTCGGTCTGTCAGTTTTACTGACAGACTTTTTTTTAC | | |
| 2451 | ---------+---------+------\overline{---}+---------+---------+ | 2500 | |
| | GAAAAAATTAATCATAATAGTTAAAAACTATTGTTTTTAGTTTAAGAAAG | | |
| 2501 | ---------+---------+---------+---------+---------+ | 2550 | |
| | TTAAATTTTATGCTAAAATAGATGAATGAAAATGGTAATTGGATTGACAG | | |
| 2551 | ---------+---------+---------+---------+---------+ | 2600 | |
| | GCGGAATTGCGATGGGAAATCAACGGTGGTTGATTTTTTGATTCTGAGGG | | |
| 2601 | ---------+---------+---------+---------+---------+ | 2650 | |
| | TTATCAAGTGATTGATGCTGACAAAGTTGTCCGTCAATTTACAAGAACCT | | |
| 2651 | ---------+---------+---------+---------+---------+ | 2700 | |
| | GGCGGAAAACTTTACAAGGCAATATTAGAAACTTACGGTTTAGATTTTAT | | |
| 2701 | ---------+---------+---------+---------+---------+ | 2750 | |
| | TGCTGACAATTGGACAGTTAAATCGTGAAAAATTAGGAGCTTTAGTTTTT | | |
| 2751 | ---------+---------+---------+---------+---------+ | 2800 | |
| | TCTGATTCAAAAGAGCGCGAGAAATTATCAAACTTACAAGATGAAATTAT | | |
| 2801 | ---------+---------+---------+---------+---------+ | 2850 | |
| | TCGTACAGAATTATATGATAGACGTGATGACTTATTAAAAAAAATGACTG | | |
| 2851 | ---------+---------+---------+---------+---------+ | 2900 | |
| | ACAAGTCTGTCAGTAAAAATTTTGATTCAAAGAGTCAAGGAAAAAATCTG | | |
| 2901 | ---------+---------+---------+---------+---------+ | 2950 | |
| | TCAGTAAATAAGCCAATATTTATGGATATTCCGTTATTAATTGAATACAA | | |
| 2951 | ---------+---------+---------+---------+---------+ | 3000 | |
| | TTATACCGGATTTGATGAAATATGGTTGGTCAGCTTACCTGAAAAAATAC | | |
| 3001 | ---------+---------+---------+---------+---------+ | 3050 | |
| | AATTAGAAAGACTGATGGCAAGAAATAAGTTTACGGAAGAAGAAGCTAAA | | |
| 3051 | ---------+---------+---------+---------+---------+ | 3100 | |
| | AAACGAATTTCTTCACAAATGCCATTGTCAGAAAAACAAAAAGTCGCTGA | | |
| 3101 | ---------+---------+---------+---------+---------+ | 3150 | |
| | TGTCATTCTGGATAATTCTGGAAAGATTGAAGCACTAAAAAAACAAATCC | | |
| 3151 | ---------+---------+---------+---------+---------+ | 3200 | |
| | AGCGAGAACTAGCTAGGATAGAAGAACAGAAATAGAGGTGAATCGCACGA | | |
| 3201 | ---------+---------+---------+---------+---------+ | 3250 | |
| | AAACAGTTAATTGGAAAGGAATTTATTTATAACATGGATTGGCTGCTTTT | | |
| 3251 | ---------+---------+---------+---------+---------+ | 3300 | |
| | TTGTAGGTTCATCATTTTCACTCGTCATGCCTTTCTCCCCTTGTATATTC | | |
| 3301 | ---------+---------+---------+---------+---------+ | 3350 | |
| | AAGGACTGGGTGAAGCGGTGGGAATTTGAACTTTACTCAGGGTTACTTTT | | |
| 3351 | ---------+---------+---------+---------+---------+ | 3400 | |
| | TCTTTGCCAGCCTTA | | |
| 3401 | ---------+----- 3415 | | |

TABLE 3.3

DNA homology (FASTA, GCG Wisconsin Package Version 8, Genetics Computer Group)
using the complete L. lactis DB1341 pf1 sequence shown in TABLE 3.2
Only the two highest scores (S. mutans and H. influenzae pf1 genes, designated
smpf1 and hi3281, respectively) are shown.

```
(Nucleotide) FASTA of: dbpf1.seq from: 1 to: 3415 Jul. 19, 1996 10:11
The best scores are: init1 initn opt . . .
empro: smpf1 D50491 Streptococcus mutans pf1 . . . 4335 5345 4996
empro: hi32812/rev U32812 Hae. influenzae focA 652 1077 1299
empro: ecpf1 X08035 E. coli pf1 (pyruvate form. 429 735 1214
empro: cppf1act X93463 C. pasteurianum pf1 and act 309 487 744
emnew: cef13b12/rev Z70683 Caenorhabditis eleg. 94 168 128
dbpf1.seq
empro: smpf1
ID SMPFL standard; DNA; PRO; 3067 BP.
AC D50491;
NI g1129081
DT 23 DEC. 1995 (Rel. 46, Created)
DE Streptococcus mutans pf1 gene for pyruvate formate-lyase . . . .
SCORES Init1: 4335 Initn: 5345 Opt: 4996
                                        71.8% identity in 2608 bp overlap
                                              10 20 30 40 50
dbpf1. GAATTCTGTTTGCTATTCTCAAACTGTATGATATAATGAAGTTGTAATTTGA
                                                                          ||||||||||| ||| | | |||| |||
smpf1 AAGCAAGTTCTTTCGCTTGTGTAACCGGTTACTGTATGATAGAATATAATCGTAAATTGT
          200 210 220 230 240 250
                                                60 70 80 90
dbpf1. AACAGA-----------AAGAACAAAGGAGATTTCAA-AATGAAAAC---CGAAGTTACG
              |||||| |||| | ||| || |||| ||| ||| | || ||
smpf1 AACAGATTAACTGTTACTAGAATAGAGGGGAACTCAATTATGGCAACTGTCAAAACTAAC
          260 270 280 290 300 310
              100 110 120 130 140 150
dbpf1. GAAAATATCTTTGAACAAGCTTGGGATGGTTTTAAAGGAACCAACTGGCGCGATAAAGCA
                      | | |||||| |||| ||||| || ||||||||||| ||||| || | ||||
smpf1 ACTGACGTTTTTGAAAAAGCCTGGGAAGGCTTTAAAGGAACTGACTGGAAAGACAGAGCA
          320 330 340 350 360 370
              160 170 180 190 200 210
dbpf1. AGCGTTACTCGCTTTGTACAAGAAAACTACAAACCATATGATGGTGATGAAAGCTTTCTT
              ||| || |||||||||| ||||| ||||||| |||||||| || | ||||| ||||||
smpf1 AGCATTTCTCGCTTTGTTCAAGACAACTACACTCCATATGACGGAGGCGAAAGTTTTCTT
          380 390 400 410 420 430
              220 230 240 250 260 270
dbpf1. GCTGGGCCAACAGAACGTACACTTAAAGTAAAGAAAATTATTGAAGATACAAAAAATCAC
              || || || || |||||| ||||| | | || ||| | | ||||| || ||| ||
smpf1 GCCGGCCCTACTGAACGTTCACTTCACATCAAAAAAGTCGTAGAAGAAACTAAAGCGCAT
          440 450 460 470 480 490
              280 290 300 310 320 330
dbpf1. TACGAAGAAGTAGGATTTCCCTTCGATACTGACCGCGTAACCTCTATTGATAAAATCCCT
              ||||||||| | | ||||| | ||||| || | || ||||||| | | |||||
smpf1 TACGAAGAAACACGTTTTCCAATGGATAC---ACGTATTACATCTATTGCTGATATCCCA
          500 510 520 530 540 550
              340 350 360 370 380 390
dbpf1. GCTGGATATATCGATGCTAATGATAAAGAACTTGAACTCATCTATGGGATGCAAAATAGC
              || || ||||| ||| || ||| |||| | || | ||| || |||||
smpf1 GCAGGTTATAT---------TGACAAGGAAAATGAATTGATTTTTGGTATCCAAAACGAT
                560 570 580 590 600
              400 410 420 430 440 450
dbpf1. GAACTTTTCCGCTTGAATTTCATGCCAAGAGGTGGACTTCGTGTTGCTGAAAAGATTTTG
              |||||||| |||| |||||||||| ||| || |||| | ||||||| ||||
smpf1 GAACTTTTTAAGCTGAACTTCATGCCAAAAGGCGGTATTCGCATGGCTGAAACAGCTTTG
              610 620 630 640 650 660
              460 470 480 490 500 510
dbpf1. ACAGAACACGGTCTCTCAGTTGACCCAGGCTTGCATGATGTTTTGTCACAAACAATG--A
              | |||||| ||| | ||||| | | | ||||| | | | || || ||| |
smpf1 AAAGAACATGGTTATGAACCAGACCCTGCCGTTCATGAAATCT--TTACCAAATATGCAA
              670 680 690 700 710 720
                  520 530 540 550 560 570
dbpf1. CTTCTGTAAATGATGGAATCTTTCGTGCTTATACTTCAGCAATTCGTAAAGCACGTCATG
              | | || |||||||| |||||||||||||| |||||| ||||| ||||||||||
smpf1 CAACCGTTAATGATGGTATCTTTCGTGCTTACACTTCAAACATTCGCCGTGCACGTCATG
                  730 740 750 760 770 780
                  580 590 600 610 620 630
dbpf1. CTCATACTGTAACAGGTTTGCCAGATGCTTACTCTCGTGGACGTATCATTGGTGTCTATG
              | || |||||||| ||| | |||||||| |||||||| |||||||| ||||| || ||||
smpf1 CCCACACTGTAACTGGTCTCCCAGATGCATACTCTCGCGGACGTATTATTGGAGTTTATG
                  790 800 810 820 830 840
                  640 650 660 670 680 690
dbpf1. CACGTCTTGCCCTTTACGGTGCTGATTACCTTATGAAGGAAAAAGCAAAAGAATGGGATG
              | |||||||| || || |||||||| ||| | ||| | ||||||| || || ||| |
smpf1 CCCGTCTTGCTCTCTATGGTGCTGACTACTTGATGCAAGAAAAAGTGAACGACTGGAACT
                  850 860 870 880 890 900
                  700 710 720 730 740 750
dbpf1. CAATCACTGAAATTAACGAAGAAAACATTCGTCTTAAAGAAGAAATTAATATGCAATACC
              |||| |||||||| | |||||| ||||||||| |||||||| ||| | ||||| |
smpf1 CAATTGCTGAAATTGATGAAGAATCAATTCGTCTTCGTGAAGAAATCAATCTTCAATATC
                  910 920 930 940 950 960
                  760 770 780 790 800 810
dbpf1. AAGCTTTGCAAGAAGTTGTAAACTTTGGTGCCTTATATGGTCTTGATGTTTCACGTCCAG
              | || | ||||| || || |||| | ||||||||||||||| || |
smpf1 AGGCACTTGGCGAAGTAGTGCGGTTGGGTGATCTGTATGGTCTTGATGTTCGCAAACCTG
                  970 980 990 1000 1010 1020
                  820 830 840 850 860 870
dbpf1. CTATGAACGTAAAAGAAGCAATCCAATGGGTTAACATCGCTTATATGGCAGTATGTCGTG
              ||||||| || || ||||| ||||||||| |||| ||||| | |||||| || || || |
smpf1 CTATGAATGTTAAGGAAGCTATCCAATGGATTAATATCGCCTTTATGGCTGTCTGCCGCG
                1030 1040 1050 1060 1070 1080
                  880 890 900 910 920 930
dbpf1. TCATTAATGGAGCTGCAACTTCACTTGGACGTGTTCCAATCGTTCTTGATATCTTTGCAG
              | || ||||| ||||||||||| ||||||||||| |||||||||||||||||||||||||
smpf1 TTATCAATGGTGCTGCAACTTCTCTTGGACGTGTCCCAATCGTTCTTGATATCTTTGCAG
                1090 1100 1110 1120 1130 1140
                  940 950 960 970 980 990
dbpf1. AACGTGACCTTGCTCGTGGAACATTTACTGAACAAGAAATTCAAGAATTTGTTGATGATT
              ||||||||||||||||||| || || |||||| |||||| |||||||| |||||||| |
smpf1 AACGTGACCTTGCTCGTGGCACTTTCACTGAATCAGAAATCCAAGAATTCGTTGATGACT
                1150 1160 1170 1180 1190 1200
                1000 1010 1020 1030 1040 1050
dbpf1. TCGTTTTGAAGCTTCGTACAATGAAATTTGCTCGTGCAGCTGCTTATGATGAACTTTATT
              ||||| |||| |||||||| | |||||||| ||| | |||||||| ||||||||||
smpf1 TCGTTATGAAACTTCGTACGGTTAAATTTGCACGTACTAAGGCTTATGACGAACTTTATT
                1210 1220 1230 1240 1250 1260
                1060 1070 1080 1090 1100 1110
dbpf1. CTGGTGACCCAACATTCATCACAACATCTATGGCTGGTATGGGTAATGACGGACGTCACC
              | |||||||||||||| || || || ||||||||||||||||| ||| ||||||||||
smpf1 CAGGTGACCCAACATTTATTACGACTTCTATGGCTGGTATGGGAGCTGATGGACGTCACC
                1270 1280 1290 1300 1310 1320
                1120 1130 1140 1150 1160 1170
dbpf1. GTGTCACTAAAATGGACTACCGTTTCTTGAACACACTTGATACAATCGGAAATGCTCCAG
              |||| ||||| ||||||||||||||||| || || ||||||| || || ||||||||||
smpf1 GTGTTACTAAGATGGACTACCGTTTCTTAAATACGCTTGATAATATTGGCAATGCTCCAG
                1330 1340 1350 1360 1370 1380
                1180 1190 1200 1210 1220 1230
dbpf1. AACCAAACTTGACAGTCCTTTGGGATTCTAAACTTCCTTACTCATTCAAACGTTATTCAA
              |||| ||||| || || |||||| |||| | |||||||| ||| | ||||| |
smpf1 AACCTAACTTAACCGTTCTTTGGTCAAGTAAATTGCCTTACTCTTTCCGTCATTATTGTA
                1390 1400 1410 1420 1430 1440
                1240 1250 1260 1270 1280 1290
dbpf1. TGTCTATGAGCCACAAGCATTCTTCTATTCAATATGAAGGTGTTGAAACAATGGCTAAAG
              ||||||||||||||||||||||||| ||||||||||||||||| ||| ||||||||||
smpf1 TGTCTATGAGCCACAAGCATTCTTCAATTCAATATGAAGGTGTCACAACTATGGCTAAAG
                1450 1460 1470 1480 1490 1500
                1300 1310 1320 1330 1340 1350
dbpf1. ATGGATATGGCGAAATGTCATGTATCTCTTGTTGTGTCTCACCACTTGATCCAGAAAATG
              | || ||||| ||||||||||||||||| || ||||| || || |||||||| ||||| |
smpf1 AAGGTTATGGTGAAATGTCATGTATCTCATGCTGTGTATCTCCGCTTGATCCTGAAAACG
                1510 1520 1530 1540 1550 1560
                1360 1370 1380 1390 1400 1410
dbpf1. AAGAAGGACGTCATAACCTCCAATACTTTGGTGCGCGTGTAAACGTCTTGAAAGCAATGT
              |||| | || || || || |||||||||||||| ||||| ||||| | |||||| |
smpf1 AAGATCGTCGCCACAATCTACAATACTTTGGTGCTCGTGTTAACGTTCTTAAAGCACTTC
                1570 1580 1590 1600 1610 1620
                1420 1430 1440 1450 1460 1470
dbpf1. TGACTGGTTTGAACGGTGGTTATGATGACGTTCATAAAGATTATAAAGTATTCGACATCG
              | || ||| | || || ||||| || || ||||| ||||| || |||||||| || |||
smpf1 TTACAGGTCTTAATGGCGGTTACGACGATGTTCACAAAGACTACAAAGTATTTGATGTCG
                1630 1640 1650 1660 1670 1680
                1480 1490 1500 1510 1520 1530
dbpf1. AACCTGTTCGTGACGAAATTCTTGACTATGATACAGTTATGGAAAACTTTGACAAATCTC
              ||||| | ||||| ||| | ||||| | ||| || |||| | || ||||| ||| | |
smpf1 AACCTATCCGTGATGAAGTCCTTGATTTTGAAACGGTTAAAGCTAATTTTGAAAAAGCAC
                1690 1700 1710 1720 1730 1740
                1540 1550 1560 1570 1580 1590
dbpf1. TCGACTGGTTGACTGATACTTATGTTGATGCAATGAATATCATTCATTACATGACTGATA
              | || ||||||||||||||||| || || ||||||||||||||||| || ||||||||||
smpf1 TTGATTGGTTGACTGATACTTACGTGGACGCAATGAATATCATTCACTATATGACTGATA
                1750 1760 1770 1780 1790 1800
                1600 1610 1620 1630 1640 1650
dbpf1. AATATAACTATGAAGCAGTTCAAATGGCCTTCTTGCCTACTAAAGTTCGTGCTAACATGG
              |||||||||||||||| ||||||||||||||||| || || ||| || || ||||
smpf1 AATATAACTATGAAGCCGTTCAAATGGCCTTCTTACCAACACGTGTTAAAGCCAATATGG
                1810 1820 1830 1840 1850 1860
                1660 1670 1680 1690 1700 1710
dbpf1. GATTTGGTATCTGTGGATTCGCAAATACAGTTGATTCACTTTCAGCAATTAAATATGCTA
              |||||||||| || |||||| | ||||||||||||||| | ||||| |||||||||||||
smpf1 GATTTGGTATTTGCGGATTCTCTAATACAGTTGATTCATTATCAGCTATTAAATATGCTA
                1870 1880 1890 1900 1910 1920
                1720 1730 1740 1750 1760 1770
dbpf1. AAGTTAAAACATTGCGTGATGAAAATGGCTATATCTACGATTACGAAGTAGAAGGTGATT
                  || ||| | | ||||||||| |||| || || ||||| || ||| | ||| | |
smpf1 CTGTAAAACCTATTCGTGATGAAGATGGTTACATTTACGACTATGAAACTGTTGGTAACT
                1930 1940 1950 1960 1970 1980
                1780 1790 1800 1810 1820 1830
dbpf1. TCCCTCGTTATGGTGAAGATGATGATCGTGCTGATGATATTGCTAAA--CTTGTCATGAA
              |||||||||| || ||||||||||| |||| || || ||| || ||| | ||||
smpf1 TCCCTCGTTACGGAGAAGATGATGACCGTGTAGACTCAATCGCTGAATGGTTG-CTTGAA
                1990 2000 2010 2020 2030 2040
                    1840 1850 1860 1870 1880 1890
dbpf1. AATGTACCATGAAAAATTAGCTTCACACAAACTTTACAAAAATGCTGAAGCTACTGTTTC
                  | | |||| | || || ||||| |||||| || | ||||||||||| ||
smpf1 GCT-TTCCATACTCGTCTTGCACGTCATAAACTGTACAAAGATTCCGAAGCTACTGTATC
                    2050 2060 2070 2080 2090 2100
                    1900 1910 1920 1930 1940 1950
dbpf1. ACTTTTGACAATTACATCTAACGTTGCTTACTCTAAACAAACTGGTAATTCTCCAGTACA
              | | | ||||| || ||||| |||||||| |||||||||||||||||||||||||| ||
smpf1 ATTGCTTACAATCACTTCTAATGTTGCTTATTCTAAACAAACTGGTAATTCTCCAGTTCA
                    2110 2120 2130 2140 2150 2160
                    1960 1970 1980 1990 2000 2010
dbpf1. TAAAGGAGTATTCCTCAATGAAGATGGTACAGTAAATAAATCTAAACTTGAATTCTTCTC
                || || || | |||||||||||||||| | || || |||||| | |||||||||||
smpf1 CAAGGGTGTTTACCTCAATGAAGATGGTTCTGTGAACTTGTCTAAAGTAGAATTCTTCTC
                    2170 2180 2190 2200 2210 2220
                    2020 2030 2040 2050 2060 2070
dbpf1. ACCAGGTGCTAACCCATCTAATAAAGCTAAGGGTGGTTGGTTGCAAAACCTTCGCTCATT
              |||||||||||||||||| ||||||||| || || |||||||||||| | ||||||
smpf1 ACCAGGTGCTAACCCATCAAATAAAGCTTCCGGCGGCTGGTTGCAAAACTTGAACTCATT
                    2230 2240 2250 2260 2270 2280
                    2080 2090 2100 2110 2120 2130
dbpf1. GGCTAAGTTGGAATTCAAAGATGCAAATGATGGTATTTCATTGACTACTCAAGTTTCACC
              | || | || || | |||||||||||||| |||||||| ||||||||||||||
smpf1 GAAGAAACTTGACTTTGCTCACGCAAATGATGGTATCTCATTGACAACTCAAGTTTCACC
                    2290 2300 2310 2320 2330 2340
                    2140 2150 2160 2170 2180 2190
dbpf1. TCGTGCACTTGGTAAAACTCGTGATGAACAAGTGGATAACTTGGTTCAAATTCTTGATGG
                      || |||||||| || ||||||||||| | |||||| || ||||||||||||
smpf1 AAAAGCTCTTGGTAAGACATTCGATGAACAAGTTGCTAACTTAGTAACAATTCTTGATGG
                    2350 2360 2370 2380 2390 2400
                    2200 2210 2220 2230 2240 2249
dbpf1. ATACTTCACACCAGGTGCT----TTGATTAATGGTACTGAATTTGCAGGTCAACACGTTA
                ||||| | || | | | | | | | || | | | | || | |||
smpf1 TTACTTTGAAGGCGGCGGTCAACACGTTAACTTGAAC-GTTATGGATCTTAAAGATGTTT
                    2410 2420 2430 2440 2450 2460
        2250 2260 2270 2280 2290 2300 2309
dbpf1. ACTTGAACGTAATGGACCTTAAAGATGTTTACGATAAAATCATGCGTGGTGAAGATGTTA
              | || | ||| | | |||||||| || | ||| ||| | |||||
smpf1 ATGACAAGATCATGAATGGTGAAGATGTTATCGTTCGTATC---TCAGGTTACTGTGTTA
                      2470 2480 2490 2500 2510
        2310 2320 2330 2340 2350 2360 2369
dbpf1. TCGTTCGTATCTCTGGTTACTGTGTCAATACTAAATACCTCACACCAGAACAAAAACAAG
                | | | | | | || ||| ||| | |||| | || |
smpf1 ACACTAAATACCTTACTAAAGAACAAAAGACTGAAT---TGACACAACGTGTTTTCCATG
        2520 2530 2540 2550 2560 2570
        2370 2380 2390 2400 2410 2420
dbpf1. AA-TTAACTGAACGTGTCTTCCA--TGAAGTTCTTTCAAACGATGATGAAGAAGTAA--T
              || || || || | | | || | || | ||| | | |||| |||
smpf1 AAGTTCTCTCAATGGATGATGCAGCTACAGACTTGGTTAACAACAAGTAAGAGTTAAACA
              2580 2590 2600 2610 2620 2630
                    2430 2440 2450 2460 2470
dbpf1. GCATA-CTTCAAACATCTAATTCTTAAAA--------TTTAATGAATATTCGG--TCTGT
              | || || ||| | || | || ||||| |||| | ||||| | |
smpf1 GTTTAGTTTAAAAGACCTCACTCATAAAAGTGAGGTCTTTACTTTGCTTTCGGGTACGAT
              2640 2650 2660 2670 2680 2690
                    2480 2490 2500 2510 2520 2530
dbpf1. CAGTTTTACTGACAGACTTTTTTTTACGAAAAAATTAATCATAAT-AGTTAAAAACTATT
              || | ||| || |||| | | | ||||| | | || | | || |||||| |
smpf1 CA-AAGCAGTGAGAGCTTTTTATATTCTAAAAACTCA--CAAATTCAGAAAAAAACAGCT
                2700 2710 2720 2730 2740 2750
                      2540 2550 2560 2570 2580 2590
dbpf1. GTTTTTAGTTTAAGAAAGTTAAATTTTATGCTAAAATAGATGAATGAAAATGGTAATTGG
                || | | || |||| | ||||| |||| |||| |||||||| |||||
smpf1 CTTGTGATTT---GAAA---AGCTTTTA-GCTACAATAATATTATGAAAAT--TAATTAT
                    2760 2770 2780 2790 2800
                      2600 2610 2620 2630 2640 2650
dbpf1. ATTGACAGGCGGAATTGCGATGGGAAATCAACGGTGGTTGATTTTTTGATTCTGAGGGTT
smpf1 ACTCGCGACACACTGTCATCCACCTATCTTGATGCAGTAAAAATTAGACACCTTGTCTTC
                  2810 2820 2830 2840 2850 2860
dbpf1.: corresponding to nucleotides 1-2653 of SEQ ID NO:15;
smpf1: SEQ ID NO:17
dbpf1.seq/rev
empr6: hi32812
ID HI32812 standard; DNA; PRO; 10817 BP.
AC U32812; L42023;
NI g1222092
DT Aug. 9, 1995 (Rel. 44, Created)
DE Haemophilus influenzae focA, pf1A, pf1B, rspB, yaaJ, yajF, yeiG . . .
SCORES Init1: 652 Initn: 1077 Opt: 1299
                                        55.4% identity in 1961 bp overlap
        1979 1969 1959 1949 1939 1929 1920
dbpf1. CATCTTCATTGAGGAATACTCCTTTATGTACTGGAGAATTACCAGTTTGTTTAGAGTAAG
                                                                          | || | ||||| |||| ||| |||
hi3281 GTCCGAATGGTGCACCAGCACGACGACCATCAGGGGTGTTACCCGTTTTCTTACCATAAA
                        2730 2740 2750 2760 2770 2780
        1919 1909 1899 1889 1879 1869 1860
dbpf1. CAACGTTAGATGTAATTGTCAAAAGTGAAACAGTAGCTTCAGCATTTTTGTAAAGTTTGT
              | |||||||| ||||| || || | || |||| | ||||| |||| |||
hi3281 CTACGTTAGAAGTAATGGTTAATACAGATTGTGTAGGCACTGCATTGCGGTAAGTTTTAA
                        2790 2800 2810 2820 2830 2840
        1859 1849 1839 1829 1819 1809
dbpf1. GTGAAGCTAATTT-TTCATGGTACATTTTCATGACAAGTTTAGCAATATCATCAGCACGA
              || | ||| ||||| || || | | ||| || |||||| ||||
hi3281 GTTTTTGAATTTTCTTCATAAAAC-GTTCAACTAAGTCACAAGCGATGTCATCAACACGG
                        2850 2860 2870 2880 2890 2900
          1799 1789 1779 1769 1759
dbpf1. TCATCATCTTCACCATAACGAGGGAAATCACCTTCTACTTCGTAATCG----------TA
              | ||||| | |||||| | || | |||||||| | ||| | ||| ||
hi3281 TTATCATTGTTACCATATTGTGGATATTCACCTTCGATTTCAAAGTCGATTGCTACGTTA
                          2910 2920 2930 2940 2950 2960
        1750 1749 1739 1729 1719
dbpf1. G--------ATATAGCCATTTTCAT------------CACGCAATGTTTTAACTTTAGCA
              | | || ||||| || || |||| | || ||||||||| |||
hi3281 GTTGCAACAACATTGCCATCTTTATCTTTGATGTCGCCACGAACTGGTTTAACTTTCGCA
                          2970 2980 2990 3000 3010 3020
          1709 1699 1689 1679 1669 1659
dbpf1. TATTTAATTGCTGAAAGTGAATCAACTGTATTTGCGAATCCACAGATACCAAATCCCATG
              ||||| |||||||||||||| ||| | | | | | || ||||||| | ||||
hi3281 TATTTGATTGCTGAAAGTGAGTCAGCCGCAACAGAAAGACCTGCGATACCACAAGCCATA
                          3030 3040 3050 3060 3070 3080
          1649 1639 1629 1619 1609 1599
dbpf1. TTAGCACGAACTTTAGTAGGCAAGAAGGCCATTTGAACTGCTTCATAGTTATATTTATCA
                || || | | | || |||||| | ||||| || |||||||||
hi3281 GTACGGTATACATCACGATCATGTAATGCCATTAATGCGGCTTCGTATGAATATTTATCG
                          3090 3100 3110 3120 3130 3140
          1589 1579 1569 1559 1549 1539
dbpf1. GTCATGTAATGAATGATATTCATTGCATCAACATAAGTATCAGTCAACCAGTCGAGAGAT
                  ||| || || || | || | ||| ||||| | | |||||| || | | |
hi3281 TGCATATAGTGGATTACGTTTAAGGCAGTCACATATTGTTTTGCCAACCAATCCATAAAG
                          3150 3160 3170 3180 3190 3200
          1529 1519 1509 1499 1489 1479
dbpf1. TTGTCAAAGTTTTCCATAACTGTATCATAGTCAAGAATTTCGTCACGAACAGGTTC-GAT
                | || | ||| |||||||| | || | | ||| ||| || ||| | | |
hi3281 CTATCCATACGAGTCATTACTGTATCGAAATCTAATACTTCATCAGTAATTGGTGCAGTT
                          3210 3220 3230 3240 3250 3260
            1469 1459 1449 1439 1429 1419
dbpf1. GTCGAATACTTTATAATCTTTATGAACGTCATCATAACCACCGTTCAAACCAGTCAACAT
                ||| | | || || ||||| ||| ||||| | | ||||
hi3281 TTCGGACCTACTTGCATACCTA-ATTTTTCATCGATACCGCCGTTGATTGCGTATAACAA
                          3270 3280 3290 3300 3310
            1409 1399 1389 1379 1369 1359
dbpf1. TGCTTTCAAGACGTTTACACGCGCACCAAAGTATTGGAGGTTATGACGTCCTTCTTCATT
              || |||| | |||| |||| ||||| ||| |||| | | | || || ||||
hi3281 TGTTTTCGCTAAGTTTGCACGTGCACCGAAGAATTGCATTTGTTTAC--CCACAATCAT-
        3320 3330 3340 3350 3360 3370
            1349 1339 1329 1319 1309 1299
dbpf1. TTCTGGATCAAGTGGTGAGACACAACAAGAGATACATGACATTTCGCCATATCCATCTTT
                                      |||||| |||||||| | ||| | ||| | |||
hi3281 ------------TGGTGATACACAACATGCGATTGCGTAGTCATCGTTGTTGAAGTCTGG
                                      3380 3390 3400 3410 3420
            1289 1279 1269 1259 1249 1239
dbpf1. AGCCATTGTTTCAACACCTTCATATTGAATAGAAGA-ATGCTTGTGGCT-CATAGACATT
              | |||| ||| | ||| ||||||| || || | | | | | | ||
hi3281 ACGCATTAAATCATCGTTTTCGTATTGAACTGATGAGGTATCAATCGATACTTTTGCACA
                  3430 3440 3450 3460 3470 3480
                1229 1219 1209 1199 1189 1179
dbpf1. GAATAACGTTTGAATGAGTAAGGAAGTTTAGAATCCCAAAGGACTGTCAAGTTTGGTTCT
              || |||||||||| | ||| | || | |||||| | || |||||||| |||
hi3281 GA--AACGTTTGAAGTTTTCAGGTAATTGTTCAGACCAAAGAATGGTTAAGTTTGGCTCT
                      3490 3500 3510 3520 3530 3540
                1169 1159 1149 1139 1129 1119
dbpf1. GGAGCATTTCCGATTGTATCAAGTGTGTTCAAGAAACGGTAGTCCATTTTAGTGACACGG
              |||| | | || || | | ||| |||| || | |||| | |||| || ||
hi3281 GGAGAAGTACCCATGTTGTAAAGGGTGTGTAAAATACGGAATGTATTTTTGGTTACTAAT
                      3550 3560 3570 3580 3590 3600
                1109 1099 1089 1079 1069 1059
dbpf1. TGACGTCCGTCATTACCCATACCAGCCATAGATGTTGTGATGAATGTTGGGTCACCAGAA
                  ||| || || ||||||||| || || | | || | |||||||||||||
hi3281 GTACGACCATCTAAACCCATACCTGCGATGGTTTCAGTTGCCCACATTGGGTCACCAGAG
                      3610 3620 3630 3640 3650 3660
                1049 1039 1029 1019 1009 999
dbpf1. TAAAGTTCATCATAAGCAGCTGCACGAGCAAATTTCATTGTACGAAGCTTCAAAACGAAA
                | | || ||| || ||| || ||| || | ||||||| |||| ||| ||
hi3281 AATAATTGATCGTATTCAGGTGTACGTAAGAAACGAACCATACGAAGTTTCATAACTAAG
                      3670 3680 3690 3700 3710 3720
                  989 979 969 959 949 939
dbpf1. TCATCAACAAATTCTTGAATTTCTTGTTCAGTAAATGTTCCACGAGCAAGGTCACGTTCT
              | ||||| |||||||| ||| |||||||| | |||| | ||||||||
hi3281 TGGTCAACTAATTCTTGCGCTTCAGTTTCAGTAATTTTTCCTGCTTTTAAATCACGTTCG
                      3730 3740 3750 3760 3770 3780
                  929 919 909 899 889 879
dbpf1. GCAAAGATATCAAGAACGATTGGAACACGTCCAAGTGAAGTTGCAGCTCCA-TTAATGAC
                      | | |||| || | ||| ||| || | ||| ||||||| ||| || |||
hi3281 ATGTACACGTCAATAAAGGTTGCGGTACGACCGAATGACATTGCAGCACCATTTTGTGAT
                      3790 3800 3810 3820 3830 3840
                    869 859 849 839 829 819
dbpf1. ACGACATACTGCCATATAAGCGATGTTAACCCATTGGATTGCTTCTTTTACGTTCATAGC
                    | | | || | |||||| | || | |||||| || ||||||| | || | ||
hi3281 TTTA-TTGCAGCAAGATAAGCAAAGTACATCCATTGAATGGCTTCTTGAGCATTAGTTGC
                        3850 3860 3870 3880 3890 3900
                    809 799 789 779 769 759
dbpf1. TGGACGTGAAACATCAAGACCATATAAGGCACCAAAGTTTACAACTTCTTGCAAAGCTTG
              ||| |||| |||| |||||| || | | | | | || || || |
hi3281 TGGGTTAGAAATATCATAACCATAGCTTGCTGCCATTTGTTTTAATTGACCTAATGCACG
                        3910 3920 3930 3940 3950 3960
                    749 739 729 719 709 699
dbpf1. GTATTGCATATTAATTTCTTCTTTAAGACGAATGTTTTCTTCGTTAATTTCAGTGATTGC
              || ||| |||||||| | |||||| || |||| | || | |
hi3281 GTGTTGTTCTGCGATTTCTTCACGTAAACGAATTGTTGCTTCAAGATTT------ACGCC
                        3970 3980 3990 4000 4010
                    689 679 669 659 649 639
dbpf1. ATCCCATTCTTTTGCTTTTTCCTTCATAAG-GTAATCAGCACCGTAAAGGGCAAGACGTG
              ||| |||| |||||| | | ||| | | | || | | |
hi3281 ATC---TTCTAAATCTTTTT-GTAAAGAAGAGAATTGTGCGTATTTATCTTTCATTAAGA
                      4020 4030 4040 4050 4060 4070
                      629 619 609 599 589 579
dbpf1. CATAGACACCAATGATACGTCCACGAGAGTAAGCATCTGGCAAACCTGTTACAGTATGAG
                || |||||| | | ||||| ||| || | | || | || ||
hi3281 AATCTACACCATAAAGTGCTACACGACGGTAGTCACCGATGATACGACCACGACCATAAG
                        4080 4090 4100 4110 4120 4130
                            569 559 549 539 529 520
dbpf1. CAT---GACGTGCTTTACGAATTGCTGAAGTATAAGCACGAAAGAT-TCCATCATTTACA
              ||| || | | | | | || || ||| || || || | | || |
hi3281 CATCTGGAAGACCAGTTAATACCCCAGATTTACGGCAACGTAAAATATCTGGCGTGTAAA
                        4140 4150 4160 4170 4180 4190
                  519 509 499 489 479
dbpf1. ----GAAGTCA--TTGTTTGTG------ACAAAACATCATGCAAGCCTGGGTCAACTGAG
                      ||| || ||| || || || | ||| ||| | | |||
hi3281 CATCGAATACACCTTGGTTATGTGTTTTACGGTA-TTCAGTGAAGATTTTTTTCACTTTT
                        4200 4210 4220 4230 4240 4250
              469 459 449 439 429 419
dbpf1. AGACCGTGTT--CTGTCAAAATCTTTTCAGCAACACGAAGTCCACCTCTTGGCATGAAAT
                || | ||| | || || |||| || ||| |||||| || | || |
hi3281 GGATCAAGTTCACGACCATAAACTTTACAAGAAC----CTTCCACCATTTTG-ATACCAC
                          4260 4270 4280 4290 4300
                  409 399 389 379 369 359
dbpf1. TCAAGCGGAAAAGTTCGCTATTT-----TGCATCCCATAGATGAGTTCAAGTTCTTTATC
                  || | | || | | ||| | |||| | || || ||| ||| ||
hi3281 CGAATGGCATAATGGCACGTTTTAAAGGTTCATCAGTTTGA--AGACCAACGATTTTTTC
                4310 4320 4330 4340 4350 4360
                            349 339 329 319 309
dbpf1. ATTAGCATCGATATATCCAGCAGGGATTTTATCAATAGAGGT-TACGCGGTCAGT--ATC
                    | | | | || | |||| | | ||| ||| | | || || |||
hi3281 TAAATCTTTGTTAATGTAACCAGGTGCGTGAGAGATAATGGTAGATGGTGTATGTTCATC
                    4370 4380 4390 4400 4410 4420
              299 289 279 269 259 249
dbpf1. GAAGGGAAATCCTACTT--CTTC-GTAGTGATTTTTTGTATCTTCAAT---AATTTTCTT
                || ||| | | | | || | | |||| || |||| || | || |
hi3281 AAAATCTAATGGCGCGTGAGTACGGTTTTCAATTTTAATACCTTCCATCACAGATTCCCA
                    4430 4440 4450 4460 4470 4480
                          239 229 219 209 199 189
dbpf1. TACTTTAAGTGTACGTTCTGTTGGCCCAGC-AAGAAAGCTTTCATCACCATCATATGGTT
                | || ||| ||| ||||| || || ||||||| ||||| || ||||| ||
hi3281 AAGCTTGGTTGTTGCTTCGGTTGGACCTGCTAAGAAAG-AGTCATCGCCTTCATAAGGGG
                    4490 4500 4510 4520 4530 4540
                            179 169 159 149 139 129
dbpf1. TGTAGTTTTCTTGTACAAAGCGAGTAACGCTTGCTTTATCGCGCCAGTTGGTTCCTTTAA
              | ||||||| ||| | |||| | || | | || || |||| | | || ||
hi3281 TATAGTTTTTTTGGATAAAGTCACGTACATTGACATTTTCTTGCCAATCGCCACCAGCAA
                      4550 4560 4570 4580 4590 4600
                            119 109 99 89 79 69
dbpf1. AACCATCCCAAGCTTGTTCAAAGATATTTTCCGTAACTTCGGTTTTCATTTTGAAATCTC
              ||||| |||| || || || ||| | | || ||| | | | | || ||
hi3281 AACCAGCCCACGC-----CA---ATTTTTGCATTTCATTAAGTTCTGACATAGTCATTTC
                      4610 4620 4630 4640 4650
                              59 49 39 29 19 9
dbpf1. CTTTGTTCTTTCTGTTTCAAATTACAACTTCATTATATCATACAGTTTGAGAATAGCAAA
              ||||||| ||
hi3281 CTTTGTTAATTAATAAATAAATCTTTAATGTGTTTTGGTTAAATAACGTTGGAATACACC
                  4660 4670 4680 4690 4700 4710
dbpf1: complementary strand corresponding to nucleotides 1979-9 of SEQ ID NO:15;
hi3281: SEQ ID NO:18
```

TABLE 3.4

Protein homology (FASTA, GCG Wisconsin Package Version 8. Genetics Computer Group)
using the complete protein sequence derived from the L. lactis DB1341 pf1 sequence
shown in TABLE 3.2
Only alignment of the L. lactis Pf1 protein (dbpf1.pep) with the best four scores
is shown.
The Pf1 protein of Streptococcus mutans was not recorded in the searched protein
databases.

(Peptide) FASTA of: dbpf1.pep from: 1 to: 788 Jul. 19, 1996 09:11

| | | | |
|---|---|---|---|
| The best scores are: | init1 | initn | Opt . . . |

| | | | | |
|---|---|---|---|---|
| sw:pf1b_ecoli | P09373 escherichia coli. formate ac. | 560 | 1498 | 1502 |
| sw:pf13_ecoli | P42632 escherichia coli. probable f. | 558 | 1358 | 1487 |
| sw:pf1b_haein | P43753 haemophilus influenzae. form. | 545 | 1228 | 1521 |
| sw:pf1_chlre | P37836 chiamydomonas reinhardtii. f. | 163 | 259 | 306 |
| sw:fasd_ecoli | P46000 escherichia coli. outer memb. | 53 | 113 | 75 |
| sw:gtf2_strdo | P27470 streptococcus downei (strept. | 46 | 110 | 75 |
| sw:frap_rat | P42346 rattus norvegicus (rat). fkb | 42 | 101 | 53 |
| sw:frap_human | P42345 homo sapiens (human). fkbp-r. | 42 | 101 | 53 |

```
dbpf1.pep
sw:pf1b_ecoli
ID PFLB_ECOLI STANDARD; PRT; 759 AA.
AC P09373;
DE FORMATE ACETYLTRANSFERASE 1 (EC 2.3.1.54) (PYRUVATE FORMATE-LYASE 1) . . .
SCORES Init1: 560 Initn: 1498 Opt: 1502
                                      42.2% identity in 732 aa overlap
                                  10 20 30 40 50 59
dbpf1. MKTEVTENIFEQAWDGFKGTNWRDKASVTRFVQENYKPYDGDESFLAGPTERTLKV-KKI
                                      :: ||:||: ::|:::::| |:|:||:||:||||||||:|| | :: :|:
pf1b_e SELNEKLATAWEGFTKGDWQNEVNVRDFIQKNYTPYEGDESFLAGATEATTTLWDKV
                                          10 20 30 40 50
                  60 70 80 90 100 110
dbpf1. IEDTK-NHYEEVGFPFDTDRVTSIDKIPAGYIDANDKELELIYGMQNSELFRLNFMPRGG
                    :|::| :: ::: |||: :::|:: |||| :|:|| | |:|::: :: :::| ||
pf1b_e MEGVKLENRTHAPVDFDTAVASTITSHDAGYI---NKQLEKIVGLQTEAPLKRALIPFGG
                      60 70 80 90 100 110
                  120 130 140 150 160 170
dbpf1. LRVAEKILTEHGLSVDPGLHDVLSQTMTSVNDGIFRAYTSAIRKARHAHTVTGLPDAYSR
                    ::: | :::: ::|| ::::::: ::: |:|:| :||::| : |:: ::|||||||:|
pf1b_e IKMIEGSCKAYNRELDPMIKKIFTEYRKTHNQGVFDVYTPDILRCRKSGVLTGLPDAYGR
                          120 130 140 150 160 170
                  180 190 200 210 220 230
dbpf1. GRIIGVYARLALYGADYLMKEKAKEWDAI-TEIN-----EENIRLKEEINMQYQALQEVV
                    ||||| | |:|||| |||||:| ::::: :::: |::|||:|||: |::|| ::
pf1b_e GRIIGDYRRVALYGIDYLMKDKLAQFTSLQADLENGVNLEQTIRLREEIAEQHRALGQMK
                          180 190 200 210 220 230
                              240 250 260 270 280 290
dbpf1. NFGALYGLDVSRPAMNVKEAIQWVNIAYMAVCRVINGAATSLGRVPIVLDIFAERDLARG
                    :::| || |:| || |::|||||: ::|:|: : |||| |:||::: ||:: |||| |
pf1b_e EMAAKYGYDISGPATNAQEAIQWTYFGYLAAVKSQNGAAMSFGRTSTFLDVYIERDLKAG
                          240 250 260 270 280 290
                              300 310 320 330 340 350
dbpf1. TFTEQEIQEFVDDFVLKLRTMKFARAAAYDELYSGDPTFITTSMAGMGNDGRHRVTKMDY
                    ::|||| ||:||::|:||| ::| |:::||||:||||:: |:|::||| ||| ||| ::
pf1b_e KITEQEAQEMVDHLVMKLRMVRFLRTPEYDELFSGDPIWATESIGGMGLDGRTLVTKNSF
                          300 310 320 330 340 350
                              360 370 380 390 400 410
dbpf1. RFLNTLDTIGNAPEPNLTVLWDSKLPYSFKRYSMSMSHKHSSIQYEGVETMAKDGYGEMS
                    |||||| |:| :||||:|:||::||| :||::: ::| : ||:|||: : | | ::
pf1b_e RFLNTLYTMGPSPEPNMTILWSEKLPLNFKKFAAKVSIDTSSLQYENDDLMRPDFNNDDY
                          360 370 380 390 400 410
                              420 430 440 450 460 470
dbpf1. CISCCVSPLDPENEEGRHNLQYFGARVNVLKAMLTGLNGGYDDVHKDYKVFDIEPVRDEI
                      |:|||||: ::: :|:||||:|: |:|| ::||| |: | : ||:::::
pf1b_e AIACCVSPMIVGKQ-----MQFFGARANLAKTMLYAINGGVDEKLKMQVGPKSEPIKGDV
                          420 430 440 450 460
                              480 490 500 510 520 530
dbpf1. LDYDTVMENFDKSLDWLTDTYVDAMNIIHYMTDKYNYEAVQMAFLPTKVRANMGFGICGF
                  |:||:|||::|: :|||:: |::|:|||||| |||:||| ||: :| :|: || |:
pf1b_e LNYDEVMERMDHFMDWLAKQYITALNIIHYMHDKYSYEASLMALHDRDVIRTMACGIAGL
          470 480 490 500 510 520
                              540 550 560 570 580 590
dbpf1. ANTVDSLSAIKYAKVKTLRDENGYIYDYEVEGDFPRYGEDDDRADDIAKLVMKMYHEKLA
                    : ::||||||||||||::|||:| |:|:||::|::|::| |:||:| ::::: :|::
pf1b_e SVAADSLSAIKYAKVKPIRDEDGLAIDFEIEGEYPQFGNNDPRVDDLAVDLVERFMKKIQ
          530 540 550 560 570 580
                              600 610 620 630 640 650
dbpf1. SHKLYKNAEATVSLLTITSNVAYSKQTGNSPVHKGVFLNEDGTVNKSKLEFFSPGANPSN
                    : : |::| :| |:|||||||:|:|:|||:| || ::: | : :: :
pf1b_e KLHTYRDAIPTQSVLTITSNVVYGKKTGNTP---------DG--RRAGAPFGPGANPMHG
          590 600 610 620 630
                              660 670 680 690 700 710
dbpf1. KAKGGWLQNLRSLAKLEFKDANDGISLTTQVSPRALGKTRDEQVDNLVQILDGYFTPGAL
                    ::: | :::| |:||| | |:|||| | : |:||||: : : :||: ::|||| ::|
pfLb_e RDQKGAVASLTSVAKLPFAYAKDGISYTFSIVPNALGKDDEVRKTNLAGLMDGYFHHEAS
            640 650 660 670 680 690
                              720 730 740 750 760 770
dbpf1. INGTEFAGQHVNLNVMDLKDVYDKIMRGEDVIVRISGYCVNTKYLTPEQKQELTERVFHE
                    |:|:: : :| : | |:::
pf1b_e IEGGQHLNVNVMNREMLLDAMENPEKYPQLTIRVSGYAVRFNSLTKEQQQDVITRTFTQS
            700 710 720 730 740 750
dbpf1: corresponds to amino acid residues 1-772 of SEQ ID NO:16;
pf1b_e: corresponds to amino acid residues of SEQ ID NO:14
dbpf1.pep
sw:pf13_ecoli
ID PFL3_ECOLI STANDARD; PRT; 746 AA.
AC P42632;
DE PROBABLE FORMATE ACETYLTRANSFERASE 3 (EC 2.3.1.54) (PYRUVATE FORMATE- . . .
SCORES Init1: 558 Initn: 1358 Opt: 1487
                                      39.8% identity in 741 aa overlap
                                    10 20 30 40 50
dbpf1. MKTEVTENIFEQAWDGFKGTNWRDKASVTRFVQENYKPYDGDESFLAGPTERTLKV-K
                    ::::::::::::|| |||||:|::: :| |:|:||:||:|||||||::| | :: :
pf13_e MKVDIDTSDKLYADAWLGFKGTDWKNEINVRDFIQHNYTPYEGDESFLAEATPATTELWE
                                10 20 30 40 50 60
                  60 70 80 90 100 110
dbpf1. KIIEDTK-NHYEEVGFPFDTDRVTSIDKIPAGYIDANDKELELIYGMQNSELFRLNFMPR
                |::|::: :: ::: |||: :|:|: |||| :: || | |:|::: :: :: |
pfL3_e KVMEGIRIENATHAPVDFDTNIATTITAHDAGYI---NQPLEKIVGLQTDAPLKRALHPF
                                70 80 90 100 110
                  120 130 140 150 160 170
dbpf1. GGLRVAEKILTEHGLSVDPGLHDVLSQTMTSVNDGIFRAYTSAIRKARHAHTVTGLPDAY
                ||::: :: : ::| ::|:::: :::: ::: |:|:| :|:::: : |:: ::|||||:|
pf13_e GGINMIKSSFHAYGREMDSEFEYLFTDLRKTHNQGVFDVYSPDMLRCRKSGVLTGLPDGY
                120 130 140 150 160 170
                  180 190 200 210 220 230
dbpf1. SRGRIIGVYARLALYGADYLMKEKAKEWDAI------TEINEENIRLKEEINMQYQALQE
                :|||||| | |:|||| :||::|:: ::::: :| |::|||:||:: : :|| :
pf13_e GRGRIIGDYRRVALYGISYLVRERELQFADLQSRLEKGEDLEATIRLREELAEHRHALLQ
                180 190 200 210 220 230
                              240 250 260 270 280 290
dbpf1. VVNFGALYGLDVSRPAMNVKEAIQWVNIAYMAVCRVINGAATSLGRVPIVLDIFAERDLA
                : :::| ||:|:|||| |::||:||: :||:|: : ||:| ||||:: |||: |||:
pf13_e IQEMAAKYGFDISRPAQNAQEAVQWLYFAYLAAVKSQNGGAMSLGRTASFLDIYIERDFK
                240 250 260 270 280 290
                              300 310 320 330 340 350
dbpf1. RGTFTEQEIQEFVDDFVLKLRTMKFARAAAYDELYSGDPTFITTSMAGMGNDGRHRVTKM
                  |:::||: ||::|:|::|:| ::| |::::|:|:||||:: |: ::||| ||| |||
pf13_e AGVLNEQQAQELIDHFIMKIRMVRFLRTPEFDSLFSGDPIWATEVIGGMGLDGRTLVTKN
                300 310 320 330 340 350
                              360 370 380 390 400 410
dbpf1. DYRFLNTLDTIGNAPEPNLTVLWDSKLPYSFKRYSMSMSHKHSSIQYEGVETMAKDGYGE
                ::|:|:||:|:| |||||||:||:::|| :||:|: :| ||:|||: : | :| ::
pf13_e SFRYLHTLHTMGPAPEPNLTILWSEELPIAFKKYAAQVSIVTSSLQYENDDLMRTDFNSD
                360 370 380 390 400 410
                              420 430 440 450 460 470
dbpf1. MSCISCCVSPLDPENEEGRHNLQYFGARVNVLKAMLTGLNGGYDDVHKDYKVFDIEPVRD
                    |:|||||: ::: :|:||||:|: |::| ::||| |: | :::|::|
pf13_e DYAIACCVSPMVIGKQ-----MQFFGARANLAKTLLYAINGGVDEKLKIQVGPKTAPLMD
                420 430 440 450 460 470
                              480 490 500 510 520 530
dbpf1. EILDYDTVMENFDKSLDWLTDTYVDAMNIIHYMTDKYNYEAVQMAFLPTKVRANMGFGIC
                ::||||:||:::|: :|||: |::|:|||||| |||:||| ||: :| :|: ||
pf13_e DVLDYDKVMDSLDHFMDWLAVQYISALNIIHYMHDKYSYEASLMALHDRDVYRTMACGIA
                          480 490 500 510 520 530
                              540 550 560 570 580 590
dbpf1. GFANTVDSLSAIKYAKVKTLRDENGYIYDYEVEGDFPRYGEDDDRADDIAKLVMKMYHEK
                |:: ::|||||||||:||::||||| |:|::|::|:||::|:|:|:|| ::::: :|
pf13_e GLSVATDSLSAIKYARVKPIRDENGLAVDFEIDGEYPQYGNNDERVDSIACDLVERFMKK
                          540 550 560 570 580 590
                              600 610 620 630 640 650
dbpf1. LASHKLYKNAEATVSLLTITSNVAYSKQTGNSPVHKGVFLNEDGTVNKSKLEFFSPGANP
                : : |:|| :| |:|||||||:|:::|||:| || ::: | : ::
pf13_e IKALPTYRNAVPTQSILTITSNVVYGQKTGNTP---------DG--RRAGTPFAPGANPM
                          600 610 620 630 640
                              660 670 680 690 700 710
dbpf1. SNKAKGGWLQNLRSLAKLEFKDANDGISLTTQVSPRALGKTRDEQVDNLVQILDGYFTPG
                  :::: | :::| |:||| |: |:|||| | : | ||||: : :||| :||||| ::
pf13_e HGRDRKGAVASLTSVAKLPFTYAKDGISYTFSIVPAALGKEDPVRKTNLVGLLDGYFHHE
                            650 660 670 680 690 700
                              720 730 740 750 760 770
dbpf1. ALINGTEFAGQHVNLNVMDLKDVYDKIMRGEDVIVRISGYCVNTKYLTPEQKQELTERVF
                | ::|:: : :| : | |:::
pf13_e ADVEGGQHLNVNVMNREMLLDAIEHPEKYPNLTIRVSGYACASTH
                            710 720 730 740
dbplf: corresponds to amino acid residues 1-770 of SEQ ID NO:16;
pf113_e: SEQ ID NO:19
dbpl.pep
sw:pf1b_haein
ID PFLB_HAEIN STANDARD; PRT; 769 AA.
AC P43753;
DE FORMATE ACETYLTRANSFERASE (EC 2.3.1.54) (PYRUVATE FORMATE-LYASE) . . .
SCORES Init1: 545 Initn: 1228 Opt: 1521
                                      42.1% identity in 781 aa overlap
```

| | |
|---|---|
| 10 20 30 40 50 59 | |
| dbpf1. MKTEVTENIFEQAWDGFKGTNWRDKASVTRFVQENYKPYDGDESFLAGPTERTLKV-KKI | (SEQ ID NO:16) |
| \|\|:\|\| \|::\|:::::\| \|:\|:\|\|:\|\|:\|\|:\|\|\|\|\|\|\|\| \| \|: ::: | |
| pf1b_h SELNEMQKLAWAGFAGGDWQENVNVRDFIQKNYTPYEGDDSFLAGPTEATTKLWESV | (SEQ ID NO:20) |
| 10 20 30 40 50 | |
| 60 70 80 90 100 110 | |
| dbpf1. IEDTK-NHYEEVGFPFDTDRVTSIDKIPAGYIDANDKELELIYGMQNSELFRLNFMPRGG | |
| :\|::\| :: ::: : \|\|:: ::\| : ::\|\|\| :\|:\|\| \| \|:\|::\| :: ::\|\| \|\| | |
| pf1b_h MEGIKIENRTHAPLDFDEHTPSTIISHAPGYI---NKDLEKIVGLQTDEPLKRAIMPFGG | |
| 60 70 80 90 100 110 | |
| 120 130 140 150 160 170 | |
| dbpf1. LRVAEKILTEHGLSVDPGLHDVLSQTMTSVNDGIFRAYTSAIRKARHAHTVTGLPDAYSR | |
| ::::\| : :\| ::\|\| ::::::: ::: \|:\|:\| :\|\|::\| : \|:: ::\|\|\|\|\|\|\|:\| | |
| pf1b_h IKMVEGSCKVYGRELDPKVKKIFTEYRKTHNQGVFDVYTPDILRCRKSGVLTGLPDAYGR | |
| 120 130 140 150 160 170 | |
| 180 190 200 210 220 230 | |
| dbpf1. GRIIGVYARLALYGADYLMKEKAKEWDAI-----TEIN-EENIRLKEEINMQYQALQEVV | |
| \|\|\|\|\| \| \|:\|\|\|\|:\|:\|\|\|:\| ::::: :::\| \|::\|\|\|:\|\|\|: \|::\|\| :: | |
| pf1b_h GRIIGDYRRVALYGVDFLMKDKYAQFSSLQKDLEDGVNLEATIRLREEIAEQHRALGQLK | |
| 180 190 200 210 220 230 | |
| 240 250 260 270 280 290 | |
| dbpf1. NFGALYGLDVSRPAMNVKEAIQWVNIAYMAVCRVINGAATSLGRVPIVLDIFAERDLARG | |
| :::\| \|\| \|:\|:\|\| \|::\|\|\|\|\|: :\|\|:\|: : \|\|\|\| \|:\|\|::: :\|:: \|\|\|\| \| | |
| pf1b_h QMAASYGYDISNPATNAQEAIQWMYFAYLAAIKSQNGAAMSFGRTATFIDVYIERDLKAG | |
| 240 250 260 270 280 290 | |
| 300 310 320 330 340 350 | |
| dbpf1. TFTEQEIQEFVDDFVLKLRTMKFARAAAYDELYSGDPTFITTSMAGMGNDGRHRVTKMDY | |
| ::\|\| \| \|\|:\|\|::\|:\|\|\| ::\| \|:::\|\|:\|:\|\|\|\| : \|:::\|\|\|\| \|\|\| \|\|\| :: | |
| pf1b_h KITETEAQELVDHLVMKLRMVRFLRTPEYDQLFSGDPMWATETIAGMGLDGRTLVTKNTF | |
| 300 310 320 330 340 350 | |
| 360 370 380 390 400 410 | |
| dbpf1. RFLNTLDTIGNAPEPNLTVLWDSKLPYSFKRYSMSMSHKHSSIQYEGVETMAKDGYGEMS | |
| \|:\|:\|\| ::\|::\|\|\|\|\|\|:\|\|:::\|\| :\|\|\|:: ::\| : \|\|:\|\|\|: : \| \| :: | |
| pf1b_h RILHTLYNMGTSPEPNLTILWSEQLPENFKRFCAKVSIDTSSVQYENDDLMRPDFNNDDY | |
| 360 370 380 390 400 410 | |
| 420 430 440 450 460 470 | |
| dbpf1. CISCCVSPLDPENEEGRHNLQYFGARVNVLKAMLTGLNGGYDDVHKDYKVFDIEPVRDEI | |
| \|:\|\|\|\|\|: ::: :\|:\|\|\|\|:\|: \|::\| ::\|\|\| \|: :::\|: \|\|: | |
| pf1b_h AIACCVSPMIVGKQ-----MQFFGARANLAKTLLYAINGGIDEKLGMQVGPKTAPITDEV | |
| 420 430 440 450 460 | |
| 480 490 500 510 520 530 | |
| dbpf1. LDYDTVMENFDKSLDWLTDTYVDAMNIIHYMTDKYNYEAVQMAFLPTKVRANMGFGICGF | |
| \|\|:\|\|\|\|:::\|: :\|\|\|:: \|\|:\|:\|:\|\|\|\| \|\|\|:\|\|\|: \|\|: :\| :\|: \|\| \|: | |
| pf1b_h LDFDTVMTRMDSFMDWLAKQYVTALNVIHYMHDKYSYEAALMALHDRDVYRTMACGIAGL | |
| 470 480 490 500 510 520 | |
| 540 550 560 570 580 | |
| dbpf1. ANTVDSLSAIKYAKVKTLR----DENGYI------YDYEVEGDFPRYGEDDDRADDIAKL | |
| : ::\|\|\|\|\|\|\|\|\|\|\|\|::\| \|::\| : \|:\|:\|\|::\|:\|\|::\|:\|:\|\|\|\| | |
| pf1b_h SVAADSLSAIKYAKVKPVRGDIKDKDGNVVATNVAIDFEIEGEYPQYGNNDNRVDDIACD | |
| 530 540 550 560 570 580 | |
| 590 600 610 620 630 640 | |
| dbpf1. VMKMYHEKLASHKLYKNAEATVSLLTITSNVAYSKQTGNSPVHKGVFLNEDGTVNKSKLE | |
| ::::: :\|::: \| \|:\|\| :\| \|:\|\|\|\|\|\|\|:\|:\|:\|\|\|:\| \|\| : : | |
| pf1b_h LVERFMKKIQKLKTYRNAVPTQSVLTITSNVVYGKKTGNTP---------DGRRAGAP-- | |
| 590 600 610 620 630 | |
| 650 660 670 680 690 700 | |
| dbpf1. FFSPGANPSN-KAKGGWLQNLRSLAKLEFKDANDGISLTTQVSPRALGKTRDEQVDNLVQ | |
| \|:\|\|\|\|\| : ::: \| :::\| \|:\|\|\| \| \|:\|\|\|\| \| : \|:\|\|\|\|: ::\| \|\|: | |
| pf1b_h -FGPGANPMHGRDQKGAVASLTSVAKLPFAYAKDGISYTFSIVPNALGKDAEAQRRNLAG | |
| 640 650 660 670 680 690 | |
| 710 720 730 740 750 760 | |
| dbpf1. ILDGYFTPGALINGTEFAGQHVNLNVMDLKDVYDKIMRGEDVIVRISGYCVNTKYLTPEQ | |
| ::\|\|\|\| ::\| ::\|:: : :\| \|\| \| \|: :: : ::::\|:\|\|\| \|: : \|\| \|\| | |
| pf1b_h LMDGYFHHEATVEGGQHLNVNV-LNREMLLDAMENPDKYPQLTIRVSGYAVRFNSLTKEQ | |
| 700 710 720 730 740 750 | |
| 770 780 | |
| dbpf1. KQELTERVFHEVLSNDDEEVMHTSNIX | |
| :\|::::\|:\| \| : | |
| pf1b_h QQDVITRTFTESM | |
| 760 | |

```
dbpf1.pep
sw:pf1_chlre
ID PFL_CHLRE STANDARD; PRT; 195 AA.
AC P37836;
DE FORMATE ACETYLTRANSFERASE (EC 2.3.1.54) (PYRUVATE FORMATE-LYASE) . . .
SCORES Init1: 163 Initn: 259 Opt: 306
                                      38.0% identity in 213 aa overlap
                        540 550 560 570 580 590
dbpf1. NTVDSLSAIKYAKVKTLRDENGYIYDYEVEGDFPRYGEDDDRADDIAKLVMKMYHEKLAS
                                                                            |:||:||:||||:|:||: ::: : :|||:
pf1_ch GSFPKYGNDDDRVDEIAEWVVSTFSSKLAK
                                                                                            10 20 30
                        600 610 620 630 640 650
dbpf1. HKLYKNAEATVSLLTITSNVAYSKQTGNSPVHKGVFLNEDGTVNKSKLEFFSPGANP-SN
                :: |:|: :|:|:|||||||:|:|:||::| || ::| | |:||||| :
pf1_ch QHTYRNSVPTLSVLTITSNVVYGKKTGSTP---------DG---RKKGEPFAPGANPLHG
                                40 50 60 70
                          660 670 680 690 700 710
dbpf1. KAKGGWLQNLRSLAKLEFKDANDGISLTTQVSPRALGK-TRDEQVDNLVQILDGYFTPGA
                :: | |::|:|:||| :: |||| | : |::||: : :|:::||: ||||||: |:
pf1_ch RDAHGALASLNSVAKLPYTMCLDGISNTFSLIPQVLGRGGEHERATNLASILDGYFANGG
                80 90 100 110 120 130
                            720 730 740 750 760 770
dbpf1. LINGTEFAGQHVNLNVMDLKDVYDKIMRGEDVIVRISGYCVNTKYLTPEQKQELTERVFH
                      ::: :: : ::::: : | ::::|:||| |: ||:||: |:::|:||
pf1_ch HHINVNVLNRSMLMDAVEHPEKY------PNLTIRVSGYAVHFARLTREQQLEVIARTFH
              140 150 160 170 180 190
                            780
dbpf1. EVLSNDDEEVMHTSNIX (corresponding to amino acid residues 535-788 of SEQ ID NO:16)
                :::
pf1_ch DTM (SEQ ID NO:21)
```

The highest homology value obtained when analysing the sequence from clone pfl1 corresponds to the S. mutans pfl gene (Table 3.1), i.e. about 80% at the DNA level, in the region covered by the probe used for library screening and 68.5% for the 1.1 kb pfl fragment analyzed.

Sequence comparisons indicated that the fragment included in clone pfl1 encompasses 367 amino acids of the C-terminal region of the L. lactis pfl gene. Therefore, about 1.3 kb of the 5′-end of the pfl gene was lacking.

A 0.6 kb PstI-EcoRI fragment of clone pfl1, spanning from the polylinker (PstI site) and including a fragment spanning from positions 1342-2003 in the sequence shown in Table 3.2, was randomly labelled and used for screening a λZAP genomic library of strain DB1341 (Sambrook et al., 1989) to get the upstream region of the pfl gene. High stringency hybridization (washing steps at 65° C., 2×30 min in 2×SSC, then 1×30 min in 0.1×SSC; 0.1% SDS) resulted in the isolation of twelve positive clones.

Sequence analysis of clones pfl9, pfl10, pfl19 and pfl20 showed that they included the same pfl fragment as did clone pfl1. Restriction analysis of the above clones showed that they all contained a 460 bp Sau3AI fragment identical to pfl1 (positions 1342-1798 in Table 1.2). Only clone pfl14 showed a different Sau3AI restriction pattern. This clone lacked the above Sau3AI fragment and had a 600 bp fragment that hybridized to the PstI-EcoRI pfl probe, suggesting that rearrangement of the insert occurred during in vivo excision of the plasmid. Sequence analysis of pfl14 confirmed that it included a pfl fragment that lacked the Sau3AI site at position 1 in clone pfl1, but showed sequence identity from position 30 onwards in clone pfl1 (position 1372 in Table 3.2). It is therefore likely that the presence of an intact L. lactis pfl gene is toxic in E. coli and leads to plasmid rearrangement.

4. Inverse PCR to Obtain the Complete pfl Sequence of L. lactis DB1341

To facilitate the characterization of the 5′ region of the L. lactis pfl gene from strain DB1341 inverse PCR was used. EcoRI-digested genomic DNA of strain DB1341 was religated at low concentration (Sambrook et al., 1989) and PCR was carried out using primers pfl1-250 and pfl-390 (see FIG. 4). A 1.6 kb fragment that contained the lacking 421 codons and the upstream region of the L. lactis pfl sequence (positions 1 to 1342 in Table 3.2) was amplified. This PCR fragment was re-amplified from EcoRI-digested and religated DB 1341 DNA using modified primers pfl1-250 (including an XhoI site at the 5′-end) and pfl1-390 (including a BamHI site at the 5′-end) and the amplified product was digested with XhoI and BamHI and ligated into vector pGEM digested with the same enzymes and transformation of E. coli DH5α resulted in strain pflup-1. The L. lactis DB1341 pfl gene encodes a 787 amino acid protein (Tables 3.2, 3.4 and 3.6) with a deduced molecular weight of 89.1 kDa.

A sample of E. coli DH5α strain pflup-1 was deposited under the Budapest Treaty with the German Collection of Microorganisms and Cell Cultures, Mascheroder Weg 1b, D-38 124 Braunschweig, Germany on 18 Jul. 1996 under the accession No. DSM 11087.

5. Cloning of the pfl Upstream Sequence from L. lactis DB1341

Inverse PCR was carried out on HhaI-digested and religated chromosomal DNA of strain DB1341, using primers derived from the above sequence (Table 3.2). The HhaI fragment spans about 1.7 kb from position 1 to 1707 in the below sequence which overlaps the sequence shown in Table 3.2 from position 1563 to 1750.

TABLE 3.5
pf1 upstream sequence from L. lactis DB1341

| | | | |
|---|---|---|---|
| | HhaI | | |
| 1 | GCGCCTAGATAAGAAACAGCAACAGCTAAAAGATAGGTATCAAAAGCACT | 50 | |
| 51 | TGATTTAAAAATAATGACTTTATCCGATTTTTTGATTCCCAACTCAGATA | 100 | |
| 101 | AGAGACTTGCCTTATCAACAATTGCTTGATGAGTCTTTTGGTAAGTCGTT | 150 | |
| 151 | TCAAGAGCTAGTTCGGGGAAAGCTCCAACAGCCTCATCAAAGATAATTGG | 200 | |
| 201 | GCTATCAGGAAACTGTTCAGCTGATTTTTTAAAGTTTAGATACAAATTTA | 250 | |
| 251 | GGGGTTCGTGTTTGAATTTCAAAAAAAATCTCCTCAAGTTAATAAGTTTA | 300 | |
| 302 | TTATATCACAAAGTATTCTTTAGACCAATAGTTAATGTAAATGTTTTCTT | 350 | |
| 351 | AAGTCGTAGAGAATAAAATTCTCGGAAAAAAAGTCTAAAATCTGCTACAA | 400 | |
| 401 | TTAAAGGGACACTAAGAGGATTCCAATCCTCTTTTATCAGGAAAAGAAGG | 450 | |
| 451 | GATAGATAGGAAAATGATTAAAAATTATGAACTATCCAACGAAAAAAAAT | 500 | |
| | orfA M I K N Y E L S N E K K L | | (SEQ ID NO:35) |
| 501 | TAATTTCAACCTCTGAAATGAAGAATTTCACCTATGTTCTCAATCCAACA | 550 | |
| | I S T S E M K N F T Y V L N P T | | |
| 551 | CGTGAAGAAATTGGGAATATTTCTGAATACTATGACTTCCCTTTTGACTA | 600 | |
| | R E E I G N I S E Y Y D F P F D Y | | |
| 601 | TTTATCAGGAATTTTGGATGACTATGAAAATGCCCGTTTTGAAACAGATG | 650 | |
| | L S G I L D D Y E N A R F E T D D | | |
| 651 | ATAATGATAATAATCTGATTCTCTTACAATATCCTCCACTCTCTAATTAT | 700 | |
| | N D N N L I L L Q Y P P L S N Y | | |
| 701 | GGAGAAGTGGCGACTTTTCCATATTCTTTGGTTTGGACTAAAAATGAATC | 750 | |
| | G E V A T F P Y S L V W T K N E S | | |
| 751 | GGTTATTTTAGCACTTAATCATGAGATTGATAATGGCTTAATTTTCGAGC | 800 | |
| | V I L A L N H E I D N G L I F E R | | |
| 801 | GTGAATATGATTATAAACGCTACAAACATCAAGTTATTTTTCAAGTGATG | 850 | |
| | E Y D Y K R Y K H Q V I F Q V M | | |
| 851 | TATCAAATGACACACACTTTCCATGATTATTTGAGAGATTTCCGAACAAG | 900 | |
| | Y Q M T H T F H D Y L R D F R T R | | |
| 901 | GCGTCGCAGACTTGAACAGGGAATCAAAAATTCAACAAAGAACGACCAAA | 950 | |
| | R R R L E Q G I K N S T K N D Q I | | |
| 951 | TTGTTGATTTGATTGCCATTCAAGCAAGTTTAATTTATTTTGAAGATGCC | 1000 | |
| | V D L I A I Q A S L I Y F E D A | | |
| 1001 | TTGCACAATAATATGCAAGTACTTCAGGATTTTATTGATTACTTGAGAGA | 1050 | |
| | L H N N M Q V L Q D F I D Y L R E | | |
| 1051 | AGATGATGAAGACGGTTTTGCTGAAAAGATTTATGATATTTTTGTCGAAA | 1100 | |
| | D D E D G F A E K I Y D I F V E T | | |
| 1101 | CAGACCAAGCTTATACAGAAACCAAGATTCAGCTCAAGTTACTAGAAAAT | 1150 | |
| | D Q A Y T E T K I Q L K L L E N | | |
| 1151 | CTCCGAGATTTGTTCTCAAACAATGTCTCTAATAACTTGAACATTGTCAT | 1200 | |
| | L R D L F S N N V S N N L N I V M | | |
| 1201 | GAAAATCATGACATCAGCTACTTTCGTTCTAGGGATTCCTGCAGTAATTG | 1250 | |
| | K I M T S A T F V L G I P A V I V | | |
| 1251 | TTGGTTTTTACGGAATGAATGTTCCAATTCCTGGTCAAAATTTTAATTGG | 1300 | |
| | G F Y G M N V P I P G Q N F N W | | |
| 1301 | ATGGTTTGGCTTATTTTAGTTCTAGGAATTTTATTATGTGTTTGGGTCAC | 1350 | |
| | M V W L I L V L G I L L C V W V T | | |
| 1351 | TTGGTGGTTACATAAAAAAGATATGTTATAAAATGGAGAAAAATCTCCAT | 1400 | |
| | W W L H K K D M L Stop | | |
| 1401 | TTTTTTGCTCTTTGTGAAAAAATTAATTAGTGATTGCAGATTATGAAGTT | 1450 | |
| 1451 | AGCAATGTTTGTTAAAACTATTTTGTGAATTATTTATGAAAACGTTTTAA | 1500 | |
| 1501 | AAAAGTATAACAGATATTAAAATAATTGGAACTGTATTAGTAAAGAATCT | 1550 | |
| | EcoRI | | |
| 1551 | GTAATTTCTCTTGAATTCTGTTTGCTATTCTCAAACTGTATGATATAATG | 1600 | |
| 1601 | AAGTTGTAATTTGAAACAGAAAGAACAAAGGAGATTTCAAAATGAAAACC | 1650 | |
| | pf1 M K T | | |
| 1651 | GAAGTTACGGAAAATATCTTTGAACAAGCTTGGGATGGTTTTAAAGGAAC | 1700 | |
| | E V T E N I F E Q A W D G F K G T | | |
| | HhaI | | |
| 1701 | CAACTGGCGCGATAAAGCAAGCGTTACTCGCTTTGTACAAGAAAACTACA | 1750 | |
| | N W R D K A S V T R F V Q E N Y K | | |

Nucleotides 1-1750: SEQ ID NO:34

The sequence included an open reading frame, designated orfA encoding a putative 37 kDa protein with no relevant homology to any sequence in available databases.

### EXAMPLE 4

### Characterization of L. lactis orfA Encoding a Putative Transporter Protein

In gram-negative bacteria, the pfl gene is located downstream of an open reading frame transcribed with focA that codes for a putative membrane-bound formate transporter (Suppmann and Sawers 1994). This genetic organization is conserved in E. coli and H. influenzae but has shown great variation in streptococci (Arnau et al. 1997). In L. lactis, the orfA gene is located immediately upstream of pfl. An open reading frame is also found upstream of the pfl gene in Streptococcus mutans that showed no homology to the L. lactis orfA.

In E. coli, growth under anaerobiosis results in the synthesis of large amounts of PFL protein, about 3% of the total protein content (Suppmann and Sawers 1994). Consequently, high amounts of formate are formed intracellularly. At physiological intracellular pH in E. coli formate (low pKa, 3.75) is not dissociated and therefore is not membrane-permeable. Thus, there is a requirement for a specific transporter to remove the excess formate in the cells.

In the following the novel orfA gene of L. lactis and its gene product is characterized.

1. The orfA Gene Structure, Protein Homology and Structure

Sequence analysis of orfA (see Table 3.5. above) showed a “weak” RBS (AGG) and a consensus −10 promoter region upstream of the ATG start codon. No −35 consensus region was identified, suggesting a low expression level for this gene. The deduced protein encoded by orfA, consisting of 306 amino acids and a size of 37 kDa, showed homology (38% identity at the C-terminus) to a 37 kDa putative lactococcal protein (Donkersloot and Thompson 1995) and to a less extent to numerous membrane-bound transporter proteins. A prediction of the structure of OrfA suggested the presence of a large intracellularly located N-terminal region followed by two transmembrane domains, Leu²⁴² to Phe²⁶⁵ and Asn²⁷⁶ to Val²⁹⁴ (FIG. 6). These features are consistent with a possible role of the protein in transport across the cell membrane, although neither sequence homology nor structural similarities with the E. coli FocA protein could be identified. A molecular prediction of the FocA protein showed the presence of six transmembrane domains, but among the related proteins a certain variation in the number of these domains is found. In fact, one of these proteins, the E. coli NirC has four and not six of these domains in its primary sequence (Suppmann and Sawers 1994).

2. Expression of orfA

RNA was isolated from aerobic and anaerobic cultures of L. lactis MG1363 grown in fermenters at 30° C. Using an orfA specific probe (FIG. 7A), Northern blot hybridization was carried out. As shown (FIG. 7B), a low level of expression was observed under the conditions used, which is in agreement with the sequence analysis (lack of −35 region, short RBS) of the upstream region of orfA and with the level of expression expected for a gene coding a membrane associated protein.

No anaerobic induction was observed in GM17 or GalM17 during exponential growth. In GM17 a lower expression of orfA was detected as compared to GalM17 and virtually no expression of the gene was observed during stationary phase.

3. Construction and Analysis of orfA Mutant Strains in L. lactis MG1363

In order to determine whether orfA is the focA analogue in L. lactis, two mutant strains of MG1363 were constructed. A null mutation was carried out by gene disruption using an internal fragment of the orfA gene (including codons 30-168, FIG. 7A), cloned into the integrative vector pSMA500 and transformed into MG1363. One transformant (MG1363ΔorfA) that formed light blue colonies on X-gal was selected. An orfA multicopy strain was constructed by cloning of the entire coding sequence and promoter region of this gene in pAK80 and transforming into MG1363. As above, a transformant giving blue colonies in X-gal was selected (MG1363 pAK80::orfA).

In E. coli, a focA null mutant strain was capable of growing at higher sodium hypophosphite concentrations than was the wild type strain. This compound is a formate analogue that is toxic. Thus, transport of hypophosphite into the cytosol via the FocA channel protein is deleterious for the cells (Suppmann and Sawers 1994). If the OrfA protein has a similar function in L. lactis as does FocA in E. coli, then a null mutant should show an increased resistance to hypophosphite and a strain containing multiple copies of the gene should be more sensitive to this compound than the wild type. As shown in FIG. 8, strain MG1363 showed reduced growth when the medium was supplemented with 500 mM of hypophosphite and it did not grow at 600 mM.

MG1363ΔorfA grew at 600 mM and was unable to grow at higher concentrations. The orfA multicopy strain, MG1363 pAK80::orfA was completely unable to grow at 500 mM hypophosphite. Thus, these results confirmed that OrfA may represent a formate transporter protein in L. lactis.

The mutant strains constructed included a translational fusion of the orfA gene to the lacLM reporter gene (Madsen et al. 1996). The effect of the addition of formate to the medium on the expression of orfA was studied. To exclude a possible toxic effect of the addition of formate to the medium, a dosis curve was studied. Growth inhibition of the wild type strain was observed at formate concentrations exceeding 10 mM. Exponentially growing cultures (OD₆₀₀ about 1) were used to measure β-galactosidase after the addition of 10 mM of formate to the growth medium. As shown in the below Table 3.6 similar levels of β-galactosidase were observed in MG1363ΔorfA independently of the addition of formate or the growth conditions.

TABLE 4.1
Analysis of orfA expression in mutant strains of L. lactis strains.^{a)}

| | Aerobic | | Anaerobic | |
|---|---|---|---|---|
| STRAIN | +Formate | −Formate | +Formate | −Formate |
| MG1363ΔorfA | 9.1 ± 0.3 | 8.2 ± 0.7 | 7.5 ± 0.7 | 6.2 ± 0.1 |
| MG1363pAK80::orfA | 14.6 ± 0.2 | 16.7 ± 0.7 | 13.2 ± 0.1 | 13.2 ± 1.3 |

^{a)}β-galactosidase activity in exponentially growing cultures. At OD₆₀₀ about 1, formate was added (+formate) and the cultures were incubated further for 15 min before cells were separated by centrifugation and frozen.

Higher levels were observed in all cases with the multicopy strain MG1363 pAK80::orfA. These levels, about 2-fold higher, did not correlate with the number of copies (5-10 per cell) expected in this strain. A degree of regulation of expression may exist for orfA in L. lactis to ensure an appropriate level of the OrfA protein.

### EXAMPLE 5

### Isolating and Characterizing the pfl Gene from L. lactis Subspecies lactis MG1363

1. Cloning of a Fragment of the pfl Gene

A pfl fragment was amplified with the above modified primers pfl1-20 and pfl1-1066 from chromosomal DNA of strain MG1363 (see FIG. 4). This fragment was digested and cloned into the vector pGEM digested with XhoI and BamHI, respectively and transformed into E. coli strain DH5α (Stratagene), resulting in strain MGpfl-1. The fragment was sequenced using the relevant primers derived from the sequence of the DB1341 pfl fragment (see FIG. 4).

The sequence of the MG1363 pfl fragment showed 48 differences (42 base changes and a 6 bp deletion) in the 1 kb region characterized when compared to the corresponding sequence of the DB1341 pfl (below Table 5.1). The deduced Pfl protein fragment encoded by the characterized pfl sequences of strains MG1363 and DB1341 showed high homology. Only four sequence differences are found in a 336 amino acid stretch (below Table 5.1): two amino acid substitutions (Pro⁴⁴⁷ to Thr⁴⁷³ and Asn⁴⁸⁶ to Asp⁴⁸⁶ in Table 5.1) and two adjacent deletions (Asp⁴⁵⁴-Asp⁴⁵⁵) encoded by the DB1341 pfl gene. The latter two residues are also present in the protein encoded by the S. mutans pfl gene.

A sample of E. coli DH5α strain MGpfl-1 was deposited under the Budapest Treaty with the German Collection of Microorganisms and Cell Cultures, Mascheroder Weg 1b, D-38 124 Braunschweig, Germany on 18 Jul. 1996 under the accession Nos DSM 11088.

TABLE 5.1

Homology between the DNA sequences of a fragment of the pf1 gene
fragment isolated from L. lactis strains DB1341 (db1341pf1)
and a fragment of the pf1 gene of MG1363 (mg1363-pf1)
The comparison starts at the position of the Sau3AI site
in the L. lactis DB 1341 pf1 gene (position 1342 in TABLE 3.2).

| | | |
|---|---|---|
| | 1 50 | |
| mg1363pf1 | .......... .......... .......... .......... .......... | (SEQ ID NO:22) |
| db1341pf1 | GATCCAGAAA ATGAAGAAGG ACGTCATAAC CTCCAATACT TTGGTGCGCG | |
| consensus | .......... .......... .......... .......... .......... | |
| | 51 100 | |
| mg1363pf1 | .......... .......... TGTTACCTGG TTTGAACGGT GGTTAC.... | |
| db1341pf1 | TGTAAACGTC TTGAAAGCAA TGTTGACTGG TTTGAACGGT GGTTATGATG | |
| consensus | .......... .......... TGTT..CTGG TTTGAACGGT GGTTA..... | |
| | 101 150 | |
| mg1363pf1 | ..GTTCATAA AGATTATAAA GTATTCGATA TTGAACCTGT TCGTGATGAA | |
| db1341pf1 | ACGTTCATAA AGATTATAAA GTATTCGACA TCGAACCTGT TCGTGACGAA | |
| consensus | ..GTTCATAA AGATTATAAA GTATTCGA.A T.GAACCTGT TCGTGA.GAA | |
| | 151 200 | |
| mg1363pf1 | ATTCTTGACT ATGATACAGT TATGGAAAAC TTCGACAAAT CACTCAACTG | |
| db1341pf1 | ATTCTTGACT ATGATACAGT TATGGAAAAC TTTGACAAAT CTCTCGACTG | |
| consensus | ATTCTTGACT ATGATACAGT TATGGAAAAC TT.GACAAAT C.CTC.ACTG | |
| | 201 250 | |
| mg1363pf1 | GTTGACAGAT ACTTATGTTG ATGCAATGAA TATCATTCAC TACATGACTG | |
| db1341pf1 | GTTGACTGAT ACTTATGTTG ATGCAATGAA TATCATTCAT TACATGACTG | |
| consensus | GTTGAC.GAT ACTTATGTTG ATGCAATGAA TATCATTCA. TACATGACTG | |
| | 251 300 | |
| mg1363pf1 | ACAAATATAA CTATGAAGCA GTTCAAATGG CCTTCTTGCC TACTAAAGTT | |
| db1341pf1 | ATAAATATAA CTATGAAGCA GTTCAAATGG CCTTCTTGCC TACTAAAGTT | |
| consensus | A.AAATATAA CTATGAAGCA GTTCAAATGG CCTTCTTGCC TACTAAAGTT | |
| | 301 350 | |
| mg1363pfL | CGTGCTAACA TGGGATTTGG TATCTGTGGT TTCGCAAATA CAGTTGATTC | |
| db1341pf1 | CGTGCTAACA TGGGATTTGG TATCTGTGGA TTCGCAAATA CAGTTGATTC | |
| consensus | CGTGCTAACA TGGGATTTGG TATCTGTGG. TTCGCAAATA CAGTTGATTC | |
| | 351 400 | |
| mg1363pf1 | ACTTTCAGCG ATTAAATATG CTAAAGTTAA AACTTTGCGT GATGAAAATG | |
| db1341pf1 | ACTTTCAGCA ATTAAATATG CTAAAGTTAA AACATTGCGT GATGAAAATG | |
| consensus | ACTTTCAGC. ATTAAATATG CTAAAGTTAA AAC.TTGCGT GATGAAAATG | |
| | 401 450 | |
| mg1363pf1 | GCTACATCTA CGATTATGAA GTAGAAGGTG ACTTCCCACG TTATGGTGAA | |
| db1341pf1 | GCTATATCTA CGATTACGAA GTAGAAGGTG ATTTCCCTCG TTATGGTGAA | |
| consensus | GCTA.ATCTA CGATTA.GAA GTAGAAGGTG A.TTCCC.CG TTATGGTGAA | |
| | 451 500 | |
| mg1363pf1 | GATGATGACC GTGCTGATGA TATCGCTAAA CTTGTCATGA AAATGTACCA | |
| db1341pf1 | GATGATGATC GTGCTGATGA TATTGCTAAA CTTGTCATGA AAATGTACCA | |
| consensus | GATGATGA.C GTGCTGATGA TAT.GCTAAA CTTGTCATGA AAATGTACCA | |
| | 501 550 | |
| mg1363pf1 | TGAAAAATTA GCTTCACACA AACTTTACAA AAATGCTGAA GCTACTGTTT | |
| db1341pf1 | TGAAAAATTA GCTTCACACA AACTTTACAA AAATGCTGAA GCTACTGTTT | |
| consensus | TGAAAAATTA GCTTCACACA AACTTTACAA AAATGCTGAA GCTACTGTTT | |
| | 551 600 | |
| mg1363pf1 | CACTTTTGAC AATCACATCT AACGTTGCTT ACTCTAAACA AACTGGTAAC | |
| db1341pf1 | CACTTTTGAC AATTACATCT AACGTTGCTT ACTCTAAACA AACTGGTAAT | |
| consensus | CACTTTTGAC AAT.ACATCT AACGTTGCTT ACTCTAAACA AACTGGTAA. | |
| | 601 650 | |
| mg1363pf1 | TCTCCAGTTC ATAAAGGAGT ATTCCTCAAT GAAGATGGTA CAGTCAACAA | |
| db1341pf1 | TCTCCAGTAC ATAAAGGAGT ATTCCTCAAT GAAGATGGTA CAGTAAATAA | |
| consensus | TCTCCAGT.C ATAAAGGAGT ATTCCTCAAT GAAGATGGTA CAGT.AA.AA | |
| | 651 700 | |
| mg1363pf1 | ATCTAAACTT GAATTCTTCT CACCAGGTGC TAACCCATCT AACAAAGCTA | |
| db1341pf1 | ATCTAAACTT GAATTCTTCT CACCAGGTGC TAACCCATCT AATAAAGCTA | |
| consensus | ATCTAAACTT GAATTCTTCT CACCAGGTGC TAACCCATCT AA.AAAGCTA | |
| | 701 750 | |
| mg1363pf1 | AAGGTGGATG GTTGCAAAAT CTTCGTTCAT TAGCTAAATT GGAATTCAAA | |
| db1341pf1 | AGGGTGGTTG GTTGCAAAAC CTTCGCTCAT TGGCTAAGTT GGAATTCAAA | |
| consensus | A.GGTGG.TG GTTGCAAAA. CTTCG.TCAT T.GCTAA.TT GGAATTCAAA | |
| | 751 800 | |
| mg1363pf1 | GATGCAAATG ACGGTATTTC ATTAACTACT CAAGTTTCTC CTCGTGCACT | |
| db1341pf1 | GATGCAAATG ATGGTATTTC ATTGACTACT CAAGTTTCAC CTCGTGCACT | |
| consensus | GATGCAAATG A.GGTATTTC ATT.ACTACT CAAGTTTC.C CTCGTGCACT | |
| | 801 850 | |
| mg1363pf1 | TGGTAAAACT CGTGATGAAC AAGTAGATAA CTTGGTTCAA ATTCTTGATG | |
| db1341pf1 | TGGTAAAACT CGTGATGAAC AAGTGGATAA CTTGGTTCAA ATTCTTGATG | |
| consensus | TGGTAAAACT CGTGATGAAC AAGT.GATAA CTTGGTTCAA ATTCTTGATG | |
| | 851 900 | |
| mg1363pf1 | GATACTTCAC ACCAGGAGCT TTGATTAATG GTACTGAATT TGCAGGTCAA | |
| db1341pf1 | GATACTTCAC ACCAGGTGCT TTGATTAATG GTACTGAATT TGCAGGTCAA | |
| consensus | GATACTTCAC ACCAGG.GCT TTGATTAATG GTACTGAATT TGCAGGTCAA | |
| | 901 950 | |
| mg1363pf1 | CACGTTAACT TGAACGTTAT GGACCTTAAA GATGTTTACG ATAAAATCAT | |
| db1341pf1 | CACGTTAACT TGAACGTAAT GGACCTTAAA GATGTTTACG ATAAAATCAT | |
| consensus | CACGTTAACT TGAACGT.AT GGACCTTAAA GATGTTTACG ATAAAATCAT | |
| | 951 1000 | |
| mg1363pf1 | GCGTGGTGAA GATGTTATCG TTCGTATCTC TGGATACTGT GTTAACACTA | |
| db1341pf1 | GCGTGGTGAA GATGTTATCG TTCGTATCTC TGGTTACTGT GTCAATACTA | |
| consensus | GCGTGGTGAA GATGTTATCG TTCGTATCTC TGG.TACTGT GT.AA.ACTA | |
| | 1001 1050 | |
| mg1363pf1 | AATACCTCAC ACCTGAACAA AAACAAGAAT TGACTGAACG TGTCTTCCAT | |
| db1341pf1 | AATACcTCAC ACCAGAACAA AAACAAGAAT TAACTGAACG TGTCTTCCAT | |
| consensus | AATACCTCAC ACC.GAACAA AAACAAGAAT T.ACTGAACG TGTCTTCCAT | |
| | 1051 1100 | |
| mg1363pf1 | GAAGTACTTT CAAACGATGA TGAAGAAGTA AT | |
| db1341pf1 | GAAGTTCTTT CAAACGATGA TGAAGAAGTA ATGCATACTT CAAACATCTA | |
| consensus | GAAGT.CTTT CAAACGATGA TGAAGAAGTA AT........ .......... | |
| | 1101 1150 | |
| db1341pf1 | ATTCTTAAAA TTTAATGAAT ATTCGGTCTG TCAGTTTTAC TGACAGACTT | |
| consensus | .......... .......... .......... .......... .......... | |
| | 1151 1200 | |
| db1341pf1 | TTTTTTACGA AAAAATTAAT CATAATAGTT AAAAACTATT GTTTTTAGTT | |
| consensus | .......... .......... .......... .......... .......... | |
| | 1201 1250 | |
| db1341pf1 | TAAGAAAGTT AAATTTTATG CTAAAATAGA TGAATGAAAA TGGTAATTGG | |
| consensus | .......... .......... .......... .......... .......... | |
| | 1251 1300 | |
| db1341pf1 | ATTGACAGGC GGAATTGCGA KTGGGAAATC AACGGTGGTT GATTTTTTGA | |
| consensus | .......... .......... .......... .......... .......... | |
| db1341pf1: | corresponding to nucleotides 1342-2641 of SEQ ID NO:15 | |

TABLE 5.2

Multialignment of the putative Pf1 protein from L. lactis strains MG1363
(partial sequence; 1) and DB134l (2) with the deduced amino acid sequences
of known cloned bacterial pf1 genes
The L. lactis Pf1 proteins were aligned with the following known Pf1 proteins:
deduced proteins of S. mutans pf1 (3); E. coli pf13 and pf1b genes
(Accession Nos. P42632 and P09373; 4 and 5); H. influenzae Pf1 (6);
C. pasteurianum Pf1 (7).
Consensus (con) shows conserved positions (bold) among all of the protein
sequences. The four amino acid differences between the MG1363 and DB1341 Pf1
are shown in underlined, bold at the top (1)

| | | |
|---|---|---|
| | 60 | |
| 2 | MKTEVTENI FEQAWDGFKG TNWRDKASVT RFVQENYKPY DGDESFLAGP TERTLKVKKI | (SEQ ID NO:16) |
| 3 | MATVKTNTDV FEKAWEGFKG TDWKDRASIS RFVQDNYTPY DGGESFLAGP TERSLHIKKV | (SEQ ID NO:24) |
| 4 | MKVDIDTSDKL YADAWLGFKG TDWKNEINVR DFIQHNYTPY EGDESFLAEA TPATTELWEK | (SEQ ID NO:19) |
| 5 | SELNEK LATAWEGFTK GDWQNEVNVR DFIQKNYTPY EGDESFLAGA TEATTTLWDK | (SEQ ID NO:14) |
| 6 | SELNEM QKLAWAGFAG GDWQENVNVR DFIQKNYTPY EGDDSFLAGP TEATTKLWES | (SEQ ID NO:20) |
| 7 | LFKQWEGFQD GEWTNDVNVR DFIQKNYKEY TGDKSFLKGP TEKTKKVWDK | (SEQ ID NO:25) |
| con | W GF W F Q NY Y G SFL T | |
| | 120 | |
| 2 | IEDTKNHYEE VGFPFDTD-- RVTSIDKIPA GYIDANDKEL ELIYGMQNSE LFRLNFMPRG | |
| 3 | VEETKAHYEE TRFPMDT--- RITSIADIPA GYID---KEN ELIFGIQNDE LFKLNFMPKG | |
| 4 | VMEGIRIENA THAPVDFDTN IATTITAHDA GYIN---QPL EKIVGLQTDA PLKRALHPFG | |
| 5 | VMEGVKLENR THAPVDFDTA VASTITSHDA GYIN---KQL EKIVGLQTEA PLKRALIPFG | |
| 6 | VMEGIKIENR THAPLDFDEH TPSTIISHAP GYIN---KDL EKIVGLQTDE PLKRAIMPFG | |
| 7 | AVS-LILEEL KKGILDVDTE TISGINSFKP GYLD---KDN EVIVGFQTDA PLKRITNPFG | |
| con | D I GY E I G Q P G | |
| | 180 | |
| 2 | GLRVAEKILT EHGLSVDPGL HDVLSQTMTS VNDGIFRAYT SAIRKARHAH TVTGLPDAYS | |
| 3 | GIRMAETALK EHGYEPDPAV HEIFTKYATT VNDGIFRAYT SNIRRARHAH TVTGLPDAYS | |
| 4 | GINMIKSSFH AYGREMDSEF EYLFTDLRKT HNQGVFDVYS PDMLRCRKSG VLTGLPDGYG | |
| 5 | GIKMIEGSCK AYNRELDPMI KKIFTEYRKT HNQGVFDVYT PDILRCRKSG VLTGLPDAYG | |
| 6 | GIKMVEGSCK VYGRELDPKV KKIFTEYRKT HNQGVFDVYT PDILRCRKSG VLTGLPDAYG | |
| 7 | GIRMAEQSLK EYGFKISDEM HNIFTNYRKT HNQGVFDAYS EETRIARSAG VLTGLPDAYG | |
| con | G G F Y R TGLPD Y | |
| | 240 | |
| 2 | RGRIIGVYAR LALYGADYLM KEKAKEWDAI ------TEIN EENIRLKEEI NMQYQALQEV | |
| 3 | RGRIIGVYAR LALYGADYLM QEKVNDWNSI ------AEID EESIRLREEI NLQYQALGEV | |
| 4 | RGRIIGDYRR VALYGISYLV RERELQFADL QSRLEKGEDL EATIRLREEL AEHRHALLQI | |
| 5 | RGRIIGDYRR VALYGIDYLM KDKLAQFTSL QADLENGVNL EQTIRLREEI AEQHRALGQM | |
| 6 | RGRIIGDYRR VALYGVDFLM KDKYAQFSSL QKDLEDGVNL EATIRLREEI AEQHRALGQL | |
| 7 | RGRIIGDYRR VALYGIDFLI QEKKKDLSNL -----KGDML DELIRLREEV SEQIRALDEI | |
| con | RGRIIG Y R ALYG L IRLREE AL | |
| | 300 | |
| 2 | VNFGALYGLD VSRPAMNVKE AIQWVNIAYM AVCRVINGAA TSLGRVPIVL DIFAERDLAR | |
| 3 | VRLGDLYGLD VRKPAMNVKE AIQWINIAFM AVCRVINGAA TSLGRVPIVL DIFAERDLAR | |
| 4 | QEMAAKYGFD ISRPAQNAQE AVQWLYFAYL AAVKSQNGGA MSLGRTASFL DIYIERDFKA | |
| 5 | KEMAAKYGYD ISGPATNAQE AIQWTYFGYL AAVKSQNGAA MSFGRTSTFL DVYIERDLKA | |
| 6 | KQMAASYGYD ISNPATNAQE AIQWMYFAYL AAIKSQNGAA MSFGRTATFI DVYIERDLKA | |
| 7 | KKMALSYGVD ISRPAVNAKE AAQFLYFGYL AGVKENNGAA MSLGRTSTFL DIYIERDLEQ | |
| con | YG D PA N E A Q A NG A S GR D ERD | |
| | 360 | |
| 2 | GTFTEQEIQE FVDDFVLKLR TMKFARAAAY DELYSGDPTF ITTSMAGMGN DGRHRVTKMD | |
| 3 | GTFTESEIQE FVDDFVMKLR TVKFARTKAY DELYSGDPTF ITTSMAGMGA DGRHRVTKMD | |
| 4 | GVLNEQQAQE LIDHFIMKIR MVRFLRTPEF DSLFSGDPIW ATEVIGGMGL DGRTLVTKNS | |
| 5 | GKITEQEAQE MVDHLVMKLR MVRFLRTPEY DELFSGDPIW ATESIGGMGL DGRTLVTKNS | |
| 6 | GKITETEAQE LVDHLVMKLR MVRFLRTPEY DQLFSGDPMW ATETIAGMGL DGRTLVTKNT | |
| 7 | GLITEDEAQE VIDQFIIKLR LVRHLRTPEY NELFAGDPTW VTESIAGVGI DGRSLVTKNS | |
| con | G QE D K R R L GDP T G G DGR VTK | |
| | 420 | |
| 2 | YRFLNTLDTI GNAPEPNLTV LWDSKLPYSF KRYSMSMSHK HSSIQYEGVE TMAKDGYGEM | |
| 3 | YRFLNTLDNI GNAPEPNLTV LWSSKLPYSF RHYCMSMSHK HSSIQYEGVT TMAKEGYGEM | |
| 4 | FRYLHTLHTM GPAPEPNLTI LWSEELPIAF KKYAAQVSIV TSSLQYENDD LMRTDFNSDD | |
| 5 | FRFLNTLYTM GPSPEPNMTI LWSEKLPLNF KKFAAKVSID TSSLQYENDD LMRPDFNNDD | |
| 6 | FRILHTLYNM GTSPEPNLTI LWSEQLPENF KRFCAKVSID TSSVQYENDD LMRPDFNNDD | |
| 7 | FRYLHTLINL GSAPEPNMTV LWSENLPESF KKFCAEMSIL TDSIQYENDD IMRPI-YGDD | |
| con | L TL G PEPN T LW LP F S S QYE M | |
| | 480 | |
| 1 | LPGLNG GY--VHKDYK VFDIEPVRDE | (SEQ ID NO:23) |
| 2 | SCISCCVSPL DPENEEGRHN LQYFGARVNV LKAMLTGLNG GYDDVHKDYK VFDIEPVRDE | |
| 3 | SCISCCVSPL DPENEDRRHN LQYFGARVNV LKALLTGLNG GYDDVHKDYK VFDVEPIRDE | |
| 4 | YAIACCVSPM VIG-----KQ MQFFGARANL AKTLLYAING GVDEKLKIQV GPKTAPLMDD | |
| 5 | YAIACCVSPM IVG-----KQ MQFFGARANL AKTMLYAING GVDEKLKMQV GPKSEPIKGD | |
| 6 | YAIACCVSPM IVG-----KQ MQFFGARANL AKTLLYAING GIDEKLGMQV GPKTAPITDE | |
| 7 | YAIACCVSAM RVG-----KD MQFFGARCNL AKCLLLAING GVDEKKGIKV VPDIEPITDE | |
| con | I CCVS Q FGAR N K L NG G P | |
| | 540 | |
| 1 | ILDYDTVMEN FDKSLNWLTD TYVDAMNIIH YMTDKYNYEA VQMAFLPTKV RANMGFGICG | |
| 2 | ILDYDTVMEN FDKSLDWLTD TYVDAMNIIH YMTDKYNYEA VQMAFLPTKV RANMGFGICG | |
| 3 | VLDFETVKAN FEKALDWLTD TYVDAMNIIH YMTDKYNYEA VQMAFLPTRV KANMGFGICG | |
| 4 | VLDYDKVMDS LDHFMDWLAV QYISALNIIH YMHDKYSYEA SLMALHDRDV YRTMACGIAG | |
| 5 | VLNYDEVMER MDHFMDWLAK QYITALNIIH YMHDKYSYEA SLMALHDRDV IRTMACGIAG | |
| 6 | VLDFDTVMTR MDSFMDWLAK QYVTALNVIH YMHDKYSYEA ALMALHDRDV YRTMACGIAG | |
| 7 | VLDYEKVKEN YFKVLEYMAG LYVNTNNIIH FMHDKYAYEA SQMALHDTKV GRLMAFGIAG | |
| con | L V Y N IH YM DKY YEA MA V M GI G | |
| | 600 | |
| 1 | FANTVDSLSA IKYAKVKTLR DEN------- ---GYIYDYE VEGDFPRYGE DDDRADDIAK | |
| 2 | FANTVDSLSA IKYAKVKTLR DEN------- ---GYIYDYE VEGDFPRYGE DDDRADDIAK | |
| 3 | FSNTVDSLSA IKYATVKPIR DED------- ---GYIYDYE TVGNFPRYGE DDDRVDSIAE | |
| 4 | LSVATDSLSA IKYARVKPIR DEN------- ---GLAVDFE IDGEYPQYGN NDERVDSIAC | |
| 5 | LSVAADSLSA IKYAKVKPIR DED------- ---GLAIDFE IEGEYPQFGN NDPRVDDLAV | |
| 6 | LSVAADSLSA IKYAKVKPVR GDIKDKDGNV VATNVAIDFE IEGEYPQYGN NDNRVDDIAC | |
| 7 | FSVAADSLSA IRYAKVKPIR -EN------- ---GITVDFV KEGDFPKYGN DDDRVDSIAV | |
| con | DSLSA IKYA VK R D G P G D R D A | |
| | 660 | |
| 1 | LVMKMYHEKL ASHKLYKNAE ATVSLLTITS NVAYSKQTGN SPVHKGVFLN EDGTVNKSKL | |
| 2 | LVMKMYHEKL ASHKLYKNAE ATVSLLTITS NVAYSKQTGN SPVHKGVFLN EDGTVNKSKL | |
| 3 | WLLEAFHTRL ARHKLYKDSE ATVSLLTITS NVAYSKQTGN SPVHKGVYLN EDGSVNLSKV | |
| 4 | DLVERFMKKI KALPTYRNAV PTQSILTITS NVVYGQKTGN TPD------- -----GRRAG | |
| 5 | DLVERFMKKI QKLHTYRDAI PTQSVLTITS NVVYGKKTGN TPD------- -----GRRAG | |
| 6 | DLVERFMKKI QKLKTYRNAV PTQSVLTITS NVVYGKKTGN TPD------- -----GRRAG | |
| 7 | EIVEKFSDEL KKHPTYRNAK HTLSVLTITS NVMYGKKTGT TPD------- -----GRKVG | |
| con | Y T S LTITS NV Y TGN P | |
| | 720 | |
| 1 | EFFSPGANPS NKA-KGGWLQ NLRSLAKLEF KDANDGISLT TQVSPRALGK TRDEQVDNLV | |
| 2 | EFFSPGANPS NKA-KGGWLQ NLRSLAKLEF KDANDGISLT TQVSPRALGK TRDEQVDNLV | |
| 3 | EFFSPGANPS NKA-SGGWLQ NLNSLKKLDF AHANDGISLT TQVSPKALGK TFDEQVANLV | |
| 4 | TPFAPGANPM HGRDRKGAVA SLTSVAKLPF TYAKDGISYT FSIVPAALGK EDPVRKTNLV | |
| 5 | APFGPGANPM HGRDQKGAVA SLTSVAKLPF AYAKDGISYT FSIVPNALGK DDEVRKTNLA | |
| 6 | APFGPGANPM HGRDQKGAVA SLTSVAKLPF AYAKDGISYT FSIVPNALGK DAEAQRRNLA | |
| 7 | EPLAPGANPM HGRDMEGALA SLNSVAKVPY VCCEDGVSNT FSIVPDALGN DHDVRINNLV | |
| con | PGANP G L S K DG S T P ALG NL | |
| | 780 | |
| 1 | QILDGYFTPG ALINGTEFAG QHVNLNVMDL KDVYDKIMRG EDV---IVRI SGYCVNTKYL | |
| 2 | QILDGYFTPG ALINGTEFAG QHVNLNVMDL KDVYDKIMRG EDV---IVRI SGYCVNTKYL | |
| 3 | TILDGYF--- ------EGGG QHVNLNVMDL KDVYDKIMNG EDV---IVRI SGYCVNTKYL | |
| 4 | GLLDGYFHHE ADV----EGG QHLNVNVMNR EMLLDAIEHP EKYPNLTIRV SGYACASTH | |
| 5 | GLMDGYFHHE ASI----EGG QHLNVNVMNR EMLLDAMENP EKYPQLTIRV SGYAVRFNSL | |
| 6 | GLMDGYFHEE ATV----EGG QHLNVNVLNR EMLLDAMENP DKYPQLTIRV SGYAVRFNSL | |
| 7 | SIMGGYF--- ------GQGA HHLNVNVLNR ETLIDAMNNP DKYPTLTIRV SGYAVNFNRL | |
| con | GYF H N NV D R SGY | |
| 1 | TPEQKQELTE RVFHEVLSND DEEV | |
| 2 | TPEQKQELTE RVFHEVLSND DEEVMHTSNI Z | |
| 3 | TKEQKTELTQ RVFHEVLSMD DAATDLVNNK Z | |
| 4 | | |
| 5 | TKEQQQDVIT RTFTQSM | |
| 6 | TKEQQQDVIT RTFTESM | |
| 7 | SKDHQKEVIS RTFHEKL | |
| con | | |

2. Cloning and Sequencing of the Entire pfl Gene of L. lactis Strain MG1363

The entire pfl gene sequence was obtained from L. lactis subsp. cremoris strain MG1363 using PCR. Like the pfl coding sequence of L. lactis strain DB1341 the coding sequence of MG1363 comprises 2363 bp and encodes a 787 amino acid PFL protein having a predicted molecular weight of 89.1 kDa.

TABLE 5.3
The complete sequence of the pf1 locus of L. lactis strain MG1363

| | | | |
|---|---|---|---|
| 1 | TTGGGCTATAAGGAAATTGTTCTGCTGATTTTTTAAAGTTTAGATATAGG | 50 | Nucleotides 1-4191: |
| 51 | TTTAGGGGTTCATGTTTGAATTTCAAAAAAAGTCTCCTCAAGTTAATAAG | 100 | SEQ ID NOS:36/38) |
| 101 | TTTATTATATCACAAAGTATTATTTAGACCAACTTCCTTCAAAAAACTTT | 150 | |
| 151 | TCGTTAAGGCTTTGAAATAAAATAATGAGAAAAAAATAGGAAAATCTGCT | 200 | |
| 201 | ACAATTAGAAGGAGAAGAAGAGGATTTAAATCCTTTTTTATTAGGAAAAG | 250 | |
| 251 | AAGGGATAGATAGGCTGATATGATAAAAAATTATGAACTATCCAATGAAA | 300 | |
| | orfA M I K N Y E L S N E K | (SEQ ID NO:37) | |
| | Sau3AI | | |
| 301 | AAAAATTGATCTCAACTTCTGAGATGAAGAATTTCACTTATGTCCTCAAT | 350 | |
| | K L I S T S E M K N F T Y V L N | | |
| 351 | CCAACACGTGAAGAAATTGGGAATATCTCAGAACACTATGATTTTCCTTT | 400 | |
| | P T R E E I G N I S E H Y D F P F | | |
| 401 | TGACTATCTATCTGGAATTTTAGATGACTATGAAAATGCCCGTTTTGAAA | 450 | |
| | D Y L S G I L D D Y E N A R F E T | | |
| 451 | CAGATGATAATGACAATAATCTGATTCTTTTGCAATATCCCGCCTTGTCC | 500 | |
| | D D N D N N L I L L Q Y P A L S | | |
| 501 | AACTATGGAGAAGTGGCCACTTTTCCATATTCTTTGGTTTGGACTAAGAA | 550 | |
| | N Y G E V A T F P Y S L V W T K N | | |
| 551 | TGAATCGGTTATTTTGGCCCTTAACCATGAAATTGATAATGGTCTCATTT | 600 | |
| | E S V I L A L N H E I D N G L I F | | |
| 601 | TTGAACGAGAATATGATTATAAACGCTATAAACACCAATTGATTTTTCAA | 650 | |
| | E R E Y D Y K R Y K H Q L I F Q | | |
| 651 | GTGATGTCACCAAATGACTCATACTTTTCATGATTATTTGAGAGACTTTAG | 700 | |
| | V M Y Q M T H T F H D Y L R D F R | | |
| 701 | AACAAGGCGCCGCCGGCTTGAAGTTGGTATCAAAAATTCAACAAAAAATG | 750 | |
| | T R R R L E V G I K N S T K N D | | |
| 751 | ACCAAATTGTTGACTTAATTGCCATTCAAGCGAGTTTGATTTATTTTGAA | 800 | |
| | Q I V D L I A I Q A S L I Y F E | | |
| 801 | GATGCGCTGCACAATAATATGCAAGTTCTCCAGAATTTTATTGATTACTT | 850 | |
| | D A L H N N M Q V L Q N F I D Y L | | |
| 851 | ACGAGAAGATGATGAAGATGGTTTTGCCGAAAAAATCTATGATATTTTTG | 900 | |
| | R E D D E D G F A E K I Y D I F V | | |
| 901 | TCGAAACAGACCAAGCTTATACAGAAACCAAGATTCAGCTCAAGTTACTA | 950 | |
| | E T D Q A Y T E T K I Q L K L L | | |
| 951 | GAAAATCTCCGAGATTTGTTCTCAAACATTGTCTCTAATAATTTGAATAT | 1000 | |
| | E N L R D L F S N I V S N N L N I | | |
| 1001 | CGTCATGAAAATTATGACCTCAGCAACATTTGTTCTAGGTATTCCGGCGG | 1050 | |
| | V M K I M T S A T F V L G I P A V | | |
| 1051 | TTATTGTCGGCTTTTATGGAATGAATGTTCCGATTCCTGGTCAAAATTTT | 1100 | |
| | I V G F Y G M N V P I P G Q N F | | |
| 1101 | AATTGGATGGTCTGGCTCATTTTGGTGTTTGGAATTTTATTATGTGTTTG | 1150 | |
| | N W M V W L I L V F G I L L C V W | | |
| 1151 | GGTTACTTGGTGGCTACACAAAAAAGATATGTTATGAATGGAGAAAATTT | 1200 | |
| | V T W W L H K K D M L Stop | | |
| 1201 | CTCCGTTTTTTTATCTTTGTGAAAAAATTAATTAGTGATAATAAATCATG | 1250 | |
| 1251 | AAGTTAGCAATGTTTGTCAAAGCTATTTAGTGAATTAATTATGAAAACGT | 1300 | |
| 1301 | TTTAAAAAAGTATAACAGATATTAAAATAATTGAAACTGTATTAGTAAAG | 1350 | |
| | EcoRI | | |
| 1351 | AATCTGTAATTTCTCTTGAATTCTGTTTGCTATTATCAAACTGTATGATA | 1400 | |
| 1401 | TAATGAAGTTGTAATTTGAAACAGAAAGAACAAAGGAGATTTCAAAATGA | 1450 | |
| | pf1 M K | (SEQ ID NO:39) | |
| 1451 | AAACCGAAGTTACGGAAAATATCTTTGAACAAGCTTGGGATGGTTTTAAA | 1500 | |
| | T E V T E N I F E Q A W D G F K | | |
| 1501 | GGAACTAACTGGCGCGATAAAGCAAGCGTTACTCGCTTTGTACAAGAAAA | 1550 | |
| | G T N W R D K A S V T R F V Q E N | | |
| 1551 | CTACAAACCATATGATGGTGATGAAAGCTTTCTTGCTGGGCCAACAGAAC | 1600 | |
| | Y K P Y D G D E S F L A G P T E R | | |
| 1601 | GTACACTTAAAGTAAAGAAAATTATTGAAGATACAAAAAATCACTACGAA | 1650 | |
| | T L K V K K I I E D T K N H Y E | | |
| 1651 | GAAGTAGGATTTCCCTTTGATACTGACCGCGTAACCTCTATCGATAAAAT | 1700 | |
| | E V G F P F D T D R V T S I D K I | | |
| 1701 | TCCTGCTGGATATATTGATGCTAATGATAAAGAACTTGAACTCATCTATG | 1750 | |
| | P A G Y I D A N D K E L E L I Y G | | |
| 1751 | GGATGCAAAATAGCGAACTTTTCCGCTTAAACTTCATGCCAAGAGGTGGT | 1800 | |
| | M Q N S E L F R L N F M P R G G | | |
| 1801 | CTTCGTGTTGCTGAAAAGATTTTGACAGAACACGGTCTTTCAGTTGACCC | 1850 | |
| | L R V A E K I L T E H G L S V D P | | |
| 1851 | AGGTTTGCATGATGTTTTGTCACAAACAATGACTTCTGTAAATGATGGAA | 1900 | |
| | G L H D V L S Q T M T S V N D G I | | |
| 1901 | TCTTCCGTGCTTATACTTCAGCAATTCGTAAAGCACGTCACGCTCACACT | 1950 | |
| | F R A Y T S A I R K A R H A H T | | |
| 1951 | GTAACAGGTTTGCCTGATGCATACTCTCGTGGACGTATCATCGGGGTATA | 2000 | |
| | V T G L P D A Y S R G R I I G V Y | | |
| 2001 | TGCACGTCTTGCTCTTTATGGAGCTGACTACCTTATGAAGGAAAAAGCAA | 2050 | |
| | A R L A L Y G A D Y L M K E K A K | | |
| 2051 | AAGAATGGGATGCAATCACTGAAATTAATGATGATAACATTCGTCTTAAA | 2100 | |
| | E W D A I T E I N D D N I R L K | | |
| 2101 | GAAGAAATTAACATGCAATACCAAGCTTTGCAAGAAGTTGTAAACTTTGG | 2150 | |
| | E E I N M Q Y Q A L Q E V V N F G | | |
| 2151 | TGCTTTGTATGGTCTTGACGTTTCTCGTCCAGCGATGAACGTAAAAGAAG | 2200 | |
| | A L Y G L D V S R P A M N V K E A | | |
| 2201 | CAATCCAATGGGTTAATATTGCATACATGGCAGTTTGTCGTGTTATCAAT | 2250 | |
| | I Q W V N I A Y M A V C R V I N | | |
| 2251 | GGTGCTGCAACTTCACTTGGACGTGTGCCAATCGTTCTTGACATCTTTGC | 2300 | |
| | G A A T S L G R V P I V L D I F A | | |
| 2301 | AGAACGTGACCTTGCTCGTGGAACATTTACTGAGCAAGAAATCCAAGAAT | 2350 | |
| | E R D L A R G T F T E Q E I Q E F | | |
| 2351 | TTGTTGATGATTTCATTTTAAAACTTCGTACAATGAAATTTGCTCGTGCT | 2400 | |
| | V D D F I L K L R T M K F A R A | | |
| 2401 | GCTGCTTATGATGAACTTTATTCTGGTGACCCCACGTTCATCACAACATC | 2450 | |
| | A A Y D E L Y S G D P T F I T T S | | |
| 2451 | TATGGCTGGTATGGGTAATGACGGACGCCACCGTGTCACTAAAATGGACT | 2500 | |
| | M A G M G N D G R H R V T K M D Y | | |
| 2501 | ATCGTTTCTTGAACACACTTGATACAATCGGAAATGCTCCAGAACCAAAC | 2550 | |
| | R F L N T L D T I G N A P E P N | | |
| 2551 | TTGACAGTTCTTTGGGACTCTAAACTCCCATATTCATTCAAACGTTATTC | 2600 | |
| | L T V L W D S K L P Y S F K R Y S | | |
| 2601 | AATGTCTATGAGTCACAAACACTCATCTATCCAATATGAAGGTGTTGAAA | 2650 | |
| | M S M S H K H S S I Q Y E G V E T | | |
| 2651 | CAATGGCTAAAGATGGATATGGCGAAATGTCATGTATCTCTTGTTGTGTC | 2700 | |
| | M A K D G Y G E M S C I S C C V | | |
| 2701 | TCACCACTTGACCCAGAAAATGAAGAAGGACGTCATAATCTCCAATTACTT | 2750 | |
| | S P L D P E N E E G R H N L Q Y F | | |
| 2751 | TGGTGCGCGTGTAAACGTCTTGAAAGCAATGTTGACTGGTTTGAACGGTG | 2800 | |
| | G A R V N V L K A M L T G L N G G | | |
| 2801 | GTTACGATGACGTTCATAAAGATTATAAAGTATTCGATATTGAACCTGTT | 2850 | |
| | Y D D V H K D Y K V F D I E P V | | |
| 2851 | CGTGATGAAATTCTTGACTATGATACAGTTATGGAAAACTTCGACAAATC | 2900 | |
| | R D E I L D Y D T V M E N F D K S | | |
| 2901 | ACTCAACTGGTTGACAGATACTTATGTTGATGCAATGAATATCATTCACT | 2950 | |
| | L N W L T D T Y V D A M N I I H Y | | |
| 2951 | ACATGACTGACAAATATAACTATGAAGCAGTTCAAAGTGGCCTTCTTGCCT | 3000 | |
| | M T D K Y N Y E A V Q M A F L P | | |
| 3001 | ACTAAAGTTCGTGCTAACATGGGATTTGGTATCTGTGGTTTCGCAAATAC | 3050 | |
| | T K V R A N M G F G I C G F A N T | | |
| 3051 | AGTTGATTCACTTTCAGCGATTAAATATGCTAAAGTTAAAACTTTGCGTG | 3100 | |
| | V D S L S A I K Y A K V K T L R D | | |
| 3101 | ATGAAAATGGCTACATCTACGATTATGAAGTAGAAGGTGACTTCCCACGT | 3150 | |
| | E N G Y I Y D Y E V E G D F P R | | |
| 3151 | TATGGTGAAGATGATGACCGTGCTGATGATATCGCTAAACTTGTCATGAA | 3200 | |
| | Y G E D D D R A D D I A K L V M K | | |
| 3201 | AATGTACCATGAAAAATTAGCTTCACACAAACTTTACAAAAATGCTGAAG | 3250 | |
| | M Y H E K L A S H K L Y K N A E A | | |
| 3251 | CTACTGTTTCACTTTTGACAATCACATCTAACGTTGCTTACTCTAAACAA | 3300 | |
| | T V S L L T I T S N V A Y S K Q | | |
| 3301 | ACTGGTAACTCTCCAGTTCATAAAGGAGTATTCCTCAATGAAGATGGTAC | 3350 | |
| | T G N S P V H K G V F L N E D G T | | |
| | EcoRI | | |
| 3351 | AGTCAACAAATCTAAACTTGAATTCTTCTCACCAGGTGCTAACCCATCTA | 3400 | |
| | V N K S K L E F F S P G A N P S N | | |
| 3401 | ACAAAGCTAAAGGTGGATGGTTGCAAAATCTTCGTTCATTAGCTAAATTG | 3450 | |
| | K A K G G W L Q N L R S L A K L | | |
| | EcoRI | | |
| 3451 | GAATTCAAAGATGCAAATGACGGTATTTCATTAACTACTCAAGTTTCTCC | 3500 | |
| | E F K D A N D G I S L T T Q V S P | | |
| 3501 | TCGTGCACTTGGTAAAACTCGTGATGAACAAGTAGATAACTTGGTTCAAA | 3550 | |
| | R A L G K T R D E Q V D N L V Q I | | |
| 3551 | TTCTTGATGGATACTTCACACCAGGAGCTTTGATTAATGGTACTGAATTT | 3600 | |
| | L D G Y F T P G A L I N G T E F | | |
| 3601 | GCAGGTCAACACGTTAACTTGAACGTTATGGACCTTAAAGATGTTTACGA | 3650 | |
| | A G Q H V N L N V M D L K D V Y D | | |
| 3651 | TAAAATCATGCGTGGTGAAGATGTTATCGTTCGTATCTCTGGATACTGTG | 3700 | |
| | K I M R G E D V I V R I S G Y C V | | |
| 3701 | TTAACACTAAATACCTCACACCTGAACAAAAACAAGAATTGACTGAACGT | 3750 | |
| | N T K Y L T P E Q K Q E L T E R | | |
| 3751 | GTCTTCCATGAAGTACTTTCAAATGATGATGAAGAAGTAATGCACACTTC | 3800 | |
| | V F H E V L S N D D E E V M H T S | | |
| 3801 | AAATATCTAATTCTTAGTATTAAAAAATATAAGGTCTGTCAGTTCTACTG | 3850 | |
| | N I Stop | | |
| 3851 | ACAGACTTTTTTTCTATAAATTAATTATAATAGTTAAAAACTATTATTTT | 3900 | |
| 3901 | TAGTTTAAGAAAAATAAAATTTGTGCTAAAATAGATGAATGATAAAGGTA | 3950 | |
| 3951 | ATTGGATTAACAGGCGGAATTGCGAGTGGGAAATCAACGGTGGTTGATTT | 4000 | |
| 4001 | TTTGATTTCTGAAGGTTATCAAGTAATTGATGCTGACAAAGTTGTTCGTC | 4050 | |
| 4051 | AGTTGCAAGAACCTGATGGGAAACTTTTTAATGCAATAATGGAAACTTTC | 4100 | |
| 4101 | GGTTCAGATTTTACTGACGAAAATGGGAAATTAAACCGATGCAAAATTGA | 4150 | |
| 4151 | GTGCTTAAGTTTTGCTGACCCAAATCAACGTCAAAAATTAT 4191 | | |

Homology searches using the above deduced PFL protein revealed a 790 overall protein sequence identity with the S. mutans PFL and higher than 40% with the E. coli, C. pasteurianum and H. influenzae PFL.

In the promoter region of the MG1363 pfl gene canonical lactococcal ribosome binding site (AAAGGAG, position +21 to +27). −35 and −10 promoter regions (TTGCTA and TATAAT, respectively were found. A putative rho-dependent transcription terminator was located 24 bp downstream of the pfl stop codon (position 2432 to 2445). Additionally, two sequences (FNR-1 and FNR-2 with significant homology to E. coli FNR-boxes having consensus sequence TTGAT-N₄-ATCAA (SEQ ID NO:40) and being involved in regulation of the expression of pfl in E. coli were identified. The MG1363 FNR-1 (GGAGT-N₄ATCAA) (SEQ ID NO:41) was also present in strain DB1341. FNR-2 (TTTGC-N₄-ATCAA) (SEQ ID NO:42); position −36 to −23 overlaps with the −35 hexamer of the promoter region of the pfl gene.

The coding sequence of the MG1363 pfl gene showed 102 basepair changes when compared to the corresponding sequence of strain DB1341, but these changes resulted only in four amino acid changes in the PFL primary structure. The lactococcal PFL includes the conserved Gly residue at position 749, flanked by Ser and Tyr residues, which is involved in activation and deactivation of the enzyme in E. coli via free radical formation. This region is present in all PFL proteins characterized to date. The L. lactis sequence ISCCVSP is highly conserved and includes two adjacent Cys residues.

### EXAMPLE 6

### Construction of pfl Mutant Strains of L. lactis Strains DB1341 and MG1363 by Gene Inactivation and Physiological Characterization of pfl⁻ Strains

A 460 bp Sau3AI internal fragment (positions 1343 to 1799 in Table 3.2) of the L. lactis DB1341 pfl gene was cloned into BamHI-digested pSMA500 (Madsen et al., 1996), resulting in plasmid pSMAKAS7, and transformed into E. coli MC1000 by electroporation (Sambrook et al., 1989). A transformant (SMAKAS7) containing the recombinant plasmid was isolated. The orientation of the pfl fragment in pSMAKAS7 was confirmed by sequencing. Homologous recombination of pSMAKAS7 into the L. lactis pfl gene allows translational fusion of the reporter lacLM gene (Madsen et al., 1996).

Plasmid pSMAKAS7 was used to transform L. lactis strains DB1341 and MG1363 by electroporation (Holo and Nes 1989). Two single transformants were isolated (DBKAS7 and MGKAS7, respectively). DBKAS7 became blue on X-gal plates, as expected if homologous integration at the chromosomal pfl locus had occurred, and was further characterized. Integration of pSMAKAS7 by homologous recombination into the DB1341 chromosome would result in a truncated pfl gene, where the N-terminal region of the protein (residues Met¹-Asp⁵⁷⁴) would be separated from the C-terminal domain (residues Asp⁴²²-Ile⁷⁷⁸). PCR analysis was used to confirm that DBKAS7 carries a disrupted pfl gene. The activation site of the E. coli Pfl, a glycine residue at position Gly⁷³⁴ flanked by serine and tyrosine is conserved in all bacterial Pfl proteins characterized (Weidner and Sawers, 1996; Yamamoto et al., 1996), including the L. lactis Pfl (position 2321-2329 of the nucleotide sequence in Table 3.2; Table 3.6). The truncated Pfl protein in strain DBKAS7 would lack an activation site.

A sample of Lactococcus lactis subspecies lactis biovar diacetylactis strain DBKAS7 and of Lactococcus lactis subspecies lactis strains MGKAS7, respectively were deposited under the Budapest Treaty with the German Collection of Microorganisms and Cell Cultures, Mascheroder Weg 1b, D-38 124 Braunschweig, Germany on 18 Jul. 1996 under the accession Nos DSM 11086 and DSM 11083, respectively.

A 495 bp PCR fragment was amplified from MG1363 using primers pfl1-P1MG1363 (5′-GGCCGCTCGA GTTGTGTCTC ACCACTTGAC CC-3′ (SEQ ID NO:43); XhoI site underlined) and pflP2MG1363 (5′-TAGTAGGATC CCATCATCTT CACCATAACG TGG-3′ (SEQ ID NO:44); BamHI site underlined) and cloned into XhoI+BamHI digested pSMA500 and transformed into strain MG1363, resulting in strain MGKAS13.

MGKAS13 was deposited under the Budapest Treaty with the German Collection of Microorganisms and Cell Cultures, Mascheroder Weg 1b, D-38 124 Braunschweig, Germany on 10 Jul. 1997 under the accession No. DSM 11653.

DBKAS7 and MGKAS13 formed blue colonies on X-gal-containing plates. Plasmid integration through homologous recombination was confirmed via PCR in both strains.

Physiological Analysis of the L. lactis pfl⁻ Strain

A colorimetric assay (Voges-Proskauer, VP; Westerfeld 1945) was used to study acetoin and diacetyl production in strain DBKAS7. The presence of acetoin and diacetyl in the samples results in the formation of red colour which is monitored by measuring OD₅₂₀. Overnight cultures of strain DBKAS7 (pfl⁻) and wild type strain DB1341, grown at 30° C. without aeration in GM17 were used. The VP assay was performed by mixing 200 μl bacterial culture, 100 μl 0.3% (w/v) creatine, 100 μl 5 M NaOH, and 50 μl 5% α-naphthol (dissolved in 2.5 M NaOH immediately before use). The mixture was incubated for 10 min at room temperature, with constant stirring to provide aeration. The reaction was stopped by adding 1 ml 4 mM DTT. After centrifugation to remove cellular debris, OD₅₂₀ was measured. As shown in Table 6.1. DBKAS7 had approximately a 2-fold increase in the production of acetoin/diacetyl as compared to strain DB1341.

TABLE 6.1
Voges-Proskauer assay for aroma compounds produced by
DB1341 and DBKAS7, respectively

| Strain | OD₆₀₀ | OD₅₂₀ |
|---|---|---|
| DB1341 | 2.40 | 0.082 |
| DBKAS7 | 2.22 | 0.155 |

Overnight cultures were grown at 30° C., without shaking in GM17.
The OD₆₀₀ values represent a measure for growth. The OD₅₂₀ values are the results of the production of acetoin and diacetyl (Westerfeld 1945).

Thus, gene inactivation of the pfl gene in the L. lactis strain DB1341 results in an enhanced production of aroma compounds, without affecting the ability to grow.

Similar levels of formate were obtained in strain DB1341 as in MG1363, and no formate was detected in DBKAS7 under anaerobic conditions, confirming the pfl mutant phenotype in this strain.

L. lactis biovar diacetylactis strains are used as starter cultures due to their ability to produce diacetyl during milk fermentation. A mutation in the pfl gene of DB1341 should result in increased pyruvate levels under anaerobic growth. Thus, if excess pyruvate is directed towards the production of diacetyl and acetoin, a higher level of these metabolites would be expected in strain DBKAS7 grown under anaerobiosis. As shown in Table 6.2. a 7-fold increase in the production of aroma compounds was observed in strain DBKAS7 grown in GM17 and a more than 4-fold increase was detected in GalM17 as compared to the wild type strain, DB1341. This demonstrated the effect of a pfl mutation in the production of diacetyl and acetoin in a L. lactis biovar diacetylactis strain.

TABLE 6.2
Production of aroma compounds in the L. lactis
biovar diacetylactis pfl⁻ strain,
DBKAS7 as compared to the wild type strain

Voges-Proskauer assay (diacetyl + acetoin in mM^{a})

| Strain | Glucose | Galactose |
|---|---|---|
| DB1341 | 0.2 | ≦0.05 |
| DBKAS7 | 1.5 | 0.2 |

^{a}cell extracts from stationary culture (OD₆₀₀ about 3) were assayed according to Casabadan et al. 1980. Values shown are the mean of two independent experiments.

Inactivation of the pfl gene leads to a transcriptional fusion of the lacLM reporter gene (Madsen et al. 1996) β-galactosidase levels were measured in overnight cultures of strain MGKAS13 grown in M17 with either glucose (GM17) or galactose (GalM17) (Table 6.2). Using GM17, anaerobic growth was observed, about a 10-fold increase of β-galactosidase units, which is consistent with the induction observed at RNA level. High levels of β-galactosidase were observed under anaerobic growth when growing in the presence of galactose, and a 4-fold induction was observed under anaerobiosis in this medium which is in agreement with the RNA studies.

TABLE 6.3
Characterization of the L. lactis Mg1363 pfl⁻ strain. MGKAS13

| | | Aerobic | | Anaerobic | |
|---|---|---|---|---|---|
| | Growth | Formate | β-gal | Formate | β-gal |
| Strain | medium | (mM) | (units) | (mM) | (units) |
| MG1363 | GM17 | 0 | — | 5.3 | — |
| | GalM17 | 0 | — | 42 | — |
| MGKAS13 | GM17 | 0 | 9.5 | 0 | 150.0 |
| | GalM17 | 0 | 94.6 | 0 | 600.0 |

MGKAS13 should not produce formate under anaerbic conditions as a result of the inactivation of the pfl gene in this strain. In strain MG1363, no formate was detected under aerobic growth in GM17, as it would be expected if the lactococcal PFL is inactivated in the presence of oxygen. Relatively low levels of formate were detected under anaerobic conditions. In GalM17 a 8-fold higher amount of formate was detected in anaerobiosis.
No formate was detected in strain MGKAS13 in either of the test media, confirming that this strain carries a pfl null mutation.

### EXAMPLE 7

### Identification of pfl and adhE Homologues in Non-Lactococcus lactic Acid Bacteria Using Lactococcus lactis pfl and adhE Gene Fragments as Probes

1. Southern Hybridization of Genomic DNA from Non-Lactococcus Lactic Acid Bacteria Using a L. lactis pfl Gene Fragment as a Probe

A PCR fragment including most of the L. lactis pfl coding sequence was obtained by amplification of MG1363 genomic DNA with primers pfl89 and pfl1066 (see FIG. 4). A 2 kb DNA fragment (FIG. 9) was obtained and used as a probe in Southern hybridization experiments using EcoRI-digested total DNA from Streptococcus thermophilus ATCC 19258, Leuconostoc mesenteroides subsp. mesenteroides ATCC 10878 and Lactobacillus acidophilus ATCC 4796 (FIG. 10).

Hybridization was carried out overnight at 65° C. Filters were washed twice in 5×SSC at room temperature for 30 minutes and subsequently once in 3×SSC; 0.1% SDS at 65° C. for 30 minutes. As shown in FIG. 10C the expected EcoRI genomic fragment deduced from L. lactis pfl sequence was detected after overnight exposure. After short exposure of the filters (FIG. 10B) only hybridization was detected in S. thermophilus and only weak signals were detected in L. mesenteroides and L. acidophilus after longer exposure (FIG. 10C), indicating lower pfl sequence homology in these bacteria, as would be expected due to their taxonomic distance to L. lactis.

2. Southern Hybridisation of Genomic DNA from Non-Lactococcus Lactic Acid Bacteria Using L. lactis adhE Gene Fragment as a Probe

Two Sau3AI fragments including most of the L. lactis subsp. lactis biovar diacetylactis DB 1341 adhE coding sequence (FIG. 11) were used as a probe in Southern hybridization experiments using EcoRI-digested total DNA from Streptococcus thermophilus ATCC 19258, Leuconostoc mesenteroides subsp. mesenteroides ATCC 10878 and Lactobacillus acidophilus ATCC 4796.

Hybridization was carried out overnight at 65° C. Filters were washed twice in 5×SSC at room temperature for 30 minutes and subsequently once in 3×SSC; 0.1% SDS at 65° C. for 30 minutes. As shown in FIG. 12, the expected EcoRI genomic fragment (about 5 kb) deduced from the L. lactis MG1363 adhE sequence was detected. Strongly hybridizing bands were also detected in S. thermophilus (5 kb) and L. mesenteroides (5 and 0.4 kb). Weaker hybridizing bands were also detected in L. acidophilus (4.2 and 2 kb, and two minor bands, 2.3 and 5 kb).

3. Conclusions

Using the above L. lactis DNA probes, preliminary restriction maps of the pfl and adhE genes, respectively in the three non-Lactococcus lactic acid bacterial species could be carried out using different restriction digests of the genomic DNA. Two strategies for the cloning of these non-Lactococcus genes can be followed: (i) cloning of DNA fragments isolated from agarose gels corresponding in size to the hybridizing bands detected in Southern analysis; (ii) PCR of conserved regions using primers derived from the corresponding L. lactis sequence.

### REFERENCES

1. Arnau, J., F. Jørgensen, S. Madsen, A. Vrang and H. Israelsen. 1997. Cloning, expression and characterization of the Lactococcus lactis pfl gene, encoding pyruvate formate-lyase. Submitted for publication.

2. Chen, Y.-M. and C. C. Lin. 1991. Regulation of the adhE gene, which encodes ethanol dehydrogenase in Escherichia coli. J. Bacteriol. 173:8009-8013.

3. Chippaux, M., F. Casse and M.-C. Pascal. 1972. Isolation and phenotypes of mutants from Salmonella typhimurium defective in formate hydrogenlyase activity. J. Bacteriol. 110:766-768.

4. Christiansen, L. and S. Pedersen. 1981. Cloning, restriction endonuclease mapping and post-transcriptional regulation of rspA, the structural gene for ribosomal protein S1. Mol. Gen. Genet. 181:548-551.

5. Crow, V. L. and G. G. Pritchard. 1977. Fructose 1,6-diphosphate-activated L-lactate dehydrogenase from Streptococcus lactis: kinetic properties and factors affecting activation. J. Bacteriol. 131:82-91.

6. Donkersloot, J. A. and J. Thompson. 1995. Cloning, expression, sequence analysis, and site-directed mutagenesis of the Tn5306-encoded N5-(Carboxyethyl)ornithine synthase from Lactococcus lactis K1. J. Biol Chem 270:12226-12234.

7. Fleischmann, R. D., M. D. Adams, O. White, R. A. Clayton, E. F. Kirkness, A. R. Kerlavage, C. J. Bult, J. F. Tomp, B. A. Dougherty and J. M. Merrick et al. 1995. Whole-genome random sequencing and assembly of Haemophilus influenzae Rd. Science 269:496-512.

8. Frey, M., Rothe, M., Volker Wagner, A. F. and Knappe, J. 1994. Adenosylmethionine-dependent synthesis of the glycyl radical in pyruvate formate-lyase by abstraction of the glycine C-2 pro-S hydrogen atom. J. Biol. Chem. 269: 12432-12437.

9. Goodlove, P. E., P. R. Cunningham, J. Parker, and D. P. Clark. 1989. Cloning and sequence analysis of the fermentative alcohol-dehydrogenase-encoding gene of Escherichia coli. Gene 85:209-214.

10. Holo, H. and I. F. Ness. 1989. High-frequency transformation by electroporation of Lactococcus lactis subsp. cremoris grown with glycine in osmotically stabilized media. Appl. Environ. Microbial. 55:3119-3123.

11. Kessler, D., I. Leibrecht and J. Knappe. 1991. Pyruvate-formate-lyase-deactivase and acetyl CoA reductase activities of Escherichia coli reside on a polymeric protein particle encoded by adhE. FEBS Lett. 281:59-63.

12. Kessler, D., W. Herth and J. Knappe. 1992. Ultrastructure and pyruvate formate-lyase radical quenching property of the multienzymic AdhE protein of Escherichia coli. J. Biol. Chem. 267:18073-18079.

13. Madsen, S. M., B. Albrechtsen, E. B. Hansen and H. Israelsen. 1996. Cloning and transcriptional analysis of two threonine biosynthetic genes from Lactococcus lactis MG1614. J. Bacteriol 178:3689-3694.

14. Mat-Jan, F., K. Y. Alam and D. P. Clark. 1989. Mutants of Escherichia coli deficient in the fermentative lactate dehydrogenase. J. Bacteriol. 171:342-348.

15. Nair, R. V., G. N. Bennett and E. T. Papoutsakis. 1994. Molecular characterization of an aldehyde/alcohol dehydrogenase from Clostridium acetobutylicum ATCC 824. J. Bacteriol. 176:871-881.

16. Pecher, A., H. P. Blaschkowski, K. Knappe and A. Böck. 1982. Expression of pyruvate formate-lyase of Escherichia coli from the cloned structural gene. Arch. Microbiol. 132:365-371.

17. Platteeuw, C., J. Hugenholtz, M. Starrenburg, I. van Alen-Boerrigter and W. M. de Vos. 1995. Metabolic engineering of Lactococcus lactis: influence of the overproduction of α-acetolactate synthase in strains deficient in lactate dehydrogenase as a function of culture conditions. Appl. Environ. Microbiol. 61:3967-3971.

18. Sambrook, J., E. F. Fritsch and T. Maniatis. 1989. Molecular cloning: a laboratory manual, 2nd ed. Cold Spring Harbor Laboratory Press, Cold Spring Harbor, N.Y.

19. Sauter, M. and Sawers, R. G. 1990. Transcriptional analysis of the gene encoding pyruvate formate-lyase-activating enzyme of Escherichia coli. Mol. Microbiol. 4: 355-363.

20. Sawers, G., A. F. Wagner and A. B\ddot{ock}. 1989. Transcription initiation at multiple promoters of the pfl gene by Eσ70-dependent transcription in vitro and heterologous expression in Pseudomonas putida in vivo. J. Bacteriol. 171: 4930-4937.

21. Sawers, G. and A. Böck. 1988. Anaerobic regulation of pyruvate formate-lyase from Escherichia coli K-12. J. Bacteriol. 170:5330-5336.

22. Sawers, G. and A. Böck. 1989. Novel transcriptional control of the pyruvate formate-lyase gene: upstream regulatory sequences and multiple promoters regulate anaerobic expression. J. Bacteriol. 171:2485-2498.

23. Snoep, J. L., M. J. T. de Mattos, M. J. C. Starrenburg and J. Hugenholtz. 1992. Isolation, characterization, and physiological role of the pyruvate dehydrogenase complex and α-acetolactate synthase of Lactococcus lactis subsp. lactis bv. diacetylactis. J. Bacteriol. 174: 4838-4841.

24. Suppmann, B. and G. Sawers. 1994. Isolation and characterization of hypophosphite-resistant mutants of E. coli: identification of the FocA protein, encoded by the pfl operon, as a putative formate transporter. Mol. Microbiol 11:965-982.

25. Takahashi, S., K. Abbe and T. Yamada. 1982. Purification of pyruvate formate-lyase from Streptococcus mutans and its regulatory properties. J. Bacteriol. 149:1034-1040.

26. Varenne, S., F. Casse, M. Chippaux and M. C. Pascal. 1975. A mutant of Escherichia coli deficient in pyruvate formate-lyase. Mol. Gen. Genet. 141:181-184.

27. deVos, W. M. and G. Simons. 1994. Gene cloning and expression systems in lactococci. In: Gasson, M., Vos W. de (eds) Genetics and biotechnology of lactic acid bacteria, Chapman and Hall, London, pp. 52-105.

28. Weidner, G. and G. Sawers. 1996. Molecular characterization of the genes encoding formate-lyase and its activating enzyme of Clostridium pasteurianum. J. Bacteriol. 178:2440-2444.

29. Westerfeld, W. W. 1945. A calorimetric determination of blood acetoin. J. Biol. Chem. 161: 495-502.

30. Wong, K. K., K. L. Suen and H. S. Kwan. 1989. Transcription of pfl is regulated by anaerobiosis, catabolite repression, pyruvate, and oxrA: pfl::Mu dA operon fusions of Salmonella typhimurium. J. Bacteriol. 171:4900-4905.

31. Yamamoto, Y., Y. Sato, S. Takahashi-Abbe, K. Abbe, T. Yamada and H. Kizaki. 1996. Cloning and sequence analysis of the pfl gene encoding pyruvate formate-lyase from Streptococcus mutans. Infect. Immun. 64:385-391.

1. An isolated DNA sequence comprising a sequence derived from a lactic acid bacterium, said sequence coding for a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

2. A DNA sequence according to claim 1 further comprising sequences regulating the expression of the coding sequence and/or the activity of its gene product.

3. A DNA sequence according to claim 1 which is derived from a lactic acid bacterium selected from the group consisting of a Lactococcus species, a Lactobacillus species, a Streptococcus species, a Pediococcus species, a Bifidobacterium species and a Leuconostoc species.

4. A DNA sequence according to claim 3 which is derived from Lactococcus lactis.

5. A DNA sequence according to claim 1 coding for a polypeptide which is at least 30% identical with a polypeptide which is selected from the group consisting of the gene product of the adhE gene of E. coli as recorded in FASTA, GCG Wisconsin under the accession No. P17547, the gene product of the aad gene of Clostridium acetobutylicum as recorded in FASTA, GCG Wisconsin under the accession No. P33744 and of the DNA sequence of SEQ ID NO:3.

6. A DNA sequence according to claim 1 which comprises the coding sequence of SEQ ID NO:3 or SEQ ID NO:30, or a mutant or variant hereof which codes for a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

7. A recombinant replicon comprising the DNA sequence of claim 1.

8. A replicon according to claim 7 which is selected from a plasmid capable of replicating in a lactic acid bacterium and a lactic acid bacterial chromosome.

9. A recombinant lactic acid bacterial cell comprising the replicon of claim 7.

10. A lactic acid bacterial cell according to claim 9 which is selected from the group consisting of a Lactococcus species, a Lactobacillus species, a Streptococcus species, a Pediococcus species, a Bifidobacterium species and a Leuconostoc species.

11. A lactic acid bacterial cell according to claim 9 which is in the form of a starter culture composition for the production of a food product or an animal feed, or in the form of a culture for the production of an aroma or antimicrobially active compound.

12. A lactic acid bacterial cell according to claim 9 wherein the DNA sequence comprising the sequence coding for the multi-functional polypeptide is modified so as to inactivate or reduce the production of or the activity of at least one of the enzymatic activities selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

13. A lactic acid bacterial cell according to claim 12 wherein said modification of the DNA sequence results in the cell producing increased amounts of a metabolite selected from the group consisting of acetaldehyde, acetate and ethanol.

14. A lactic acid bacterial cell according to claim 9 wherein the DNA sequence comprising the sequence coding for the multi-functional polypeptide is modified so as to enhance the production of or the activity of at least one of the enzymatic activities selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity.

15. A lactic acid bacterial cell according to claim 14 wherein said modification of the DNA sequence results in the cell producing an increased amount of a metabolite selected from the group consisting of acetaldehyde, ethanol, formate, acetate, α-acetolactate, acetoin, diacetyl and 2,3 butylene glycol.

16. An isolated DNA sequence comprising a sequence derived from a lactic acid bacterium, said sequence coding for a polypeptide having pyruvate formate-lyase activity, subject to the limitation that the sequence is not derived from an oral Streptococcus species.

17. A DNA sequence according to claim 16 comprising at least one regulatory sequence regulating the expression of the pyruvate formate-lyase polypeptide or coding for a gene product regulating the pyruvate formate-lyase activity of the polypeptide.

18. A DNA sequence according to claim 17 wherein the regulating gene product is selected from a pyruvate formate-lyase activase and a pyruvate formate-lyase deactivase.

19. A DNA sequence according to claim 18 wherein the deactivase is a polypeptide having at least one enzymatic activity selected from the group consisting of (i) acetaldehyde dehydrogenase (ACDH) activity whereby acetyl CoA is converted into acetaldehyde, (ii) alcohol dehydrogenase (ADH) activity whereby acetaldehyde is converted into ethanol, (iii) capability of converting acetyl CoA into ethanol and (iv) pyruvate formate-lyase deactivase activity as defined in claim 1.

20. A DNA sequence according to claim 16 which is derived from a lactic acid bacterium selected from the group consisting of a Lactococcus species, a Lactobacillus species, a Streptococcus species, a Pediococcus species, a Bifidobacterium species and a Leuconostoc species.

21. A DNA sequence according to claim 20 which is derived from Lactococcus lactis.

22. A DNA sequence according to claim 16 which comprises the coding sequence of SEQ ID NO:15 or SEQ ID NO:30, or a mutant or variant hereof which codes for a polypeptide having pyruvate formate-lyase activity.

23. A recombinant replicon comprising the DNA sequence of claim 16.

24. A replicon according to claim 23 which is selected from a plasmid capable of replicating in a lactic acid bacterium and a lactic acid bacterial chromosome.

25. A recombinant lactic acid bacterial cell comprising the replicon of claim 23.

26. A lactic acid bacterial cell according to claim 25 which is selected from the group consisting of a Lactococcus species, a Lactobacillus species, a Streptococcus species, a Pediococcus species, a Bifidobacterium species and a Leuconostoc species.

27. A lactic acid bacterial cell according to claim 25 which is in the form of a starter culture composition for the production of a food product or an animal feed.

28. A lactic acid bacterial cell according to claim 25 wherein the DNA sequence is modified whereby its production of pyruvate formate-lyase is reduced or inhibited or whereby the enzyme is produced in a modified form having a reduced pyruvate formate-lyase activity.

29. A lactic acid bacterial cell according to claim 28 wherein said modification of the DNA sequence results in that the cell produces increased amounts of a metabolite selected from the group consisting of α-acetolactate, acetoin, diacetyl and 2,3 butylene glycol.

30. A lactic acid bacterial cell according to claim 25 wherein the DNA sequence is modified whereby its production of pyruvate formate-lyase is enhanced or whereby the enzyme is produced in a modified form having an increased pyruvate formate-lyase activity.

31. A lactic acid bacterial cell according to claim 30 wherein said modification of the DNA sequence results in the cell producing increased amounts of formate.

32. A recombinant lactic acid bacterial cell comprising the DNA sequence of claim 1 and the DNA sequence of claim 16.

33. A recombinant lactic acid bacterial cell according to claim 32 wherein at least one of said DNA sequences is modified so as to modify the expression of pyruvate formate-lyase or the activity hereof.

34. A method of producing a lactic acid bacterial metabolite, the method comprising cultivating a lactic acid bacterium according to any of claims 12, 15, 30 or 32 under conditions where the metabolite is produced and isolating the metabolite from the culture.

36. A method of producing an animal feed, the method comprising the step of admixing to the feed starting materials a starter culture of a lactic acid bacterium according to claim 9 or 26 and keeping the mixture under conditions allowing the starter culture to be metabolically active.

37. An isolated DNA sequence derived from a lactic acid bacterium, said sequence coding for a product having a formate transporter activity.

38. A DNA sequence according to claim 37 which is the open reading frame orfA isolated from Lactococcus lactis strain DB1341 where it is located upstream of the pfl gene (SEQ ID NO:34).
